(19) United States
(12) Patent Application Publication
(10) Pub. No.: US 2023/0372093 A1
(43) Pub. Date: Nov. 23, 2023

(54) 3-D SHAPED SKIRTS FOR PROSTHETIC HEART VALVES

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Noam Nir, Pardes-Hanna (IL); Tomer Saar, Pardes Hanna-Karkur (IL); Tamir S. Levi, Zikhron Yaakov (IL)

(21) Appl. No.: 18/358,845

(22) Filed: Jul. 25, 2023

Related U.S. Application Data
(63) Continuation of application No. PCT/US22/13724, filed on Jan. 25, 2022.
(60) Provisional application No. 63/141,811, filed on Jan. 26, 2021.

(51) Int. Cl. A61F 2/24

(52) U.S. Cl. CPC A61F 2/2418; A61F 2250/0069

(57) ABSTRACT

The present invention relates to implantable prosthetic devices, and more particularly, to 3D-shaped skirts having various coatings and/or configurations thereof.

### RELATED APPLICATIONS

This is a continuation of PCT application no. PCT/US2022/013724 filed on Jan. 25, 2022, which claims the benefit of and priority to U.S. provisional patent application No. 63/141,811 filed on Jan. 26, 2021, the entire disclosure of each of which being incorporated herein by this specific reference.

### FIELD OF THE INVENTION

The present invention relates to the field of implantable prosthetic heart valves, and more particularly, to sealing members having first layers with various 3D-shaped coatings thereon, method of preparation thereof and implantable prosthetic heart valves comprising the same.

### BACKGROUND OF THE INVENTION

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Since surgeries are prone to an abundance of clinical complications, alternative less invasive techniques of delivering a prosthetic heart valve over a catheter and implanting it over the native malfunctioning valve have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter. In some cases, explant of the valves is required, in which case the originally implanted valve is surgically removed from the patient's body.

Paravalvular leakage (PVL) is a complication that is related to the replacement of a prosthetic heart valve. It may occur when blood flows through a channel or gap located between the structure of an implanted prosthetic heart valve in an expanded state and the site of implantation (e.g., the cardiac or arterial tissue surrounding it), due to a lack of appropriate sealing therebetween.

Thus, there is an ongoing need to provide a prosthetic heart valve which enables appropriate sealing with the surrounding tissue at the site of implantation, so as to substantially fill in the gaps or channels that may result in PVL, but will enable simple extraction thereof from the site of implantation when required.

### SUMMARY OF THE INVENTION

The present disclosure is directed toward prosthetic heart valves and methods for the manufacture and/or utilization thereof, especially for 3D-shaped prosthetic heart valves which can enable appropriate sealing with the surrounding tissue at the site of implantation, so as to substantially fill in the gaps or channels that may result in PVL, and can also enable simple extraction thereof from the site of implantation when an explant procedure is performed.

According to a first aspect of the present invention, there is provided a prosthetic heart valve comprising: a frame, a leaflet assembly mounted within the frame, and a sealing member coupled to an outer surface of the frame. The frame comprises a plurality of intersecting struts and is movable between a radially compressed state and a radially expanded state. The sealing member extends from an inflow edge toward an opposing outflow edge, and comprises a first layer and a second layer coating the first layer. A nonfibrous outer surface of the sealing member is formed of a material inherently shaped to define a plurality of elevated portions with peaks and a plurality of non-elevated portions. The first and second layers are disposed externally to the outer surface of the frame.

According to some examples, each one of the plurality of non-elevated portions is defined by adjacent pairs of the plurality of elevated portions.

According to some examples, the elevated portions are configured to deform when an external pressure exceeding a predefined threshold is applied thereto in a direction configured to press them against the frame, and to revert to a relaxed state thereof when the external pressure is no longer applied thereto. The distance of the peaks from the frame is greater than the distance of the non-elevated portions from the frame in the relaxed state.

According to some examples, the predefined threshold of the external pressure is 300 mmHg.

According to some examples, the distance of the peaks from the frame is at least 1000% greater than the distance of the non-elevated portions from the frame, in the absence of an external force applied to press the elevated portions against the frame (i.e., in the relaxed state). According to some examples, the distance of the peaks from the frame is at least 2000% greater than the distance of the non-elevated portions therefrom. According to some examples, the distance of the peaks from the frame is at least 3000% greater than the distance of the non-elevated portions therefrom.

According to some examples, the nonfibrous outer surface is a smooth surface.

According to some examples, the sealing member comprises a third layer. According to some examples, the second layer and the third layer collectively form a coating which covers the first layer.

According to some examples, the first layer comprises at least one tear resistant fabric. According to some examples, the tear resistant fabric comprises a ripstop fabric. According to some examples, the first layer comprises a biocompatible material. According to some examples, the first layer comprises at least one elastic material. According to some examples, the first layer comprises a PET fabric. According to some examples, the first layer is having a tear resistance of at least 5N. According to some examples, the first layer is having a tear resistance of at least 15N.

According to some examples, the second layer comprises a biocompatible material. According to some examples, the second layer comprises at least one thromboresistant material. According to some examples, the second layer is made of a thermoplastic material. According to some examples, the second layer is made of a thermoplastic material selected from the group consisting of: polyamides, polyesters, polyethers, polyurethanes, polyolefins, polytetrafluoroethylenes, and combinations and copolymers thereof. According to some examples, the second layer is made of a thermoplastic elastomer. According to some examples, the second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the second layer comprises TPU.

According to some examples, the third layer comprises a biocompatible material. According to some examples, the third layer comprises at least one thromboresistant material. According to some examples, the third layer is made of a thermoplastic material. According to some examples, the third layer is made of a thermoplastic material selected from the group consisting of: polyamides, polyesters, polyethers, polyurethanes, polyolefins, polytetrafluoroethylenes, and combinations and copolymers thereof. According to some examples, the third layer is made of a thermoplastic elastomer. According to some examples, the third layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the third layer comprises TPU.

According to some examples, the second layer and the third layer are made from the same material.

According to some examples, the elevated portions of the sealing member comprise a plurality of ridges, wherein the plurality of ridges are spaced apart from each other along a first surface of the sealing member. According to some examples, the second layer forms the first surface of the sealing member. According to some examples, each one of the plurality of ridges extends outward from the outer surface of the frame. According to some examples, the plurality of ridges are compressible.

According to some examples, the sealing member comprises a plurality of inner channels, wherein each channel is formed at a second surface of the sealing member. According to some examples, the number of channels is identical to the number of ridges, wherein each one of the plurality of channels is formed by a respective one of the plurality of ridges at an opposing surface of the sealing member. According to some examples, each one of the plurality of channels is facing inward.

According to some examples, the non-elevated portions of the sealing member comprise a plurality of inter-ridge gaps formed over the surface of the first layer between each two adjacent ridges of the sealing member.

According to some examples, the plurality of ridges follow parallel path-lines extending around and/or along the first surface of the sealing member.

According to some examples, the plurality of ridges follow parallel path-lines extending substantially in parallel to at least one of the inflow edge and/or the outflow edge.

According to some examples, the plurality of ridges follow parallel path-lines extending substantially perpendicular to at least one of the inflow edge and the outflow edge.

According to some examples, the plurality of ridges follow parallel path-lines extending substantially diagonally with respect to at least one of the inflow edge and the outflow edge.

According to some examples, the sealing member has a total layer thickness measured between the first surface and the second surface of the sealing member, at one of the inter-ridge gaps, and a sealing member thickness measured by the height of the ridges of the sealing member, wherein the sealing member thickness is greater by at least 1000% than the total layer thickness. According to some examples, the sealing member thickness is greater by at least 2000% than the total layer thickness. According to some examples, the sealing member thickness is greater by at least 3000% than the total layer thickness.

According to some examples, the sealing member as disclosed herein above is prepared by a method comprising: (i) providing a tear resistant flat sheet extending from a first lateral edge to a second lateral edge; (ii) treating the sheet in a thermal shape-forming process to assume a resilient structure comprising a plurality of elevated portions and a plurality of non-elevated portions, in a spread relaxed state; and (iii) connecting two opposite edges of the sheet to form a cylindrical sealing member in a cylindrical folded state.

According to some examples, the thermal shape-processing of the sheet at step (ii) comprises thermoforming.

According to some examples, the sheet comprises a tear resistant first layer and a thermoplastic second layer.

According to some examples, the tear resistant first layer comprises a ripstop fabric. According to some examples, the tear resistant first layer comprises a biocompatible material. According to some examples, the tear resistant first layer comprises at least one elastic material. According to some examples, the tear resistant first layer comprises a PET fabric. According to some examples, the tear resistant first layer is having a tear resistance of at least 5N. According to some examples, the tear resistant first layer is having a tear resistance of at least 15N.

According to some examples, the thermoplastic second layer comprises at least one thromboresistant material. According to some examples, the thermoplastic second layer is made of a biocompatible material. According to some examples, the thermoplastic second layer is made of a thermoplastic material selected from the group consisting of: polyamides, polyesters, polyethers, polyurethanes, polyolefins, polytetrafluoroethylenes, and combinations and copolymers thereof. According to some examples, the thermoplastic second layer is made of a thermoplastic elastomer. According to some examples, the thermoplastic second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic second layer comprises TPU.

According to some examples, the tear resistant flat sheet at step (i) further comprises a thermoplastic third layer. According to some examples, the second layer and the third layer are made from the same material. According to some examples, the thermoplastic third layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic third layer comprises TPU.

According to some examples, step (ii) comprises placing the flat sheet on a mold at an elevated temperature, thereby forming a plurality of ridges thereon, and lowering the temperature, thereby maintaining a resilient structure of the thermoplastic second layer.

According to some examples, the thickness of sealing member in its spread relaxed state is at least 1000% greater than the initial thickness of the sheet provided in step (i).

According to some examples, step (ii) comprises placing the flat sheet on a mold at an elevated temperature and gravitationally submerging the heated sheet, thereby forming a plurality of ridges thereon. According to some examples, the mold is selected from a plurality of rods, tubes, pipes, and combinations thereof.

According to some examples, the mold comprises a base, a plurality of protrusions and a vacuum source comprising apertures. According to some examples, step (ii) comprises placing the flat sheet over the plurality of protrusions at an elevated temperature and applying vacuum, using the vacuum source and the apertures, thereby thermoforming the sheet to a resilient structure in a spread relaxed state.

According to some examples, step (ii) includes application of force using mold over two opposite edges of the flexible sheet. According to some examples, the mold comprises a first mold and a second mold, wherein the first mold comprises a first base and plurality of first mold protrusions and the second mold comprises a second base and plurality of second mold protrusions. According to some examples, step (ii) comprises placing the flat sheet between the plurality first mold protrusions and the plurality of second mold protrusions, so that the plurality first mold protrusions and the plurality second mold protrusions are disposed at a zipper-like configuration. According to some examples, step (ii) further comprises pressing the second mold against the first mold at an elevated temperature, thereby effectively engaging the flat sheet therebetween to enable the sheet to conform to said molds.

According to some examples, step (ii) comprises placing the flat sheet comprising a tear resistant first layer, as disclosed herein above, on a mold at an elevated temperature and coating the shaped sheet with a second layer over the mold, thereby forming a plurality of ridges thereon. According to some examples, the mold comprises a base and a plurality of protrusions. According to some examples, step (ii) involves heat-coating the shaped sheet with the second layer at an elevated thermoformable temperature.

According to some examples, the second layer is made of a thermoplastic material. According to some examples, the thermoplastic material is selected from the group consisting of: polyamides, polyesters, polyethers, polyurethanes, polyolefins, polytetrafluoroethylenes, and combinations and copolymers thereof. According to some examples, the thermoplastic second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic second layer comprises TPU.

According to some examples, the elevated portions of the sealing member of the present invention comprise a plurality of protrusions extending around and/or outward from a first surface of the sealing member. According to some examples, said plurality of protrusions are spaced apart from each other along the first surface. According to some examples, each one of the plurality of protrusions is compressible. According to some examples, the sealing member comprises a flat second surface located opposite to the first surface, when in its spread relaxed state.

According to some examples, the non-elevated portions of the sealing member comprise a plurality of inter-protrusion gaps, wherein each gap is located between two adjacent protrusions. According to some examples, the plurality of inter-protrusion gaps are facing the same direction as the protrusions face.

According to some examples, each one of the plurality of protrusions extends around and/or away from the first surface and forms 3D shapes thereon. According to some examples, the 3D shapes can be selected from the group consisting of: inverse U-shapes, half-spheres, domes, cylinders, pyramids, triangular prisms, pentagonal prisms, hexagonal prisms, flaps, polygons, and combinations thereof.

According to some examples, the plurality of protrusions form elongated 3D shapes and extend substantially in parallel to at least one of the inflow edge and/or the outflow edge.

According to some examples, the plurality of protrusions form elongated 3D shapes and extend substantially perpendicular to at least one of the inflow edge and/or the outflow edge.

According to some examples, the plurality of protrusions form elongated 3D shapes and extend substantially diagonally with respect to at least one of the inflow edge and/or the outflow edge.

According to some examples, the sealing member has a total layer thickness measured between the first surface and the second surface at one of the inter-protrusion gaps, and a sealing member thickness defined as the distance between the protrusions to the second surface, wherein the sealing member thickness is greater by at least 1000% than the total layer thickness. According to some examples, the sealing member thickness is greater by at least 2000% than the total layer thickness. According to some examples, the sealing member thickness is greater by at least 3000% than the total layer thickness.

According to some examples, the plurality of protrusions comprises the same material as the second layer. According to some examples, each protrusion is made of a biocompatible material. According to some examples, each protrusion is made of at least one thromboresistant material. According to some examples, each protrusion is made of a thermoplastic material. According to some examples, each protrusion is made of a thermoplastic material selected from the group consisting of: polyamides, polyesters, polyethers, polyurethanes, polyolefins, polytetrafluoroethylenes, and combinations and copolymers thereof. According to some examples, each protrusion is made of thermoplastic elastomer. According to some examples, each protrusion is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, each protrusion is made of TPU.

According to some examples, each one of the plurality of protrusions of the sealing member of the present invention defines a non-hollow structure, thereby forming non-hollow protrusions.

According to some examples, the sealing member as disclosed herein above, comprising the non-hollow protrusions, is prepared by a method comprising: (i) providing a tear resistant flat sheet extending from a first lateral edge to a second lateral edge, and from an inflow edge to an outflow edge; (ii) treating the sheet in a thermal shape-forming process to assume a resilient structure comprising a plurality of elevated portions and a plurality of non-elevated portions, in a spread relaxed state; and (iii) connecting two opposite edges of the sheet to form a cylindrical sealing member in a cylindrical folded state. The tear resistant flat sheet comprises a tear resistant first layer and a thermoplastic second layer, as disclosed herein above.

According to some examples, step (ii) entails an extrusion-based shape-forming process, comprising extruding a plurality of members on the thermoplastic second layer of the flat sheet. Each member comprises a molten composition at an elevated temperature, wherein the members are spaced from each other. Step (ii) further entails lowering the temperature, resulting in the transition of each extruded member to a resilient state, thereby forming the plurality of protrusions thereon.

According to some examples, the molten composition comprises at least one thromboresistant biocompatible material. According to some examples, the molten composition comprises at least one thermoplastic material selected from the group consisting of polyamides, polyesters, polyethers, polyurethanes, polyolefins, polytetrafluoroethylenes, and combinations and copolymers thereof. According to some examples, the molten composition comprises at least one thermoplastic elastomer material selected from the group consisting of thermoplastic polyurethane (TPU), styrene block copolymers (TPS), thermoplastic polyolefinelastomers (TPO), thermoplastic vulcanizates (TPV), thermoplastic copolyester (TPC), thermoplastic polyamides (TPA), and combinations and variations thereof. According to some examples, the molten composition comprises TPU.

According to some examples, step (ii) entails an injection molding process, comprising inserting the flat sheet into a mold at an elevated temperature, and injecting a molten composition into said mold on top of at least one surface of the flat sheet. The molten composition is configured to conform to the shape of the mold when the temperature is lowered. The mold is configured to be removed after the cooling thereof, thereby forming the resilient structure of the sealing member in the spread relaxed state. The molten composition comprises a thermoplastic material as disclosed herein above.

According to some examples, step (ii) entails placing a mold comprising a plurality of masking elements, spaced apart one from the other on the thermoplastic second layer of the flat sheet, and depositing a thermoplastic material, as disclosed herein above, in the spaces formed between adjacent masking elements, at an elevated temperature. Step (ii) further entails lowering the temperature, resulting in the transition of the thermoplastic material to a resilient state, thereby forming the plurality of protrusions thereon.

According to some examples, the deposition of the thermoplastic material at step (ii) is performed by a technique selected from the group consisting of extrusion, brushing, spray-coating, chemical deposition, liquid deposition, vapor deposition, chemical vapor deposition, physical vapor deposition, roller printing, stencil printing, screen printing, inkjet printing, lithographic printing, 3D printing, and combinations thereof.

According to some examples, a thickness of sealing member in its spread relaxed state, is at least 1000% greater following step (ii) than an initial thickness of the sheet provided in step (i).

According to some examples, each one of the plurality of protrusions of the sealing member of the present invention defines a hollow lumen therein, thereby forming hollow protrusions. According to some examples, each hollow lumen comprise two lumen edges, wherein each hollow lumen is open at one or both lumen edges.

According to some examples, each one of the plurality of protrusions comprises a plurality of apertures spaced from each other therealong. According to some examples, each aperture is configured to provide fluid communication between the hollow lumen and an external environment outside of the apertures.

According to some examples, each one of the plurality of apertures is sealed by a biodegradable membrane, configured to enable a controlled release of a pharmaceutical composition from within the each one of the hollow lumens therethrough.

According to some examples, each one of the hollow lumens contains a pharmaceutical composition disposed therein.

According to some examples, each one of the hollow lumens contains an elastic porous element disposed therein. According to some examples, the elastic porous element comprises a pharmaceutical composition disposed therein. According to some examples, the elastic porous element is a sponge.

According to some examples, the pharmaceutical composition comprises at least one pharmaceutical active agent selected from the group consisting of antibiotics, antivirals, antifungals, antiangiogenics, analgesics, anesthetics, anti-inflammatory agents including steroidal and non-steroidal anti-inflammatories (NSAIDs), corticosteroids, antihistamines, mydriatics, antineoplastics, immunosuppressive agents, anti-allergic agents, metalloproteinase inhibitors, tissue inhibitors of metalloproteinases (TIMPs), vascular endothelial growth factor (VEGF) inhibitors or antagonists or intraceptors, receptor antagonists, RNA aptamers, antibodies, hydroxamic acids and macrocyclic anti-succinate hydroxamate derivatives, nucleic acids, plasmids, siRNAs, vaccines, DNA binding compounds, hormones, vitamins, proteins, peptides, polypeptides and peptide-like therapeutic agents, anesthetizers and combinations thereof.

According to some examples, each one of the plurality of protrusions is a divided protrusion, wherein each one of the plurality of divided protrusions forms an inner space between the divided protrusions. According to some examples, said inner space extends between an opening of each divided protrusion toward the first surface of the sealing member. According to some examples, said inner space extends between an opening of each divided protrusion toward a first surface of the first layer. According to some examples, the opening of each one of the plurality of divided protrusions is symmetric relative to an axis extending through the middle of each divided protrusion, thereby forming a symmetric inner space therein. According to some examples, the opening of each one of the plurality of divided protrusions is diverted at an angle relative to an axis extending through the middle of each divided protrusion, thereby forming an asymmetric inner space therein.

According to some examples, the sealing member as disclosed herein above, comprising the hollow protrusions, is prepared by a method comprising: (i) providing a tear resistant flat sheet extending from a first lateral edge to a second lateral edge, and from an inflow edge to an outflow edge; (ii) treating the sheet in a thermal shape-forming process, comprising placing a plurality of elongated molding members on the tear resistant flat sheet. Step (ii) further comprises depositing a thermoplastic layer, at an elevated temperature, on the plurality of elongated molding members, thereby forming a plurality of protrusions (i.e., elevated portions) thereon, which causes the sheet to assume a 3D structure comprising a plurality of elevated portions and a plurality of non-elevated portions. Step (ii) further comprises lowering the temperature, thereby forming a resilient 3D structure of the thermoplastic layer, comprising the plurality of elevated portions.

According to some examples, the method further comprises step (iii) connecting two opposite edges of the sheet to form a cylindrical sealing member in a cylindrical folded state.

According to some examples, the thickness of sealing member in its spread relaxed state following step (ii) is at least 1000% greater than the initial thickness of the sheet provided in step (i).

According to some examples, the tear resistant flat sheet comprises a tear resistant first layer, as disclosed herein above. According to some examples, the tear resistant flat sheet further comprises a thermoplastic second layer, as disclosed herein above. According to some examples, the tear resistant flat sheet further comprises a thermoplastic third layer, as disclosed herein above.

According to some examples, each elongated molding member is made of a temperature resilient metal or a metal alloy.

According to some examples, step (ii) comprises removing the plurality of elongated molding members from within the plurality of protrusions after the formation thereof.

According to some examples, removing the plurality of elongated molding members from within the plurality of protrusions, in step (ii), comprises extracting each elongated molding member through at least one protrusion edge located at the first lateral edge or the second lateral edge of the sheet, thereby forming a plurality of hollow lumens therein.

According to some examples, step (ii) further comprise perforating a plurality of apertures in the plurality of protrusions. According to some examples, step (ii) further comprise inserting a pharmaceutical composition into at least part of said hollow lumens.

According to some examples, the plurality of elongated molding members are a plurality of elastic porous members. According to some examples, step (ii) further comprises impregnating the plurality of elastic porous members with a pharmaceutical composition.

According to some examples, each one of the plurality of elongated molding members comprise a sharp tip. According to some examples, depositing the thermoplastic layer on the plurality of elongated molding members at step (ii) entails contacting the thermoplastic layer with the sharp tips of the elongated molding members. According to some examples, step (ii) further comprises removing the plurality of elongated molding members through the plurality of protrusions, thereby forming a plurality of divided protrusions.

According to some examples, step (ii) comprises pulling the sharp tip of each elongated molding member through the thermoplastic layer. According to some examples, the sharp tip of each elongated molding member is pulled along an axis extending through the middle of each divided protrusion, in a direction perpendicular to the flat sheet, thereby forming a symmetric inner space therein. According to some examples, the sharp tip of each elongated molding member is pulled in the direction of a pulling arrow which is diverted at the angle relative to a direction perpendicular to the flat sheet, thereby forming an asymmetric inner space therein.

According to another aspect of the present invention, there is provided a prosthetic heart valve, comprising: a frame, a leaflet assembly mounted within the frame, and a sealing member coupled to an outer surface of the frame. The frame comprises a plurality of intersecting struts and is movable between a radially compressed state and a radially expanded state. The sealing member is in a folded state. The sealing member extends from an inflow edge toward an opposing outflow edge. The sealing member comprises a first layer and a second layer coating the first layer. A nonfibrous outer surface of the sealing member is formed of a material inherently shaped to define at least one helical protrusion, extending radially outward in a helical configuration around the second layer, between the inflow edge and the outflow edge of the sealing member. The first and second layers are disposed externally to the outer surface of the frame.

According to some examples, the at least one helical protrusion is configured to deform when an external pressure exceeding a predefined threshold is applied thereto in a direction configured to press it against the frame, and to revert to a relaxed state thereof when the external pressure is no longer applied thereto. The distance of the at least one helical protrusion from the frame is greater than the distance of the second layer from the frame in the relaxed state. According to some examples, the predefined threshold of the external pressure is 300 mmHg.

According to some examples, the distance of the at least one helical protrusion from the frame is at least 1000% greater than the distance of the second layer from the frame, in the absence of an external force applied to press the helical protrusion against the frame (i.e., in the relaxed state). According to some examples, the distance of the helical protrusion from the frame is at least 2000% greater than the distance of the second layer therefrom. According to some examples, the distance of the helical protrusion from the frame is at least 3000% greater than the distance of the second layer therefrom.

According to some examples, the nonfibrous outer surface is a smooth surface.

According to some examples, the first layer comprises at least one tear resistant fabric. According to some examples, the tear resistant fabric comprises a ripstop fabric. According to some examples, the first layer comprises a biocompatible material. According to some examples, the first layer comprises at least one elastic material. According to some examples, the first layer comprises a PET fabric. According to some examples, the first layer is having a tear resistance of at least 5N. According to some examples, the first layer is having a tear resistance of at least 15N.

According to some examples, the second layer comprises a biocompatible material. According to some examples, the second layer comprises at least one thromboresistant material. According to some examples, the second layer is made of a thermoplastic material. According to some examples, the second layer is made of a thermoplastic material selected from the group consisting of: polyamides, polyesters, polyethers, polyurethanes, polyolefins, polytetrafluoroethylenes, and combinations and copolymers thereof. According to some examples, the second layer is made of a thermoplastic elastomer. According to some examples, the second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the second layer comprises TPU.

According to some examples, the sealing member comprises a third layer. According to some examples, the second layer and the third layer collectively form a coating which covers the first layer.

According to some examples, the third layer comprises a biocompatible material. According to some examples, the third layer comprises at least one thromboresistant material. According to some examples, the third layer is made of a thermoplastic material. According to some examples, the third layer is made of a thermoplastic material selected from the group consisting of: polyamides, polyesters, polyethers, polyurethanes, polyolefins, polytetrafluoroethylenes, and combinations and copolymers thereof. According to some examples, the third layer is made of a thermoplastic elastomer. According to some examples, the third layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the third layer comprises TPU.

According to some examples, the second layer and the third layer are made from the same material.

According to some examples, the sealing member as disclosed herein above, comprising the at least one helical protrusion, is prepared by a method which comprises: (i) providing a tear resistant flat sheet in a folded cylindrical state, which extends from an inflow edge toward an outflow edge; and (ii) placing at least one helical mandrel around the tear resistant flat sheet. Step (ii) further comprises depositing a thermoplastic layer, at an elevated temperature, on the at least one helical mandrel, thereby forming the at least one helical protrusion thereon, extending radially away at a helical configuration therearound. Step (ii) further comprises lowering the temperature, thereby forming a resilient 3D structure of the thermoplastic layer. Step (ii) further comprises removing the at least one helical mandrel from within the at least one helical protrusion, through at least one helical protrusion edge, located at the inflow edge or the outflow edge, thereby forming a helical hollow lumen therein.

According to some examples, the tear resistant flat sheet comprises a tear resistant first layer, as disclosed herein above.

According to some examples, the thermoplastic layer at step (ii) comprises at least one thromboresistant material. According to some examples, the thermoplastic layer is made of a biocompatible material. According to some examples, the thermoplastic layer is made of a thermoplastic material selected from the group consisting of: polyamides, polyesters, polyethers, polyurethanes, polyolefins, polytetrafluoroethylenes, and combinations and copolymers thereof. According to some examples, the thermoplastic layer is made of a thermoplastic elastomer. According to some examples, the thermoplastic layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic layer comprises TPU.

According to some examples, the tear resistant flat sheet at step (i) further comprises a thermoplastic third layer. According to some examples, the second layer and the third layer comprise the same material.

According to some examples, step (ii) further comprise perforating a plurality of apertures in the helical protrusion. According to some examples, step (ii) further comprise inserting a pharmaceutical composition into at least a part of the helical hollow lumen.

According to another aspect of the present invention, there is provided a prosthetic heart valve, comprising: a frame, a leaflet assembly mounted within the frame, and a sealing member coupled to an outer surface of the frame. The frame comprises a plurality of intersecting struts defining a plurality of junctions, and is movable between a radially compressed state and a radially expanded state. The sealing member extends from an inflow edge toward an opposing outflow edge. The sealing member comprises a tear resistant first layer, and a thermoplastic second layer coating the first layer and defining a first surface of the sealing member. A nonfibrous outer surface of the sealing member is formed of a material inherently shaped to define a single compressible protrusion extending away and around said first surface of the sealing member, in parallel to any one of the outflow and the inflow edges. The first and second layers are disposed externally to the outer surface of the frame.

The length of the single protrusion in a direction extending between the outflow and inflow edges of the sealing member is at least as great as the distance between two junctions of the frame. The junctions are aligned and distanced axially from each other along the frame of the valve.

According to some examples, the distance of the protrusion from the frame is greater than the distance of the first surface of the sealing member from the frame, in the absence of an external force applied to press the protrusion against the frame. According to some examples, the distance of the protrusion from the frame is greater by at least 1000% than the distance of the first surface of the sealing member from the frame. According to some examples, the distance of the protrusion from the frame is greater by at least 2000% than the distance of the first surface from the frame. According to some examples, the distance of the protrusion from the frame is greater by at least 3000% than the distance of the first surface from the frame.

According to some examples, the nonfibrous outer surface is a smooth surface.

According to some examples, the tear resistant first layer comprises a ripstop fabric. According to some examples, the first layer comprises a biocompatible material. According to some examples, the first layer comprises at least one elastic material. According to some examples, the first layer comprises a PET fabric. According to some examples, the first layer is having a tear resistance of at least 5N. According to some examples, the first layer is having a tear resistance of at least 15N.

According to some examples, the thermoplastic second layer comprises a biocompatible material. According to some examples, the second layer comprises at least one thromboresistant material. According to some examples, the second layer is made of a thermoplastic material selected from the group consisting of: polyamides, polyesters, polyethers, polyurethanes, polyolefins, polytetrafluoroethylenes, and combinations and copolymers thereof. According to some examples, the second layer is made of a thermoplastic elastomer. According to some examples, the second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the second layer comprises TPU.

According to some examples, the sealing member comprises a thermoplastic third layer. According to some examples, the thermoplastic second layer and the thermoplastic third layer collectively form a thermoplastic coating which covers the tear resistant first layer.

According to some examples, the thermoplastic third layer comprises a biocompatible material. According to some examples, the third layer comprises at least one thromboresistant material. According to some examples, the third layer is made of a thermoplastic material selected from the group consisting of: polyamides, polyesters, polyethers, polyurethanes, polyolefins, polytetrafluoroethylenes, and combinations and copolymers thereof. According to some examples, the third layer is made of a thermoplastic elastomer. According to some examples, the third layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the third layer comprises TPU.

According to some examples, the thermoplastic second layer and the thermoplastic third layer are made from the same material.

According to some examples, the single compressible protrusion defines a single hollow lumen therein.

According to some examples, the single compressible protrusion comprises a plurality of apertures spaced from each other therealong. According to some examples, each one of the plurality of apertures is sealed by a biodegradable membrane, configured to enable a controlled release of a pharmaceutical composition from within the single hollow lumen therethrough. According to some examples, the single hollow lumen contains a pharmaceutical composition disposed therein. According to some examples, at least a portion of the apertures are sealed with a semi permeable membrane.

According to another aspect of the present invention, there is provided a method for producing a perivalvular leakage (PVL) skirt, the method comprises: (i) providing a tear resistant flat sheet; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

According to some examples, the tear resistant flat sheet at step (i) comprises a tear resistant first layer and a thermoplastic second layer. The tear resistant flat sheet extends between a first lateral edge and a second lateral edge, and between an inflow edge and an outflow edge. According to some examples, the treatment at step (ii) comprises contacting the flat sheet with a mold at an elevated temperature. Step (ii) further comprises lowering the temperature, thereby maintaining a resilient structure of the thermoplastic second layer, wherein the second layer is located distally to the mold. Step (ii) further comprises removing the mold from the sheet, after the temperature was lowered.

According to some examples, the flat sheet in step (i) comprises a tear resistant first layer located between a thermoplastic second layer and a thermoplastic third layer of the flat sheet. According to some examples, step (ii) entails contacting the flat sheet with the mold, wherein the third layer is contacting the mold.

According to some examples, step (ii) comprises contacting the flat sheet with the mold at an elevated temperature, thereby forming a plurality of ridges thereon.

According to some examples, the second layer is thermally shape-formable at the elevated temperature and resilient at the lowered temperature. According to some examples, the elevated temperature in step (ii) is at least 60° C. According to some examples, the lowered temperature in step (ii) is below 40° C.

According to some examples, step (ii) entails placing the flat sheet on a mold, wherein the second layer is located distally to the mold. According to some examples, step (ii) entails placing the flat sheet on the mold, wherein the third layer is contacting the mold.

According to some examples, step (ii) comprises placing the flat sheet on a mold at an elevated temperature and gravitationally submerging the heated sheet, thereby forming a plurality of ridges thereon. According to some examples, the mold is selected from a plurality of rods, tubes, pipes, and combinations thereof.

According to some examples, the mold comprises a base, a plurality of protrusions and a vacuum source comprising a plurality of apertures. According to some examples, the plurality of protrusions extend away from the base and are spaced from each other along the base. According to some examples, the plurality of apertures are formed at the base, at the protrusions, or at both.

According to some examples, step (ii) comprises positioning the flat sheet above the mold. Step (ii) further comprises heating the flat sheet to a thermoformable temperature. Step (ii) further comprises bringing the sheet towards said mold, to effectively engage said flat sheet with the protrusions of mold, thereby to enable the sheet to conform to said protrusions. The engagement of the sheet with the plurality of protrusions forms a plurality of ridges, while the engagement of the sheet with the base forms a plurality of inter-ridge gaps of the sealing member.

According to some examples, step (ii) includes application of force using mold over two opposite edges of the flexible sheet. According to some examples, the mold comprises a first mold and a second mold. According to some examples, the first mold comprises a first base and plurality of first mold protrusions and the second mold comprises a second base and plurality of second mold protrusions. According to some examples, step (ii) comprises placing the flat sheet between the plurality first mold protrusions and the plurality of second mold protrusions, so that the plurality first mold protrusions and the plurality second mold protrusions are disposed at a zipper-like configuration. According to some examples, step (ii) further comprises pressing the second mold against the first mold at an elevated temperature, thereby effectively engaging the flat sheet therebetween to enable the sheet to conform to said molds.

According to another aspect of the present invention, there is provided a method for producing a perivalvular leakage (PVL) skirt, the method comprises: (i) providing a tear resistant flat sheet consisting of a tear resistant first layer; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

The tear resistant flat sheet extends between a first lateral edge and a second lateral edge, and between an inflow edge and an outflow edge. According to some examples, the treatment at step (ii) comprises placing the flat sheet on a mold, thereby forming a plurality of ridges thereon over the mold, wherein the mold comprises a base and a plurality of protrusions. Step (ii) further comprises heat-coating the sheet at an elevated thermoformable temperature with a thermoplastic material, thereby forming a thermoplastic second layer thereon. Step (ii) further comprises lowering the temperature, thereby forming a resilient structure of the thermoplastic second layer.

According to some examples, the elevated thermoformable temperature in step (ii) is at least 60° C. According to some examples, the lowered temperature in step (ii) is below 40° C.

According to another aspect of the present invention, there is provided a method for producing a perivalvular leakage (PVL) skirt, the method comprises: (i) providing a tear resistant flat sheet; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

According to some examples, the tear resistant flat sheet at step (i) comprises a tear resistant first layer and a thermoplastic second layer. The tear resistant flat sheet extends between a first lateral edge and a second lateral edge, and between an inflow edge and an outflow edge. According to some examples, the treatment at step (ii) comprises extruding a plurality of members on the thermoplastic second layer of the flat sheet, wherein the members are spaced from each other. Each member comprises a molten composition at an elevated temperature. Step (ii) further comprises lowering the temperature, resulting in the transition of each extruded member to a resilient state, thereby forming a plurality of protrusions thereon.

According to some examples, the flat sheet in step (i) comprises a tear resistant first layer located between a thermoplastic second layer and a thermoplastic third layer of the flat sheet.

According to some examples, the molten composition comprises at least one thromboresistant material. According to some examples, the molten composition is made of a biocompatible thermoplastic material selected from the group consisting of: polyamides, polyesters, polyethers, polyurethanes, polyolefins, polytetrafluoroethylenes, and combinations and copolymers thereof. According to some examples, the molten composition is made of a thermoplastic elastomer. According to some examples, the molten composition is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the molten composition comprises TPU.

According to some examples, the elevated temperature in step (ii) is at least 60° C. According to some examples, the lowered temperature in step (ii) is below 40° C.

According to some examples, each one of the plurality of protrusions formed in step (ii) is in a 3D shape selected from the group consisting of: inverse U-shapes, half-spheres, domes, cylinders, pyramids, triangular prisms, pentagonal prisms, hexagonal prisms, flaps, polygons, and combinations thereof.

According to some examples, each one of the plurality of protrusions formed in step (ii) is elongated and extends substantially in parallel to at least one of the inflow edge and/or the outflow edge of the sheet. According to some examples, each one of the plurality of protrusions formed in step (ii) is elongated and extends substantially perpendicular to at least one of the inflow edge and the outflow edge of the sheet. According to some examples, each one of the plurality of protrusions formed in step (ii) is elongated and extends substantially diagonally with respect to at least one of the inflow edge and the outflow edge of the sheet.

According to another aspect of the present invention, there is provided a method for producing a perivalvular leakage (PVL) skirt, the method comprises: (i) providing a tear resistant flat sheet; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

According to some examples, the tear resistant flat sheet at step (i) comprises a tear resistant first layer and a thermoplastic second layer. The tear resistant flat sheet extends between a first lateral edge and a second lateral edge, and between an inflow edge and an outflow edge. According to some examples, the treatment at step (ii) comprises placing a mold comprising a plurality of masking elements spaced apart one from the other on the thermoplastic second layer of the flat sheet. Step (ii) further comprises depositing a thermoplastic material at an elevated temperature in the spaces formed between adjacent masking elements. Step (ii) further comprises lowering the temperature, resulting in the transition of the thermoplastic material to a resilient state, thereby forming a plurality of protrusions on the flat sheet.

According to some examples, the flat sheet in step (i) comprises a tear resistant first layer located between a thermoplastic second layer and a thermoplastic third layer of the flat sheet.

According to some examples, the thermoplastic material at step (ii) is biocompatible. According to some examples, the thermoplastic material at step (ii) is selected from the group consisting of: polyamides, polyesters, polyethers, polyurethanes, polyolefins, polytetrafluoroethylenes, and combinations and copolymers thereof. According to some examples, the thermoplastic material is thromboresistant. According to some examples, the thermoplastic material is a thermoplastic elastomer. According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic material comprises TPU.

According to some examples, each one of the plurality of protrusions formed in step (ii) is in a 3D shape selected from the group consisting of: inverse U-shapes, half-spheres, domes, cylinders, pyramids, triangular prisms, pentagonal prisms, hexagonal prisms, flaps, polygons, and combinations thereof.

According to some examples, each one of the plurality of protrusions formed in step (ii) is elongated and extends substantially in parallel to at least one of the inflow edge and/or the outflow edge of the sheet. According to some examples, each one of the plurality of protrusions formed in step (ii) is elongated and extends substantially perpendicular to at least one of the inflow edge and the outflow edge of the sheet. According to some examples, each one of the plurality of protrusions formed in step (ii) is elongated and extends substantially diagonally with respect to at least one of the inflow edge and the outflow edge of the sheet.

According to some examples, the deposition of the thermoplastic material at step (ii) is performed by a technique selected from the group consisting of extrusion, brushing, spray-coating, chemical deposition, liquid deposition, vapor deposition, chemical vapor deposition, physical vapor deposition, roller printing, stencil printing, screen printing, inkjet printing, lithographic printing, 3D printing, and combinations thereof.

According to some examples, the deposition of the thermoplastic material at step (ii) comprises depositing a monomer composition in the spaces formed between adjacent masking elements, and polymerizing the composition, resulting in a transition of the monomer composition to a polymerized resilient state, thereby forming a plurality of protrusions on the flat sheet.

According to some examples, the elevated temperature in step (ii) is at least 60° C. According to some examples, the lowered temperature in step (ii) is below 40° C.

According to another aspect of the present invention, there is provided a method for producing a perivalvular leakage (PVL) skirt, the method comprises: (i) providing a tear resistant flat sheet; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

The tear resistant flat sheet extends between a first lateral edge and a second lateral edge, and between an inflow edge and an outflow edge. According to some examples, the treatment at step (ii) comprises placing a plurality of elongated molding members on the tear resistant flat sheet. Step (ii) further comprises depositing a thermoplastic layer, at an elevated temperature on the plurality of elongated molding members, thereby forming a plurality of protrusions thereon. Step (ii) further comprises lowering the temperature, thereby forming a resilient 3D structure of the protrusions. Step (ii) further comprises removing the plurality of elongated molding members from within the plurality of protrusions.

According to some examples, the flat sheet in step (i) consists of a single tear resistant first layer. According to some examples, the flat sheet in step (i) further comprises a thermoplastic second layer. According to some examples, the flat sheet in step (i) comprises a tear resistant first layer located between a thermoplastic second layer and a thermoplastic third layer of the flat sheet.

According to some examples, step (ii) comprises placing the plurality of elongated molding members on the tear resistant flat sheet; and depositing the thermoplastic layer, at the elevated temperature, on the tear resistant flat sheet, such that the plurality of elongated molding members are positioned between the tear resistant flat sheet and the thermoplastic layer, thereby forming a plurality of 3D shaped protrusions.

According to some examples, the elevated temperature in step (ii) is at least 60° C. According to some examples, the lowered temperature in step (ii) is below 40° C.

According to some examples, the thermoplastic layer at step (ii) is biocompatible. According to some examples, the thermoplastic layer at step (ii) is selected from the group consisting of: polyamides, polyesters, polyethers, polyurethanes, polyolefins, polytetrafluoroethylenes, and combinations and copolymers thereof. According to some examples, the thermoplastic layer is thromboresistant. According to some examples, the thermoplastic layer is a thermoplastic elastomer. According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic layer comprises TPU.

According to some examples, the plurality of elongated molding members are made of a temperature resilient metal or a metal alloy. According to some examples, the plurality of elongated molding members are selected from rods, tubes, pipes, and combinations thereof.

According to some examples, removing the plurality of elongated molding members from within the plurality of protrusions in step (ii) comprises extracting each elongated molding member through at least one protrusion edge, located at the first lateral edge or the second lateral edge of the sheet, thereby forming a plurality of hollow lumens therein.

According to some examples, step (ii) further comprises perforating a plurality of apertures in the plurality of protrusions. According to some examples, step (ii) further comprise inserting a pharmaceutical composition into at least part of said hollow lumens.

According to another aspect of the present invention, there is provided a method for producing a perivalvular leakage (PVL) skirt, the method comprises: (i) providing a tear resistant flat sheet; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

The tear resistant flat sheet extends between a first lateral edge and a second lateral edge, and between an inflow edge and an outflow edge. According to some examples, the treatment at step (ii) comprises placing a plurality of elastic porous members on the tear resistant flat sheet. Step (ii) further comprises depositing a thermoplastic layer, at an elevated temperature on the plurality of elastic porous members, thereby forming a plurality of protrusions. Step (ii) further comprises lowering the temperature, thereby forming a resilient 3D structure of the protrusions.

According to some examples, the flat sheet in step (i) consists of a single tear resistant first layer. According to some examples, the flat sheet in step (i) further comprises a thermoplastic second layer. According to some examples, the flat sheet in step (i) comprises a tear resistant first layer located between a thermoplastic second layer and a thermoplastic third layer of the flat sheet.

According to some examples, step (ii) comprises placing the plurality of elastic porous members on the tear resistant flat sheet; and depositing the thermoplastic layer, at the elevated temperature, on the tear resistant flat sheet, such that the plurality of elastic porous members are positioned between the tear resistant flat sheet and the thermoplastic layer, thereby forming a plurality of 3D shaped protrusions comprising the elastic porous members there-within.

According to some examples, the elevated temperature in step (ii) is at least 60° C. According to some examples, the lowered temperature in step (ii) is below 40° C.

According to some examples, the thermoplastic layer at step (ii) is biocompatible. According to some examples, the thermoplastic layer at step (ii) is selected from the group consisting of: polyamides, polyesters, polyethers, polyurethanes, polyolefins, polytetrafluoroethylenes, and combinations and copolymers thereof. According to some examples, the thermoplastic layer is thromboresistant. According to some examples, the thermoplastic layer is a thermoplastic elastomer. According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic layer comprises TPU.

According to some examples, each elastic porous member is made of a temperature resilient biocompatible sponge.

According to some examples, step (ii) further comprises perforating a plurality of apertures in the plurality of protrusions.

According to some examples, step (ii) further comprises impregnating the plurality of elastic porous members with a pharmaceutical composition.

According to another aspect of the present invention, there is provided a method for producing a perivalvular leakage (PVL) skirt, the method comprises: (i) providing a tear resistant flat sheet; (ii) treating the sheet in a thermal shape-forming process to assume a resilient structure comprising a plurality of elevated portions and a plurality of non-elevated portions, in a spread relaxed state, and (iii) connecting two opposite edges of the sheet to form a cylindrical sealing member in a cylindrical folded state.

The tear resistant flat sheet extends between a first lateral edge and a second lateral edge, and between an inflow edge and an outflow edge. According to some examples, the treatment at step (ii) comprises placing a plurality of elongated molding members on the tear resistant flat sheet, wherein each of the plurality of elongated molding members comprises a sharp tip. Step (ii) further comprises depositing a thermoplastic layer, at an elevated temperature on the plurality of elongated molding members, thereby forming a plurality of protrusions. Step (ii) further comprises lowering the temperature, thereby forming a resilient 3D structure thereof. Step (ii) further comprises removing the plurality of elongated molding members through the plurality of protrusions, thereby forming a plurality of divided protrusions.

According to some examples, the flat sheet in step (i) consists of a single tear resistant first layer. According to some examples, the flat sheet in step (i) further comprises a thermoplastic second layer. According to some examples, the flat sheet in step (i) comprises a tear resistant first layer located between a thermoplastic second layer and a thermoplastic third layer of the flat sheet.

According to some examples, depositing the thermoplastic layer on the plurality of elongated molding members at step (ii) entails contacting the thermoplastic layer with the sharp tips of the elongated molding members.

According to some examples, the plurality of elongated molding members and sharp tips are made of a temperature resilient metal or a metal alloy.

According to some examples, step (ii) comprises pulling the sharp tip of each elongated molding member through the thermoplastic layer.

According to some examples, the sharp tip of each elongated molding member is pulled along an axis extending through the middle of each divided protrusion, in a direction perpendicular to the flat sheet, thereby forming a symmetric inner space therein.

According to some examples, the sharp tip of each elongated molding member is pulled in the direction of a pulling arrow which is diverted at the angle relative to a direction perpendicular to the flat sheet, thereby forming an asymmetric inner space therein.

According to some examples, the elevated temperature in step (ii) is at least 60° C. According to some examples, the lowered temperature in step (ii) is below 40° C.

According to some examples, the thermoplastic layer at step (ii) is biocompatible. According to some examples, the thermoplastic layer at step (ii) is selected from the group consisting of: polyamides, polyesters, polyethers, polyurethanes, polyolefins, polytetrafluoroethylenes, and combinations and copolymers thereof. According to some examples, the thermoplastic layer is thromboresistant. According to some examples, the thermoplastic layer is a thermoplastic elastomer. According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic layer comprises TPU.

According to another aspect of the present invention, there is provided a method for producing a perivalvular leakage (PVL) skirt, the method comprises: (i) providing a tear resistant flat sheet in a folded cylindrical state, extending from an inflow edge towards an outflow edge; and (ii) treating the sheet in a thermal shape-forming process to assume a resilient structure comprising a plurality of elevated portions and a plurality of non-elevated portions, in the folded cylindrical state.

According to some examples, the treatment at step (ii) comprises placing at least one helical mandrel around the tear resistant flat sheet. Step (ii) further comprises depositing a thermoplastic layer, at an elevated temperature, on the at least one helical mandrel, thereby forming at least one helical protrusion thereon extending radially away at a helical configuration therearound. Step (ii) further comprises lowering the temperature, thereby maintaining a resilient structure of the thermoplastic layer. Step (ii) further comprises removing the at least one helical mandrel from within the at least one helical protrusion, through at least one helical protrusion edge located at the inflow edge or the outflow edge, thereby forming a helical hollow lumen therein.

According to some examples, the flat sheet in step (i) consists of a single tear resistant first layer. According to some examples, the flat sheet in step (i) further comprises a thermoplastic second layer. According to some examples, the flat sheet in step (i) comprises a tear resistant first layer located between a thermoplastic second layer and a thermoplastic third layer of the flat sheet.

According to some examples, step (ii) entails placing the at least one helical mandrel around the thermoplastic second layer of the flat sheet.

According to some examples, the elevated temperature in step (ii) is at least 60° C. According to some examples, the lowered temperature in step (ii) is below 40° C.

According to some examples, the thermoplastic layer at step (ii) is biocompatible. According to some examples, the thermoplastic layer at step (ii) is selected from the group consisting of: polyamides, polyesters, polyethers, polyurethanes, polyolefins, polytetrafluoroethylenes, and combinations and copolymers thereof. According to some examples, the thermoplastic layer is thromboresistant. According to some examples, the thermoplastic layer is a thermoplastic elastomer. According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic layer comprises TPU.

According to some examples, step (ii) further comprise perforating a plurality of apertures in the helical protrusion.

According to some examples, step (ii) further comprise inserting a pharmaceutical composition into at least a part of the helical hollow lumen.

According to some examples, at any one of the above methods, the thickness of sealing member, optionally in the spread relaxed state, following step (ii) is at least 1000% greater than the initial thickness of the sheet provided in step (i). According to further examples, the thickness of sealing member following step (ii) is at least 2000% greater than the initial thickness of the sheet provided in step (i). According to still further examples, the thickness of sealing member following step (ii) is at least 3000% greater than the initial thickness of the sheet provided in step (i).

According to some examples, the tear resistant first layer of the flat sheet, at any one of the above methods, comprises a ripstop fabric. According to some examples, the tear resistant first layer comprises a biocompatible material. According to some examples, the tear resistant first layer comprises at least one elastic material. According to some examples, the tear resistant first layer comprises a PET fabric. According to some examples, the tear resistant first layer is having a tear resistance of at least 5N. According to some examples, the tear resistant first layer is having a tear resistance of at least 15N.

According to some examples, the thermoplastic second layer of the flat sheet, at any one of the above methods, comprises at least one thromboresistant material. According to some examples, the thermoplastic second layer is made of a biocompatible material. According to some examples, the thermoplastic second layer is made of a thermoplastic material selected from the group consisting of: polyamides, polyesters, polyethers, polyurethanes, polyolefins, polytetrafluoroethylenes, and combinations and copolymers thereof. According to some examples, the thermoplastic second layer is made of a thermoplastic elastomer. According to some examples, the thermoplastic second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic second layer comprises TPU.

According to some examples, the thermoplastic third layer of the flat sheet, at any one of the above methods, is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof. According to some examples, the thermoplastic third layer comprises TPU. According to some examples, the thermoplastic second layer and the thermoplastic third layer are made from the same material.

According to another aspect of the present invention, there is provided a perivalvular leakage (PVL) skirt, produced by any one of the methods as disclosed herein above.

According to another aspect of the present invention, there is provided a prosthetic heart valve, comprising: a frame, a leaflet assembly mounted within the frame, and a sealing member coupled to an outer surface of the frame. The frame comprises a plurality of intersecting struts and is movable between a radially compressed state and a radially expanded state. The sealing member produced according to any one of the methods as disclosed herein above.

Certain examples of the present invention may include some, all, or none of the above advantages. Further advantages may be readily apparent to those skilled in the art from the figures, descriptions, and claims included herein. Aspects and examples of the invention are further described in the specification herein below and in the appended claims.

Unless otherwise defined, all technical and scientific terms used herein have the same meaning as commonly understood by one of ordinary skill in the art to which this invention pertains. In case of conflict, the patent specification, including definitions, governs. As used herein, the indefinite articles “a” and “an” mean “at least one” or “one or more” unless the context clearly dictates otherwise.

The following examples and aspects thereof are described and illustrated in conjunction with systems, tools and methods which are meant to be exemplary and illustrative, but not limiting in scope. In various examples, one or more of the above-described problems have been reduced or eliminated, while other examples are directed to other advantages or improvements.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 show a prosthetic valve, including various components thereof, according to some examples.

FIGS. 2A-2B show the prosthetic valve, in a crimped state (FIG. 2A), and in an expanded state disposed over an inflated balloon (FIG. 2B), according to some examples.

FIGS. 3A-3B show a side view and a top view, respectively, of the prosthetic valve positioned at a target implantation site, according to some examples.

FIG. 4A shows a view in perspective of a sealing member in a spread relaxed state, according to some examples.

FIGS. 4B and 4C show cross sectional views of the sealing member in a spread relaxed state, according to some examples.

FIGS. 4D-4F show views in perspective of various configurations of the sealing member, in a cylindrical folded state, according to some examples.

FIGS. 5A-5C show various configurations of sealing member mounted on the frame of the prosthetic valve, according to some examples.

FIGS. 6A-6B show exemplary thermal shape-processing steps utilizing thermoforming, for the fabrication of the sealing member in a spread state, according to some examples.

FIGS. 6C-6D show thermal processing steps of a flat flexible sheet, utilizing placing, heating and vacuum-thermoforming over a mold, for the fabrication of the sealing member in a spread state, according to some examples.

FIG. 6E shows thermal processing steps of a flat flexible sheet, utilizing thermoforming, which includes application of force using mold over two opposite surface thereof, for the fabrication of the sealing member in a spread state, according to some examples.

FIG. 7A shows a flexible sheet at a spread relaxed state, according to some examples.

FIG. 7B shows the flexible sheet of FIG. 7A placed over a mold, such that the flexible sheet flexibly alters its shape to assume the shape of the mold, according to some examples.

FIG. 7C shows a coating process of the shaped-altered flexible sheet of FIG. 7B, according to some examples.

FIG. 8A shows a view in perspective of a sealing member in a spread relaxed state, according to some examples.

FIGS. 8B and 8C show cross-sectional views of the sealing member in a spread relaxed state, according to some examples.

FIGS. 8D-8F shows views in perspective of various configurations of the sealing member, in a cylindrical folded state, according to some examples.

FIGS. 9A-9C show various configurations of the sealing member mounted on the frame of the prosthetic valve, according to some examples.

FIGS. 10A-10C show processing steps utilizing extrusion for the fabrication of the sealing member, according to some examples.

FIGS. 11A-11E show processing steps utilizing a plurality of making elements, for the fabrication of the sealing member, according to some examples.

FIG. 12A shows a view in perspective of a sealing member in a spread relaxed state, according to some examples.

FIGS. 12B-12E show various cross-sectional views of the sealing member in a spread relaxed state, according to some examples.

FIG. 12F shows a view in perspective of a sealing member in a spread relaxed state comprising a plurality of apertures, according to some examples.

FIG. 12G shows a cross section of the sealing member of FIG. 12F, according to some examples.

FIG. 12H shows a view in perspective of a sealing member in a spread relaxed state comprising a plurality of flaps, according to some examples.

FIGS. 13A-13C show views in perspective of various configurations of the sealing member, in a cylindrical folded state, according to some examples.

FIG. 13D shows a view in perspective of a folded sealing member, according to some examples.

FIGS. 14A-14C show various configurations of the sealing member mounted on the prosthetic valve, according to some examples.

FIG. 14D shows the folded sealing member mounted on the frame of prosthetic valve, according to some examples.

FIG. 15 shows the configurations of the sealing member comprising the plurality of apertures, mounted on the frame of prosthetic valve, according to some examples.

FIGS. 16A-16E show various stages of processing steps for the manufacture of sealing member utilizing a plurality of mandrels, according to some examples.

FIGS. 17A-17F show various stages of processing steps for the manufacture of sealing member, utilizing a plurality of mandrels comprising sharp tips, according to some examples.

FIGS. 18A-18D show various stages of processing steps for the manufacture of sealing member utilizing a plurality of mandrels, according to some examples.

FIGS. 19A-19D show various stages of processing steps for the manufacture of sealing member, utilizing a plurality of mandrels comprising sharp tips, according to some examples.

FIG. 20 shows a view in perspective of various configurations of the sealing members of the present invention, during a folding transition from a spread to a cylindrical folded state, according to some examples.

FIGS. 21A-21B show a side view and a top view, respectively, of the prosthetic valve comprising various sealing members at a specific configuration, positioned at a target implantation site, according to some examples.

FIGS. 22A-22B show a side view and a top view, respectively, of the prosthetic valve comprising various sealing members at a specific configuration, positioned at a target implantation site, according to some examples.

FIGS. 23A-23B show an additional configuration of a sealing member comprising a single protrusion, mounted on the frame of prosthetic valve, in an expanded state (FIG. 23A), and in a crimped state (FIG. 23B), according to some examples.

FIG. 24 shows an additional configuration of a sealing member comprising a single protrusion provided with a plurality of apertures, according to some examples.

### DETAILED DESCRIPTION OF SOME EXAMPLES

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

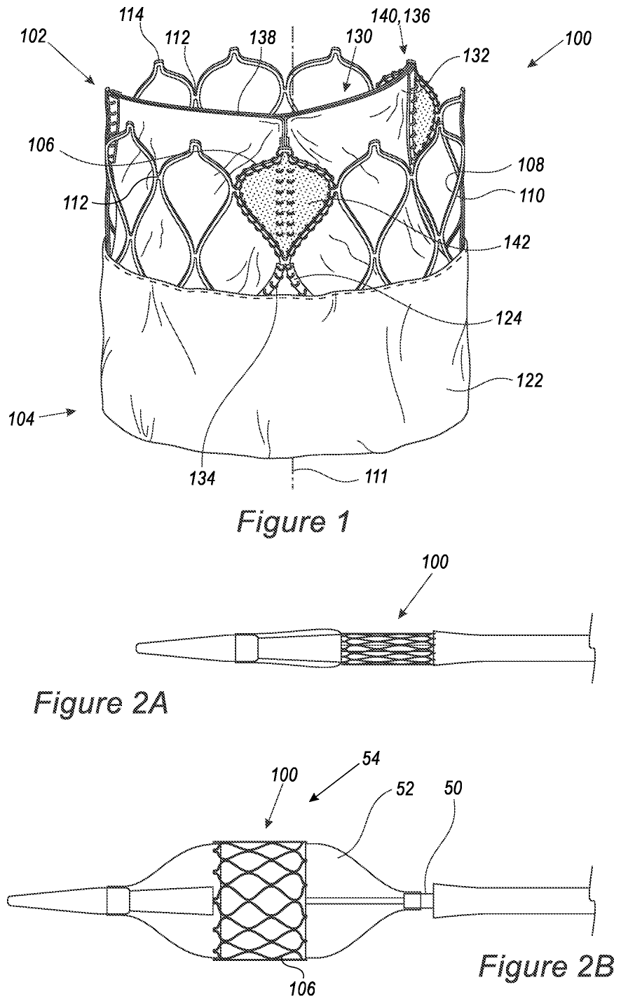

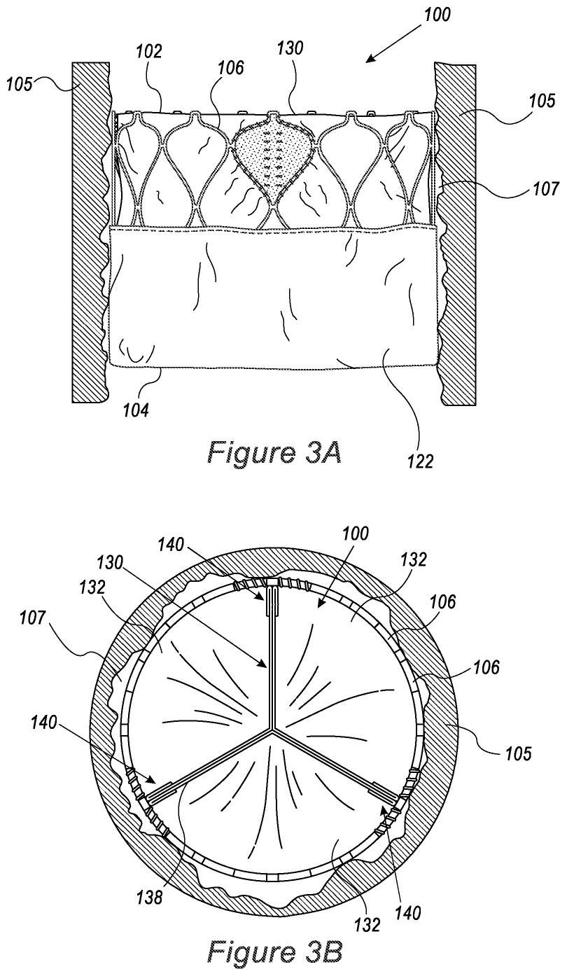

Reference is now made to FIGS. 1-3B. FIG. 1 shows a prosthetic heart valve 100, including various components thereof, according to some examples. FIG. 2A shows the prosthetic valve 100 in a crimped state, and FIG. 2B shows the prosthetic heart valve 100 in an expanded state disposed over an inflated balloon, according to some examples. FIGS. 3A-3B show a side view and a top view, respectively, of the prosthetic heart valve 100 positioned at a target implantation site, according to some examples.

The prosthetic heart valve 100 is deliverable to a subject's target site over a catheter 50 (shown, for example, in FIGS. 2A-2B), and is radially expandable and compressible between a radially compressed, or crimped, state (as shown, for example, in FIG. 2A), and a radially expanded state (as shown, for example, in FIGS. 1 and 2B). It is to be understood by the skilled in the art that the subject's target sites for implantation of prosthetic heart valves include a native aortic valve, a native mitral valve, a native pulmonary valve, and a native tricuspid valve of a subject. The term “prosthetic valve”, as used herein, refers to any type of a prosthetic valve deliverable to a patient's target site over a catheter, which is radially expandable and compressible between a radially compressed, or crimped, state, and a radially expanded state.

The expanded state may include a range of diameters to which the valve 100 may expand, between the compressed state and a maximal diameter reached at a fully expanded state. Thus, a plurality of partially expanded states may relate to any expansion diameter between a radially compressed or crimped state, and maximally expanded state. It is thus to be understood that when the term “expanded state” is used herein, both the maximally and the partially expanded states are referred. A prosthetic valve 100 of the current disclosure may include any prosthetic valve configured to be mounted within the native aortic valve, the native mitral valve, the native pulmonary valve, and the native tricuspid valve of a human subject.

As used herein, the terms “compressed” and “crimped” are interchangeable, and refer to the state of valve 100 as shown in FIG. 2A.

The term “plurality”, as used herein, means more than one.

As stated above, the prosthetic heart valve 100 can be delivered to the site of implantation via a delivery assembly carrying the valve 100 in a radially compressed or crimped state, toward the target site, to be mounted against the native anatomy, by expanding the valve 100 via various expansion mechanisms. FIG. 1 shows an example of a balloon expandable prosthetic valve 100. Processes for implanting balloon expandable prosthetic valves generally involve a procedure of inflating a balloon within a prosthetic valve, thereby expanding the prosthetic valve 100 within the desired implantation site. Once the valve is sufficiently expanded, the balloon is deflated and retrieved along with the delivery assembly.

Other types of valves may include other expansion mechanisms, such as mechanical expansion mechanisms or self-expandable mechanisms (not shown). Mechanically expandable valves are a category of prosthetic valves that rely on a mechanical actuation mechanism for expansion. The mechanical actuation mechanism usually includes a plurality of expansions and locking assemblies, releasably coupled to respective actuation assemblies of a delivery apparatus, controlled via a handle for actuating the actuation assemblies to expand the prosthetic valve to a desired diameter. The expansion and locking assemblies may optionally lock the valve's position to prevent undesired recompression thereof, and disconnection of the actuation assemblies from the expansion and locking assemblies, to enable retrieval of the delivery apparatus once the prosthetic valve is properly positioned at the desired site of implantation.

Self-expandable valves include a frame that is shape-set to automatically expand as soon as an outer retaining structure, such as a capsule or a portion of a shaft, is withdrawn proximally relative to the prosthetic valve.

A prosthetic valve 100 can comprise an inflow end 104 and an outflow end 102. The prosthetic valve 100 can define a centerline 111 extending through the inflow end 104 and the outflow end 102. In some instances, the outflow end 102 is the distal end of the prosthetic valve 100, and the inflow end 104 is the proximal end of the prosthetic valve 100. Alternatively, depending for example on the delivery approach of the valve, the outflow end can be the proximal end of the prosthetic valve, and the inflow end can be the distal end of the prosthetic valve.

The term “proximal”, as used herein, generally refers to a position, direction, or portion of any device or a component of a device, which is closer to the user (e.g., medical personnel) and further away from the implantation site.

The term “distal”, as used herein, generally refers to a position, direction, or portion of any device or a component of a device, which is further away from the user (e.g., medical personnel) and closer to the implantation site.

The term “outflow”, as used herein, refers to a region of the prosthetic valve through which the blood flows through and out of the valve 100.

The term “inflow”, as used herein, refers to a region of the prosthetic valve through which the blood flows into the valve 100.

It is thus to be understood that upon implantation of the prosthetic heart valve 100 in a subject's implantation site, blood is flowing through the prosthetic heart valve 100 in the direction from the inflow end 104, where blood enters the valve 100 to outflow end 102, where blood exits the valve 100.

The valve 100 comprises an annular frame 106 movable between a radially compressed state and a radially expanded state, and a leaflet assembly 130 mounted within the frame 106.

The frame 106 can be made of various suitable materials, including plastically-deformable materials such as, but not limited to, stainless steel, a nickel-based alloy (e.g., a cobalt-chromium or a nickel-cobalt-chromium alloy such as MP35N alloy), polymers, or combinations thereof. When constructed of a plastically-deformable material(s), the frame 106 can be crimped to a radially compressed state on a delivery shaft 50 (e.g., catheter 50), for example by using a crimping device (not shown), and then expanded inside a patient by an inflatable balloon 52 (see FIGS. 2A-B) or an equivalent expansion mechanism. Alternatively or additionally, the frame 106 can be made of shape-memory materials such as, but not limited to, nickel titanium alloy (e.g., Nitinol). When constructed of a shape-memory material, such as the case for self-expandable valves, the frame 106 can be crimped to a radially compressed state and restrained in the compressed state by insertion into a shaft 50 or an equivalent mechanism of a delivery apparatus (not shown).

In the example illustrated in FIG. 1, the frame 106 is an annular, stent-like structure comprising a plurality of intersecting struts 110. The frame 106 can have one or more rows of openings or cells 108 defined by intersecting struts, such as the angled struts 110 shown in FIG. 1. The struts 110 can intersect at junctions 112, such as for example, struts 110 can intersect at an upper junction defining an outflow apex 114. The frame 106 can have a cylindrical or substantially cylindrical shape having a constant diameter from the inflow end 104 to the outflow end 102 of the frame as shown, or the frame can vary in diameter along the height of the frame, as disclosed in U.S. Pat. No. 9,155,619, which is incorporated herein by reference.

The struts 110 may be pivotable or bendable relative to each other, so as to permit frame expansion or compression. In some implementations, the frame 106 can be formed from a single piece of material, such as a metal tube, via various processes such as, but not limited to, laser cutting, electroforming, and/or physical vapor deposition, while retaining the ability to collapse/expand radially.

The leaflet assembly 130 comprises a plurality of leaflets 132 (e.g., three leaflets), positioned at least partially within the frame 106, and configured to regulate flow of blood through the prosthetic valve 100 from the inflow end 104 to the outflow end 102. While three leaflets 132 arranged to collapse in a tricuspid arrangement are shown in the example illustrated in FIG. 1, it will be clear that a prosthetic valve 100 can include any other number of leaflets 132. The lower edge 134 of the leaflet assembly 130 preferably has an undulating, curved scalloped shape. By forming the leaflets with this scalloped geometry, stresses on the leaflets 130 are reduced, which in turn improves durability of the valve 100. The scalloped geometry also reduces the amount of tissue material used to form leaflet assembly 130, thereby allowing a smaller, more even crimped profile at the inflow end of the valve.

In the context of the prosthetic aortic valve 100 disclosed herein, the terms “lower” and “upper” are used interchangeably with the terms “inflow” and “outflow”, respectively, for convenience.

The leaflets 132 are made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, which are incorporated by reference herein.

Each leaflet 132 can be coupled to the frame 106 along its inflow edge (the lower edges of the leaflets, also referred to as “cusp edges”) and/or at commissures 140 of the leaflet assembly 130 where adjacent portions of two leaflets 130 are connected to each other.

According to some examples, the prosthetic valve 100 further comprises a sealing member 122 that can be mounted on the outer surface of the frame 106. According to some examples, the sealing member 122 is configured to function, for example, as a sealing member retained between the frame 106 and the surrounding tissue of the native annulus against which the prosthetic valve 100 is mounted. Advantageously such incorporation of the sealing member 122 reduces the risk of paravalvular leakage (PVL) past the prosthetic valve 100. The sealing member 122 can be connected to the frame 106 using suitable techniques or mechanisms. For example, the sealing member 122 can be sutured to the frame 106 utilizing sutures that can extend around the struts 110. Thus, sealing members, such as sealing member 122, are conventionally referred as PVL skirts.

In some implementations, the inflow or cusp edges 134 of the leaflets 132 can be secured to the frame 106 using one or more connecting skirts 124. Each connecting skirt 124 can comprise an elongated, generally rectangular strip, that can be formed with slits (not shown) to partially separate between different portions thereof, and can be made of suitable synthetic material (e.g., PET) or natural tissue. The cusp edges 134 can be attached to the connecting skirts 124 which are secured, in turn, to the frame 106 along diagonal lines extending along the curved surface of the frame 106 defined by diagonally extending rows of struts 110 extending from the inflow end 104 of the frame toward the outflow end 102. In alternative examples, the cusp edges 134 can be directly coupled, for example utilizing a series of suture stitches, to the struts 110 of a frame 106, or to other types of connecting members such as an inner skirt mounted over the inner surface of the frame. Further examples and methods of attaching seal members to a frame, as well as method and techniques for coupling leaflets 132 to the frame 106, with or without connecting skirts, are disclosed in US Pat. Publication No. 2018/0028310, which is incorporated herein by reference.

Each leaflet 132 typically comprises opposing tabs 136. Each tab 136 can be secured to an adjacent tab 136 of an adjacent leaflet 132 to form a commissure 140 that is secured to the frame 106.

During valve cycling, the leaflets 132 can articulate at the inner most edges of the tab layers, which helps space the leaflets away from the frame 106 during normal operation of the prosthetic valve. This may be advantageous in cases where the prosthetic valve 100 is not fully expanded to its maximum nominal size when implanted in a patient. As such, the prosthetic valve 100 can be implanted in a wider range of patient annulus sizes.

According to some examples, the prosthetic valve 100 further comprises a plurality of support members 142 that can be made of relatively flexible and soft materials, including synthetic materials (e.g., PET fabric) or natural tissue (e.g. bovine pericardium), attached to struts 110 of cells 108. The number of support members 142 can match the number of commissures 140, wherein each commissure 140 can be mounted to the frame 106 by a plurality of sutures.

Each support member 142 can be sutured to the struts 110 defining a cell 108. In some examples, each support member 142 is attached (e.g., sutured) to each strut of a set of struts 110 forming a cell 108 of the frame 106. In the example illustrated in FIGS. 1 and 3A, for example, the support member 142 can be sutured to each strut of a cell 108 comprised of four struts 110.

A commissure 140 can be formed by folding the tabs 136 and stitching them to each other, and/or to additional components of the commissure, such as reinforcement members, fabrics and the like, according to various configurations disclosed in US Pat. Publication No. 2018/0028310, which is incorporated herein by reference. The commissure 140 can be then attached to the respective support member 142, for example by suturing it to the support member 142.

FIGS. 2A-2B show the transition between states a prosthetic valve 100 conventionally goes through prior to and/or during deployment within the implantation site. A prosthetic valve 100 may be assembled in a radially expanded state, as shown in FIG. 1. Prior to insertion into the patient's body, a crimping device (not shown) can be used to crimp the prosthetic valve 100 to the compressed configuration, which can be then stored in this configuration up to utilization thereof for implantation into the patient's body. During an implantation procedure, the prosthetic valve 100 can be advanced through the patient's vasculature in a crimped or compressed state thereof, as shown in FIG. 2A.

Once the valve 100 is positioned at the target implantation site (e.g., the aortic annulus in the case of aortic valve replacement), the balloon 52 can be inflated, thereby expanding the valve 100 to its expanded state, as shown in FIGS. 2B, 3A and 3B, so as to mount it against the surrounding tissue, such as the annular or arterial wall 105. Once the valve is fully expanded, the balloon can be deflated and retrieved from the patient's body, leaving the prosthetic valve in place.

In some cases, the prosthetic valve 100 can recoil radially inward to an expanded diameter that is slightly smaller than the diameter defined by the inflated balloon 52, once the balloon 52 is deflated and no longer exerts an expanding force on the frame 106. The recoil is preferably in the range of less than 5% of the diameter of the valve when expanded over the inflated balloon 52.

The leaflet assembly 130 constantly transitions between an open state during systole (not shown) and a closed state in diastole, as shown in FIG. 3B. The leaflets 132 define a non-planar coaptation plane (not annotated) when their coaptation edges 138 co-apt with each other to seal blood flow through the prosthetic valve 100 in the closed state shown in FIG. 3B. Specifically, during diastole, the leaflets 132 collapse radially inward to effectively seal blood flow through the prosthetic valve 100, optionally defining a non-planar coaptation plane (not annotated) when their coaptation edges 138 move toward each other. This collapse exerts pull forces oriented radially inward in the commissures 140. During diastole, once the pull forces of the leaflets 132 are relieved, the commissures 140 resiliently revert back (radially outward) to their free-state positions.

In FIGS. 3A-3B, the external peripheral surface of the prosthetic valve 100 is shown to be in discontinuous engagement with the inner surface of the arterial wall 105 as shown by the gaps 107 (or voids or channels), which may result in a lack of appropriate sealing therebetween. These gaps 107 are formed due to the fact that the inner surface of the arterial wall 105 may have an irregular surface shape while the outer surface of the frame 106 of the prosthetic heart valve 100 is typically circular, and therefore may cause paravalvular leakage (PVL) around the valve 100.

Paravalvular leakage (PVL) is a complication that is related to the implantation of prosthetic heart valves. It may occur when blood flows through a channel or gap located between the structure of an implanted prosthetic heart valve in an expanded state and the site of implantation (e.g., the cardiac or arterial tissue surrounding it), due to a lack of appropriate sealing therebetween. PVL has been previously shown to greatly affect the clinical outcome of transcatheter aortic valve implantation procedures, and the severity of PVL has been correlated with patient mortality.

In order to address this issue, adaptive seal components can be provided around the external peripheral surface of the prosthetic heart valve, in order to provide improved sealing thereto, as previously disclosed, for example, in U.S. Pat. No. 10,722,316, which is incorporated herein by reference. Typically, these seal components (also known as external skirts, or PVL skirts) can be configured to improve PVL sealing around the implanted prosthetic heart valves. In addition, several PVL skirts were designed to promote tis sue ingrowth (for example, utilizing textured yarns over the external surface of the skirt).

In some cases, explanation of the valves is required, in which case the originally implanted valve is surgically removed from the patient's body. However, explanation of conventional implantable prosthetic heart valves can be challenging in cases in which neointimal tissue has been formed between the seal component and the surrounding anatomy, preventing the valve from being removable from the site of implantation without surgically cutting the surrounding tissue, which is a delicate procedure that may entail significant risks to the patient.

Advantageously, the present invention discloses for the first-time sealing members (or PVL skirts) having three-dimensional (3D) shapes adapted to enable a conforming fit or engagement between prosthetic heart valves in which they are incorporated and the inner surface of the annular or arterial wall 105 at the implantation site, thereby improving PVL sealing around the implanted prosthetic heart valves. Moreover, the sealing members of the present invention can be adapted to prevent and/or reduce tissue ingrowth around the prosthetic heart valve, thereby enabling easier and safer explanation thereof from the surrounding tissue when required. Advantageously, minimization of tissue ingrowth reduces the risks associated with the complex surgical procedures required when ingrown tissues connect between the implant and the anatomy.

Sealing members which comprise a first tear resistant layer and a second cushioning layer attached thereto and extending radially outward therefrom have been previously disclosed, for example in US Pub. No. 2019/0374337 which is incorporated herein by reference. US Pub. No. 2019/0374337 discloses a second layer comprising pile strands or pile yarns woven or knitted into loops attached to the first layer. Such strands or yarns may be spaced from each other, in a manner that can encourage tissue ingrowth. Thus, for applications in which tissue ingrowth is to be avoided, it may be preferable to form a second layer from a continuous material that is devoid of strands and yarns that can be interspaced from each other. However, the formation of a desired resilient 3D continuous layer which is different from such strands or yarns may prove to be challenging, as it requires significant adaptation of the manufacturing procedures, so as to from such a layer in a manner that is bonded to (or coating) the first layer. The current specification provides several fabrication procedures and sealing members resulting from such procedures that may address such challenges.

Thus, according to certain aspects, the present invention provides a prosthetic heart valve 100 comprising a frame 106 and a leaflet assembly 130 mounted within the frame, the frame comprising a plurality of intersecting struts 110, wherein the frame is movable between a radially compressed state and a radially expanded state, as disclosed herein above, wherein the valve 100 further comprises a sealing member 222 coupled to an outer surface of the frame 106, and wherein the sealing member 222 has a three-dimensional (3D) shape in a spread relaxed state thereof.

The terms coupled, engaged, connected and attached, as used herein, are interchangeable.

According to some examples, the present invention provides a prosthetic heart valve 100 comprising a frame 106 and a leaflet assembly 130 mounted within the frame, the frame comprising a plurality of intersecting struts 110, wherein the frame is movable between a radially compressed state and a radially expanded state, as disclosed herein above, wherein the valve 100 further comprises a sealing member 222 coupled to an outer surface of the frame 106, and wherein the sealing member 222 has a resilient three-dimensional (3D) shape in a spread relaxed state thereof.

According to some examples, there is provided a prosthetic heart valve 100 comprising a frame 106 and a leaflet assembly 130 mounted within the frame, the frame comprising a plurality of intersecting struts 110, wherein the frame is movable between a radially compressed state and a radially expanded state, wherein the valve 100 further comprises a sealing member 222 coupled to an outer surface of the frame 106, and wherein the sealing member 222 is formed by a process comprising at least one thermal shape-forming step.

The term “shape-forming” refers to a process, a procedure or a step thereof by which an object assumes a different shape compared to its original shape prior to the shape-forming. Shape-forming, as referring to the processes and products of the present invention, are processes in which a two-dimensional object is shaped into a three-dimensional object. The 3D shapes are formed to resiliently maintain their shapes when not subjected to heat or physical pressure (e.g. in a successive shape-forming process).

Thus, the term “thermal shape-forming” refers to a process, a procedure or a step thereof by which shape-forming is assisted by heating the object to be shaped above ambient temperature. It is to be understood that thermal shape-forming processes and step(s) thereof according to the present invention are shape-forming processes and step(s), which are impossible or difficult under ambient temperatures. Each possibility represents a separate example. The 3D shapes are formed to resiliently maintain their shapes when not subjected to heat and physical pressure (e.g. in a successive shape-forming process).

It is to be understood that simple coating of objects with a coating material are not considered to be shape-forming, according to some examples, unless measures are taken during the coating to form the shape of the coated object. In other words, according to some examples, a coating process in which the coated object has substantially the same shape before and after the coating, is in not considered to be shape-forming.

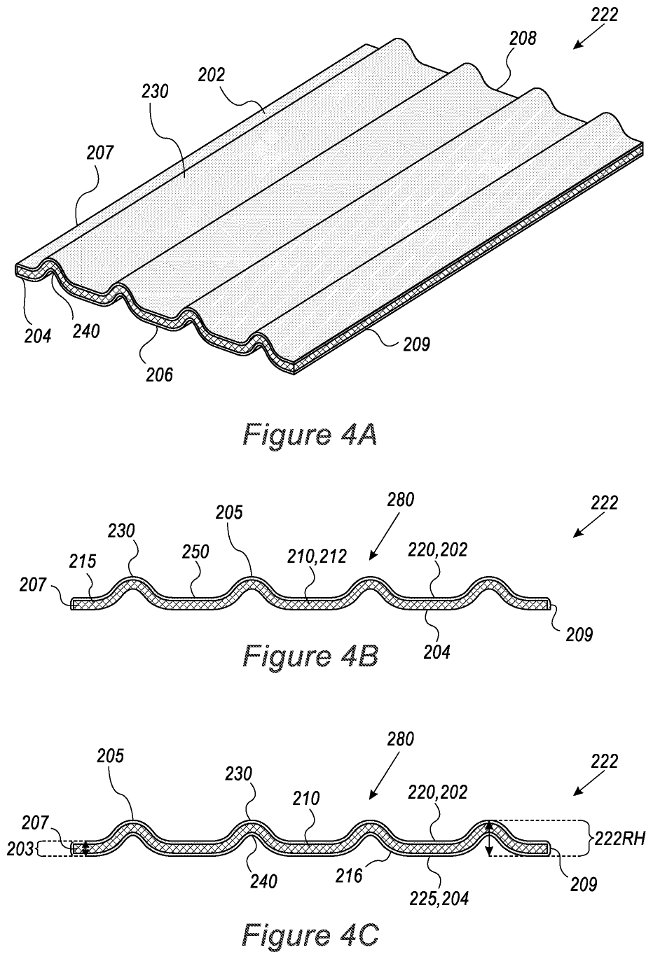

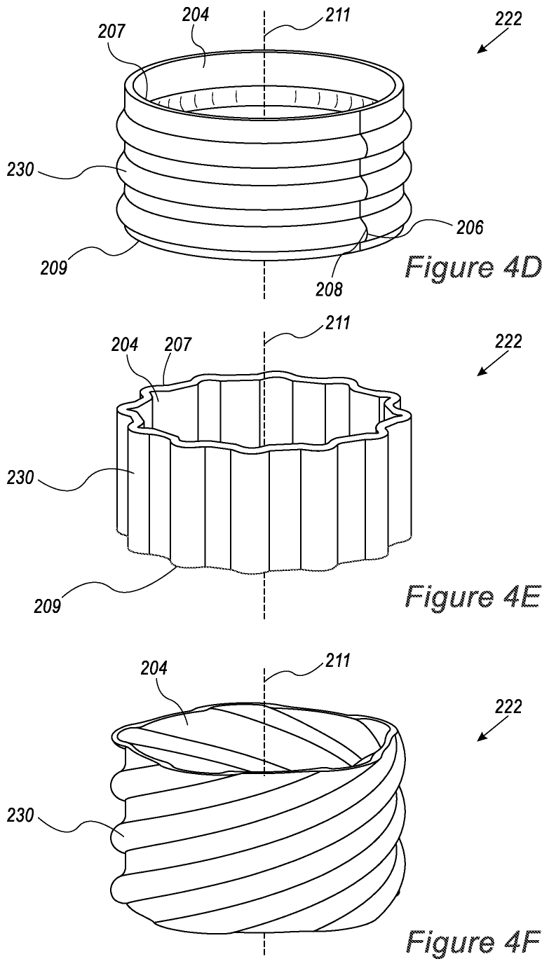

The term “spread”, as used herein, refers to a state of a foldable sheet, which is substantially flat. For quadrilateral objects, which have four edges (e.g. typical PVL skirts, according to some examples) a spread state is assumed when two opposite edges are distanced from each other. For example, FIG. 4A present sealing member 222 in a spread state.

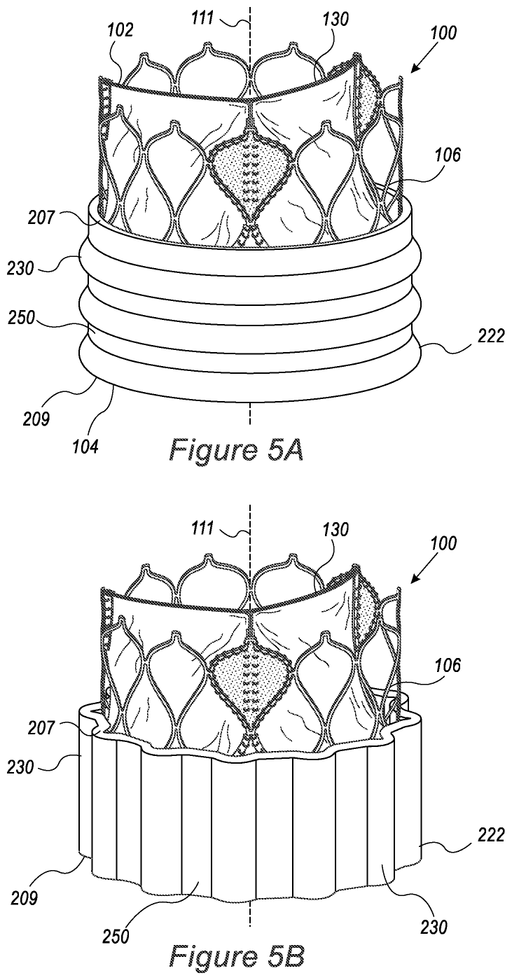

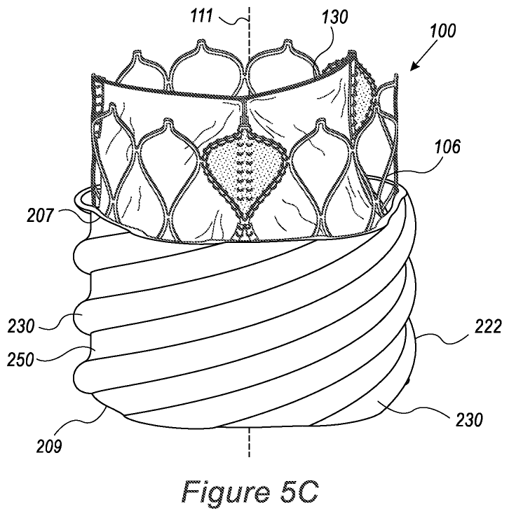

Conversely, the term “folded”, as used herein, with reference to PVL skirts, refers to the state of the skirts of the present invention, in which they are in a substantially cylindrical 3-dimensional shape, and optionally coupled to an object (e.g., a frame of a prosthetic heart valve). For example, FIG. 5A shows sealing member (or PVL skirt) 222 in a folded state surrounding prosthetic heart valve 100, and FIG. 4D shows sealing member 222 in a folded state separate from a heart valve.

It is to be understood that typical PVL skirts may transform from a spread state to a folded state upon connecting, linking or attaching two opposite edges thereof.

The term “relaxed”, as used herein refers to a state of matter, which is substantially devoid of application of physical force or pressure thereto.

The term “spread relaxed state” as used herein, refers to a state of a material (e.g., sealing member 222) which is substantially both relaxed and spread, as disclosed herein above. According to some examples, in the spread relaxed state, the sealing member(s) of the present invention (e.g., sealing member 222) is substantially devoid of application of physical force or pressure thereon, and has two opposite edges which are substantially distanced from each other.

Reference is now made to FIGS. 4A-5C. FIG. 4A shows a view in perspective of a sealing member 222, according to some examples. FIGS. 4B and 4C show cross-sectional views of the sealing member 222, according to some examples. FIGS. 4D-4F shows views in perspective of various configurations of sealing member 222, in a cylindrical folded state, according to some examples. FIGS. 5A-5C show various configurations of sealing member 222 mounted on the frame 106 of the prosthetic valve 100, according to some examples.

According to a certain aspect, there is provided a sealing member 222, adapted to be mounted on (or coupled to) the outer surface of the frame 106 of the prosthetic valve 100 (see for example FIGS. 5A-5C), or any other similar prosthetic valve known in the art. The sealing member 222 can be connected/mounted to the frame 106 using suitable techniques or mechanisms. For example, the sealing member 222 can be sutured to the frame 106 utilizing sutures that can extend around the struts 110. The sealing member 222 can be provided in a spread state, and connected/mounted to the frame 106 by folding it over the frame 106, thereby transforming it from the spread to the folded state. Alternatively, the sealing member 222 may be provided in an already folded state prior to attachment to the frame 106. For example, the frame 106 may be inserted into the already cylindrically folded sealing member 222 and sutured thereto. The sealing member 222 can be configured to form a snug fit with the frame 106 such that it lies against the outer surface of the frame 106 when the prosthetic valve 100 is in the radially expanded state, as illustrated.

According to some examples, the sealing member 222 has a 3D shape in a spread relaxed state thereof, as can be appreciated for example from FIGS. 4A-4C. According to some examples, the sealing member 222 inherently has a 3D shape in a cylindrical folded state thereof (FIGS. 4D-4F and 5A-5C).

According to some examples, the sealing member 222 has a 3D resilient structure such that a nonfibrous outer surface 280 of the sealing member 222 exhibits a plurality of elevated portions 230 with peaks 205 and a plurality of non-elevated portions 250. In further examples, each one of the plurality of non-elevated portions 250 is defined by adjacent pairs of the plurality of elevated portions 230. In further examples, the nonfibrous outer surface 280 is a smooth surface. In further examples, the nonfibrous outer surface 280 is a unitary/continuous surface.

In some examples, the elevated portions 230 are ridges 230 and the non-elevated portions 250 are inter-ridge gaps 250. As used herein, the terms “elevated portions 230” and “ridges 230” are interchangeable, and refer to the same plurality of elevated portions of the sealing member 222, as illustrated in FIGS. 4B-4C. As used herein, the terms “non-elevated portions 250” and “inter-ridge gaps 250” are interchangeable, and refer to the same plurality of non-elevated portions of the sealing member 222, as illustrated in FIGS. 4B-4C.

Specifically, as can be appreciated for example from FIG. 4A, the sealing member 222 includes ridges 230, which cause its shape to be 3-dimensional, in contrast to the substantially flat two-dimensional (2D) shape it would assume in the absence of such ridges 230. It is thus to be understood that the 3-dimensions of the 3-dimensional (3D) sealing member 222 include: (i) a spatial length dimension extending between an outflow edge 207 and an inflow edge 209 of the sealing member 222 (see FIGS. 4B and 4C); (ii) a spatial length dimension extending between a first lateral edge 206 and an second lateral edge 208 of the sealing member 222; and (iii) a spatial length dimension defined by the sealing member's ridges height (or thickness) 222RH of ridges 230 (see FIG. 4C). It is further to be understood that the 3D structure of the sealing member 222 is attributed to the ridges height 222RH of ridges 230, which is greater by at least 1000%, preferably at least 2000%, than the thickness of the flat 2D structure thereof, prior to the formation of the ridges 230 thereon.

The terms “including” and/or “having”, as used herein, are defined as comprising (i.e., open language).

According to some examples, the sealing member 222 comprises a plurality of protrusions or ridges 230, extending away from a first surface 202 of the sealing member 222. According to some examples, the plurality of protrusions or ridges 230 are spaced apart from each other along the first surface 202 of the sealing member 222. The plurality of ridges 230 form the 3D shape of the sealing member 222 when in its spread relaxed state (as can be seen in FIGS. 4A-4C), according to some examples.

According to some examples, the sealing member 222 has four edges. According to some examples, the sealing member 222 has four vertices. According to some examples, each one of the four vertices of the sealing member 222 has a substantially right angle. The phrase “substantially right angle” refers to an angle in the range of 80° to 100°.

According to some examples, the sealing member 222 has four substantially right angle vertices, and two sets of two opposing edges (a set of first lateral edge 206 and second lateral edge 208, and a set of outflow edge 207 and an inflow edge 209), wherein in each set, the two opposing edges are substantially parallel. According to some examples, the sealing member 222 extends from a first lateral edge 206 toward a second lateral edge 208, when the sealing member 222 is in a spread state. According to some examples, the sealing member 222 extends around a sealing member centerline 211, when the sealing member 222 is in a folded state. According to some examples, the sealing member centerline 211 and the centerline 111 of valve 100 are coaxial and may coincide when the sealing member 222 is connected to heart valve 100. According to some examples, the sealing member 222 extends from an inflow edge 209 toward an outflow edge 207. According to some examples, the sealing member 222 extends from an inflow edge 209 toward an outflow edge 207 in both the folded state and the spread state thereof.

According to some examples, in the spread state, sealing member 222 is substantially rectangular. According to some examples, the distance from first lateral edge 206 and second lateral edge 208 is greater that the distance from inflow edge 209 to outflow edge 207.

According to some examples, the plurality of ridges 230 extend radially outward, away from the sealing member centerline 211, in a folded state of the sealing member 222 (see FIGS. 4D-4F). According to some examples, the plurality of ridges 230 extend outward, radially away from the frame 106 of valve 100 (and outward relative to centerline 111 thereof), when the sealing member 222 is mounted on the frame 106 (see FIGS. 5A-5C). According to some examples, the sealing member 222 is folded by connecting first lateral edge 206 and second lateral edge 208, such that the plurality of ridges 230 are oriented radially away from the sealing member centerline 211 (see for example, FIG. 4D). According to some examples, the sealing member 222 in a folded state is coupled to the outer surface of the frame 106 of the prosthetic valve 100 so that the plurality of ridges 230 are oriented to extend radially away from the centerline 111.

In some examples, the sealing member 222 comprises a plurality of inner channels 240, wherein each channel 240 is formed at a second surface 204 of the sealing member 222. In further examples, the plurality of channels 240 correspond to the plurality of ridges 230, wherein each ridge 230 comprise a corresponding channel 240 at an opposite surface of the sealing member 222. In further examples, the number of channels 240 is identical to the number of ridges 230, wherein each one of the plurality of channels 240 is formed by a respective one of the plurality of ridges 230 at an opposing surface of the sealing member 222.

According to some examples, each one of the plurality of channels 240 is facing sealing member centerline 211, in a folded state of the sealing member 222 (see FIGS. 4A-4C). According to some examples, each one of the plurality of channels 240 is facing inward, in a folded state of the sealing member 222 (see FIGS. 4A-4C).

It is to be understood that in the context of the sealing member(s) of the present invention, such as sealing member 222, the term “inward” refers to the radial direction facing from the surface of the sealing member toward a sealing member centerline (e.g., sealing member centerline 211), whereas the term “outward” refers to the opposite radial direction. According to some examples, the term “outward” refers to a direction facing the surrounding tissue of the native annulus, against which the prosthetic valve 100 is configured to be mounted.

According to some examples, each one of the plurality of channels 240 is facing centerline 111 of valve 100, when the sealing member 222 is mounted on the frame 106 (see FIGS. 5A-5C). According to some examples, the sealing member 222 is folded by connecting first lateral edge 206 and second lateral edge 208, such that the plurality of channels 240 are oriented inward. According to some examples, the sealing member 222 is folded by connecting first lateral edge 206 and second lateral edge 208, such that the plurality of channels 240 are oriented to face sealing member centerline 211.

According to some examples, the plurality of inter-ridge gaps 250 are formed over the surface of the first layer 210 between each two adjacent ridges 230 of the sealing member 222. According to further examples, one inter-ridge gap 250 is formed between the outflow edge 207 and one of the ridges 230, while another inter-ridge gap 250 is formed between the inflow edge 209 and one of the other ridges 230. It is to be understood that the inter-ridge gaps 250 are spaces formed due to the 3-dimensional shape of the sealing member 222, according to some examples. Specifically, according to some examples, the plurality of inter-ridge gaps 250 are facing the same direction, which the ridges 230 face. According to some examples, each one of the inter-ridge gaps 250 is facing outward from the folded sealing member 222.

According to some examples, the prosthetic heart valve 100 comprising the sealing member 222 is configured to be positioned (i.e., implanted) at the target implantation site (e.g., the aortic annulus in the case of aortic valve replacement) so as to form contact between the arterial wall 105 and the plurality of ridges 230. Advantageously, the plurality of ridges 230 of the sealing member 222 are adapted to contact the arterial wall 105 following the expansion of the prosthetic heart valve 100 at the site of implantation, and thus to enable a conforming fit or engagement between the prosthetic heart valve 100 and the inner surface of the arterial wall 105, thereby improving PVL sealing around the implanted prosthetic heart valve.

According to some examples, the sealing member 222 is configured to transition from the spread relaxed state to the cylindrical folded state, due to its elastic and/or flexible characteristics, in order to form a cylindrical folded PVL skirt. A folded PVL skirt 222 may become coupled to outer surface of the frame 106 of the prosthetic valve 100, for example during a procedure of valve assembly. Alternatively, a spread sealing member 222 may be folded around the outer surface of the frame 106 and coupled thereto to achieve a similar product.

In FIGS. 4D-4F, plurality of ridges 230 are portrayed to follow parallel path-lines extending in different directions. These may be vertical, horizontal or diagonal with respect to the centerline 211 of the cylindrically shaped sealing member 222 in its folded state. It is to be understood that the orientation of the ridges 230 in the folded state of the sealing member 222 may be dictated by their construction prior to the folding, i.e. when the sealing member 222 is in a spread state. For example, a sealing member 222, which has plurality of ridges 230 follow parallel path-lines extending from first lateral edge 206 to second lateral edge 208 (as shown in FIG. 4A), may be folded by connecting first lateral edge 206 to second lateral edge 208 such that a cylindrical shape of the sealing member 222 is formed. In such an exemplary situation, upon said folding the sealing member 222 in its folded shape will have plurality of circumferentially extending ridges 230, which are substantially parallel to inflow edge 209 and to outflow edge 207 (as shown in FIG. 4D). In a second example, a sealing member 222, which has plurality of ridges 230 following parallel path-lines extending from inflow edge 209 to outflow edge 207 (not specifically shown in spread relaxed state), may be folded by connecting first lateral edge 206 to second lateral edge 208 such that a cylindrical shape of the sealing member 222 is formed. In such a second exemplary configuration, upon said folding, the sealing member 222 in its folded shape will have plurality of vertically oriented ridges 230, which are substantially perpendicular to inflow edge 209 and to outflow edge 207 (as shown in FIG. 4E). Similarly, angled or diagonal ridges in the spread state will lead to diagonally oriented ridges 230 in the folded state of the sealing member 222, as shown in FIG. 4F.

As detailed herein the shape-forming process of creating the ridges 230 in the sealing member 222 is not limited to be performed prior to the folding, and ridges 230 may be formed on the first surface 202 of the sealing member 222 after the folding, according to some examples. In such cases the orientation of the ridges 230 path-lines is straightforward. Furthermore, the ridges of the present sealing member 222 are not required to form parallel path-lines with respect to each other.

According to some examples, each one of the plurality of ridges 230 follows a path-line extending from the first lateral edge 206 to the second lateral edge 208 in a spread state of the sealing member 222. According to some examples, each one of the plurality of ridges 230 follows a path-line perpendicular to any one of the first lateral edge 206 and/or the second lateral edge 208 in a spread state of the sealing member 222. According to some examples, each one of the plurality of ridges 230 follows a path-line parallel to any one of the outflow edge 207 and/or the inflow edge 209 in a spread state of the sealing member 222.

According to some examples, each one of the plurality of ridges 230 follows a path-line circumferentially extending around the sealing member centerline 211, in a folded state of the sealing member 222. According to some examples, each one of the plurality of ridges 230 follows a path-line circumferentially extending around the centerline 111 when the sealing member 222 is in a folded state and mounted on the frame 106 of the prosthetic heart valve 100. According to some examples, each one of the plurality of ridges 230 follows a path-line parallel to any one of the outflow edge 207 and/or the inflow edge 209, circumferentially around the sealing member centerline 211, in a folded state of the sealing member 222 (see FIG. 4D).

According to some examples, each one of the plurality of ridges 230 follows a path-line extending from the inflow edge 209 to the outflow edge 207 in a spread state of the sealing member 222. According to some examples, each one of the plurality of ridges 230 follows a path-line parallel to any one of the first lateral edge 206 and/or the second lateral edge 208 in a spread state of the sealing member 222. According to some examples, each one of the plurality of ridges 230 follows a path-line perpendicular to any one of the outflow edge 207 and/or the inflow edge 209 in a spread state of the sealing member 222.

According to some examples, each one of the plurality of ridges 230 follows a path-line extending parallel to the sealing member centerline 211 in a folded state of the sealing member 222. According to some examples, each one of the plurality of ridges 230 follows a path-line extending parallel to the centerline 111 when the sealing member 222 is in a folded state and mounted on the frame 106 of the prosthetic heart valve 100. According to some examples, each one of the plurality of ridges 230 follows a path-line perpendicular to any one of the outflow edge 207 and/or the inflow edge 209 in a folded state of the sealing member 222 (see FIG. 4E).

According to some examples, each one of the plurality of ridges 230 follows a path-line extending diagonally along the surface of the sealing member 222, in a spread state thereof. According to some examples, each one of the plurality of ridges 230 follows a path-line extending diagonally along the surface of the sealing member 222, in a folded state thereof. According to some examples, each one of the plurality of ridges 230 follows a path-line extending diagonally with respect to the centerline 111 when the sealing member 222 is in a folded state and mounted on the frame 106 of the prosthetic heart valve 100 (see FIG. 4F).

Various configurations and orientations as described above may be advantageous for different physiological and implantation-related requirements. For example, the configuration of FIGS. 4D and 5A may be advantageous due to the generally perpendicular orientation of the plurality of ridges 230 relative to the axial orientation of the direction of the flow, when the valve 100 is mounted against the annular or arterial wall 105, and therefore can thereby potentially improving PVL sealing therebetween.

According to some examples, the sealing member 222 comprises a first layer 210.

According to some examples, the sealing member 222 comprises a first layer 210 and a second layer 220. According to further examples, said first and second layers 210 and 220, respectively, are disposed externally to the outer surface of the frame 106, when the sealing member 222 is coupled thereto. According to further examples, the sealing member 222 can comprise additional layer(s), as detailed herein.

According to some examples, the second layer 220 is in contact with a first surface 215 of the first layer 210. According to some examples, the second layer 220 is in contact with a first surface 215 of the first layer 210 both when the sealing member 222 is in a spread state and when it is in a folded state. According to some examples, the second layer 220 is attached to and/or is coating a first surface 215 of the first layer 210. According to some examples, said first surface 215 of the first layer 210 is oriented in the outward direction when the sealing member 222 is in a folded state. According to some examples, said first surface 215 is oriented toward the implantation site (e.g., the annular or arterial wall 105) when the sealing member 222 is mounted on the frame 106 of the prosthetic heart valve 100 and implanted in the implantation site. According to further examples, the second layer 220 is forming a first surface 202 of the sealing member 222, as illustrated in FIG. 4B. According to some examples, the first surface 202 of the sealing member 222 is oriented in the outward direction when the sealing member 222 is in a folded state. According to some examples, the first surface 202 of the sealing member 222 is oriented toward the implantation site when the sealing member 222 is mounted on the frame 106 of the prosthetic heart valve 100 and implanted in the implantation site.

Without wishing to be bound by any theory or mechanism of action, various sealing members 222 as disclosed herein assume a three-dimensional shape, which may be a result of a thermal shape-processing procedure. Such procedure is enabled or facilitated by the employment of thermoplastic material, which can be shaped at elevated temperature as detailed herein. To enable thermoplastic materials to be molded or shaped into a desired structure with thin sheet-like objects, it is advantageous that the thermoplastic materials constitute or cover the objects. This may be achieved, e.g., utilizing coating with a thermoplastic coating layer or forming the object with a thermoplastic layer. Although one thermoplastic layer may be sufficient for enabling the shape-forming process, it may be advantageous, according to some examples, to include a plurality of thermoplastic layers, such as two layers. Specifically, a configuration in which the two external layers of the sealing member 222 include a thermoplastic material may be advantageous.

According to some examples, the sealing member 222 comprises a third layer 225.

According to some examples, the third layer 225 is in contact with a second surface 216 of the first layer 210. According to some examples, the third layer 225 is in contact with a second surface 216 of the first layer 210 both when the sealing member 222 is in a spread state and when it is in a folded state. According to some examples, the third layer 225 is attached to and/or is coating a second surface 216 of the first layer 210. According to some examples, said second surface 216 of the first layer 210 is oriented in the inward direction when the sealing member 222 is in a folded state. According to some examples, said second surface 216 is oriented in the direction opposite to the implantation site (e.g., the arterial wall 105) when the sealing member 222 is mounted on the frame 106 of the prosthetic heart valve 100 and implanted in the implantation site. According to further examples, the third layer 225 is forming a second surface 204 of the sealing member 222, as illustrated in FIG. 4C. According to some examples, the second surface 204 of the sealing member 222 is oriented in the inward direction when the sealing member 222 is in a folded state. According to some examples, the second surface 204 of the sealing member 222 is oriented in the direction opposite to the anatomical wall at the implantation site when the sealing member 222 is mounted on the frame 106 of the prosthetic heart valve 100 and implanted in the implantation site.

According to some examples, sealing member 222 comprises both the second layer 220 and the third layer 225. According to some examples, the second layer 220 is connected to the third layer 225. According to some examples, the second layer 220 and the third layer 225 are unified to cover the first layer 210, as illustrated in FIG. 4C. According to some examples, the second layer 220 and the third layer 225 collectively form a coating which covers both the first and second surfaces 202 and 204, respectively, of the sealing member 222. According to some examples, the second layer 220 and the third layer 225 collectively form a coating which covers the sealing member 222.

It is to be understood based on the above that the spread sealing member 222 is folded into its folded state through connecting its first lateral edge 206 and its second lateral edge 208, over the second surface 204 thereof, such that when the sealing member 222 is in a folded state, its second surface 204 faces inward (toward the sealing member centerline 211) and its first surface 202 faces outward, according to some examples. Therefore, when the folded sealing member 222 is mounted on the frame 106 of the prosthetic heart valve 100 and implanted at the implantation site, the second layer 220 is in contact with the anatomical wall at the implantation site (e.g., the inner surface of the annular or arterial wall 105).

According to some examples, the sealing member 222 extends between a first surface 202 and a second surface 204, wherein the sealing member 222 has a total layer thickness 203 measured between the first surface 202 and the second surface 204 at one of the inter-ridge gaps 250, as illustrated at FIG. 4C. According to some examples, said total layer thickness 203 is measured from the first surface 202 of the sealing member 222 to the second surface 216 of the first layer 210 (not shown). According to some examples, the total layer thickness 203 is measured from the first surface 202 of the sealing member 222 (e.g., the second layer 220) to the second surface 204 (e.g., the third layer 225), as shown in FIG. 4C. According to some examples, the sealing member's 222 ridges height 222RH (e.g., the thickness measured by the height of the ridges 230) is at least 1000% greater than the total layer thickness 203. In further examples, the ridges height 222RH is at least 2000%, at least 3000%, at least 4000%, at least 5000%, or at least 6000% greater than the total layer thickness 203 of the sealing member 222. In still further examples, the ridges height 222RH is no greater than 6000%, 7000%, 8000%, 9000%, 10,000%, 20,000%, 30,000%, 40,000% or 50,000% compared to the total layer thickness 203 of the sealing member 222. Each possibility represents a different example.

It is to be understood that the present invention including each of the specified elements is not limited to the examples described in the figures. Specifically, dimensions may be drawn in the figures so that the elements are clear and comprehensible rather than reflecting the actual dimensions and dimension rations. For example, the thickness ratio between ridges height 222RH and total layer thickness 203 in FIGS. 4B-C is moderate, whereas, as described above, the actual ratio is greater (e.g. the ridges height 222RH is 10-60 times greater than the total layer thickness 203). For example, in some non-binding implementations, the total layer thickness 203 can be in the range of 0.02 to 0.1 mm, while the ridges height 222RH can be in the range of 0.5-3 mm.

According to some examples, the sealing member 222 has a resilient 3D structure such that the nonfibrous outer surface 280 of the sealing member 222 exhibits the plurality of elevated portions 230 with peaks 205 and the plurality of non-elevated portions 250, as disclosed herein above (see for example FIGS. 4B-C). According to some examples, the nonfibrous outer surface 280 of the sealing member 222 is defined as an outer surface combining the first surface 202 and an outer surface of each one of the plurality of elevated portions 230 (i.e., ridges 230). According to some examples, the peaks 205 are defined as the highest point along the outer surface of each one of the plurality of elevated portions 230, extending away from the first surface 202 of the sealing member 222. According to some examples, the height of each peak 205 is defined as the distance of the highest point along the outer surface of each one of the plurality of elevated portions 230, relative to the frame 106, when the sealing member 222 is coupled to the outer surface of the frame 106 of the prosthetic valve 100 (e.g., the ridges height 222RH).

According to some examples, the non-elevated portions 250 are defined as the inter-ridge gaps 250. In further such examples, the height of each non-elevated portion 250 is defined as the distance of the first surface 202 relative to the frame 106, when the sealing member 222 is coupled to outer surface of the frame 106 of the prosthetic valve 100 (e.g., the total layer thickness 203 in the examples illustrated in FIGS. 4A-C). According to some examples, the distance of the peaks 205 from the frame 106 is at least 1000% greater than the distance of the non-elevated portions 250 from the frame 106, in the absence of an external force applied to press the elevated portions 230 against the frame (also referred to as the “relaxed state” for convenience). According to further examples, the distance of the peaks 205 from the frame 106 is at least 2000%, at least 3000%, at least 4000%, at least 5000%, or at least 6000% greater than the distance of the non-elevated portions 250 therefrom. Each possibility represents a different example.

The term “external force”, with respect to a force applied to deform the 3D shape of a sealing member, may relate to the force applied by the surrounding tissue (e.g., annular or arterial wall 105) when the prosthetic valve 100 is deployed there-against, or to the force applied by an internal wall of a sheath or a capsule in which the valve 100 is retained during storage or delivery to the implantation site.

The term “resilient”, as used herein with respect to the 3D shape of the sealing member, refers to the sealing member, and more specifically, the peaks or peak portions thereof, being resistant to permanent deformation when such external force is applied thereto, and having a tendency to return to return to its relaxed state, when the external force is no longer applied thereto.

The term “nonfibrous” as used herein with respect to the nonfibrous outer surface(s) of the sealing member(s) of the present invention (e.g., nonfibrous outer surface 280 of the sealing member 222), refers to an outer surface of the sealing member which is devoid of yarns and/or strands (including being devoid of texturized yarns and/or strands). Thus, a second layer defining a nonfibrous outer surface is necessarily a nonfibrous layer, which is to be understood as being a non-woven and non-braided layer.

According to some examples, the first layer 210 is made from a flexible and/or elastic material(s) adapted to provide mechanical stability, and optionally tear resistance (or tear strength), to the sealing member 222. In further examples, the first layer 210 is configured to enable the continuous durable attachment of the sealing member 222 to the outer surface of the frame 106 of the prosthetic valve 100, optionally by preventing the formation of irreversible deformation thereto (e.g., resist tearing), thus providing mechanical stability to the structure Furthermore, it may be advantageous thereof.

As used herein, the terms “tear resistance” and “tear strength” are interchangeable, and refer to a material's ability to resists the formation of extent tears, when the material is subjected to the application of stress. The tear refers to the extent of a notch or incision in the material under stress. A tear resistant material is capable of resisting significant stress and/or deformation applied thereto without experiencing loss of integrity. According to some examples, the tear resistant layer(s) (e.g., the first layer 210) of the present invention can be relatively thin and yet strong enough to allow any covering or coating layer attached thereto to be sutured to the frame, and to allow the prosthetic valve 100 to be crimped, without tearing.

According to some examples, the tear resistant layer(s) (e.g., the first layer 210) of the present invention may include a ripstop fabric. The term “ripstop” as used herein, refers to a woven reinforced fabric which is resistant to tearing and ripping. A ripstop fabric typically refers to a woven fabric in which a reinforcing yarn has been interwoven at designated intervals in a crosshatch pattern, wherein the designated interval can vary from one fabric to another and optionally vary within a single fabric. Depending on how the reinforcing yarn is incorporated, the woven fabric can take on a variety of textures, such as for example, a box pattern. According to some examples, the first layer(s) (e.g., the first layer 210) of the sealing member(s) of the present invention comprises a ripstop fabric, optionally comprising fibers made from polyethylene terephthalate (PET). In further examples, the first layer(s) (e.g., the first layer 210) of the sealing member(s) comprises a tear resistant ripstop fabric comprising PET.

According to some examples, the first layer 210 comprises at least one tear resistant material. According to further examples, the first layer 210 is made from at least one tear resistant material.

The first layer 210 can be made of various suitable materials, optionally biocompatible, such as, but not limited to: various synthetic materials (e.g., polyethylene terephthalate (PET), polyester, polyamide (e.g., Nylon), polypropylene, polyetheretherketone (PEEK), polytetrafluoroethylene (PTFE), etc.), natural tissue and/or fibers (e.g. bovine pericardium, silk, cotton, etc.), metals (e.g., a metal mesh or braid comprising gold, stainless steel, titanium, nickel, nickel titanium (Nitinol), etc.), and combinations thereof. Each possibility represents a different example.

The first layer 210 can be a metallic or polymeric member, such as a shape memory metallic or polymeric member. The first layer 210 can be a woven textile. It is to be understood that the first layer 210 is not limited to a woven textile. Other textile constructions, such as knitted textiles, braided textiles, fabric webs, fabric felts, filament spun textiles, and the like, can be used. The textiles of first layer 210 can comprise at least one suitable material, selected from various synthetic materials, natural tissue and/or fibers, metals, and combinations thereof, as described herein above.

According to some examples, the first layer 210 comprises a tough, tear resistant material such as, but not limited to, polyethylene terephthalate (PET). According to further examples, the first layer 210 comprises a tear resistant PET fabric. According to further examples, the first layer 210 comprises at least one tear resistant knit/woven PET fabric.

The tear resistant material (e.g., PET fabric) of the present invention may be woven from yarns using any known weave pattern, including simple plain weaves, basket weaves, twill weaves, velour weaves and the like, according to some examples. Weave patterns include warp yarns running along the longitudinal length of the woven tear resistant material (e.g., the sealing member 222) and weft also known as fill yarns running around the width or circumference of the woven tear resistant material.

According to some examples, the first layer 210 comprises at least one flexible material. According to further examples, the first layer 210 is made from at least one flexible material. According to some examples, the first layer 210 is flexible.

According to some examples, the first layer 210 comprises at least one elastic material. According to further examples, the first layer 210 is made from at least one elastic material. According to some examples, the first layer 210 is elastic.

According to some examples, the tear resistant layer(s) (e.g., the first layer 210) of the sealing member(s) (e.g., sealing member 222) of the present invention comprises at least one tear resistant and flexible material, which is able to withstand loads of above about 3N of force before tearing. According to some examples, the first layer 210 comprises at least one tear resistant and flexible material, which is able to withstand loads of above about 5N of force before tearing, thereby enabling the sealing member 222 to reliably operate without tearing during regular use thereof. According to some examples, the first layer 210 comprises at least one tear resistant and flexible material, which is able to withstand loads of above about 7N of force before tearing.

According to further examples, the at least one tear resistant and flexible material of the first layer 210 is able to withstand loads of above about 10N of force before tearing. According to still further examples, the at least one tear resistant and flexible material of the first layer 210 is able to withstand loads of above about 15N of force before tearing. According to yet still further examples, the at least one tear resistant and flexible material of the first layer 210 is able to withstand loads of above about 20N of force before tearing. According to still further examples, the at least one tear resistant and flexible material of the first layer 210 is able to withstand loads of above about 25N of force before tearing. According to yet still further examples, the at least one tear resistant and flexible material of the first layer 210 is able to withstand loads of above about 30N of force before tearing. According to a preferred example, the at least one tear resistant and flexible material of the first layer 210 comprises a PET fabric and is able to withstand loads of up to about 20N of force before tearing.

It is to be understood that having a tear resistance of at least 5N means that the layer is able to be stretched at least in an axial direction (i.e., having its inflow edge 209 and outflow edge 207 stretched away from each other), without tearing.

According to some examples, the first layer 210 comprises at least one biocompatible material. According to further examples, the first layer 210 is made from at least one biocompatible material. According to some examples, the first layer 210 is biocompatible.

The term “biocompatible” as used herein means that the implantable valve and the sealing member thereof are capable of being in contact with living tissues or organisms without causing harm to the living tissue or the organism. Biocompatible materials and objects are substantially non-toxic in the in vivo environment of the implantation site, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing”. Typically, these tests measure a material's toxicity, infectivity, pyrogenicity, irritation potential, reactivity, hemolytic activity, carcinogenicity and/or immunogenicity.

It is to be understood that when the first layer 210 is covered by the second layer 220 and third layer 225, as shown in FIG. 4C, it should not come in contact with tissues when implanted, and thus, in this case first layer 210 may be made of non-biocompatible materials. Nevertheless, it may be preferable to form the first layer 210 from biocompatible materials in such cases as well, to prevent risks of abrasive damage or tears of any of the second layer 220 or third layer 225, which may in turn expose portions of the first layer 210.

The sealing members of the present invention, such as for example sealing member 222, may further comprise silicone or other lubricious materials or polymers that could assist in explant procedures for removal of the prosthetic valve from its initial site of implantation, according to some examples. Such lubricants are typically incorporated into and/or onto the outermost surface or surfaces, which is to come in contact with the surrounding tissue (e.g., the first surface 202 and/or the second layer 220 of the present example sealing member 222). Additionally or alternatively, the outermost surfaces of the sealing members of the present invention (e.g., the first surface 202 and/or the second layer 220) may be smooth and/or comprise a low-friction or lubricious material. The lubricious material of the outermost surfaces can also reduce friction with tissue of the native valve in contact with the inflow end 104 (or other portions) of the prosthetic valve 100, thereby preventing damage to the tissue.

According to some examples, the first surface 202 and/or the second layer 220 are continuous in a manner which is devoid of yarns and/or strands (including being devoid of texturized yarns and/or strands).

According to some examples, the second layer 220 is adapted to contact the implantation site tissue (i.e., the inner surface of the annular or arterial wall 105) and therefore is made from at least one elastic biocompatible material. Furthermore, it may be advantageous for the second layer 220 to be made of materials that may prevent/resist and/or reduce the extent of tissue ingrowth around or over the sealing member 222, according to some examples, such that if and when an explant procedure is required, the valve 100 can be easily removed from the site of implantation, as detailed above.

According to some examples, the second layer 220 can be made of various suitable biocompatible synthetic materials, such as, but not limited to, a thermoplastic material. Suitable thermoplastics biocompatible materials are selected from, but not limited, polyamides, polyesters, polyethers, polyurethanes, polyolefins (such as polyethylene and/or polypropylenes), polytetrafluoroethylenes, and combinations and copolymers thereof. Each possibility represents a different example. Thus, according to some examples, the second layer 220 is made of a thermoplastic material. According to some examples, second layer 220 comprises a thermoplastic material. According to some examples, second layer 220 consists of a thermoplastic material. According to some examples, the thermoplastic material is selected from the group consisting of: polyamides, polyesters, polyethers, polyurethanes, polyolefins, polytetrafluoroethylenes, and combinations and/or copolymers thereof.

According to some examples, the second layer 220 can be made of various suitable biocompatible synthetic materials, such as, but not limited to, thermoplastic material, including thermoplastic elastomers (TPE). According to some examples, the thermoplastic material is a thermoplastic elastomer. According to some examples, the thermoplastic material comprises a thermoplastic elastomer (TPE).

As used herein, the terms “thermoplastic elastomer” or TPE are interchangeable, and refer to a type of copolymers or a physical mix of polymers having thermoplastic and elastomeric properties, characterized by having elastic properties while being able to undergo thermal shape-forming (i.e., under the application of heat, similar to thermoplastic polymers) in order to form 3D geometrical shapes from substantially 2D counterparts. Thermoplastic polyurethane (TPU) is an example of a TPE consisting of linear segmented block copolymers composed of hard and soft sections. TPEs can be thermally processed to form various shapes utilizing various known methods, such as injection molding, extrusion, 3D printing, thermoforming, and the like.

According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example. According to some examples, the thermoplastic elastomer is TPU. According to some examples, the thermoplastic elastomer comprises TPU.

According to some examples, the second layer 220 comprises at least one thromboresistant material, adapted to prevent the formation of blood clots (thrombus) therearound, in order to prevent and/or reduce tissue ingrowth around the implanted prosthetic heart valve, thereby enabling easily and safe explant thereof from the surrounding tissue when required, preferably devoid of complex surgical procedures. According to some examples, the second layer 220 comprises at least one thermoplastic elastomer thromboresistant material. According to some examples, the second layer 220 comprises at least one thermoplastic elastomer thromboresistant material, which is adapted to prevent and/or reduce tissue ingrowth therearound. Such material include TPU, according to some examples.

The term “thromboresistant”, as used herein, refers to a material's resistance to platelet adhesion and subsequent thrombus formation and/or tissue ingrowth in vitro and/or in vivo.

According to some examples, the second layer 220 comprises TPU.

The third layer 225, when incorporated into the sealing member 222, may be united with the second layer 220 as detailed herein, according to some examples. When the third and second layers 225 and 220, respectively, are formed as a united coating covering the first layer 210, they preferably are made of the same material, according to some examples. Even if the third and second layers 225 and 220, respectively, are separated, according to some examples, they may have similar or the same composition. According to some examples, the third and second layers 225 and 220, respectively, are made of the same material.

According to some examples, the third layer 225 is made of a thermoplastic material. According to some examples, the third layer 225 comprises a thermoplastic material. According to some examples, the third layer 225 consists of a thermoplastic material. According to some examples, the thermoplastic material is selected from the group consisting of: polyamides, polyesters, polyethers, polyurethanes, polyolefins, polytetrafluoroethylenes, and combinations and copolymers thereof.

Suitable thermoplastic materials for producing the third layer 225 are detailed herein with respect to the composition of the second layer 220.

According to some examples, the third layer 225 comprises at least one thermoplastic elastomer thromboresistant material. According to some examples, the third layer 225 comprises at least one thermoplastic elastomer thromboresistant material, which is adapted to prevent and/or reduce tissue ingrowth therearound.

According to some examples, the third layer 225 comprises TPU.

According to some examples, the sealing member 222 comprises the first layer 210 and the second layer 220, wherein the first layer 210 comprises at least one tear resistant material, and wherein the second layer 220 comprises at least one thermoplastic thromboresistant material. According to some examples, the sealing member 222 comprises the first layer 210 and the second layer 220 and the third layer 225, wherein the first layer 210 comprises at least one tear resistant material, and wherein each one of the second layer 220 and the third layer 225 comprises at least one thermoplastic thromboresistant material. According to further examples, the second layer 220 assumes a 3D configuration in a relaxed spread state. According to further examples, the third layer 225 assumes a 3D configuration in a relaxed spread state. According to some examples, the second layer 220 and the third layer 225 assume a similar 3D configuration in a relaxed spread state.

According to some examples, the second layer 220 in configured to resiliently retain its 3D shape as detailed herein (i.e. with ridges 230). According to some examples, the third layer 225 in configured to resiliently hold its similar 3D shape as detailed herein (i.e. with channels 240).

According to some examples, the sealing member 222 comprises the first layer 210 and the second layer 220, wherein the first layer 210 is configured to provide mechanical stability and tear resistance and support the structure thereof, while the second layer 220 is configured to form and maintain the 3D shape thereof and optionally prevent and/or reduce tissue ingrowth thereover. According to some examples, the sealing member 222 comprises the first layer 210 and the second layer 220, wherein the first layer 210 is configured to provide mechanical stability and tear resistance and support the structure thereof, while the second and third layers 220 and 225 are configured to form and maintain the 3D shape thereof, wherein the second layer 220 is optionally configured to prevent and/or reduce tissue ingrowth thereover.

It is contemplated that the second layer 220 on its own or together with the optional third layer 225, may lack the ability to maintain a successful durable attachment of the sealing member 222 to the outer surface of the frame 106. Specifically, the second layer 220, and optionally the third layer 225, may have low tear resistance, which does not enable sewing it to the frame 106 in a durable manner. Advantageously, the combination between the first layer 210 and the second layer 220 enables to provide the required features of the sealing member 222. While TPU can potentially reduce tissue ingrowth and maintain the 3D shape of the sealing member 222, it can tear when sutured to the frame. According to some examples, the second layer 220 comprising TPU is reinforced by the first layer 210 comprising PET to provide the strength required to retain the sutures.

Sealing members comprising the thermoplastic elastomeric (TPE) materials of the present invention (e.g., TPU) possess excellent elasticity, exceptional resilience, exhibit minimal tissue ingrowth thereon and enable to maintain the 3D shape thereof, yet remain non-toxic and biocompatible. This unique combination of mechanical and biological properties results in a structure that is ideally suited for its medical uses.

It is contemplated that the utilization of thermoplastic elastomer material(s), such as TPU, as a layer of sealing member 222, enables to fabricate it in a manner which allows formation of a desired 3D-shaped sealing member 222 having a plurality of elastic ridges 230. In some examples, advantageously, the plurality of elastic ridges 230 of the sealing member 222 are adapted to contact, and become compressed against, the annular or arterial wall 105 at the implantation site, following expansion of the prosthetic heart valve 100 therein, thereby improving PVL sealing between the prosthetic heart valve 100 and the inner surface of the annular or arterial wall 105. Thus, according to some examples, each one of the plurality of ridges 230 is elastic and compressible. The elastic and compressible characteristics of the plurality of ridges 230 can improv retention of the sealing member 222 against the tissues of the native heart valve at the implantation site.

According to some examples, the sealing member 222 has a resilient 3D shape, wherein said resilient 3D shape is configured to deform when an external force is applied thereto (e.g., when compressed against the annular or arterial wall 105, or against inner walls of a shaft or a retaining capsule), and further configured to revert to its original shape (i.e., the shape of its relaxed state) when the external force is no longer applied thereto (e.g., when a valve is released from the shaft or capsule prior to expansion thereof).

According to some examples, the sealing member(s) of the present invention (e.g., sealing member 222) has a resilient 3D structure/shape, which is configured to deform when an external force exceeding a predefined threshold is applied thereto, and to revert to a relaxed state thereof when the external pressure is no longer applied thereto. According to some examples, the predefined threshold of the external pressure is 300 mmHg.

It is to be understood that the compressibility of the ridges 230 does not contradict the resilient 3D structure of the second layer 220, on which the ridges 230 are formed, as upon the ceasing of compression on the ridges 230 (e.g. if the sealing member 222 reverts back to a relaxed state), the ridges 230 structure of the second layer 220 will be reinstated.

According to some examples, the sealing member 222 comprises at least the first layer 210 comprising a tear resistant material and the second layer 220 comprising a thermoplastic thromboresistant material. According to some examples, the sealing member 222 further comprises the third layer 225 comprising a thermoplastic thromboresistant material. According to further examples, the sealing member 222 comprises the first layer 210 comprising a tear resistance material comprising a PET fabric, and the second layer 220 comprising thermoplastic thromboresistant material comprising TPU, wherein the TPU is thermally processed to assume a 3D geometrical shape along the first surface 202 of the sealing member 222, thereby forming a plurality of ridges 230 as described herein above. According to further examples, the sealing member 222 comprises the third layer 225 comprising a thermoplastic thromboresistant material comprising TPU, wherein the TPU is thermally processed to assume a 3D geometrical shape along the second surface 204 of the sealing member 222, thereby forming a plurality of channels 240 as described herein above.

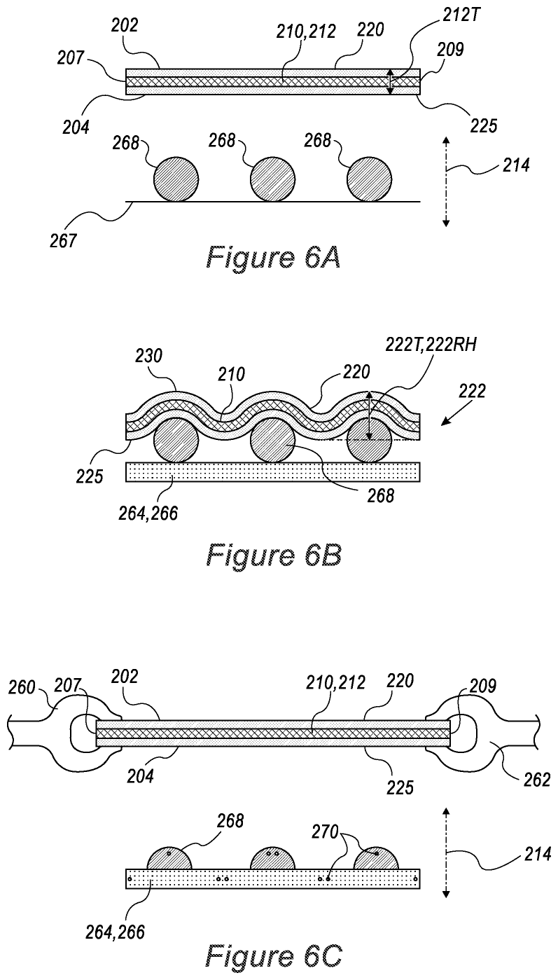

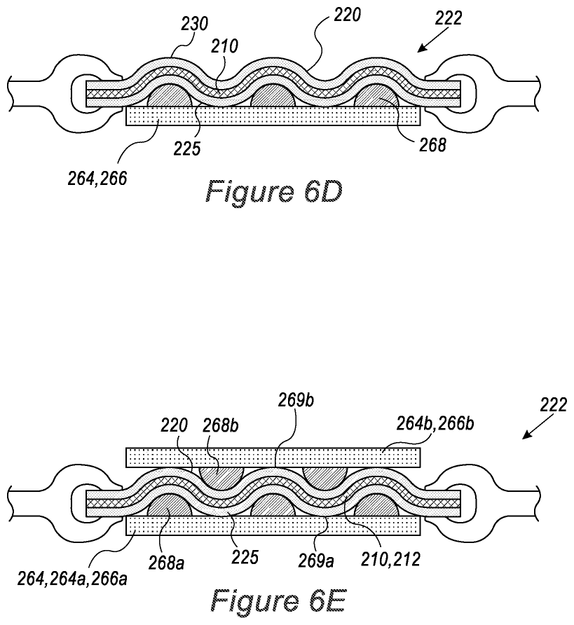

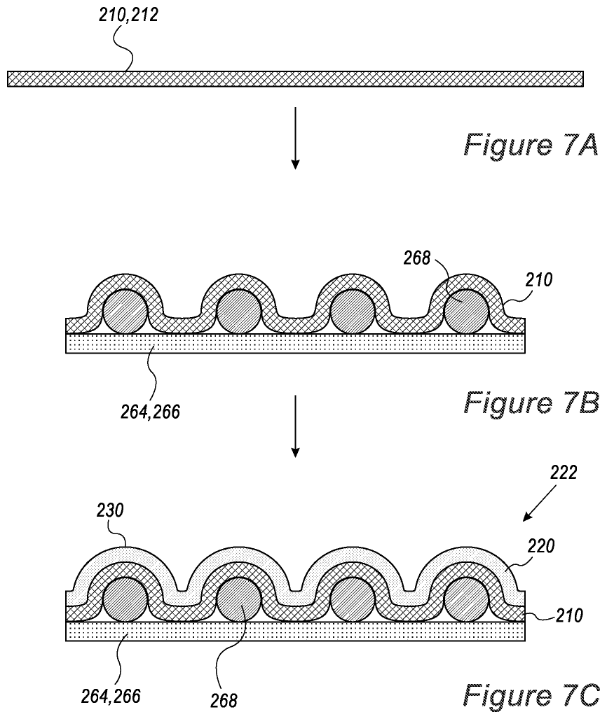

Reference is now made to FIGS. 6A-6E and 7A-7C. FIGS. 6A-6B show exemplary thermal shape-processing steps utilizing thermoforming, for the fabrication of the sealing member 222 in a spread state, according to some examples. Specifically, FIGS. 6A-6B show thermal processing steps of a flat flexible sheet 212, utilizing placing and heating over mold 264, for the fabrication of the sealing member 222 in a spread state, according to some examples. FIGS. 6C-6D show thermal processing steps of a flat flexible sheet 212, utilizing placing, heating and vacuum-thermoforming over a mold 264, for the fabrication of the sealing member 222 in a spread state, according to some examples. FIG. 6E shows thermal processing steps of a flat flexible sheet 212, utilizing thermoforming, which includes application of force using mold 264 over two opposite surface thereof, for the fabrication of the sealing member 222 in a spread state, according to some examples.

According to some examples, there is provided a PVL skirt 222 prepared by the methods of the present invention. According to some examples, there is provided a PVL skirt 222 in a folded state prepared by the methods of the present invention.

According to some examples, there is provided a method of fabricating a sealing member, such as the sealing member 222 as described herein above, in a cost-effective and simple manner. According to some examples, the method comprising: (i) providing a tear resistant flat sheet 212; (ii) treating the sheet in a thermal shape-forming process to assume a 3D shape in a spread relaxed state; and (iii) connecting two opposite edges of the sheet 212 of step (ii) to form a cylindrical sealing member (or PVL skirt) in a cylindrical folded state.

According to some examples, the method comprises (i) providing a flat flexible sheet 212, which comprises a tear resistant first layer 210 and a thermoplastic second layer 220; (ii) placing the flat flexible sheet 212 on a mold 264 at an elevated temperature, thereby forming a plurality of ridges 230 thereon, and lowering the temperature, thereby maintaining a resilient 3D structure of the thermoplastic second layer 220; and (iii) connecting two opposite edges of the sheet 212 of step (ii) to form a cylindrical sealing member 222.

According to some examples, the method comprises (i) providing a flat flexible sheet 212, which comprises a tear resistant first layer 210 disposed between a thermoplastic second layer 220 and a thermoplastic third layer 225 of the flat flexible sheet 212; (ii) placing the flat flexible sheet 212 on a mold 264 at an elevated temperature thereby forming a plurality of ridges 230 on the second layer 220, and lowering the temperature thereby maintaining a resilient 3D structure of the thermoplastic second layer 220; and (iii) connecting two opposite edges of the sheet 212 of step (ii) to form a cylindrical sealing member 222 (i.e., folding the sheet 212). According to some examples, the thermal processing of the sheet 212 utilizing a mold 264 at step (ii) comprises thermoforming.

It is to be understood that any of the properties introduced above for each one of the layers (i.e. the first layer 210, the second layer 220 and the third layer 225) similarly apply for the respective layers when referring to the method of the present invention. Specifically, according to some examples, the first layer 210 comprises at least one biocompatible material. According to some examples, the first layer 210 comprises at least one elastic material. According to some examples, the first layer 210 comprises at least one flexible material. According to further examples, the first layer 210 comprises a tear resistant PET fabric. According to some examples, the first layer 210 comprises at least one tear resistant material. According to some examples, the second layer 220 is made of a thermoplastic material. According to some examples, the third layer 225 is made of a thermoplastic material. According to some examples, the thermoplastic material is selected from the group consisting of: polyamides, polyesters, polyethers, polyurethanes, polyolefins, polytetrafluoroethylenes, and combinations and copolymers thereof. According to some examples, the thermoplastic material is a thermoplastic elastomer.

According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example. According to some examples, the thermoplastic elastomer is TPU. According to some examples, the second layer 220 comprises at least one thromboresistant material. According to some examples, the second layer 220 comprises TPU. According to some examples, the third layer 225 comprises at least one thromboresistant material. According to some examples, the third layer 225 comprises TPU. According to some examples, the second layer 220 and the third layer 225 are made from the same material. According to some examples, the third layer 225 is united with the second layer 220 as detailed herein.

According to some examples, step (ii) entails placing the flat flexible sheet 212 on a mold 264, wherein the second layer 220 is positioned opposite to the mold 264. According to some examples, step (ii) entails placing the flat flexible sheet 212 on a mold 264, wherein the third layer 225 is positioned in proximity to the mold 264. According to some examples, step (ii) entails placing the flat flexible sheet 212 on a mold 264, wherein the third layer 225 is contacting the mold 264. According to some examples, step (ii) entails placing the flat flexible sheet 212 on a mold 264, wherein the first layer 210 is contacting the mold 264.

According to some examples, the ridges 230 formed in step (ii) are formed over the second layer 220, thereby forming corresponding channels 240 at the third layer 225. According to some examples, the ridges 230 formed in step (ii) are formed over the second layer 220, thereby forming corresponding channels 240 at the first layer 210.

It is to be understood that the thermoplastic properties of the second layer 220 (and optionally of the third layer 225) enable the thermal shape-forming process described above. Specifically, thermoplastic materials are converted from a resilient relatively rigid state at lower temperatures to a pliable relatively soft state when heated. In step (ii) the thermoplastic second layer 220 is heated to its pliable state, according to some examples, thereby allowing the mold 264 to form a 3D shape comprising ridges 230 of the thermoplastic second layer 220. This thermal shape-forming process may be facilitated by application of external force, but it was exemplary found that placing simple mandrels (as mold 264) below the sheet 212 and heating in an oven is sufficient to allow thermal shape-forming via gravitation alone.

This example is illustrated in FIGS. 6A-6B. FIG. 6A illustrates, separately, the flat flexible sheet 212 when originally provided and mold 264 shown to include a plurality of mandrels 268 spaced apart from each other, optionally equally spaced apart from each other, according to some examples.

The term “mandrel”, as used herein, refers to an elongated member, such as a rod or a pipe, that may serve as a core over which thermoplastic material may be molded or otherwise shaped at an elevated temperature. A mandrel, as used herein, may relate to an elongated member, such as a rod or a pipe, having a uniformly sized cross-sectional profile along its length.

According to some examples, step (i) further includes providing a mold. FIG. 6A also shows that the mandrels 268 are placed over a ground surface 267, which in this case is to be heated at step (ii) of the method, and therefore, may be, the floor of and oven, according to some examples. In some implementations, the plurality of mandrels may be integrally formed with a base plate (e.g., ground surface 267), serving as protrusions extending therefrom. In other implementations, the mandrels 268 may be separate components attached to, or removable placed over, a base plate (e.g., ground surface 267). FIG. 6B shows the thermal shape-processing of the sheet 212 into the 3-dimensional shape of sealing member 222.

Specifically, in the example illustrated in FIGS. 6A-6B, the flat flexible sheet 212 is positioned over mandrels 268. As seen in this figure, the thermoplastic second and third layers 210 and 225 respectively are in their resilient state, according to some examples. Then, when the heating of the sheet 212 over the mandrels 268 is taking place, the portions of the sheet 212 which are not located above a mandrel 268 are gravitationally submerging (e.g. until contacting the oven floor), whereas the portions of the sheet 212 which are located above a mandrel 268 are not submerging due to the interference by the mandrels 268 (FIG. 6B). According to some examples, in step (ii), each ridge 230 is formed over each corresponding mandrel 268. After the 3D shape is assumed, the sheet 212 may be allowed to cool, so that thermoplastic second layer 220 reverts back to its resilient non-pliable state. The mandrels are then removed to obtain the seal member 222 in its spread state, according to some examples (FIG. 4A). Lastly, two opposite edges of the flexible sheet 212 are attached (e.g., sewn) to each other to obtain the seal member 222 in its folded state, according to some examples.

As used herein, the term “gravitationally submerging” refers to a material which is submerging in the direction of the gravitational force.

As further seen in FIGS. 6A and 6B, the flat flexible sheet 212 may also include the third layer 225, which is elaborated herein and undergoes a similar shape-processing as the second layer 220.

According to some examples, the sheet 212 of step (i) has a first surface 202 and a second surface 204, wherein the distance between the first surface 202 and a second surface 204 of the sheet 212 of step (i) constitutes the initial thickness 212T of the sheet 212 of step (i). In addition, according to some examples, the sheet 212 of step (i) has a first lateral edge 206 and a second lateral edge 208, wherein the distance between the first lateral edge 206 and a second lateral edge 208 of the sheet 212 of step (i) constitutes the initial width 212W (not shown) of the sheet 212 of step (i). Lastly, according to some examples, the sheet 212 of step (i) has an inflow edge 209 and an outflow edge 207, wherein the distance between the inflow edge 209 and the outflow edge 207 of the sheet 212 of step (i) constitutes the initial length 212L of the sheet 212 of step (i) (not shown). According to some examples, the initial thickness 212T corresponds to, or is identical to, the total layer thickness 203, as described above.

Specifically, according to some examples, the sheet 212 of step (i) is flat and substantially two dimensional. This means that the initial thickness 212T of the sheet 212 of step (i) is substantially shorter that the initial width 212W and the initial length 212L thereof.

According to some examples, the sheet 212 produced in step (ii) is the sealing member 222 in its spread, non-folded state. According to some examples, the first lateral edge 206 and the second lateral edge 208 of the sheet 212 of step (i) are the same the first lateral edge 206 and the second lateral edge 208 of the spread the sealing member 222 produced in step (ii). According to some examples, the inflow edge 209 and the outflow edge 207 of the sheet 212 of step (i) are the same the inflow edge 209 and the outflow edge 207 of the spread the sealing member 222 produced in step (ii).

According to some examples, upon performing the method of the present invention, ridges 230 are formed, wherein the ridges 230 have ridge height 222RH, being the thickness 222T of sealing member 222 in its spread relaxed state. It is to be understood that any reference to the ridge height 222RH or thickness 222T is equivalent to the distance of the peaks 205 from the external surface of the frame 106, in a relaxed state of the sealing member 222 when coupled to the frame 106. Similarly, and reference to the initial thickness 212T is equivalent to the distance of the non-elevated portions 250 from the external surface of the frame 106, when the sealing member 222 is coupled thereto.

According to some examples, the thickness 222T of sealing member 222 in its spread relaxed state, following the thermal shape-forming step (ii) configured to assume the 3D shape thereof, is at least 1000% greater than the initial thickness 212T of the sheet 212. According to some examples, the thickness 222T of sealing member 222 in its spread relaxed state is at least 2000%, at least 3000%, at least 4000%, at least 5000%, or at least 6000% greater than the initial thickness 212T of the sheet 212.

It is to be understood that the width 212W and length 212L of the sheet 212 may also be somewhat modified upon performance of the present process, however, the significant dimension modification is of the thickness (212T to 222T), which convert the initial 2D structure of the sheet 212 to a 3D structure in sealing member 222. In some implementations, the resulting sheet 212 after step (ii) has dimensions that are greater than any of the desired width 212W and/or the desired length 212L, and the method can include an additional step of cutting the sheet 212 to the desired width 212W and/or the desired length 212L, after step (ii) and prior to step (iii).

According to some examples, the tear resistant flat sheet 212 of step (i) comprises the first layer 210 as described herein above. According to some examples, the sheet 212 of step (i) comprises the second layer 220 as described herein above. According to some examples, the sheet 212 of step (i) comprises the third layer 225 as described herein above. According to further examples, the tear resistant flat sheet of step (i) comprises a PET fabric.

According to some examples, the method of the present invention comprises coating at least one surface of a flat tear resistant sheet with a thermoplastic polymeric coating layer to obtain the sheet 212 of step (i).

According to some examples, treating the sheet to assume a 3D shape in step (ii) comprises simultaneously coating at least one surface of the flat tear resistant sheet while thermally shape-forming processing the sheet, to form a 3D coated shape in a spread relaxed state as described herein above. According to further examples, coating at least one surface of the flat tear resistant sheet comprises coating the tear resistant first layer 210 with at least one of the thermoplastic second layer 220 and the thermoplastic third layer 225.

According to some examples, coating at least one surface of the flat tear resistant sheet with a thermoplastic polymeric coating layer is performed by a coating technique selected from brushing, spray-coating, dip coating, dipping or immersing, and combinations thereof. The present invention, however is not limited to such coating techniques, and other coating techniques, such as chemical deposition, vapor deposition, chemical vapor deposition, physical vapor deposition, printing and the like, may suitably be used. These techniques are generally suitable for medical textiles. Moreover, printing techniques, such as roller printing, stencil printing, screen printing, inkjet printing, lithographic printing, 3D printing, and the like may be also used with the present invention for applying the thermoplastic polymeric coating.

According to some examples, step (ii) comprises placing the flat flexible sheet 212 on a mold 264 at an elevated temperature thereby forming a plurality of ridges 230 on the second layer 220, and lowering the temperature thereby maintaining a resilient 3D structure of the thermoplastic second layer 220.

It is to be understood that the elevated temperature in step (ii) refers to a temperature in which the thermoplastic material of the second layer 220 (and of the third layer 225) is pliable and soft, so that the sheet 212 is thermally shape-formable into a 3D configuration, according to some examples. Thus, the temperature is dependent on the specific thermoplastic material used. It is further to be understood, according to some examples, that the lowering of the temperature in step (ii) refers to a temperature in which the thermoplastic material of the second layer 220 (and of the third layer 225) is resilient, so that it maintains its 3D structure. Since step (iii) of folding the sealing member 222 into a cylindrical shape is typically done at ambient temperatures, the lowering of the temperature in step (ii) may entail lowering to ambient (e.g. room) temperature.

According to some examples, the elevated temperature in step (ii) is at least 50° C. According to some examples, the elevated temperature in step (ii) is at least 60° C. According to some examples, the elevated temperature in step (ii) is at least 70° C. According to some examples, the elevated temperature in step (ii) is at least 80° C. According to some examples, the elevated temperature in step (ii) is at least 90° C. According to some examples, the elevated temperature in step (ii) is at least 100° C. According to some examples, the elevated temperature in step (ii) is at least 120° C. According to some examples, heating the flat sheet to the elevated temperature comprises heating at least one surface of the sheet 212 or preferably at least two surfaces of the sheet, to a temperature selected from about 100° C. to about 250° C., or preferably from about 120° C. to 200° C.

According to some examples, the lowering of the temperature in step (ii) comprises cooling the sheet 212 to a temperature below 40° C. According to some examples, the lowering of the temperature in step (ii) comprises cooling the sheet 212 to room temperature.

According to some examples, the mold 264 is made of a temperature resilient material. According to some examples, the mold 264 comprises a temperature resilient material. According to some examples, the mold 264 is made of a metal or a metal alloy. Each possibility represents a separate example. According to some examples, the mold 264 comprises a metal or a metal alloy.

It is to be understood that a thermally resistant mold 264 may be required for thermal shape-processing methods, which involve molds, since the structure of the mold 264 is to remain substantially unchanged during the method.

According to some examples, the mold 264 has an elongated structure. According to some examples, the mold 264 has an elongated mold. Specifically, as shown in FIG. 6B, the formed shape of the sheet 212 produced in step (ii) includes line-shaped ridges 230, which are formed to follow path-lines due to the elongated shape of the mold 264. The present method, however, is not limited to elongated mold 264, according to some examples, as other types of mold 264 would lead to other types of ridges 230, as can be appreciated by those skilled in the art.

Specific types of elongated mold include, but are not limited to, pipes, shafts, rods and mandrels. According to some examples, the mold 264 comprises at least one rod. According to some examples, the mold 264 comprises a plurality of rods. According to some examples, the mold 264 comprises mandrels.

According to some examples, step (ii) comprises placing the flat flexible sheet 212 on elongated mold 264, wherein the mold 264 extends at least from the first lateral edge 206 to the second lateral edge 208 of the sheet 212, at an elevated temperature thereby forming a plurality of ridges 230 on the second layer 220, wherein the plurality of ridges 230 extend from the first lateral edge 206 to the second lateral edge 208 of the sheet 212, and lowering the temperature thereby maintaining a resilient 3D structure of the thermoplastic second layer 220. According to some examples, the plurality of ridges 230 are perpendicular to any one of the first lateral edge 206 and/or the second lateral edge of the sheet 212 produced in step (ii). According to some examples, the plurality of ridges 230 are parallel to any one of the inflow edge 209 and/or the outflow edge 207 of the sheet 212 produced in step (ii). According to some examples, the plurality of ridges 230 are parallel to any one of the inflow edge 209 and/or the outflow edge 207 of the sealing member produced in step (iii). Such configurations are shown in FIGS. 4A and 4D.

According to some examples, step (ii) comprises placing the flat flexible sheet 212 on elongated mold 264, wherein the mold 264 extends from the inflow edge 209 to the outflow edge 207, at an elevated temperature thereby forming a plurality of ridges 230 on the second layer 220, wherein the plurality of ridges 230 extend between inflow edge 209 to the outflow edge 207 of the sheet 212, and lowering the temperature thereby maintaining a resilient 3D structure of the thermoplastic second layer 220.

According to some examples, the plurality of ridges 230 are parallel to the first lateral edge 206 and to the second lateral edge of the sheet 212 produced in step (ii). According to some examples, the plurality of ridges 230 are perpendicular to the inflow edge 209 and to the outflow edge 207 of the sheet 212 produced in step (ii). According to some examples, the plurality of ridges 230 are perpendicular to the inflow edge 209 and to the outflow edge 207 of the sealing member produced in step (iii). Such a configuration is shown in FIG. 4E.

According to some examples, step (ii) comprises placing the flat flexible sheet 212 on elongated mold 264, wherein the mold 264 extends diagonally along at least a portion of the second surface 204 of the sheet 212, at an elevated temperature thereby forming a plurality of diagonal ridges 230 on the second layer 220, wherein the plurality of ridges 230 extend from inflow edge 209 to the outflow edge 207 of the sheet 212, and lowering the temperature thereby maintaining a resilient 3D structure of the thermoplastic second layer 220. Such a configuration is shown in FIG. 4F.

According to some examples, step (ii) further comprises removing the mold 264 from the sheet after the temperature was lowered.

According to some examples, step (ii) further comprises cooling the processed sheet 212, thereby stabilizing the desired 3D shape thereof.

Once the resilient 3D structure of the thermoplastic second layer 220 was obtained at step (ii), the resulting 3D sheet 212 can be folded and sutured into a cylindrical shape, thereby forming a cylindrical sealing member 222.

According to some examples, step (iii) comprises connecting the two opposite edges (i.e., first lateral edge 206 and a second lateral edge 208) of the sheet of step (ii) to form a cylindrical sealing member 222 (or PVL skirt) in a cylindrical folded state. The connection between the opposite edges can be performed by using at least one of adhesives, clipping, sutures, or heating and optionally melting the edges thereof. Alternatively, step (iii) comprises coupling the sealing member 222 to an outer surface of the frame 106, utilizing at least one of adhesives, sutures, or heating and optionally melting the edges of the sealing member 222 therearound. Such coupling results in a cylindrical folded shape the sealing member 222, which is forced by the cylindrical shape of the frame 106 (see FIGS. 5A-5C).

Reference is now made to FIGS. 6C-6D. FIGS. 6C-6D show thermal processing steps of a flat flexible sheet 212, utilizing placing, heating and vacuum-thermoforming over a mold 264, for the fabrication of the sealing member 222 in a spread state, according to some examples.

According to some examples, there is provided a method of fabricating the sealing member 222 as described herein above, the method comprising: (i) providing (a) a flat sheet 212 comprising a tear resistant first layer 210 and a thermoplastic second layer 220 as described herein above, and (b) a mold 264 comprising a base 266, a plurality of protrusions, wherein each one is in a form of a mandrel 268, and a vacuum source comprising apertures 270; (ii) placing the flat sheet 212 over the plurality of elongated rods or mandrels 268 and applying vacuum using the vacuum source utilizing apertures 270 thereto at an elevated temperature, thereby thermoforming the sheet 212 to a 3D shape in a spread relaxed state; and (iii) connecting two opposite edges of the sheet 212 of step (ii) to form a cylindrical sealing member (or PVL skirt) in a cylindrical folded state.

According to some examples, the mandrels 268 are provided in the form of elongated rods or protrusions. According to some examples, there is provided a method of fabricating the sealing member 222 as described herein above, the method comprising: (i) providing (a) a flat sheet 212 comprising a tear resistant first layer 210, a thermoplastic second layer 220, and a third layer 225, as described herein above, and (b) a mold 264 comprising a base 266, a plurality of protrusions 268 and a vacuum source comprising apertures 270; (ii) placing the flat sheet 212 over the plurality of protrusions 268 and applying vacuum using the vacuum source utilizing apertures 270 thereto at an elevated temperature, thereby thermoforming the sheet 212 to a 3D shape in a spread relaxed state; and (iii) connecting two opposite edges of the sheet 212 of step (ii) to form a cylindrical sealing member (or PVL skirt) in a cylindrical folded state.

The properties of each of the first layer 210, second layer 220 and third layer 225 are as described herein above.

According to some examples, step (i) further comprises providing a mold 264 comprising a base 266 and a plurality of protrusions 268 extending away therefrom in parallel to an axis 214 (see FIG. 6C), and spaced from each other along the base 266. According to further examples, the base 266, the plurality of protrusions 268, or both, comprise a plurality of apertures 270. According to some examples, the plurality of apertures 270 are formed at the base 266. According to some examples, the plurality of apertures 270 are part of a vacuum source. According to some examples, the plurality of apertures 270 are connected (e.g., fluidly connected) to a vacuum pump.

According to further examples, step (ii) further comprises supporting the sheet 212 by at least one holder. According to further examples step (ii) further comprises supporting the sheet 212 by at least two holders, wherein a first holder 260 is configured to secure/support the outflow edge 207 and a second holder 262 is configured to secure/support the inflow edge 209 of the sheet. According to some examples, the flat sheet is secured/supported by a plurality of holders (not shown). It is to be understood that the holders may similarly secure/support the opposite lateral edges.

According to some examples, step (ii) of the method comprises thermally shape-processing the sheet utilizing thermoforming to assume a 3D shape in a spread relaxed state utilizing the mold 264 (see FIG. 6D).

According to some examples, step (ii) comprises: supporting the flat sheet 212 utilizing at least the first and second holders 260 and 262, respectively positioning the flat sheet 212 above the mold 264; heating the flat sheet to a thermoformable temperature; and bringing the sheet 212 toward said mold 264, by moving the first and second holders and 262 respectively in its direction, to effectively engage said flat sheet with the protrusions 268 of mold 264 to thereby enable the sheet 212 to conform to said protrusions 268.

According to some examples, step (ii) comprises: supporting the flat sheet 212 utilizing at least the first and second holders 260 and 262; positioning the flat sheet 212 above the mold 264; heating the flat sheet to a thermoformable temperature; approximating the sheet 212 toward said mold 264, by moving the first and second holders and 262 in its direction to effectively engage said flat sheet 212 with the protrusions 268 of mold 264; and applying vacuum through the apertures 270, to facilitate conformation of the sheet 212 to said protrusions 268.

According to some examples, step (ii) comprises: supporting the flat sheet utilizing at least the first and second holders 260 and 262 respectively; positioning the flat sheet above the mold 264; heating the flat sheet to a thermoformable temperature; and lifting the mold 264 toward the flat sheet, while the first and second holders 260 and 262, respectively, remain stationary relative to the movement of the mold, or concurrently approximated toward the mold 264 as well, to effectively engage the protrusions 268 of the mold 264 with the sheet 212 to facilitate conformation of the sheet to said mold 264.

According to some examples, step (ii) comprises: supporting the flat sheet utilizing at least the first and second holders 260 and 262 respectively; positioning the flat sheet above the mold 264; heating the flat sheet to a thermoformable temperature; lifting the mold 264 toward the flat sheet, while the first and second holders 260 and 262, respectively, remain stationary relative to the movement of the mold, or concurrently approximated toward the mold 264 as well, to effectively engage the protrusions 268 of the mold 264 with the sheet 212; and applying vacuum through the apertures 270, to facilitate conformation of the sheet to said mold 264.

According to some examples, heating the flat sheet to the thermoformable temperature can be performed after forming the engagement between the mold 264 and the flat sheet.

As used herein, the term “thermoformable temperature” refers to a temperature in which the second layer 220 (and optionally the third layer 225) comprising the thermoplastic material as described above, preferably TPU, is heated to, in order to enable comfortable handling and thermal processing thereof, to conform to the 3D shape of the mold, without igniting or undergoing degradation. According to some examples, the thermoformable temperature is above or equal to the glass transition temperature of the thermoplastic material. According to some examples, the thermoformable temperature is above the glass transition temperature of the thermoplastic material.

According to some examples, heating the flat sheet to the thermoformable temperature comprises heating at least one surface of the sheet, or preferably at least two surfaces of the sheet, to a temperature selected from about 100° C. to about 250° C., or preferably from about 120° C. to about 200° C. According to some examples, the elevated temperature in step (iii) is at least 50° C. According to some examples, the elevated temperature in step (iii) is at least 60° C. According to some examples, the elevated temperature in step (iii) is at least 70° C. According to some examples, the elevated temperature in step (iii) is at least 80° C. According to some examples, the elevated temperature in step (iii) is at least 90° C. According to some examples, the elevated temperature in step (iii) is at least 100° C. According to some examples, the elevated temperature in step (iii) is at least 120° C.

According to some examples, the engagement of the sheet 212 with the plurality of protrusions 268 forms the plurality of ridges 230, while the engagement of the sheet with the base 266 forms the plurality of inter-ridge gaps 250 in the sealing member 222.

According to some examples, step (ii) further comprises applying reduced pressure through apertures 270 (e.g., by vacuum-pumping therethrough) between the sheet 212 and the mold 264, in order to stretch and pull the sheet toward the mold 264 and to form enhanced attachment therebetween, thereby allowing the sheet 212 to successfully conform to the shape of the mold 264.

It is to be understood that, according to some examples, a part of the system for molding the 3D structure of the sealing member includes means for applying suction, e.g., a vacuum pump. The vacuum pump may be, according to some examples, connected through tubing to the apertures 270 in the base 266 (and/or protrusions 268) from the surface of the base 266, which is opposite to the side of the plurality of protrusions 268. In this configuration, upon actuation of the vacuum pump, air is sucked through the apertures 270 from the protrusions 268 side, onto which the sheet 212 is held. As the sheet is heated in step (ii), the thermoplastic properties of its thermoplastic layer(s) (second layer 220 and, optionally, third layer 225) render it pliable or thermoformable, such that upon the application of the suction force (i.e., the negative pressure), the sheet 212 is stretched and pulled toward the mold 264, according to some examples. Upon ceasing of the heating and allowing the thermoplastic layer/s to reach a temperature in which it is more rigid and resilient, the external force can no longer shape-set the formed sealing member 222, which resiliently remains in the newly formed 3D shape.

Reference is now made to FIG. 6E. FIG. 6E shows thermal processing of a flat flexible sheet 212, utilizing thermoforming. The thermoforming of FIG. 6E includes application of force using mold (264a, 264b) over two opposite sides of the flexible sheet 212, for the fabrication of the sealing member 222 in a spread state, according to some examples.

According to some examples, there is provided a method of fabricating a sealing member 222, the method comprising: (i) providing (a) a flat sheet 212 comprising a tear resistant first layer 210 and a thermoplastic second layer 220 as described herein above, and (b) a mold 264 comprising a first mold 264a and a second mold 264b, wherein the first mold 264a comprises a first base 266a and plurality of first mold protrusions 268a and the second mold 264b comprises a second base 266b and plurality of second mold protrusions 268b; (ii) placing the flat sheet 212 between the plurality of first mold protrusions 268a and the plurality of second mold protrusions 268b pressing the second mold 264b against the first mold 264a at an elevated temperature, thereby thermoforming the sheet 212 to a 3D shape; and (iii) connecting two opposite edges of the sheet 212 of step (ii) to form a cylindrical sealing member (or PVL skirt) in a cylindrical folded state.

According to some examples, there is provided a method of fabricating a sealing member 222, the method comprising: (i) providing (a) a flat sheet 212 comprising a tear resistant first layer 210, a thermoplastic second layer 220 and a third layer 225 as described herein above, and (b) a mold 264 comprising a first mold 264a and a second mold 264b, wherein the first mold 264a comprises a first base 266a and plurality of first mold protrusions 268a and the second mold 264b comprises a second base 266b and plurality of second mold protrusions 268b; (ii) placing the flat sheet 212 between the plurality of first mold protrusions 268a and the plurality of second mold protrusions 268b, and pressing the second mold 264b against the first mold 264a at an elevated temperature, thereby thermoforming the sheet 212 to a 3D shape; and (iii) connecting two opposite edges of the sheet 212 of step (ii) to form a cylindrical sealing member (or PVL skirt) in a cylindrical folded state.

The properties of each of the first layer 210, second layer 220 and third layer 225 are as described herein above. the temperatures in which step (ii) is conducted are also as described herein above.

According to some examples, second mold 264b comprises a second base 266b and a plurality of protrusions 268b extending away therefrom and spaced from each other along the second base 266.

According to some examples, step (ii) comprises placing the flat sheet 212 between the plurality first mold protrusions 268a and the plurality of second mold protrusions 268b, so that each one of the plurality first mold protrusions 268a (optionally excluding the outermost protrusions) is positioned laterally between second mold protrusions 268b, wherein the flat sheet 212 spaces therebetween. According to some examples, step (ii) further comprises pressing the second mold 264b against the first mold 264a at an elevated temperature, thereby effectively engaging the flat sheet 212 therebetween to allow the sheet 212 to conform to said the shape of molds (see FIG. 6E). The second mold 264b and the first mold 264a can be identical or different relative to one another.

According to some examples, step (ii) comprises placing the flat sheet 212 between the plurality of first mold protrusions 268a and the plurality of second mold protrusions 268b, so that the plurality first mold protrusions 268a and the plurality second mold protrusions 268b are intermittently disposed relative to each other along both opposite sides of the sheet 212, having each first mold protrusion 268a positioned laterally between a couple two second mold protrusions 268b (optionally excluding the outermost protrusions), wherein the flat sheet 212 spaces between the first mold 264a and the second mold 264b; and pressing the second mold 264b against the first mold 264a at an elevated temperature, thereby effectively engaging the flat sheet 212 therebetween to allow it to conform to the shape of said molds.

According to some examples, step (ii) comprises placing the flat sheet 212 between the plurality of first mold protrusions 268a and the plurality of second mold protrusions 268b, so that the plurality first mold protrusions 268a and the plurality second mold protrusions 268b are disposed at a zipper-like configuration; and pressing the second mold 264b against the first mold 264a at an elevated temperature, thereby effectively engaging the flat sheet 212 therebetween to allow it to conform to the shape of said molds. According to some examples, step (ii) comprises placing the flat sheet 212 between the plurality of first mold protrusions 268a and the plurality of second mold protrusions 268b, so that the plurality first mold protrusions 268a and the plurality second mold protrusions 268b are disposed at a staggered configuration.

The terms “zipper-like configuration” and “staggered configuration” as used herein can be appreciated from FIG. 6E. Specifically, as shown therein, a first mold inter-protrusion gap 269a is formed between each couple of adjacent first mold protrusions 268a. Similarly, a second mold inter-protrusion gap 269b is formed between each couple of adjacent second mold protrusions 268b, according to some examples. The zipper-like staggered configuration between the first mold 264a and second mold 264b elements is characterized by that the first mold protrusions 268a are located below and are aligned with the second mold inter-protrusion gaps 269b, and the second mold protrusions 268b are located above and are aligned with the first mold inter-protrusion gaps 269a. In addition, similar to a conventional zipper-like configuration, the external (outer-most) protrusions (which can refer to either first mold protrusions 268a or second mold protrusions 268b) may not be necessarily positioned above an inter-protrusion gap.

Vacuum can be formed between the sheet 212 and each of the molds 264a and 264b for enhanced attachment therebetween, as disclosed herein above.

According to some examples, step (ii) further comprises cooling the sheet 212 below the thermoformable temperature, thereby stabilizing the 3D shape in the spread relaxed state of the sealing member 222. According to some examples, step (ii) further comprises removing said 3D shape-set sheet 212 from the molds 264a and 264b once the desired three-dimensional shape has been assumed.

According to some examples, the method further comprises: (iii) connecting two opposite edges (i.e., first lateral edge 206 and a second lateral edge 208) of the sheet of step (ii) to form a cylindrical sealing member (or PVL skirt) in a cylindrical folded state. The connection between the opposite edges can be performed by using at least one of adhesives, sutures, or heating and optionally melting the edges thereof. Alternatively, step (iii) comprises coupling the sealing member 222 to an outer surface of the frame 106, utilizing at least one of adhesives, sutures, or heating and optionally melting the edges of the sealing member 222 therearound (see FIGS. 5A-5C).

Reference is now made to FIGS. 7A-C. FIG. 7A shows a flexible pre-coated sheet 212 at a spread relaxed state, according to some examples. FIG. 7B shows the flexible pre-coated sheet 212 of FIG. 7A placed over a mold 264, such that the flexible sheet 212 flexibly alters its shape to assume the shape of the mold 264, according to some examples. FIG. 7C shows a coating process of the shaped-altered flexible sheet 212 of FIG. 7B, according to some examples.

According to some examples, there is provided a method of fabricating a sealing member 222, the method comprising: (i) providing a flat sheet 212 comprising a tear resistant first layer 210 (see FIG. 7A), and providing mold 264 comprising a base 266, and a plurality of protrusions 268; (ii) placing the flat sheet 212 onto the mold 264, thereby engaging the flat sheet 212 with the mold 264 to thereby enable the sheet to conform to said mold 264 in a 3D shape (see FIG. 7B), and coating the shaped sheet 212 with a second layer 220 (see FIG. 7C); and (iii) connecting two opposite edges of the sheet of step (ii) to form a cylindrical sealing member (or PVL skirt) in a cylindrical folded state.

It is to be understood that the phrase “conform to said mold 264” is intended to mean that the flat sheet 212 is being shaped similarly to the shape of the mold 264. More specifically, while the initial sheet 212 is flat, upon is placing and conforming to the mold 264, the sheet 212 roughly assumes the shape of the protrusions 268 of the mold 264. This confirmation may be prompted by gravitation and/or assisted by external force, according to some examples.

According to some examples, placing the flat sheet 212 onto the mold 264, entails gravitationally conforming the sheet 212 to the shape of said mold 264.

As used herein, the term “gravitationally conforming” refers to a material which is conforming onto the mold 264 in the direction of the gravitational force.

The properties of each of the first layer 210 and second layer 220 are as described herein above.

According to some examples, the second layer 220 is made of a thermoplastic material, and the coating of the 3D shaped sheet 212 with the second layer 220 in step (ii) involves heat-coating the shaped sheet 212 with the second layer 220 at an elevated thermoformable temperature. The heat-coating can be performed via the various coating techniques disclosed herein.

According to some examples, the engagement of the pre-coated sheet 212 with the plurality of protrusions 268 of the mold 264 forms the plurality of ridges 230 of the desired 3D shape of the sealing member 222, while the engagement of the sheet with the base 266 forms the plurality of inter-ridge gaps 250. According to some examples, step (ii) further comprises cooling the sheet and/or the mold, optionally below the thermoformable temperature, thereby stabilizing the 3D shape of the sheet 212. According to some examples, step (ii) further comprises removing said formed 3D shaped sheet 212 from the mold 264. According to some examples, step (ii) further comprises removing said formed 3D shaped sheet 212 from the mold 264 one the 3D shaped has been resiliently assumed.

According to some examples, step (iii) entails connecting two opposite edges (i.e., first lateral edge 206 and second lateral edge 208) of the sheet of step (ii) to form a cylindrical sealing member (or PVL skirt) in a cylindrical folded state. The connection between the opposite edges can be performed by using at least one of adhesives, sutures, or heating and optionally melting the edges thereof. Alternatively, step (iii) comprises coupling the sealing member 222 to an outer surface of the frame 106, utilizing at least one of adhesives, sutures, or heating and optionally melting the edges of the sealing member 222 therearound.

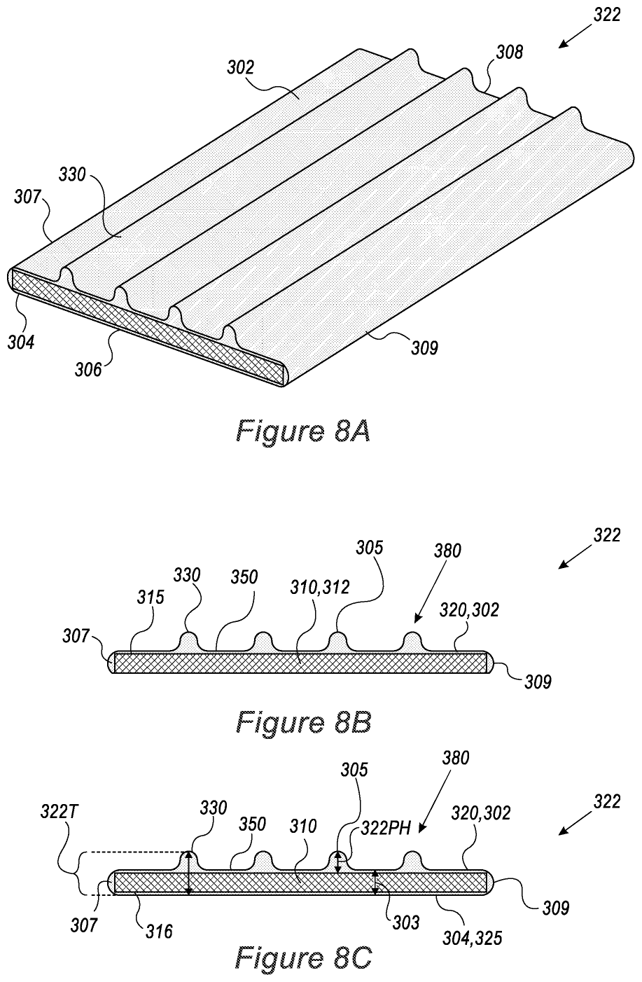

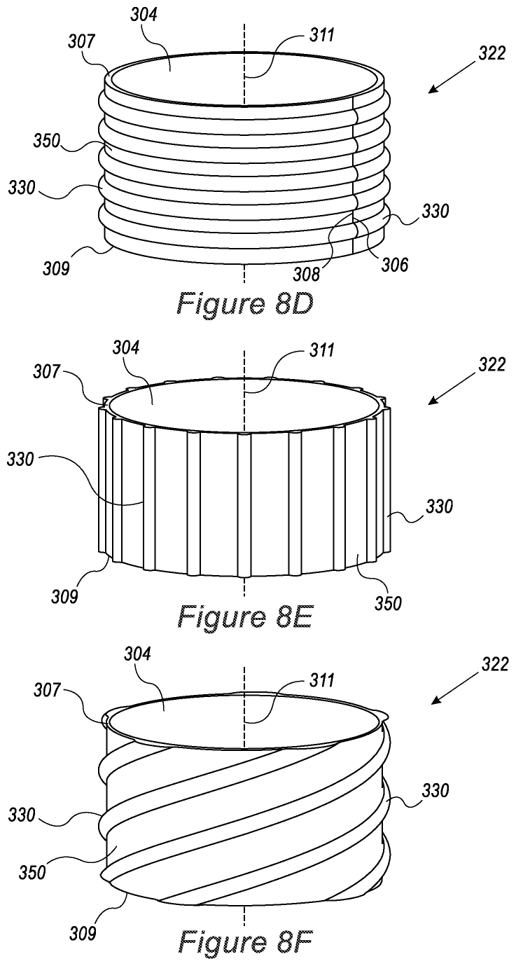

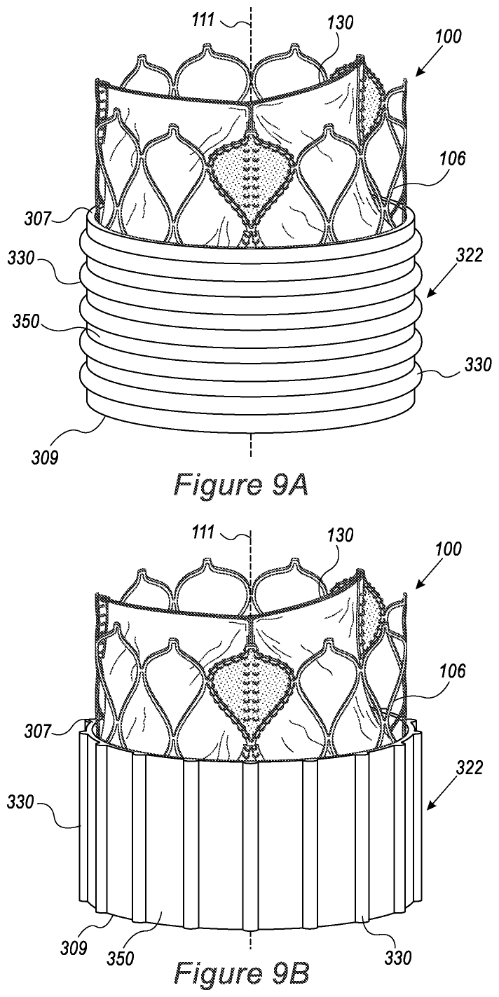

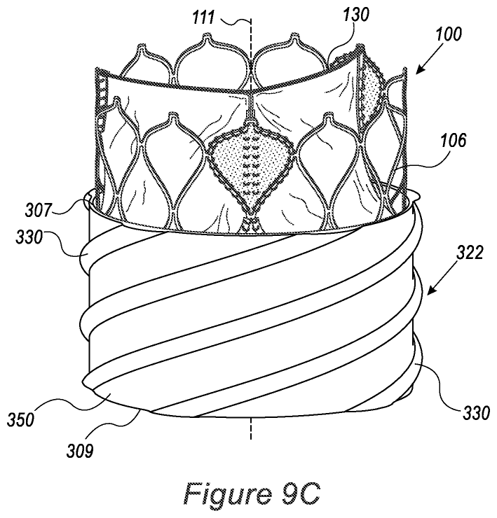

Reference is now made to FIGS. 8A-9C. FIG. 8A show a view in perspective of a sealing member 322 in a spread relaxed state, according to some examples. FIGS. 8B and 8C shows cross-sectional views of the sealing member 322, according to some examples. FIGS. 8D-8F shows views in perspective of various configurations of sealing member 322, in a cylindrical folded state, according to some examples. FIGS. 9A-9C show various configurations of the sealing member 322 mounted on the frame 106 of the prosthetic valve 100, according to some examples.

According to another aspect, there is provided a sealing member 322, adapted to be mounted on (or coupled to) the outer surface of the frame 106 of the prosthetic valve 100 (see for example, FIGS. 9A-9C), or any other similar prosthetic valve known in the art. According to some examples, the present invention provides a prosthetic heart valve 100 comprising a frame 106 and a leaflet assembly 130 mounted within the frame, the frame comprising a plurality of intersecting struts 110, wherein the frame is movable between a radially compressed state and a radially expanded state, as disclosed herein above, wherein the valve 100 further comprises a sealing member 322 coupled to an outer surface of the frame 106, and wherein the sealing member 322 has a three-dimensional (3D) shape in a spread relaxed state thereof.

The sealing member 322 can be connected/mounted to the frame 106 using suitable techniques or mechanisms. For example, the sealing member 322 can be sutured to the frame 106 utilizing sutures that can extend around the struts 110. The sealing member 322 can be provided in a spread state, and connected/mounted to the frame 106 by folding it over the frame 106, thereby transforming it from the spread to the folded state. Alternatively, the sealing member 222 may be provided in an already folded state prior to attachment to the frame 106. For example, the frame 106 may be inserted into the already cylindrically folded sealing member 322 and sutured thereto. The sealing member 222 can be configured to form a snug fit with the frame 106 such that it lies against the outer surface of the frame 106 when the prosthetic valve 100 is in the radially expanded state, as illustrated.

According to some examples, the sealing member 322 has a 3D shape in a spread relaxed state thereof, as can be appreciated for example from FIGS. 8A-8C. According to some examples, the sealing member 322 inherently has a 3D shape in a cylindrical folded state thereof (FIGS. 8D-8F and 9A-9C).

According to some examples, the sealing member 322 has a 3D resilient structure such that a nonfibrous outer surface 380 of the sealing member 322 exhibits a plurality of elevated portions 330 with peaks 305 and a plurality of non-elevated portions 350. In further examples, each one of the plurality of non-elevated portions 350 is defined by adjacent pairs of the plurality of elevated portions 330. In further examples, the nonfibrous outer surface 380 is a smooth surface. In further examples, the nonfibrous outer surface 380 is a unitary/continuous surface.

In some examples, the elevated portions 330 are protrusions 330 and the non-elevated portions 350 are inter-protrusion gaps 350. As used herein, the terms “elevated portions 330” and “protrusions 330” are interchangeable, and refer to the same plurality of elevated portions of the sealing member 322, as can be seen in FIGS. 8B-8C. As used herein, the terms “non-elevated portions 350” and “inter-protrusion gaps 350” are interchangeable, and refer to the same plurality of non-elevated portions of the sealing member 322, as can be seen in FIGS. 8B-8C.

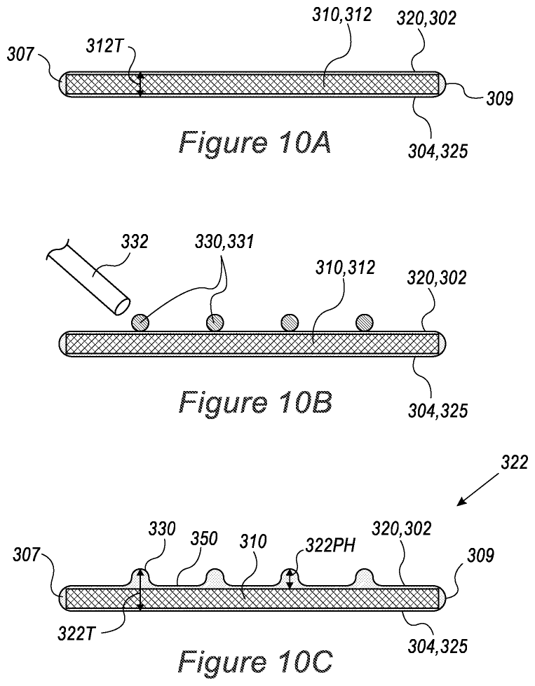

Specifically, as can be appreciated for example from FIG. 8A, the sealing member 322 comprises a plurality of protrusions 330, which cause its shape to be 3-dimensional (3D), in contrast to the substantially flat two-dimensional shape it would assume in the absence of such protrusions 330 (c.f., FIG. 10A). It is thus to be understood that the 3-dimensions of the 3-dimensional sealing member 322 include: (i) a spatial length dimension extending between an outflow edge 307 and an inflow edge 309 of the sealing member 322 (see FIGS. 8A, 8B and 8C); (ii) a spatial length dimension extending between a first lateral edge 306 and an second lateral edge 308 of the sealing member 322 (see FIG. 8A); and (iii) a spatial length (thickness) dimension 322T defined as the distance between the sealing member's protrusions 330 and its second surface 304 height (see FIG. 8C). It is further to be understood that the 3D structure of the sealing member 322 is attributed to the thickness 322T, which is greater by at least 1000%, alternatively at least 2000%, than the thickness of the flat 2D structure thereof, prior to the formation of the protrusions 330 thereon.

According to some examples, the sealing member 322 comprises a plurality of protrusions 330 extending away from a first surface 302 of the sealing member 322, and are spaced apart from each other along the first surface 302 of the sealing member 322. The plurality of protrusions 330 form the 3D shape of the sealing member 322 when in its spread relaxed state (as can be seen in the FIGS. 8A-8C), according to some examples. According to some examples, the sealing member 322 comprises a flat surface (e.g., a surface 316 or a surface 304) located opposite to the first surface 302, when in its spread relaxed state. According to some examples, an inner layer of the sealing member 322 (e.g., a first layer 310) is flat, when the sealing member 322 is in its spread relaxed state.

According to some examples, the sealing member 322 has four edges. According to some examples, the sealing member 322 has four vertices. According to some examples, each one of the four vertices of the sealing member 322 has a substantially right angle.

According to some examples, the sealing member 322 has four substantially right angle vertices, and two sets of two opposing edges (a set of first lateral edge 306 and second lateral edge 308, and a set of outflow edge 307 and an inflow edge 309), wherein in each set, the two opposing edges are substantially parallel. According to some examples, the sealing member 322 extends from a first lateral edge 306 toward a second lateral edge 308, when the sealing member 322 is in a spread state. According to some examples, the sealing member 322 extends around a sealing member centerline 311, when the sealing member 322 is in a folded state. According to some examples, the sealing member centerline 311 and the centerline 111 of valve 100 are coaxial and may coincide when the sealing member 322 is connected to heart valve 100. According to some examples, the sealing member 322 extends from an inflow edge 309 toward an outflow edge 307. According to some examples, the sealing member 322 extends from an inflow edge 309 toward an outflow edge 307 in both the folded state and the spread state thereof.

According to some examples, in the spread state, sealing member 322 is substantially rectangular. According to some examples, the distance from first lateral edge 306 and second lateral edge 308 is greater that the distance from inflow edge 309 to outflow edge 307.

According to some examples, each one of the plurality of protrusions 330 extends radially outward, away from the sealing member centerline 311, when the sealing member 322 is in a folded state (see FIGS. 8D-8F). According to some examples, each one of the plurality of protrusions 330 extends outward, radially away from the centerline 111 of valve 100, when the sealing member 322 is mounted thereon (see FIGS. 9A-9C). According to some examples, the sealing member 322 is folded by connecting first lateral edge 306 and second lateral edge 308, such that the plurality of protrusions 330 are oriented radially away from the sealing member centerline 311 (see for example, FIG. 8D). According to some examples, the sealing member 322 in a folded state is coupled to the outer surface of the frame 106 of the prosthetic valve 100 so that the plurality of protrusions 330 are oriented to extend radially away from the centerline 111 (see for example, FIG. 9A).

According to some examples, the sealing member 322 further comprises the plurality of inter-protrusion gaps 350, wherein each gap 350 is located (or spaced) between two adjacent protrusions 330. According to further examples, one inter-protrusion gap 350 is formed between the outflow edge 307 and one of the protrusions 330, while another inter-protrusion gaps 350 is formed between the inflow edge 309 and one of the other protrusions 330. The plurality of protrusions 330, and the corresponding plurality of inter-protrusion gaps 350 spacing between each two adjacent protrusions 330, form the 3D shape of the sealing member 322 when in its spread relaxed state, according to some examples. According to some examples, the plurality of inter-protrusion gaps 350 and the protrusions 330 are facing the same direction.

Although the 3D shape of the sealing member 322 is not identical to the 3D shape of the sealing member 222, it is to be understood that sealing member 322 may contain similar materials and/or have similar functionality and uses as those described herein above in conjunction with sealing member 222. According to some examples, unlike the 3D shape of the sealing member 222, the sealing member 322 comprises a flat surface (e.g., a surface 316 or a surface 304) located opposite to the first surface 302, when in its spread relaxed state.

According to some examples, the prosthetic valve 100 comprising the sealing member 322 is configured to be positioned (i.e., implanted) at the target implantation site (e.g., the aortic annulus in the case of aortic valve replacement) so as to form contact between the arterial wall 105 and the plurality of protrusions 330, similar to contact formed between the arterial wall 105 and the plurality of ridges 230 of sealing member 222, as disclosed herein above. Advantageously, the plurality of protrusions 330 of the sealing member 322 are adapted to contact the arterial wall 105 following expansion of the prosthetic heart valve 100 at the site of implantation, and thus to enable a conforming fit or engagement between the prosthetic heart valve 100 and the inner surface of the annular or arterial wall 105, thereby improving PVL sealing around the implanted prosthetic heart valve.

According to some examples, the sealing member 322 is configured to be able to transition from the spread relaxed state to the cylindrical folded state, due to its elastic and/or flexible characteristics, in order to form a cylindrical PVL skirt. A folded PVL skirt 322 may become coupled to outer surface of the frame 106 of the prosthetic valve 100, for example during a procedure of valve assembly. Alternatively, a spread sealing member 322 may be folded around the outer surface of the frame 106 and coupled thereto to achieve a similar product.

According to some examples, the plurality of protrusions 330 extends in different directions from the surface 302, and can form 3D shapes thereon, wherein the 3D shapes can be selected from: inverse U-shape, half-sphere, dome, cylinder, pyramid, triangular prism, pentagonal prism, hexagonal prism, flaps, any other polygon, and combinations thereof. Each possibility represents a different example. According to further examples, the plurality of protrusions 330 extends in different directions from the surface 302, and can form parallel elongated 3D shapes thereon, wherein the elongated 3D shapes can be selected from elongated U-shape, elongated prism, elongated cuboid, any other elongated polyhedron, and combinations thereof. Each possibility represents a different example.

As used herein, the term “elongated 3D shapes” refers to the elongated 3D shapes of the protrusions of the sealing member of the present invention (e.g., protrusions 330), which can be characterized by having various cross-sectional shapes, selected from: inverse U-shape, square, rectangle, any other polygon, and combinations thereof. Each possibility represents a different example.

In FIGS. 8D-8F, the plurality of protrusions 330 can extend in different directions from the surface 302, and can form parallel elongated 3D shapes thereon. The different directions may be vertical, horizontal or diagonal with respect to the centerline 311 of the cylindrically shaped sealing member 322 in its folded state. It is to be understood that the orientation of the protrusions 330 in the folded state of the sealing member 322 may be dictated by their construction prior to the folding, i.e. when the sealing member 322 is in a spread state. According to some examples, the sealing member 322 has a resilient 3D shape, wherein said resilient 3D shape comprise the plurality of protrusions 330 which form an overall wave-like configuration on the surface 302 thereof.

For example, a sealing member 322 has a plurality of protrusions 330, wherein the plurality of protrusions 330 form parallel elongated 3D shapes, and are extending from first lateral edge 306 to second lateral edge 308 (as shown in FIG. 8A), may be folded by connecting first lateral edge 306 to second lateral edge 308 such that a cylindrical shape of the sealing member 322 is formed. In such an exemplary situation, upon said folding, the sealing member 322 in its folded shape will have plurality of circumferentially extending protrusions 330, which are substantially parallel to inflow edge 309 and to outflow edge 307 (as shown in FIG. 8D).

In a second example, a sealing member 322 has a plurality of protrusions 330, wherein the plurality of protrusions 330 form parallel elongated 3D shapes, and are extending from inflow edge 309 to outflow edge 307 (not specifically shown in spread relaxed state). The sealing member 322 may be folded by connecting first lateral edge 306 to second lateral edge 308 such that a cylindrical shape of the sealing member 322 is formed. In such a second exemplary configuration, upon said folding, the sealing member 322 in its folded shape will have plurality of vertically oriented protrusions 330, which are substantially perpendicular to inflow edge 309 and to outflow edge 307 (as shown in FIG. 8E).

Similarly, angled or diagonal protrusions in the spread state will lead to diagonally oriented protrusions in the folded state of the sealing member 322, as shown in FIG. 8F.

As detailed herein, the shape-forming process of creating the protrusions 330 in the sealing member 322 is not limited to be performed prior to the folding, and the protrusions 330 may be formed on the first surface 302 of the sealing member 322 after the folding, according to some examples. Furthermore, the protrusions 330 of the present sealing member 322 are not required to form parallel elongated 3D shapes with respect to each other.

According to some examples, each one of the plurality of protrusions 330 follows a path-line extending from the first lateral edge 306 to the second lateral edge 308 when the sealing member 322 is in a spread state. According to some examples, each one of the plurality of protrusions 330 follows a path-line parallel to any of the first lateral edge 306 and/or the second lateral edge 308 when the sealing member 322 is in a spread state. According to some examples, each one of the plurality of protrusions 330 follows a path-line parallel to any of the outflow edge 307 and/or the inflow edge 309 when the sealing member 322 is in a spread state.

According to some examples, each one of the plurality of protrusions 330 follows a path-line circumferentially extending around the sealing member centerline 311, in a folded state of the sealing member 322 (see FIG. 8D). According to further examples, the plurality of protrusions 330 extend substantially perpendicularly to the sealing member centerline 311, or an axis parallel to the centerline 311, in a folded state of the sealing member 322. According to some examples, each one of the plurality of protrusions 330 follows a path-line circumferentially extending around the centerline 111, substantially perpendicularly to the centerline 111 or an axis parallel to centerline 111, when the sealing member 322 is in a folded state and mounted on the frame 106 of the prosthetic heart valve 100 (see FIG. 9A). According to some examples, each one of the plurality of protrusions 330 follows a path-line parallel to any one of the outflow edge 307 and/or the inflow edge 309, circumferentially around to the sealing member centerline 311, in a folded state of the sealing member 322.

According to some examples, each one of the plurality of protrusions 330 follows a path-line extending from the inflow edge 309 to the outflow edge 307, in a spread state of the sealing member 322. According to some examples, each one of the plurality of protrusions 330 follows a path-line parallel to any one of the first lateral edge 306 and/or to the second lateral edge 308, in a spread state of the sealing member 322. According to some examples, each one of the plurality of protrusions 330 follows a path-line perpendicular to any of the outflow edge 307 and/or the inflow edge 309, in a spread state of the sealing member 322.

According to some examples, each one of the plurality of protrusions 330 follows a path-line extending parallel to the sealing member centerline 311, in a folded state of the sealing member 322 (see FIG. 8E). According to some examples, each one of the plurality of protrusions 330 follows a path-line extending parallel to the centerline 111, when the sealing member 322 is in a folded state and mounted on the frame 106 of the prosthetic heart valve 100 (see FIG. 9B). According to some examples, each one of the plurality of protrusions 330 follows a path-line perpendicular to any one of the outflow edge 307 and/or the inflow edge 309, in a folded state of the sealing member 322.

According to some examples, each one of the plurality of protrusions 330 follows a path-line extending diagonally along the surface of the sealing member 322, in a spread state thereof. According to some examples, each one of the plurality of protrusions 330 follows a path-line extending diagonally along the surface of the sealing member 322 relative to the centerline 111, in a folded state thereof (see FIG. 8F). According to some examples, each one of the plurality of protrusions 330 follows a path-line extending diagonally with respect to the centerline 111, when the sealing member 322 is in a folded state and mounted on the frame 106 of the prosthetic heart valve 100 (see FIG. 9C).

According to some examples, the sealing member 322 comprises the plurality of protrusions 330 extending around and/or away from the first surface 302, wherein each protrusion is in an elongated 3D shape selected from: a half-sphere, a line (e.g., a ridge or a band), a dome, a cube, a cylinder, a pyramid and any other suitable polyhedron. Each possibility represents a different example. According to further examples, each one of the plurality of protrusions 330 forms a 3D shape extending along the surface of the sealing member 322, in a folded state thereof (not shown). According to further examples, each one of the plurality of protrusions 330 forms an elongated 3D shape extending radially around and/or away to the centerline 111 along the surface of the sealing member 322, when the sealing member 322 is in a folded state and mounted on the frame 106 of the prosthetic heart valve 100 (see FIGS. 9A-C). It is to be understood that the term “elongated”, with reference to elevated portions (e.g., ridges 230 or protrusion 330, 430), refers to a shape having a length considerably greater than a width thereof, such that each elevated portions either circumscribes the complete perimeter of the frame 106 when the sealing member is mounted thereon, or extends between an inflow edge and an outflow edge of the sealing member.

Various configurations and orientations as described above may be advantageous for different physiological and implantation-related requirements. For example, the configuration of FIGS. 8D and 9A may be advantageous due to the generally perpendicular orientation of the plurality of protrusions 330 relative to the axial direction of the flow, when the valve 100 is mounted against the annular or arterial wall 105, thereby potentially improving PVL sealing therebetween.

According to some examples, the sealing member 322 comprises a first layer 310. According to some examples, the first layer 310 is flat spread relaxed state of the sealing member 322.

According to some examples, the sealing member 322 comprises a first layer 310 and a second layer 320. According to further examples, said first and second layers 310 and 320, respectively, are disposed externally to the outer surface of the frame 106, when the sealing member 322 is coupled thereto. According to further examples, the sealing member 322 may comprise additional layer(s).

According to some examples, the second layer 320 is in contact with a first surface 315 of the first layer 310 (see FIG. 8B). According to some examples, the second layer 320 is in contact with a first surface 315 of the first layer 310 both in the spread and folded state of the sealing member 322. According to some examples, the second layer 320 is attached to and/or is coating a first surface 315 of the first layer 310. According to some examples, said first surface 315 of the first layer 310 is oriented outward in a folded state of the sealing member 322. According to some examples, said first surface 315 is oriented toward of the implantation site (e.g., the annular or arterial wall 105) when the sealing member 322 is mounted on frame 106 of the prosthetic heart valve 100 and implanted in the implantation site. According to further examples, the second layer 320 is forming a first surface 302 of the sealing member 322, as illustrated at FIG. 8B. According to some examples, the first surface 302 of the sealing member 322 is oriented outward in a folded state of the sealing member 322. According to some examples, the first surface 302 of the sealing member 322 is oriented toward the implantation site when the sealing member 322 is mounted on the frame 106 of the prosthetic heart valve 100 and implanted at the implantation site.

According to some examples, the plurality of protrusions 330 are extending away from the second layer 320 of the sealing member 322 and are spaced from each other therealong, wherein the second layer 320 is attached to and/or is coating the first surface 315 of the first layer 310.

Without wishing to be bound by any theory or mechanism of action, various sealing members 322 as disclosed herein assume a three-dimensional shape, which may be a result of a thermal shape-processing procedure. Such a procedure is enabled or facilitated by the employment of thermoplastic materials, which can be shaped at elevated temperature as detailed herein. To enable thermoplastic materials to be molded or shaped into a desired structure with thin sheet-like objects, it is advantageous that the thermoplastic materials constitute or cover the objects. This may be achieved, e.g., utilizing coating with a thermoplastic coating layer or forming the object with a thermoplastic layer. Although one thermoplastic layer may be sufficient for enabling the shape-forming process, it may be advantageous, according to some examples, to include a plurality of thermoplastic layers, such as two layers. Specifically, a configuration in which the two external layers of the sealing member 322 include a thermoplastic material may be advantageous.

According to some examples, the sealing member 322 comprises a third layer 325. According to some examples, the third layer 325 is in contact with a second surface 316 of the first layer 310 (see FIG. 8C). According to some examples, the third layer 325 is in contact with a second surface 316 of the first layer 310 both in the spread and folded state of the sealing member 322. According to some examples, the third layer 325 is attached to and/or is coating a second surface 316 of the first layer 310. According to some examples, said second surface 316 of the first layer 310 is oriented inward in a folded state of the sealing member 322.

According to some examples, the second surface 316 is oriented in the direction opposite to the implantation site (e.g., the arterial wall 105) when the sealing member 322 is mounted on the frame 106 of the prosthetic heart valve 100 and implanted at the implantation site. According to further examples, the third layer 325 is defines a second surface 304 of the sealing member 222, as illustrated at FIG. 8C. According to some examples, the second surface 304 of the sealing member 322 is oriented inward in a folded state of the sealing member 322. According to some examples, the second surface 304 of the sealing member 322 is oriented in the direction opposite to the anatomical wall at the implantation site when the sealing member 322 is mounted on the frame 106 of the prosthetic heart valve 100 and implanted at the implantation site.

According to some examples, the second surface 304 of the sealing member 322 is a flat surface (see FIG. 8C). According to other examples, the second surface 304 of the sealing member 322 comprises a plurality of additional protrusions 330 (not shown).

According to some examples, sealing member 322 comprises both the second layer 320 and the third layer 325. According to some examples, the second layer 320 is connected to the third layer 325. According to some examples, the second layer 320 and the third layer 325 are unified to cover the first layer 310, as illustrated in FIG. 8C. According to some examples, the second layer 320 and the third layer 325 collectively form a coating which covers both the first and second surfaces 302 and 304, respectively, of the sealing member 322. According to some examples, the second layer 320 and the third layer 325 collectively form a coating which covers the sealing member 322.

It is to be understood, based on the above, that the spread sealing member 322 may be folded to its folded state by connecting its first lateral edge 306 and its second lateral edge 308, over the second surface 304 thereof, such that in a folded state of the sealing member 322, its second surface 304 faces inwardly toward the sealing member centerline 311, and its first surface 302 faces outwardly, according to some examples. Therefore, when the folded sealing member 322 is mounted on the frame 106 of the prosthetic heart valve 100 and implanted at the implantation site, the second layer 320 and the plurality of protrusions 330 which are extending away therefrom are in contact with the anatomical wall at the implantation site (e.g., the inner surface of the annular or arterial wall 105).

According to some examples, the sealing member 322 extends between the first surface 302 and the second surface 304, wherein the sealing member 322 has a total layer thickness 303 measured between the first surface 302 and the second surface 304 at one of the inter-protrusion gaps 350, as illustrated at FIG. 8C. According to some examples, said total layer thickness 303 is measured from the first surface 302 of the sealing member 322 to the second surface 316 of the first layer 310 (not shown). According to some examples, the total layer thickness 303 is measured from the first surface 302 of the sealing member 322 (e.g., the second layer 320) to the second surface 304 (e.g., the third layer 325), as shown in FIG. 8C.

According to some examples, the thickness 322T of sealing member 322 (defined as the distance between the sealing member's protrusions 330 and its second surface 304) is at least 1000% greater than the total layer thickness 303. In further examples, the thickness 322T is at least 2000%, at least 3000%, at least 4000%, at least 5000%, or at least 6000% greater than the total layer thickness 303 of the sealing member 322. Each possibility represents a different example. In still further examples, the thickness 322T is no greater than 6000%, 7000%, 8000%, 9000%, 10,000%, 20,000%, 30,000%, 40,000% or 50,000% compared to the total layer thickness 303 of the sealing member 322. Each possibility represents a different example.

It is to be understood that the thickness ratio between thickness 322T and total layer thickness 303 in FIGS. 8B-C is moderate, whereas, as described above, the actual ratio is greater (e.g. the thickness 322T is 10-60 times greater than the total layer thickness 303). For example, in some non-binding implementations, the total layer thickness 303 can be in the range of 0.02 to 0.1 mm, while the thickness 322T can be in the range of 0.5-3 mm.

According to some examples, the 3D shape in the spread relaxed state of the sealing member 322 comprises protrusions 330, each having a protrusion height 322PH, being a part of the thickness 322T thereof. In further examples, each protrusion height 322PH and the total layer thickness 303 together define the thickness 322T of sealing member 322.

According to some examples, the sealing member 322 has a resilient 3D structure such that the nonfibrous outer surface 380 of the sealing member 322 exhibits the plurality of elevated portions 330 with peaks 305 and the plurality of non-elevated portions 350, as disclosed herein above (see for example FIGS. 8B-C). According to some examples, the nonfibrous outer surface 380 of the sealing member 322 is defined as an outer surface combining the first surface 302 and an outer surface of each one of the plurality of elevated portions 330 (i.e., protrusions 330). According to some examples, the peaks 305 are defined as the highest point along the outer surface of each one of the plurality of elevated portions 330, extending away from the first surface 302 of the sealing member 322. According to some examples, the height of each peak 305 is defined as the distance of the highest point along the outer surface of each one of the plurality of elevated portions 330, relative to the frame 106, when the sealing member 322 is coupled to the outer surface of the frame 106 of the prosthetic valve 100 (e.g., the thickness 322T).

According to some examples, the non-elevated portions 350 are defined as the inter-protrusion gaps 350. In further such examples, the height of each non-elevated portion 350 is defined as the distance of the first surface 302 relative to the frame 106, when the sealing member 322 is coupled to the outer surface of the frame 106 of the prosthetic valve 100 (e.g., the total layer thickness 303). According to some examples, the distance of the peaks 305 from the frame 106 is at least 1000% greater than the distance of the non-elevated portions 350 from the frame 106, in the absence of an external force applied to press the elevated portions 330 against the frame. According to further examples, the distance of the peaks 305 from the frame 106 is at least 1500%, at least 2000%, at least 3000%, at least 4000%, at least 5000%, or at least 6000% greater than the distance of the non-elevated portions 350 therefrom. Each possibility represents a different example.

It is to be understood that any reference to the thickness 322T of sealing member 322 is equivalent to the distance of the peaks 305 of the elevated portions 330 from the external surface of the frame 106, in a relaxed state of the sealing member 322 when coupled to the frame 106. Similarly, any reference to the total layer thickness 303 is equivalent to the distance of the non-elevated portions 350 from the external surface of the frame 106, when the sealing member 322 is coupled thereto.

According to some examples, the first layer 310 comprises the same materials as the first layer 210, as described herein above. According to some examples, the first layer 310 is made from a flexible and/or elastic material(s) adapted to provide mechanical stability, and optionally tear resistance (or tear strength), to the sealing member 322. In further examples, the first layer 310 is configured to enable the continuous durable attachment of the sealing member 322 to the outer surface of the frame 106 of the prosthetic valve 100, optionally by preventing the formation of irreversible deformation thereto (e.g., resist tearing), thus providing mechanical stability to the structure during utilization thereof.

The first layer 310 can comprise, for example, various woven biocompatible textiles, comprising materials such as various synthetic materials (e.g., polyethylene terephthalate (PET), polyester, polyamide (e.g., Nylon), polypropylene, polyetheretherketone (PEEK), polytetrafluoroethylene (PTFE), etc.), natural tissue and/or fibers (e.g. bovine pericardium, silk, cotton, etc.), metals (e.g., a metal mesh or braid comprising gold, stainless steel, titanium, nickel, nickel titanium (Nitinol), etc.), and combinations thereof. Each possibility represents a different example. The first layer 310 can be a metallic or polymeric member, such as a shape memory metallic or polymeric member. The first layer 310 can be a woven textile. It is to be understood that the first layer 310 is not limited to a woven textile. Other textile constructions, such as knitted textiles, braided textiles, fabric webs, fabric felts, filament spun textiles, and the like, can be used. The textiles of first layer 310 can comprise at least one suitable material, selected from various synthetic materials, natural tissue and/or fibers, metals, and combinations thereof, as described herein above.

According to some examples, the first layer 310 comprises at least one tear resistant material, wherein the tear resistant material optionally comprises a PET fabric, and wherein the tear resistant material is configured to provide mechanical stability and tear resistance and support the structure thereof, similar to the properties and characteristics of the first layer 210, as described herein above. According to further examples, the first layer 310 comprises a tear resistant PET fabric. According to further examples, the first layer 310 comprises at least one tear resistant knit/woven PET fabric.

According to some examples, the first layer 310 comprises at least one tear resistant and flexible material, which is able to withstand loads of above about 3N of force before tearing, thereby enabling the sealing member 322 to reliably operate without tearing during regular use thereof. According to further examples, the at least one tear resistant and flexible material of the first layer 310 is able to withstand loads of above about 5N, 7N, 10N, 15N, 20N, 25N, 30N, or more, of force before tearing. Each possibility represents a different example. According to still further examples, the at least one tear resistant and flexible material of the first layer 310 is able to withstand loads of above about 20N of force before tearing. According to yet still further examples, the at least one tear resistant and flexible material of the first layer 310 is able to withstand loads of above about 30N of force before tearing. According to a preferred example, the at least one tear resistant and flexible material of the first layer 310 comprises a PET fabric and is able to withstand loads of at least 20N of force before tearing. According to some examples, the flexible material tear resistant material is able to withstand loads in the range of 15N to 500N. According to some examples, the flexible material tear resistant material is able to withstand loads in the range of 20N to 500N.

According to some examples, the first layer 310 is made from at least one biocompatible material, as disclosed herein above.

It is to be understood that when the first layer 310 is covered by the second layer 320 and third layer 325, as shown in FIG. 8C, it should not come in contact with tissues when implanted, and thus, in this case first layer 310 may be made of non-biocompatible materials. Nevertheless, it may be preferable to form the first layer 310 from biocompatible materials in such cases as well, to prevent risks of abrasive damage or tears of any of the second layer 320 or third layer 325, which may in turn expose portions of the first layer 310.

According to some examples, at least one of the second layer 320, the third layer 325, and the plurality of protrusions 330, comprises the same materials as the second layer 220, as described herein above. According to some examples, the second layer 320 and the plurality of protrusions 330 are adapted to contact the implantation site tissue (i.e., the inner surface of the annular or arterial wall 105) and therefore are made from at least one elastic biocompatible material. Furthermore, it may be advantageous for the second layer 320 and the plurality of protrusions 330 to be made of materials that may prevent/resist and/or reduce the extent of tissue ingrowth around or over the sealing member 322, according to some examples, such that if and when an explant procedure is required, the valve 100 can be easily removed from the site of implantation, as detailed above.

According to some examples, the first surface 302 of the sealing member 322 (i.e., the second layer 320) is characterized by having a smooth and/or a low-friction surface, adapted to reduce friction with tissue of the implantation site, thereby reducing tissue ingrowth thereon and enabling easier removal of the previously implanted valve from the site of implantation. According to some examples, each one of the plurality of protrusions 330 are characterized by having a smooth and/or a low-friction outer surface, adapted to reduce friction with tissue of the implantation site, for the reasons described herein above. According to some examples, the second layer 320 and/or each one of the plurality of protrusions 330 may comprise silicone or other lubricious materials or polymers that could assist in explant procedures for removal of the prosthetic valve from its site of implantation.

According to some examples, the second layer 320 and/or the plurality of protrusions 330 are continuous in a manner which is devoid of yarns and/or strands (including texturized yarns or strands). According to further examples, the plurality of protrusions 330 are devoid of discontinuities that may extend along the entire width thereof.

According to some examples, the second layer 320 and the plurality of protrusions 330 (and optionally the third layer 325) can be made of various suitable biocompatible synthetic materials, such as, but not limited to, a thermoplastic material. According to some examples, the thermoplastic material is selected from the group consisting of: polyamides, polyesters, polyethers, polyurethanes, polyolefins, polytetrafluoroethylenes, and combinations and copolymers thereof. According to some examples, the second layer 320 and the plurality of protrusions 330 (and optionally the third layer 325) can be made of various suitable biocompatible synthetic materials, such as, but not limited to, thermoplastic material, including thermoplastic elastomers (TPE). According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example.

According to some examples, at least one of the second layer 320, the third layer 325, and the plurality of protrusions 330 comprises at least one thermoplastic thromboresistant material, wherein the thermoplastic thromboresistant material comprises at least one thermoplastic elastomer, optionally comprising TPU. According to further examples, the second layer 320 and the plurality of protrusions 330 are configured to form the 3D shape of the sealing member 322 in a folded cylindrical state, which is adapted to enhanced PVL sealing between the prosthetic heart valve 100 and the inner surface of the annular or arterial wall 105, and optionally prevent and/or reduce tissue ingrowth thereover. According to some examples, the second layer 320, the third layer 325, and the plurality of protrusions 330, comprise TPU.

The third layer 325, when incorporated into the sealing member 322, may be united with the second layer 320 as detailed herein, according to some examples. When the third and second layers 325 and 320, respectively, are formed as a united coating covering the first layer 310, they may be preferably made of the same material, according to some examples. Even if the third and second layers 325 and 320, respectively, are separated, according to some examples, they may have similar or the same composition. According to some examples, the third and second layers 325 and 320, respectively, are made of the same material.

According to some examples, each one of the plurality of protrusions 330 is made from a full (i.e., non-hollow) material/object, comprising the at least one thermoplastic thromboresistant material as described herein above, wherein the thermoplastic thromboresistant material optionally comprises TPU. According to further examples, each one of the plurality of protrusions 330 is not hollow, and is made entirely from the at least one thermoplastic thromboresistant material as described herein above, wherein the thermoplastic thromboresistant material optionally comprises TPU. According to some examples, each one of the plurality of protrusions 330 defines a non-hollow structure.

According to some examples, the plurality of protrusions 330 and the plurality of inter-protrusion gaps 350 spacing between adjacent protrusions 330 along the second layer 320 are configured to contact the implantation site (i.e., the inner surface of the annular or arterial wall 105). According to some examples, the plurality of protrusions 330 are made from the same material(s) as the second layer 320, and therefore are made from the same elastic biocompatible material(s), adapted to prevent/resist and/or reduce tissue ingrowth around the sealing member 322, such that when an explant procedure is required, the valve 100 can be easily removed from the site of implantation.

According to some examples, the sealing member 322 comprises the first layer 310, the second layer 320, the plurality of protrusions 330 extending away from the second layer 320 that coats at least the first surface 302 thereof, and optionally the third layer 325, wherein the first layer 310 is configured to provide mechanical stability and tear resistance and support the structure thereof, while the second layer 320 and plurality of protrusions 330 (and optionally the third layer 325) are configured to form and maintain the resilient 3D shape thereof, wherein the second layer 320 and the plurality of protrusions 330 are optionally configured to prevent and/or reduce tissue ingrowth thereover.

It is contemplated that the second layer 320, on its own, lacks the ability to support the structure of the sealing member 322, is unable to maintain a successful attachment thereof to the outer surface of the frame 106, and optionally has low tear resistance. Advantageously, the combination between the first layer 310, the second layer 320 alone or together with the optional third layer 325, and the plurality of protrusions 330, provides the required features of the sealing member 322. According to some examples, the second layer 320 comprising TPU, either alone or together with the optional third layer 325, and the plurality of protrusions 330, are reinforced by the first layer 310 comprising PET to provide the strength required to retain the sutures.

It is contemplated that the utilization of thermoplastic elastomer material(s), such as TPU, as a layer of sealing member 322 and/or a component within the plurality of protrusions 330, enables formation of a desired 3D-shaped sealing member 322 having a plurality of elastic resilient protrusions 330. In some examples, advantageously, the plurality of elastic protrusions 330 of the sealing member 322 are adapted to contact, and become compressed against, the annular or arterial wall 105 at the implantation site, following expansion of the prosthetic heart valve 100 therein, so as to improve PVL sealing between the prosthetic heart valve 100 and the inner surface of the annular or arterial wall 105. Thus, according to some examples, each one of the plurality of protrusions 330 is elastic and resiliently compressible. The elastic and resilient compressibility characteristics of the plurality of protrusions 330 can potentially improve retention of the sealing member 322 against the surrounding tissues of the native heart valve at the implantation site.

According to some examples, the sealing member 322 has a resilient 3D shape, wherein said resilient 3D shape is configured to deform when an external force is applied thereto (e.g., when compressed against the annular or arterial wall 105, or when pressed against an inner wall of a sheath or a capsule), and further configured to revert to its original shape (i.e., the shape of its relaxed state) when the external force is no longer is applied thereto (e.g., when a valve is released from the shaft or capsule prior to expansion thereof).

It is to be understood that the compressibility of the protrusions 330 does not contradict the resilient 3D structure of the second layer 320, on which the protrusions 330 are connected, as upon the ceasing of compression on the protrusions 330 (e.g. if the sealing member 322 reverts back to a relaxed state), the protrusions 330 structure of the second layer 320 will be reinstated.

According to some examples, the sealing member 322 comprises at least the first layer 310 comprising a tear resistant material, the second layer 320 that coats at least the first surface 302 and comprises a thermoplastic thromboresistant material, and the plurality of protrusions 330 extending away from the second layer 320. According to some examples, the sealing member 322 further comprises the third layer 325 comprising a thermoplastic thromboresistant material. According to further examples, the sealing member 322 comprises the first layer 310 comprising a tear resistance material comprising a PET fabric, and the second layer 320 comprising the plurality of protrusions 330 extending therefrom comprising thermoplastic thromboresistant material comprising TPU. According to further examples, the sealing member 322 comprises the third layer 325 comprising a thermoplastic thromboresistant material comprising TPU.

Reference is now made to FIGS. 10A-10C, illustrating processing steps utilizing extrusion for the fabrication of the sealing member 322, according to some examples.

According to some examples, there is provided a PVL skirt 322 prepared by the methods of the present invention. According to some examples, there is provided a PVL skirt 322 in a folded state prepared by the methods of the present invention.

According to some examples, there is provided a method of fabricating a sealing member, such as the sealing member 322 described herein above, in a cost-effective and simple manner. According to some examples, the method comprises: (i) providing a tear resistant flat sheet 312; (ii) treating the sheet in a thermal shape-forming process to assume a 3D shape in a spread relaxed state; and (iii) connecting two opposite edges of the sheet 312 of step (ii) to form a cylindrical sealing member (or PVL skirt) in a cylindrical folded state.

According to some examples, step (i) comprises providing a tear resistant flat sheet 312 comprising the first layer 310, which comprises at least one tear resistant material as described herein above, wherein the tear resistant material optionally comprises a PET fabric.

According to some examples, step (i) comprises providing a flat flexible sheet 312, which comprises a tear resistant first layer 310 and a thermoplastic second layer 320. According to some examples, step (i) comprises providing a flat flexible sheet 312, which comprises a tear resistant first layer 310 disposed between a thermoplastic second layer 320 and a thermoplastic third layer 325 of the flat flexible sheet 312 (see FIG. 10A).

According to some examples, step (i) comprises providing a flat flexible sheet 312, which comprises a tear resistant first layer 310, and coating at least a first surface 315 of the first layer 310 with a thermoplastic coating, thereby forming the thermoplastic second layer 320. According to some examples, step (i) comprises providing a flat flexible sheet 312, which comprises a tear resistant first layer 310, and coating a first surface 315 and a second surface 316 of the first layer 310 with a thermoplastic coating, thereby forming the thermoplastic second and third layers 320 and 325, respectively.

The coating of the tear resistant first layer 310 can be performed by a coating technique selected from the group consisting of brushing, spray-coating, dip coating, dipping or immersing, and combinations thereof. The present method, however, is not limited to such coating techniques, and other coating techniques, such as chemical deposition, vapor deposition, chemical vapor deposition, physical vapor deposition, printing and the like, may be suitably used, according to some examples. Such techniques are generally suitable for medical textiles. Moreover, printing techniques, such as roller printing, stencil printing, screen printing, inkjet printing, lithographic printing, 3D printing, and the like, may be also used with the present invention for applying the thermoplastic polymeric coating.

The thermoplastic coating can comprise the same materials as the materials forming the second layer 320. The thermoplastic coating can comprise thermoplastic materials such as polyamides, polyesters, polyethers, polyurethanes, polyolefins, polytetrafluoroethylenes, and combinations and copolymers thereof. The thermoplastic coating, comprising the thermoplastic material, can comprise a thermoplastic elastomer material such as thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example. The thermoplastic coating can comprise TPU. The thermoplastic coating can comprise biocompatible thromboresistant materials as disclosed herein.

It is to be understood that of the properties introduced above for each one of the layers (i.e., the first layer 310, the second layer 320 and the third layer 325) similarly apply to the respective layers when referring to the methods of fabricating the sealing member. According to some examples, the first layer 310 comprises a tear resistant PET fabric. According to some examples, the second layer 320, the third layer 325, or both, comprises at least one thermoplastic material. According to some examples, the second layer 320, the third layer 325, or both, comprises at least one thromboresistant thermoplastic elastomer material comprising TPU. According to some examples, the second layer 320 and the third layer 325 are made from the same material. According to some examples, the third layer 325 is united with the second layer 320 as detailed herein.

According to some examples, step (ii) of treating the sheet in a thermal shape-forming process to assume a 3D shape in a spread relaxed state, entails an extrusion-based shape-forming process, which comprises extruding a plurality of members 331 on the surface 302 of the second layer 320 of the sheet 312. According to some examples, each member 331 comprises a molten composition comprising a thermoplastic material (optionally thromboresistant). In further examples, each member 331 is extruded utilizing an extruder comprising an extruder die 332 (see FIG. 10B). In further examples, each member 331 is an elongated member 331, which can extend from at least one of outflow edge 307 toward inflow edge 309 or first lateral edge 306 toward second lateral edge 308.

The term “extrusion” or “extruding”, as used herein, refers to a process of forcing a molten composition through a die orifice having a desired cross sectional shape corresponding to the desired shape of the extruded members 331. Said process of forcing the molten composition through the die orifice is performed under pressure and under heat. The extrusion of the thermoplastic thromboresistant material can be performed by 3D printing, wherein the die orifice is a movable printer extruder head.

The molten composition can comprise thermoplastic materials such as polyamides, polyesters, polyethers, polyurethanes, polyolefins, polytetrafluoroethylenes, and combinations and copolymers thereof. The molten composition, comprising the thermoplastic material, can comprise a thermoplastic elastomer material such as thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example. The molten composition can comprise TPU. The molten composition can comprise biocompatible thromboresistant materials as disclosed herein above.

The molten composition can further comprise various adhesives or additives, configured to enhance the attachment between the extruded composition and the surface 302 of the second layer 320 of the sheet.

The molten composition can be extruded at an elevated temperature. The elevated temperature is a temperature sufficient to enable the molten composition to be processed to a flowing molten state and extruded under pressure thought the extruder die 332, in order to become formed over and attached to, the surface 302 of the second layer 320 of the sheet 312. According to some examples, the elevated temperature in step (ii) is above about 100° C., 125° C., 150° C., 175° C., 200° C., 225° C., 250° C., 275° C., 300° C., or more. Each possibility represents a different example.

According to some examples, step (ii) comprises extruding the plurality of members 331 on the thermoplastic second layer 320 of the flat flexible sheet 312, so that each extruded member 331 extends at least from the first lateral edge 306 to the second lateral edge 308 of the sheet 312, thereby forming a plurality of 3D shapes on the sheet, configured to transition to the configuration of protrusions 330 of the sealing member 322 illustrated in FIG. 8D. According to some examples, step (ii) comprises extruding the plurality of members 331 on the thermoplastic second layer 320 of the flat flexible sheet 312, so that each extruded member 331 extends from the inflow edge 309 to the outflow edge 307 of the sheet 312, thereby forming a plurality of 3D shapes on the sheet, configured to transition to the configuration of protrusions 330 of the sealing member 322 illustrated in FIG. 8E.

According to some examples, step (ii) comprises extruding the plurality of members 331 on the thermoplastic second layer 320 of the flat flexible sheet 312, so that each extruded member 331 extends diagonally along at least a portion of the second layer 320 of the flat flexible sheet 312, thereby forming a plurality of 3D shapes on the sheet, configured to transition to the configuration of protrusions 330 of the sealing member 322 illustrated in FIG. 8F.

After extruding the elongated members 331, each comprising the molten composition, on the surface 302 of the second layer 320 of the sheet, thereby forming a 3D shape on the sheet, the 3D shape-formed sheet can be cooled, thereby stabilizing the 3D shape in the spread relaxed state of the sealing member. While cooling the 3D shaped sheet, the molten composition transitions to a semi-rigid or resilient relatively rigid state, wherein the shape of the extruded elongated members 331 can transition to assume the shape of the plurality of the protrusions 330 (see FIG. 10C). This configuration may be prompted by gravitation and/or assisted by external force, according to some examples. According to some examples, step (ii) further comprises cooling (i.e., lowering the temperature of) the sheet 312 to a temperature below 40° C. According to further examples, the lowering of the temperature in step (ii) is cooling the sheet 312 to room temperature.

According to some examples, after cooling, each extruded elongated member 331 may transition to a semi-rigid or resilient relatively rigid state, resulting in the formation of the configuration of the plurality of the protrusions 330 of the sealing member 322 as illustrated in FIGS. 9A-9C.

It is to be understood that the thermoplastic properties of each one of the plurality of the protrusions 330 enable the extrusion-based shape-forming process described above to be performed. Specifically, thermoplastic materials are converted from a resilient relatively rigid state at lower temperatures, to a pliable relatively soft state when heated and/or a flowing molten state under extrusion conditions. In step (ii), the thermoplastic molten composition is heated under pressure within the extruder to its molten state, according to some examples, thereby allowing the extruded plurality of the elongated members 331 to assume a 3D shape comprising protrusions 330, following cooling and transformation thereof to the resilient relatively rigid state.

Specifically, in the example illustrated in FIGS. 10B-10C, a plurality of elongated members 331 comprising the thermoplastic molten composition are extruded on the surface 302 of the second layer 320 of the sheet 312, wherein the thermoplastic molten composition is in the flowing molten state at an elevated temperature as disclosed above. According to some examples, in step (ii), each elongated member 331 is extruded on the surface 302 of the second layer 320, thereby forming a 3D shape on the sheet. After assuming the desired 3D shape, the sheet 312 may be allowed to cool, so that the thermoplastic molten composition reverts back to its resilient non-pliable state, thereby transitioning to the shape of the plurality of the protrusions 330 and stabilizing the seal member 322 in its spread state, according to some examples (FIG. 10C).

According to some alternative examples, step (ii) of treating the sheet in a thermal shape-forming process to assume a 3D shape in a spread relaxed state entails an injection molding process, including inserting the flat flexible sheet 312 into a mold (not shown), and adding/injecting a molten composition comprising the thermoplastic thromboresistant material as described herein above into said mold, wherein the molten composition conforms to the shape of the mold, on top of at least one surface of the flat flexible sheet 312. The molten composition can be molded at an elevated temperature, as described herein above. The molding process where the thermoplastic thromboresistant material is formed into the desired 3D shape on top of at least one surface of the coated sheet comprising the plurality of the protrusions 330 thereon, can be performed, for example, by injection molding. After the thermoplastic thromboresistant material forms the desired 3D shape inside the mold on top of at least one surface of the sheet 312, the formed 3D molded coated sheet can be cooled and removed from the mold, thereby stabilizing the 3D shape in the spread relaxed state of the sealing member 322.

According to some examples, the sheet 312 of step (i) has a first surface 302 and a second surface 304, wherein the distance between the first surface 302 and a second surface 304 of the sheet 312 of step (i) constitutes the initial thickness 312T of the sheet 312 of step (i) (see FIG. 10A). According to some examples, the sheet 312 of step (i) is flat and substantially two dimensional. This means that the initial thickness 312T of the sheet 312 of step (i) is substantially shorter that an initial width and/or an initial length of the sheet 312. According to some examples, the initial thickness 312T corresponds to, or is identical to, the total layer thickness 303, as described above.

According to some examples, upon performing the method of the present invention, protrusions 330 are formed, wherein the protrusions 330 have protrusion height 322PH, being a part of the thickness 322T of sealing member 322 in its spread relaxed state (see FIG. 10C).

According to some examples, the thickness 322T of sealing member 322 in its spread relaxed state, following the formation of the plurality of protrusions 330 at step (ii), is configured to assume the 3D shape thereof, and is at least 1000% greater than the initial thickness 312T of the sheet 312. According to further examples, the thickness 322T of sealing member 322 in its spread relaxed state is at least 2000%, at least 3000%, at least 4000%, at least 5000%, or at least 6000% greater than the initial thickness 312T of the sheet 312. Each possibility represents a different example.

It is to be understood that any reference to the thickness 312T of sealing member 322 is equivalent to the distance of the peaks 305 from the external surface of the frame 106, in a relaxed state of the sealing member 322 when coupled to the frame 106. Similarly, any reference to the initial thickness 312T of the sheet 312 is equivalent to the distance of the non-elevated portions 350 from the external surface of the frame 106, when the sealing member 322 is coupled thereto.

According to some examples, the thickness modification of the sheet 312 following the method as described herein (312T to 322T) is configured to convert the initial 2D structure of the sheet 312 to a 3D structure in sealing member 322. In some implementations, the resulting sheet 312 after step (ii) has dimensions that are greater than any of a desired final width and/or length, and the method can include an additional step of cutting the sheet 312 to a desired final width and/or length, after step (ii) and prior to step (iii).

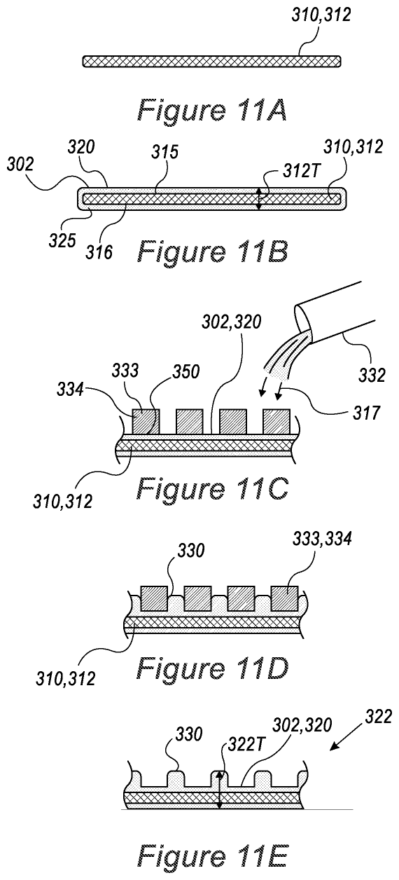

Reference is now made to FIGS. 11A-11E, showing processing steps utilizing a plurality of masking elements 333, for the fabrication of the sealing member 322, according to some examples.

According to some examples, there is provided a method of fabricating the sealing member 322 as described herein above, in a cost-effective and simple manner, the method comprising: (i) providing a tear resistant flat sheet 312 comprising the first layer 310 that comprises at least one tear resistant material, wherein the tear resistant material optionally comprises a PET fabric (FIG. 11A), and coating at least one surface of the flat tear resistant sheet with a thermoplastic polymeric coating layer, thereby forming the second layer 320 thereon; (ii) treating the sheet in a thermal shape-forming process to assume a 3D shape in a spread relaxed state utilizing a mold 334 and unevenly depositing a thermoplastic material on the second layer 320, and (iii) connecting two opposite edges of the sheet of step (ii) to form a cylindrical sealing member (or PVL skirt) in a cylindrical folded state.

According to some examples, step (i) comprises coating a first surface 315 and a second surface 316 of the first layer 310 with a thermoplastic coating as specified herein above, thereby forming the thermoplastic second and third layers 320 and 325, respectively, on opposite surfaces of the flat sheet 312 (FIG. 11B).

According to some examples, step (i) of coating at least one surface of the flat tear resistant sheet with a thermoplastic polymeric coating layer can be performed utilizing at least one coating technique, as described herein above.

It is to be understood that any of the properties introduced above for each one of the layers (i.e. the first layer 310, the second layer 320 and the third layer 325) similarly apply to the respective layers when referring to the method for fabrication of a sealing member 322 of the present invention.

According to some alternative examples, step (i) entails providing a pre-prepared tear resistant flat sheet 312, comprising the tear resistant first layer 310, the thermoplastic second layer 320, and optionally the thermoplastic third layer 325. According to further such examples, the first layer 310 comprises a PET fabric and the second and/or third layers 320 and 325, respectively, comprises TPU.

According to some examples, step (ii) entails placing a mold 334 comprising a plurality of masking elements 333 spaced apart from each other on the sheet 312, and depositing a thermoplastic material in the spaces formed between adjacent masking elements 333. According to some examples, step (ii) entails placing a mold 334 comprising a plurality of masking elements 333 spaced apart from each other on the surface 302 of the second layer 320 of the sheet 312 and depositing a thermoplastic material in the spaces formed between adjacent masking elements 333.

According to some examples, step (ii) entails providing a mold 334 comprising a plurality of masking elements 333 spaced apart from each other and depositing the plurality of masking elements 333 on the surface 302 of the second layer 320 of the sheet 312 (see FIG. 11C). According to some examples, step (ii) of treating the sheet to assume a 3D shape in a spread relaxed state initially entails providing a mold 334 comprising a plurality of masking elements 333; depositing the plurality of masking elements 333 on the surface 302 of the second layer 320 of the sheet 312 and spacing them apart from each other thereover, wherein each one of the plurality of masking elements 333 is over a corresponding inter-protrusions gap 350 (see FIG. 11C). The masking elements 333 may be equally spaced apart from each other, according to some examples.

According to some examples, step (ii) comprises placing the plurality of masking elements 333 on the surface 302 of the second layer 320 of the sheet 312, so that each masking element 333 extends from the first lateral edge 306 to the second lateral edge 308 of the sheet 312. According to some examples, step (ii) comprises placing the plurality of masking elements 333 on the surface 302 of the second layer 320 of the sheet 312, so that each masking element 333 extends from the inflow edge 309 to the outflow edge 307 of the sheet 312. According to some examples, step (ii) comprises placing the plurality of masking elements 333 on the surface 302 of the second layer 320 of the sheet 312, so that each masking element 333 extends diagonally along at least a portion of the second layer 320 of the flat flexible sheet 312.

According to some examples, step (ii) further comprises depositing a thermoplastic material in the spaces formed between adjacent masking elements 333, wherein the deposition of the thermoplastic material is performed by a technique selected from the group consisting of extrusion, brushing, spray-coating, chemical deposition, liquid deposition, vapor deposition, chemical vapor deposition, physical vapor deposition, roller printing, stencil printing, screen printing, inkjet printing, lithographic printing, 3D printing, and combinations thereof. Each possibility represents a different example.

According to some examples, step (ii) further comprises depositing a thermoplastic material in the spaces formed between adjacent masking elements 333, on the surface 302 of the second layer 320 of the sheet 312, at an elevated temperature. The thermoplastic material can comprise a thermoplastic elastomer material such as TPU, which is optionally also thromboresistant, as disclosed herein above.

According to some examples, depositing a thermoplastic material entails depositing a plurality of thermoplastic coating layers, wherein each thermoplastic coating layer can comprise thermoplastic coating as specified herein above. The plurality of thermoplastic coating layers are configured to transition to a semi-solid or solid state, thereby forming the plurality of the protrusions 330, following the deposition thereof. The deposition of the plurality of thermoplastic coating layers can be performed by a coating technique selected from brushing, spray-coating, chemical deposition, liquid deposition, vapor deposition, chemical vapor deposition, physical vapor deposition, roller printing, stencil printing, screen printing, inkjet printing, lithographic printing, 3D printing, and combinations thereof. Each possibility represents a different example. The deposition of the plurality of thermoplastic coating layers can be performed by liquid deposition.

According to some examples, step (ii) comprises depositing a molten composition comprising a thermoplastic thromboresistant material (e.g., TPU) at an elevated temperature, as described herein above (liquid deposition). According to some examples, the deposition is performed in the spaces formed between adjacent masking elements 333 (see FIG. 11C). According to some examples, step (ii) further comprises cooling the masking elements 333 and/or the disposed molten composition after the deposition.

It is to be understood that in such cooling conditions, the molten composition transitions to a semi-solid or solid state, thereby shape-forming the plurality of the protrusions 330, such that each one of the plurality of the protrusions 330 is disposed between adjacent masking elements 333 (see FIG. 11D). According to further examples, the molten composition is extruded in the direction of arrows 317 utilizing an extruder equipped with an extruder die 332 (see FIG. 11C) to form the plurality of the protrusions 330, wherein each one of the plurality of the protrusions 330 is disposed between adjacent masking elements 333.

According to some alternative examples, step (ii) comprises depositing a monomer composition in the spaces formed between adjacent masking elements 333, and polymerizing the composition in order to cause it to transition to a solid or semi solid state, thereby forming the plurality of the protrusions 330. According to some examples, each one of the plurality of the protrusions 330 is disposed between adjacent masking elements 333. According to some examples, the polymerization is initiated using a chemical initiator, thermal initiation, irradiation etc. Each possibility represents a separate example.

According to some examples, the protrusions 330 are formed of a thermoplastic elastomer. According to some examples, the thermoplastic elastomer is polyurethane. It is to be understood that polyurethane can be prepared from a reaction between a polyol (e.g. a diol, a triol and higher poly-alcohols) and a polyisocyanate (e.g. a diisocyanate, a triisocyanate and higher poly-isocyanates). Thus, according to some examples, the monomer composition comprises at least one of a polyol and a polyisocyanate, and according to some examples, polymerizing the composition entails contacting the monomer composition with a second monomer composition comprising the other monomer (polyol or polyisocyanate).

According to some alternative examples, step (iii) further comprises removing the plurality of masking elements 333 from the surface 302 of the sheet, thereby forming the 3D shape in the spread relaxed state of the sealing member, following the solidification of the plurality of the protrusions 330 as disclosed above (see FIG. 11E).

According to some alternative examples, each one of the plurality of masking elements 333 has an elongated structure, wherein each one of the plurality of the protrusions 330 is formed between adjacent elongated masking elements 333.

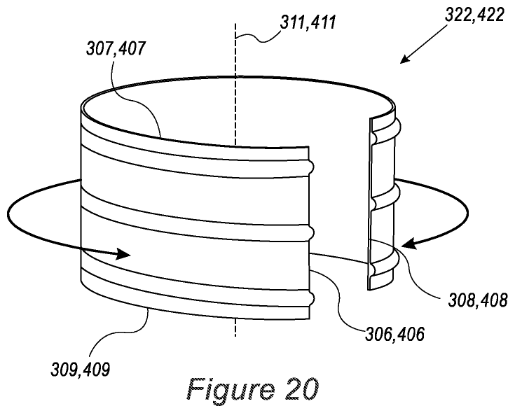

According to some examples, step (iii) comprises connecting two opposite edges (i.e., first lateral edge 306 and second lateral edge 308) of the sheet of step (ii) to form a cylindrical sealing member (or PVL skirt) in a cylindrical folded state (see for example, FIG. 20). The connection between the opposite edges can be performed by using at least one of adhesives, sutures, or heating and optionally melting the edges thereof. Alternatively, step (iii) comprises coupling the sealing member 322 to an outer surface of the frame 106, utilizing at least one of adhesives, sutures, or heating and optionally melting the edges of the sealing member 322 therearound.

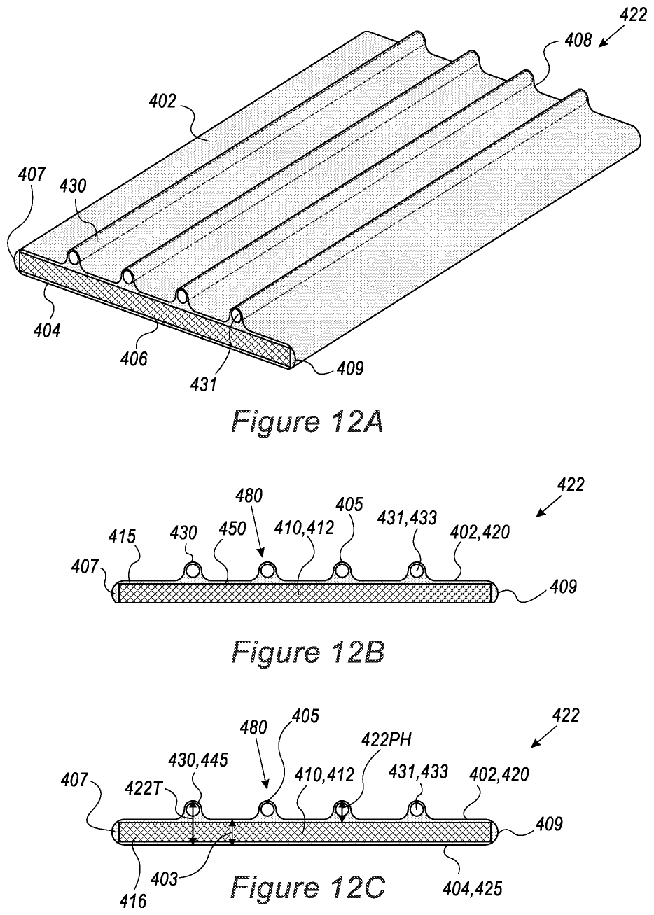

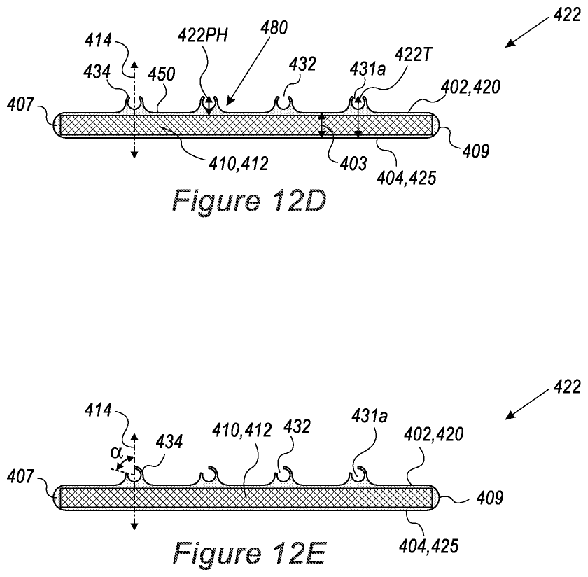

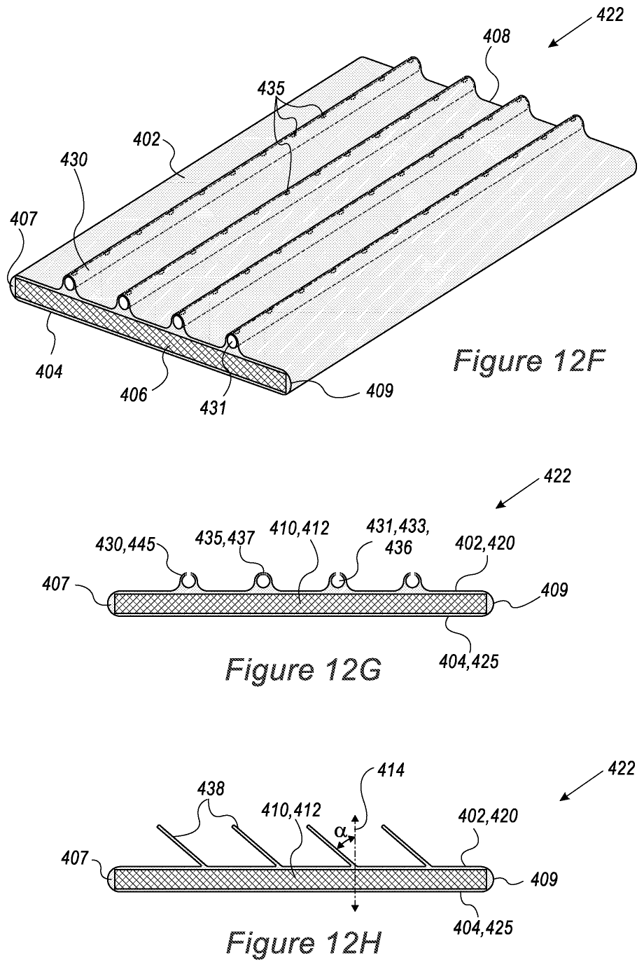

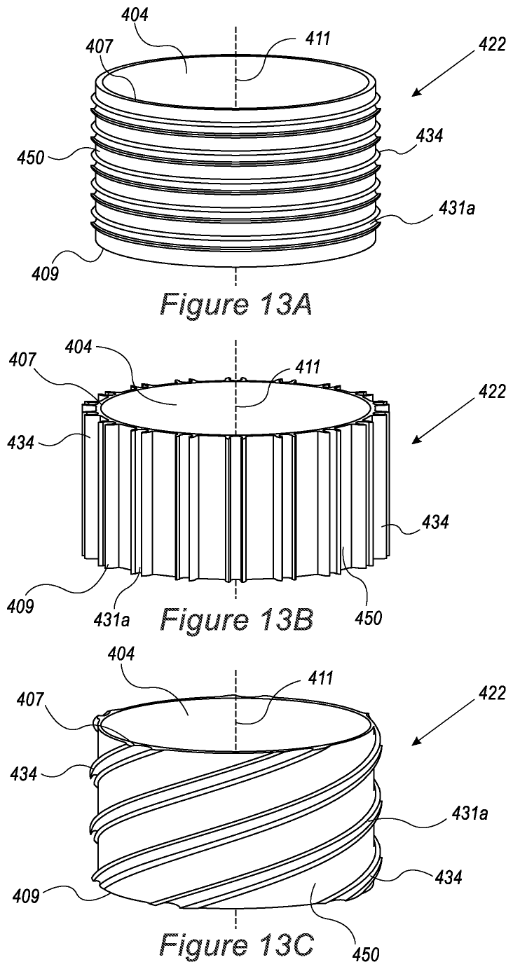

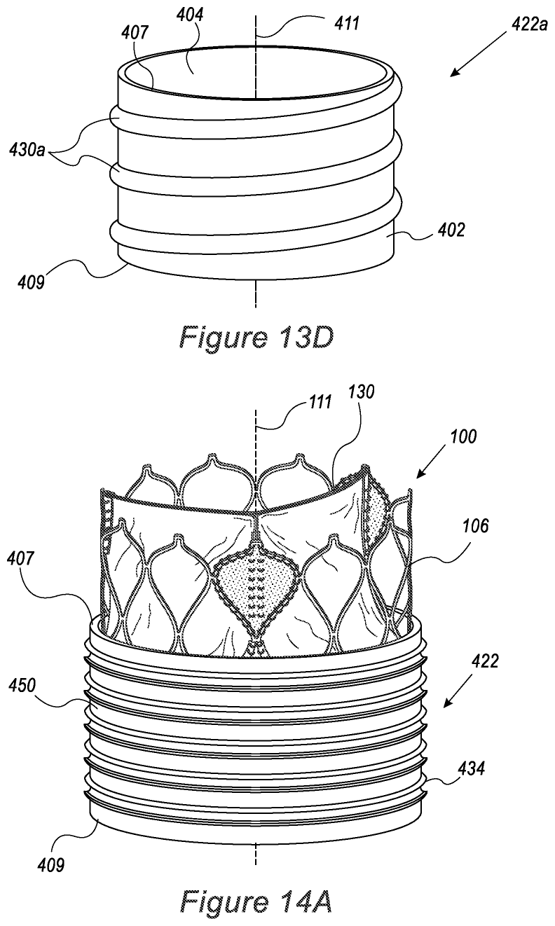

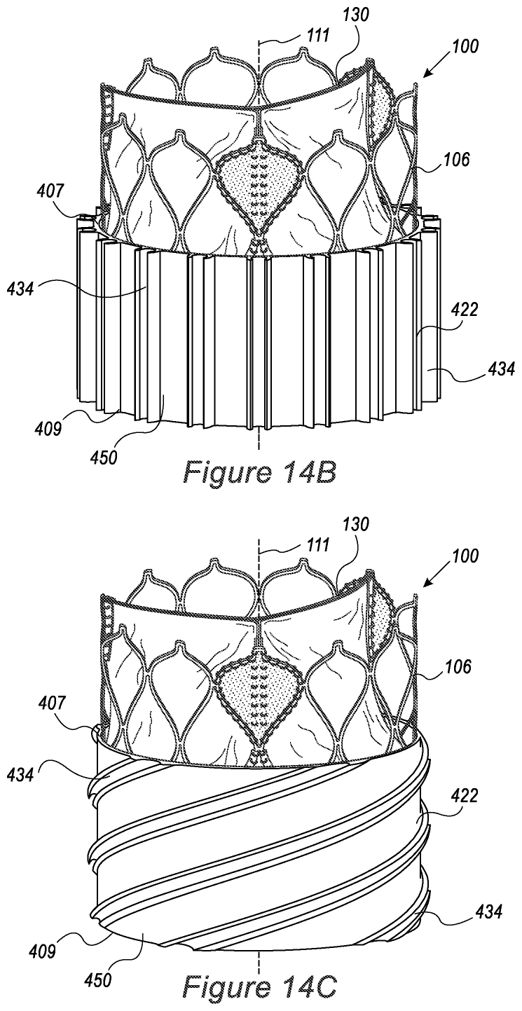

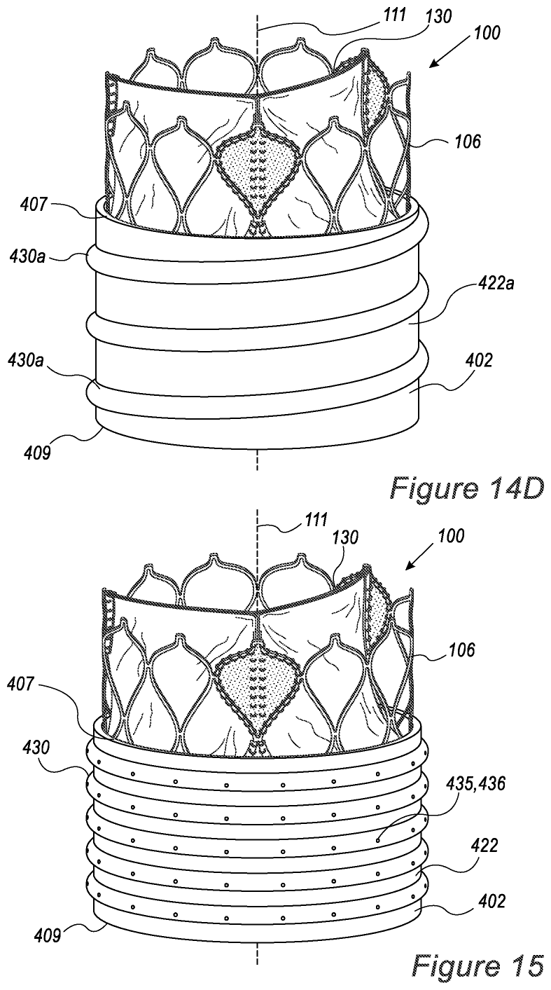

Reference is now made to FIGS. 12A-15. FIG. 12A shows a view in perspective of a sealing member 422 in a spread relaxed state, according to some examples. FIGS. 12B-12H show various cross sectional views of the sealing member 422, according to some examples. FIG. 12F shows a view in perspective of a sealing member 422 comprising a plurality of apertures 435, according to some examples. FIG. 12G shows a cross sectional view of the sealing member 422 of FIG. 12F, according to some examples. FIGS. 13A-13C show views in perspective of various configurations of sealing member 422, in a cylindrical folded state, according to some examples. FIG. 13D shows a view in perspective of a folded sealing member 422a, according to some examples. FIGS. 14A-14C show various configurations of sealing member 422 mounted on the frame 106 of the prosthetic valve 100, according to some examples. FIG. 14D shows the folded sealing member 422a mounted on the frame 106 of prosthetic valve 100, according to some examples. FIG. 15 shows the configuration of sealing member 422 comprising the plurality of apertures 435, mounted on the frame 106 of prosthetic valve 100, according to some examples.

According to another aspect, there is provided a sealing member 422, adapted to be mounted on (or coupled to) the outer surface of the frame 106 of the prosthetic valve 100 (see for example FIGS. 14A-14C), or any other similar prostatic valve known in the art. The sealing member 422 can be connected/mounted to the frame 106 using suitable techniques or mechanisms. For example, the sealing member 422 can be sutured to the frame 106 utilizing sutures that can extend around the struts 110. The sealing member 222 can be configured to form a snug fit with the frame 106 such that it lies against the outer surface of the frame 106 when the prosthetic valve 100 is in the radially expanded state, as illustrated.

According to some examples, the present invention provides a prosthetic heart valve 100 comprising a frame 106 and a leaflet assembly 130 mounted within the frame, the frame comprising a plurality of intersecting struts 110, wherein the frame is movable between a radially compressed state and a radially expanded state, as disclosed herein above, wherein the valve 100 further comprises a sealing member 422 coupled to an outer surface of the frame 106, and wherein the sealing member 422 has a three-dimensional (3D) shape in a spread relaxed state thereof.

The sealing member 422 can be provided in a spread state, and connected/mounted to the frame 106 by folding it over the frame 106, thereby transforming it from the spread to the folded state. Alternatively, the sealing member 422 may be provided in an already folded state prior to attachment to the frame 106. For example, the frame 106 may be inserted into the already cylindrically folded sealing member 422 and sutured thereto.

According to some examples, the sealing member 422 has a 3D resilient structure/shape such that a nonfibrous outer surface 480 of the sealing member 422 exhibits a plurality of elevated portions 430 with peaks 405 and a plurality of non-elevated portions 450. In further examples, each one of the plurality of non-elevated portions 450 is defined by adjacent pairs of the plurality of elevated portions 430. In further examples, the nonfibrous outer surface 480 is a smooth surface. In further examples, the nonfibrous outer surface 480 is a unitary/continuous surface.

Surface roughness is a component of surface texture. It is quantified by the deviations in the direction of the normal vector of a real surface from its ideal form. If these deviations are large, the surface is considered rough, and if they are small, the surface is considered smooth. Therefore, the term “smooth”, as used herein refers to a surface having minor deviations in the direction of the normal vector of a real surface from its ideal form. Smooth surfaces are substantially unitary/continuous surfaces, free from fibers or irregular voids. The term “smooth” is not intended to be limited to the narrow meaning of a substantially planar surface devoid of surface irregularities. Thus, none of the elevated portions, non-elevated portions and apertures of the present sealing member(s) is considered to affect the smoothness of the respective outer surface (280, 380, 480). Specifically, as can be appreciated by the skilled in the art, the outer surface(s) (280, 380, 480) of the present sealing member(s) (222, 322, 422) are to come in contact with a native tissue upon implantation. Without wishing to be bound by any theory or mechanism of action, a smooth surface coming in contact with such tissues resists or inhibits new tissue growth thereon. Therefore, it is preferable that the outer surface(s) (280, 380, 480) of the present sealing member(s) (222, 322, 422) are smooth for various implementations of the present invention.

In some examples, the elevated portions 430 are protrusions 430 and the non-elevated portions 450 are inter-protrusion gaps 450. As used herein, the terms “elevated portions 430” and “protrusions 430” are interchangeable, and refers to the same plurality of elevated portions of the sealing member 422, as can be seen in FIGS. 12B-12C. As used herein, the terms “non-elevated portions 450” and “inter-protrusion gaps 450” are interchangeable, and refers to the same plurality of non-elevated portions of the sealing member 422, as can be seen in FIGS. 12B-12C.

According to some examples, the sealing member 422 has a 3D shape in a spread relaxed state thereof, as can be appreciated for example from FIGS. 12A-12G. According to some examples, the sealing member 322 inherently has a 3D shape in a cylindrical folded state thereof (FIGS. 13A-13D and 14A-15).

Specifically, as can be appreciated for example from FIG. 12A, the sealing member 422 comprises a plurality of protrusions 430, thereby defining its 3-dimensional (3D) shape, in contrast to the substantially flat two-dimensional shape it would assume in the absence of such protrusions 430.

It is thus to be understood that the 3-dimensions of the 3-dimensional sealing member 422 include: (i) a spatial length dimension extending between an outflow edge 407 and an inflow edge 409 of the sealing member 422 (see for example, FIGS. 12B and 12C); (ii) a spatial length dimension extending between a first lateral edge 406 and an second lateral edge 408 of the sealing member 422 (see FIG. 12A); and (iii) a spatial length dimension defined by the sealing member's protrusions height (or thickness) 422T of protrusions 430 (see FIG. 12C).

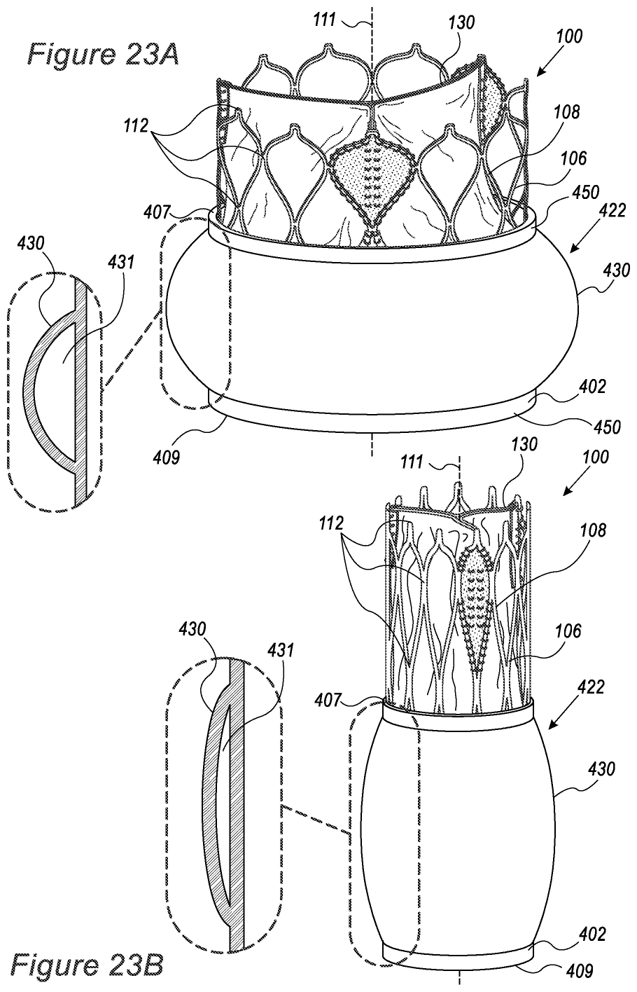

According to some examples, the sealing member 422 comprises at least one protrusion 430 extending away from a first surface 402 of the sealing member 422 (see for example, FIGS. 23A-B).

According to some examples, the sealing member 422 comprises a plurality of protrusions 430 extending away from a first surface 402 of the sealing member 422, which are spaced apart from each other along the first surface 402 of the sealing member 422. The plurality of protrusions 430 form the 3D shape of the sealing member 422 in its spread relaxed state (as can be seen in the FIGS. 12A-12G), according to some examples. According to some examples, the sealing member 422 comprises a flat surface located opposite to the first surface 402, in its spread relaxed state.

According to some examples, the sealing member 422 has four edges. According to some examples, the sealing member 422 has four vertices. According to some examples, each one of the four vertices of the sealing member 422 has a substantially right angle.

According to some examples, the sealing member 422 has four substantially right angle vertices, and two sets of two opposing edges (a set of first lateral edge 406 and second lateral edge 408, and a set of outflow edge 407 and an inflow edge 409), wherein in each set, the two opposing edges are substantially parallel. According to some examples, the sealing member 422 extends from a first lateral edge 406 toward a second lateral edge 408, when the sealing member 422 is in a spread state. According to some examples, the sealing member 422 extends around a sealing member centerline 411, in a folded state thereof. According to some examples, the sealing member centerline 411 and the centerline 111 of valve 100 are coaxial and may coincide when the sealing member 422 is connected to heart valve 100. According to some examples, the sealing member 422 extends from an inflow edge 409 toward an outflow edge 407. According to some examples, the sealing member 422 extends from an inflow edge 409 toward an outflow edge 407 in both the folded and the spread states thereof.

According to some examples, in the spread state, sealing member 422 is substantially rectangular. According to some examples, the distance from first lateral edge 406 to second lateral edge 408 is greater than the distance from inflow edge 409 to outflow edge 407.

According to some examples, each one of the plurality of protrusions 430 extends radially outward, away from the sealing member centerline 411, in a folded state of the sealing member 422 (see FIGS. 13A-13D).

According to some examples, the plurality of protrusions 430 extend in different directions from the surface 402, and can form 3D shapes thereon, wherein the 3D shapes can be selected from: inverse U-shape, half-sphere, dome, cylinder, pyramid, triangular prism, pentagonal prism, hexagonal prism, flaps, any other polygon, and combinations thereof. Each possibility represents a different example. According to further examples, the plurality of protrusions 430 extend in different directions from the surface 402, and can form parallel elongated 3D shapes thereon, wherein the elongated 3D shapes can be selected from: elongated U-shape, elongated prism, elongated cuboid, any other elongated polyhedron, and combinations thereof. Each possibility represents a different example.

According to some examples, each one of the plurality of protrusions 430 defines an elongated 3D shape and extends outward, radially away from the centerline 111 of valve 100, when the sealing member 422 is mounted on the frame 106 (see FIGS. 14A-15). According to some examples, the sealing member 422 is folded by connecting first lateral edge 406 and second lateral edge 408, such that the plurality of protrusions 430 are oriented radially away from the sealing member centerline 411. According to some examples, the sealing member 422 in a folded state thereof is coupled to the outer surface of the frame 106 of the prosthetic valve 100, such that the plurality of protrusions 430 are oriented to extend radially away from the centerline 111 (see for example, FIG. 14A).

According to some examples, the sealing member 422 is configured to transition from the spread relaxed state to the cylindrical folded state, due to its elastic and/or flexible characteristics, so as to form a cylindrical PVL skirt. A folded PVL skirt 422 may be coupled to outer surface of the frame 106 of the prosthetic valve 100, for example during a procedure of valve assembly. Alternatively, a spread sealing member 422 may be folded around the outer surface of the frame 106 and coupled thereto to achieve a similar product.

According to some examples, each one of the plurality of protrusions 430 defines a hollow lumen 431 therein (see FIGS. 12A-12C), wherein each hollow lumen 431 extends from the first lateral edge 406 toward the second lateral edge 408 of the sealing member 422. According to some examples, each hollow lumen 431 has an elongated cylindrical (including elliptic cylindrical) shape. However, it is to be understood that the cross section of each hollow lumen 431 can have a different cross-sectional shape while provided the same functionality, such as a rectangular, elliptic, triangle or any other suitable cross-sectional shape thereof. Each possibility represents a different example. It is further contemplated that the cross sectional shape of the hollow lumen 431 is not necessarily uniform along its length, according to some examples.

According to some examples, each hollow lumen 431 comprise two lumen edges.

According to some examples, each hollow lumen 431 is open ended at one or both of its lumen edges. According to further examples, each hollow lumen 431 is open ended at both lumen edges. According to some examples, one open edge is located at the first lateral edge 406 and the other one is located at the second lateral edge 408 (see FIG. 12A). According to some examples, one open edge is located at the inflow edge 409 and the other one is located at the outflow edge 407 (not shown).

According to some examples, both lateral ends of the sealing member 422 are coupled to each other in a folded state thereof, in a manner that can result in a continuous enclosed hollow lumen 431 (i.e., both open edges are fluidly connected to form a continuous lumen). In such situations, the folded sealing member 422 may be coupled to the outer surface of the frame 106 of the prosthetic valve 100 in a manner that includes trapped air within the fully enclosed hollow lumens 431. While the trapped air in such cases is fully enclosed within the hollow lumens 431 and is not exposed to the surrounding anatomy when the prosthetic valve 100 is implanted, the trapped air may still pose a risk to the patient if the protrusions 430 are degraded or accidentally torn in a manner that may release the entrapped air and result in undesirable cavitation.

According to some examples, at least one edge of the (or any other portion) of the hollow lumen 431 remains open ended or exposed to the outer environment when the sealing member 422 is coupled to the frame 106. Alternatively or additionally, protrusions 430 can include apertures (similar to apertures 435 described herein below), exposing the hollow lumen 431 to the surrounding environment. In such examples, the prosthetic valve 100 can be crimped by a crimper to the radially compressed state in a manner that flattens the protrusions 430 such that no air is trapped therein, and restrained in the crimped state as described herein above (for example, by being placed within a bounding sheath or a capsule), up until and during the implantation process, thus reducing risks of introducing entrapped air to the patient's body.

According to some examples, each one of the plurality of protrusions 430 comprises an elastic material 433 disposed therein (see FIGS. 12B-12C), wherein said elastic material 433 is different from the material the protrusions 430 are made of. According to some examples, each one of the plurality of protrusions 430 comprises a compressible material 433 disposed therein. According to some examples, each one of the hollow lumens 431 comprises an elastic material 433 disposed therein. According to some examples, each elastic material 433 is configured to be compressed without being permanently deformed (e.g., without experiencing plastic deformation) when the above-mentioned external force is applied thereto. According to some examples, the elastic material 433 comprises an elastic foam, such as an elastic sponge. According to some examples, the elastic material 433 comprises an elastic metallic cylinder forming a hollow lumen therein. According to some examples, the elastic material 433 comprises a porous elastic element/member, which is optionally elongated.

According to some examples, each one of the plurality of protrusions 430 is a divided protrusion 434, comprising at least two opposing flaps/members that together define the divided protrusion 434. According to some examples, the sealing member 422 comprises a plurality of divided protrusions 434, extending away from a first surface 402 of the sealing member 422 and spaced from each other, wherein each one of the plurality of divided protrusions 434 forms an inner space 431a therebetween. According to further examples, each inner space 431a extends from an opening 432 thereof toward the first surface 402 (see for example FIG. 12D). According to some examples, the sealing member 422 comprises a flat surface located opposite to the first surface 402, in a spread relaxed state thereof.

According to some examples, the plurality of divided protrusions 434 form the 3D shape of the sealing member 422 in its spread relaxed state (as shown in the FIGS. 12D-12E), as well as in its cylindrical folded state (FIGS. 13A-13C and 14A-14C), in contrast to a substantially flat two-dimensional shape it would have assumed in the absence of such divided protrusions 434.

The characteristics of the plurality of protrusions 430 similarly apply to the plurality of divided protrusions 434. It is thus to be understood that the 3-dimensions of the 3-dimensional sealing member 422 include: (i) a spatial length dimension extending between an outflow edge 407 and an inflow edge 409 of the sealing member 422 (see for example, FIGS. 12D and 12E); (ii) a spatial length dimension extending between a first lateral edge 406 and an second lateral edge 408 of the sealing member 422 (not shown); and (iii) a spatial length dimension defined by the sealing member's protrusions height (or thickness) 422T of divided protrusions 434 (see FIG. 12D).

According to some examples, the opening 432 of each one of the plurality of divided protrusions 434 is symmetric relative to an axis 414 extending through the middle of each divided protrusion 434 (which is a radial axis when the sealing member 422 is in its folded state), as illustrated in FIG. 12D, thereby forming a symmetric inner space 431a therein (defined as symmetry between both portions of the divided protrusion 434 across both sides of the axis 414). According to other examples, the opening 432 of each one of the plurality of divided protrusions 434 is diverted at a non-zero angle α relative to the axis 414, as can be seen at FIG. 12E, thereby forming an asymmetric inner space 431a therein. The angle α can range from about 1° to about 90°, relative to the axis 414. The angle α can range between about 1°-10°, 10°-20°, 20°-30°, 30°-40°, 40°-50°, 60°-70°, 70°-80°, or 80°-90°, relative to the axis 414. Each possibility represent a different example.

According to some examples, the sealing member 422 further comprises the plurality of inter-protrusion gaps 450, wherein each gap 450 is located (or spaces) between two adjacent protrusions 430 and/or divided protrusions 434. According to further examples, some non-elevated portions are not formed between two adjacent elevated portions, but rather between an elevated portion and an edge (e.g., an inflow edge or an outflow edge) of the sealing member. According to some examples, one inter-protrusion gap 450 is formed between the outflow edge 407 and one of the protrusions 430 and/or divided protrusions 434, while another inter-protrusion gaps 450 is formed between the inflow edge 409 and one of the other protrusions 430 and/or divided protrusions 434. It is to be understood that the non-elevated portions 450 (e.g., inter-protrusion gaps 450) are spaces formed due to the 3-dimensional shape of the sealing member 422, according to some examples. Specifically, according to some examples, the plurality of inter-protrusion gaps 450 are facing the same direction, as the protrusions 430 and/or divided protrusions 434 face.

In some implementations, attachment of the sealing member (e.g., sealing member 222, 322, 422) to the frame is accomplished by passing sutures through at least some of the non-elevated portions (e.g., non-elevated portions 250, 350, 450) and around the struts of the frame 106. Since the thickness of the first layer constitutes the major portion of the thickness of the sealing member at the non-elevated portions, the tear-resistance properties of the first layer contribute to proper retention of the sealing member when sutured to the frame, especially when the valve is crimped and elongates, optionally elongating the sealing member therewith.

Although the 3D shape of the sealing member 422 is not identical to the 3D shapes of the sealing members 222 and/or 322, it is to be understood that sealing member 422 may contain similar materials and/or have similar functionality and uses as those described above for sealing members 222 and/or 322, as presented herein above. According to some examples, unlike the 3D shape of the sealing member 222, but similar to the 3D shape of sealing member 322, the sealing member 422 comprises a flat surface (e.g., a surface 416 or a surface 404) located opposite to the first surface 402, in a spread relaxed state thereof.

According to some examples, each one of the plurality of protrusions 430 is a flap 438 (see FIG. 12H). According to some examples, the sealing member 422 comprises a plurality of flaps 438, extending away from the first surface 402 of the sealing member 422 and spaced apart from each other, wherein each one of the plurality of flaps 438 is diverted at an angle α relative to the axis 414, as illustrated in FIG. 12H. It is to be understood that the various characteristics of protrusions 430 and/or divided protrusions 434, as disclosed herein, similarly apply to flaps 438.

According to some examples, each flap 438 is resiliently elastic and comprise a thermoplastic elastomer material as disclosed herein, such as TPU, which may deflect toward or be pressed against the annular or arterial wall 105 at the implantation site, following the implantation and expansion of the prosthetic heart valve 100 therein, and thus to enable an enhanced PVL sealing between the prosthetic heart valve 100 and the inner surface of the annular or arterial wall 105.

According to some examples, the sealing member 422 comprises the plurality of flaps 438 and has a resilient 3D shape, wherein said resilient 3D shape is configured to elastically deform when an external force is applied thereto (e.g., when compressed against the annular or arterial wall 105, or against an inner wall of a sheath or a capsule), and further configured to revert to its original shape (i.e., the shape of its relaxed state) when the external force is no longer is applied thereto (e.g., when a valve is released from the shaft or capsule prior to expansion thereof). While the flaps 438 are shown to have a generally liner cross-sectional shape in FIG. 12H, it is to be understood that this is for the purpose of illustration and not limitation, and the flaps 438 may be similarly provided with an arcuate or other non-liner cross-sectional shape.

An important design parameter of a transcatheter prosthetic heart valve is the diameter of the folded or crimped state. The diameter of the crimped profile is important because it directly influences the user's (e.g., medical personnel) ability to advance the transcatheter prosthetic heart valve through the femoral artery or vein. More particularly, a smaller profile allows for treatment of a wider population of patients, with enhanced safety. Because the sealing member 422 comprising the plurality of flaps 438 is coupled to the outer surface of the frame 106 of the prosthetic valve 100, the prosthetic valve 100 can be crimped to a lower profile, within a delivery system, than would be possible if the valve 100 was crimped while including a sealing member having a different 3D structure. This lower profile permits the user to more easily navigate the delivery apparatus (including crimped valve 100) through a patient's vasculature to the implantation site. The lower profile of the crimped valve is particularly advantageous when navigating through portions of the patient's vasculature which are particularly narrow, such as the iliac artery.

According to some examples, advantageously, the prosthetic valve 100 comprising the sealing member 422 comprising the plurality of flaps 438 is characterized by having a lower profile in its crimped state within a delivery system, relative to a valve 100 comprising a sealing member having a more rigid or non-compressible 3D form in the same state. It is contemplated that the lower profile of the crimped state of valve 100 is possible due to the 3D shape of the flaps 438 made of the thermoplastic elastomer material as disclosed herein. According to some examples, the prosthetic valve 100 comprising the sealing member 422 having the plurality of flaps 438, is configured to be advanced within a delivery system in the crimped state toward the implantation site, wherein the flaps 438 are compressed against an inner wall of the sheath or capsule of the delivery system, such that the flaps 438 are diverted in the proximal direction, opposite to the distal advancement direction of the valve 100, to facilitate easier delivery.

According to some examples, the prosthetic valve 100 comprising the sealing member 422 is configured to be positioned (i.e., implanted) at the target implantation site (i.e., the aortic annulus in the case of aortic valve replacement) so as to form contact between the arterial wall 105 and the plurality of flaps 438, protrusions 430, and/or divided protrusions 434, similar to contact formed between the arterial wall 105 and the plurality of ridges 230 of sealing member 222 and/or the plurality of protrusions 330 of sealing member 322, as disclosed herein above. Advantageously, the plurality of flaps 438, protrusions 430, and/or divided protrusions 434, of the sealing member 422, are adapted to contact the arterial wall 105 following the expansion of the prosthetic heart valve 100 at the site of implantation, and thus to enable a conforming fit or engagement between the prosthetic heart valve 100 and the inner surface of the annular or arterial wall 105, which in turn improves PVL sealing around the implanted prosthetic heart valve.

Moreover, the resiliency of all peaks (or peak portions) disclosed herein, including peaks provided in the form of ridges 230, protrusions 330, flaps 438, protrusions 430, divided protrusions 434, as well as other types of peaks disclosed herein, allows them to elastically deform and be pressed or squeezed radially inward when the prosthetic valve is retained in its crimped state (for example, due to external force applied by the sheath or capsule retaining the valve 100), resulting in a favorable crimped profile, while springing radially outward to their relaxed state configuration as soon as the valve is released from the sheath or capsule, thereby extending radially outward toward the annular or arterial wall to improve PVL sealing after deployment.

According to some examples, the plurality of protrusions 430 and/or divided protrusions 434 can extend away from the first surface 402 thereof in different directions and/or configurations. These may be vertical, horizontal or diagonal with respect to the centerline 411 of the cylindrically shaped sealing member 422 in its folded state. It is to be understood that the orientation of the protrusions 430 in the folded state of the sealing member 422 may be dictated by their construction prior to the folding, i.e. when the sealing member 422 is in a spread state. According to some examples, the sealing member 422 has a resilient 3D shape, wherein said resilient 3D shape is defined by the plurality of protrusions 430 which form an overall wave-like configuration on the surface 402 thereof.

For example, a sealing member 422, which has a plurality of divided protrusions 434 extending from first lateral edge 406 to second lateral edge 408, may be folded by connecting first lateral edge 406 to second lateral edge 408 such that a cylindrical shape of the sealing member 422 is formed. In such an exemplary situation, upon said folding, the sealing member 422 in its folded shape will have the plurality of divided protrusions 434 which are substantially parallel to inflow edge 409 and to outflow edge 407 (as shown in FIG. 13A). In a second example, a sealing member 422, which has plurality of divided protrusions 434 extending from inflow edge 409 to outflow edge 407, may be folded by connecting first lateral edge 406 to second lateral edge 408 such that a cylindrical shape of the sealing member 422 is formed. In such a second exemplary situation, upon said folding, the sealing member 422 in it folded shape will have the plurality of divided protrusions 434 which are substantially perpendicular to inflow edge 409 and to outflow edge 407 (as shown in FIG. 13B). Similarly, diagonal divided protrusions 434 in the spread state will lead to diagonal divided protrusions 434 in the folded state of the sealing member 422, as shown in FIG. 13C.

According to some examples, the plurality of protrusions 430 and/or divided protrusions 434 are parallel to any one of the outflow edge 407 and/or the inflow edge 409 when the sealing member 422 is in a spread state. According to some examples, the plurality of protrusions 430 and/or divided protrusions 434 are circumferentially extending around the sealing member centerline 411, in a folded state of the sealing member 422 (see for example, FIG. 13A). According to some examples, the plurality of protrusions 430 and/or divided protrusions 434 are circumferentially extending around the centerline 111, when the sealing member 422 is in a folded state and mounted on the frame 106 of the prosthetic heart valve 100 (see for example, FIG. 14A). According to some examples, the plurality of protrusions 430 and/or divided protrusions 434 are aligned in parallel to any one of the outflow edge 407 and/or the inflow edge 409, circumferentially around the sealing member centerline 411, in a folded state of the sealing member 422.

According to some examples, the plurality of protrusions 430 and/or divided protrusions 434 extend from the inflow edge 409 to the outflow edge 407 in a spread state of the sealing member 422. According to some examples, the plurality of protrusions 430 and/or divided protrusions 434 are aligned in parallel to any one of the first lateral edge 406 and/or the second lateral edge 408 in a spread state of the sealing member 422. According to some examples, the plurality of protrusions 430 and/or divided protrusions 434 are aligned perpendicularly to any one of the outflow edge 407 and/or the inflow edge 409 in a spread state of the sealing member 422.

According to some examples, the plurality of protrusions 430 and/or divided protrusions 434 are aligned in parallel to the sealing member centerline 411 in a spread state of the sealing member 422 (see, for example, FIG. 13B). According to some examples, the plurality of protrusions 430 and/or divided protrusions 434 extend in parallel to the centerline 111 when the sealing member 422 is in a folded state and mounted on the frame 106 of prosthetic heart valve 100 (see for example, FIG. 14B). According to some examples, the plurality of protrusions 430 and/or divided protrusions 434 are aligned perpendicularly to any one of the outflow edge 407 and/or the inflow edge 409 in a folded state of the sealing member 422.

According to some examples, the plurality of protrusions 430 and/or divided protrusions 434 extend diagonally along the surface of the sealing member 422, in a spread state thereof. According to some examples, the plurality of protrusions 430 and/or divided protrusions 434 extend diagonally along the surface of the sealing member 422, in a folded state thereof (see for example, FIG. 13C). According to some examples, the plurality of protrusions 430 and/or divided protrusions 434 extend diagonally to the centerline 111 when the sealing member 422 is in a folded state and mounted on the frame 106 prosthetic heart valve 100 (see for example, FIG. 14C).

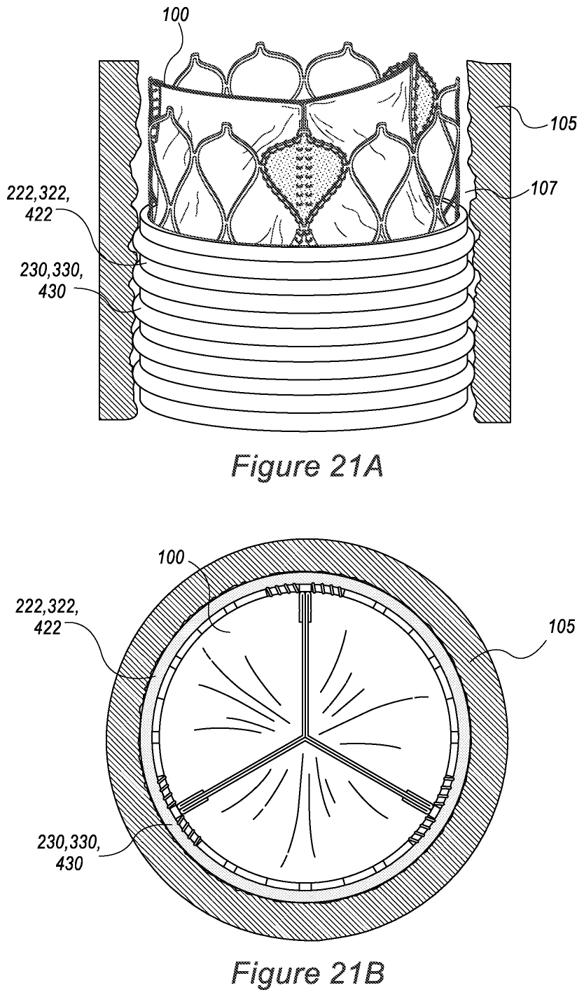

Various configurations and orientations as described above may be advantageous for different physiological and implantation-related requirements. For example, the configuration of FIGS. 13A and 14A may be advantageous due to the generally perpendicular orientation of the plurality of protrusions 430 and/or divided protrusions 434 relative to the axial direction of the flow, when the valve 100 is mounted against the annular or arterial wall 105, thereby potentially improving PVL sealing therebetween (see for example, FIGS. 21A and 21B).

Moreover, it is contemplated that the configurations of the plurality of divided protrusions 434, which may be in some examples diverted at the angle α relative to radial axes 414, may be advantageous, since such configurations, and especially asymmetric configurations forming the asymmetric inner space 431a within divided protrusions 434 (see FIGS. 13A and 14A), which can serve as a semi-closed pockets or compartments, compressible between surface 402 of the sealing member 422 and the annular or arterial wall 105, following implantation. Specifically, an asymmetric inner space 431a can prevent or significantly reduce paravalvular leakage (PVL) of blood therethrough by trapping the blood within said semi-closed pocket or compartment, thereby improving PVL sealing between the sealing members and the surrounding anatomy.

As detailed herein, the fabrication process of creating the protrusions 430 and/or divided protrusions 434 in the sealing member 422 is not limited to be performed prior to the step of folding, and in some examples, the protrusions 430 and/or divided protrusions 434 may be formed on the first surface 402 of the sealing member 422 after the step of folding. In such examples, there is provided a folded sealing member 422a, which comprises the same materials disclosed for the sealing member 422 and has a similar functionality, except that the folded sealing member 422a is manufactured in a folded cylindrical state. According to some examples, the folded sealing member 422a comprises at least one helical protrusion 430a extending radially outward, away to the centerline 411, in a helical configuration around the first surface 402, wherein the sealing member 422a is not necessarily connected to a heart valve, as illustrated in FIG. 13D.

According to some examples, the at least one helical protrusion 430a extends from the inflow edge 409 to the outflow edge 407 of the folded sealing member 422a. According to some examples, the folded sealing member 422a is coupled to the outer surface of the frame 106 of the prosthetic valve 100 so that the at least one helical protrusion 430a extend radially away from the centerline 111 in a helical configuration around the first surface 402, as illustrated at FIG. 14D.

According to some examples, the folded sealing member 422a is characterized by having a nonfibrous outer surface, comprising the at least one helical protrusion 430a, similar to the nonfibrous outer surface 480, as disclosed herein.

According to some examples, the at least one helical protrusion 430a is hollow and defines a helical hollow lumen therein (not shown). In further examples, the at least one helical protrusion 430a comprises a plurality of apertures 435 spaced from each other along a surface thereof, and are configured to provide fluid communication between the helical hollow lumen and the external environment outside of the apertures 435. In still further examples, the hollow lumen comprises pharmaceutical composition 436 disposed therein, as disclosed herein. At least a portion of the apertures 435 can be sealed with a biodegradable membrane 437, as described herein.

According to some examples, the sealing member 422 comprises a first layer 410. According to some examples, the first layer 410 is flat spread relaxed state of the sealing member 422.

According to some examples, the sealing member 422 comprises a first layer 410 and a second layer 420. According to further examples, said first and second layers 410 and 420, respectively, are disposed externally to the outer surface of the frame 106, when the sealing member 422 is coupled thereto. According to further examples, the sealing member 422 can comprise additional layer(s).

According to some examples, the second layer 420 is in contact with a first surface 415 of the first layer 410 (see FIG. 12B). According to some examples, the second layer 420 is in contact with a first surface 415 of the first layer 410 both in the spread and folded states of the sealing member 422. According to some examples, the second layer 420 is attached to and/or is coating a first surface 415 of the first layer 410. According to some examples, said first surface 415 of the first layer 410 is oriented outward in a folded state of the sealing member 422.

According to some examples, said first surface 415 is oriented toward the implantation site (e.g., the annular or arterial wall 105) when the sealing member 422 is mounted on the frame 106 of the prosthetic heart valve 100 and implanted at the implantation site. According to further examples, the second layer 420 defines a first surface 402 of the sealing member 422, as illustrated in FIG. 12B. According to some examples, the first surface 402 of the sealing member 422 is oriented outward in a folded state of the sealing member 422. According to some examples, the first surface 402 of the sealing member 422 is oriented toward the implantation site when the sealing member 422 is mounted on the frame 106 of the prosthetic heart valve 100 and implanted at the implantation site.

According to some examples, the sealing member 422 comprises a third layer 425. According to some examples, the third layer 425 is in contact with a second surface 416 of the first layer 410 (see FIG. 12C). According to some examples, the third layer 425 is in contact with a second surface 416 of the first layer 410 both in the spread and folded states of the sealing member 422. According to some examples, the third layer 425 is attached to and/or is coating a second surface 416 of the first layer 410. According to some examples, said second surface 416 of the first layer 410 is oriented inward direction in a folded state of the sealing member 422. According to some examples, said second surface 416 is oriented in a direction opposite to the annular or arterial wall 105 when the sealing member 422 is mounted on the frame 106 of the prosthetic heart valve 100 and implanted at the implantation site.

According to further examples, the third layer 425 defines a second surface 404 of the sealing member 222, as illustrated at FIG. 12C. According to some examples, the second surface 404 of the sealing member 422 is oriented in the inward direction when the sealing member 422 is in a folded state. According to some examples, the second surface 404 of the sealing member 422 is oriented in the direction opposite to the anatomical wall at the implantation site when the sealing member 422 is mounted on the frame 106 of the prosthetic heart valve 100 and implanted at the implantation site.

According to some examples, the second surface 404 of the sealing member 422 is a flat surface (FIG. 12C). According to other examples, the second surface 404 of the sealing member 422 comprises a plurality of additional protrusions 430 (not shown).

According to some examples, the sealing member 422 extends between the first surface 402 and the second surface 404, wherein the sealing member 422 has a total layer thickness 403 measured between the first surface 402 and the second surface 404 at one of the inter-protrusion gaps 450, as illustrated in FIG. 12C. According to some examples, said total layer thickness 403 is measured from the first surface 402 of the sealing member 422 to the second surface 416 of the first layer 410 (not shown). According to some examples, the total layer thickness 403 is measured from the first surface 402 of the sealing member 422 (e.g., the second layer 420) to the second surface 404 (e.g., the third layer 425), as shown in FIGS. 12C and/or 12D.

According to some examples, the thickness 422T of sealing member 422 is at least 1000% greater than the total layer thickness 403. In further examples, the thickness 422T is at least 2000%, at least 3000%, at least 4000%, at least 5000%, or at least 6000% greater than the total layer thickness 403 of the sealing member 422. In further examples, the thickness 422T is no greater than 6000%, 7000%, 8000%, 9000%, 10,000%, 20,000%, 30,000%, 40,000% or 50,000% compared to the total layer thickness 403 of the sealing member 422. Each possibility represents a different example.

It is to be understood that the thickness ratio between thickness 422T and total layer thickness 403 in FIGS. 12B-C is moderate, whereas, as described above, the actual ratio is greater (e.g. the thickness 422T is 10-60 times greater than the total layer thickness 403). For example, in some non-binding implementations, the total layer thickness 403 can be in the range of 0.02 to 0.1 mm, while the thickness 422T can be in the range of 0.5-3 mm.

According to some examples, the 3D shape of the sealing member 422 in its spread relaxed state, is achieved by protrusions 430 (FIG. 12C) or divided protrusions 434 (FIG. 12D), each having a protrusion height 422PH, being a part of thickness 422T thereof. In further examples, the protrusion height 422PH and the total layer thickness 403 together define the thickness 422T of sealing member 422.

According to some examples, any of the plurality of flaps 438, the at least one helical protrusion 430a, the plurality of protrusions 430 and/or the plurality of divided protrusions 434 extend away from the second layer 420 of the sealing member 422 and are spaced from each other, wherein the second layer 420 is attached to and/or is coating the first surface 415 of the first layer 410, wherein said surface 415 is oriented toward the implantation site (i.e., the annular or arterial wall 105) following the attachment of the sealing member 422 to valve 100 and implantation thereof.

According to some examples, sealing member 422 comprises both the second layer 420 and the third layer 425. According to some examples, the second layer 420 is connected to the third layer 425. According to some examples, the second layer 420 and the third layer 425 are unified to cover the first layer 410, as illustrated in FIG. 12C. According to some examples, the second layer 420 and the third layer 425 collectively form a coating which covers both the first and second surfaces 402 and 404, respectively, of the sealing member 422. According to some examples, the second layer 420 and the third layer 425 collectively form a coating which covers the sealing member 422.

According to some examples, the sealing member 422 further comprises a fourth layer 445. According to some examples, each one of the plurality of protrusions 430 comprises the fourth layer 445. According to some examples, the fourth layer 445 coats each one of the plurality of protrusions 430. According to some examples, the fourth layer 445 forms a coating which covers each one of the plurality of protrusions 430 and optionally the second layer 420. According to some examples, the fourth layer 445 is connected to the second layer 420.

It is to be understood based on the above that the spread sealing member 422 is folded into its folded state by connecting its first lateral edge 406 and its second lateral edge 408, over the second surface 404 thereof, such that in a folded state of the sealing member 422, its second surface 404 faces inward (toward the sealing member centerline 411) and its first surface 402 faces outward, according to some examples. Therefore, when the folded sealing member 422 is mounted on the frame 106 of the prosthetic heart valve 100 and implanted at the implantation site, the second layer 420, and any of the plurality of flaps 438, the at least one helical protrusion 430a, the plurality of protrusions 430 and/or the plurality of divided protrusions 434 (and optionally the fourth layer 445) which extend away therefrom, are in contact with the anatomical wall at the implantation site (e.g., the inner surface of the annular or arterial wall 105).

According to some examples, the sealing member 422 has a resilient 3D structure such that the nonfibrous outer surface 480 of the sealing member 422 exhibits the plurality of elevated portions 430 with peaks 405 and the plurality of non-elevated portions 450, as disclosed herein above (see for example FIGS. 12B-C). According to some examples, the nonfibrous outer surface 480 of the sealing member 422 is defined as an outer surface combining the first surface 402 and an outer surface of each one of the plurality of elevated portions 430 (i.e., protrusions 430). According to some examples, the peaks 405 are defined as the highest point along the outer surface of each one of the plurality of elevated portions 430, extending away from the first surface 402 of the sealing member 422. According to some examples, the height of each peak 405 is defined as the distance of the highest point along the outer surface of each one of the plurality of elevated portions 430, relative to the frame 106, when the sealing member 422 is coupled to the outer surface of the frame 106 of the prosthetic valve 100 (e.g., the thickness 422T).

According to some examples, the non-elevated portions 450 are defined as the inter-protrusion gaps 450. In further such examples, the height of each non-elevated portion 450 is defined as the distance of the first surface 402 relative to the frame 106, when the sealing member 422 is coupled to the outer surface of the frame 106 of the prosthetic valve 100 (e.g., the total layer thickness 403). According to some examples, the distance of the peaks 405 from the frame 106 is at least 1000% greater than the distance of the non-elevated portions 450 from the frame 106, in the absence of an external force applied to press the elevated portions 430 against the frame. According to further examples, the distance of the peaks 405 from the frame 106 is at least 2000%, at least 3000%, at least 4000%, at least 5000%, or at least 6000% greater than the distance of the non-elevated portions 450 therefrom. Each possibility represents a different example.

It is to be understood that any reference to the thickness 422T of sealing member 422 is equivalent to the distance of the peaks 405 of the elevated portions 430 from the external surface of the frame 106, in a relaxed state of the sealing member 422 when coupled to the frame 106. Similarly, any reference to the total layer thickness 403 is equivalent to the distance of the non-elevated portions 450 from the external surface of the frame 106, when the sealing member 422 is coupled thereto.

According to some examples, the first layer 410 comprises the same materials as each one of the first layers 210 and/or 310, as described herein above. According to some examples, the first layer 410 is made from a flexible and/or elastic material(s) adapted to provide mechanical stability, and optionally tear resistance (or tear strength), to the sealing member 422. In further examples, the first layer 410 is configured to enable the continuous durable attachment of the sealing member 422 to the outer surface of the frame 106 of the prosthetic valve 100, optionally by preventing the formation of irreversible deformation thereto (e.g., resist tearing), thus providing mechanical stability to the structure during utilization thereof.

The first layer 410 can contain, for example, various woven biocompatible textiles, comprising materials such as various synthetic materials (e.g., polyethylene terephthalate (PET), polyester, polyamide (e.g., Nylon), polypropylene, polyetheretherketone (PEEK), polytetrafluoroethylene (PTFE), etc.), natural tissue and/or fibers (e.g. bovine pericardium, silk, cotton, etc.), metals (e.g., a metal mesh or braid comprising gold, stainless steel, titanium, nickel, nickel titanium (Nitinol), etc.), and combinations thereof. Each possibility represents a different example.

The first layer 410 can be a metallic or polymeric member, such as a shape memory metallic or polymeric member. The first layer 410 can be a woven textile. It is to be understood that the first layer 410 is not limited to a woven textile. Other textile constructions, such as knitted textiles, braided textiles, fabric webs, fabric felts, filament spun textiles, and the like, can be used. The textiles of first layer 410 can comprise at least one suitable material, selected from various synthetic materials, natural tissue and/or fibers, metals, and combinations thereof, as described herein above.

According to some examples, the first layer 410 comprises at least one tear resistant material, wherein the tear resistant material optionally comprises a PET fabric, and wherein the tear resistant material is configured to provide mechanical stability and tear resistance and support the structure thereof, similar to the properties and characteristics of each one of the first layers 210 and/or 310, as described herein above. According to further examples, the first layer 410 comprises a tear resistant PET fabric. According to further examples, the first layer 410 comprises at least one tear resistant knit/woven PET fabric.

According to some examples, the first layer 410 comprises at least one tear resistant and flexible material, which is able to withstand loads of above about 3N of force before tearing, thereby enabling the sealing member 422 to reliably operate without tearing during regular use thereof. According to further examples, the at least one tear resistant and flexible material of the first layer 410 is able to withstand loads of above about 5N, 7N, 10N, 15N, 20N, 25N, 30N, or more, of force before tearing. Each possibility represents a different example. According to still further examples, the at least one tear resistant and flexible material of the first layer 410 is able to withstand loads of above about 20N of force before tearing. According to yet still further examples, the at least one tear resistant and flexible material of the first layer 410 is able to withstand loads of above about 30N of force before tearing. According to a preferred example, the at least one tear resistant and flexible material of the first layer 410 comprises a PET fabric and is able to withstand loads of up to about 20N of force before tearing.

According to some examples, the first layer 410 is made from at least one biocompatible material, as disclosed herein above.

It is to be understood that when the first layer 410 is covered by the second layer 420 and third layer 425, as shown in FIG. 12C, it should not come in contact with tissues when implanted, and thus, in this case first layer 410 may be made of non-biocompatible materials. Nevertheless, it may be preferable to form the first layer 410 from biocompatible materials in such cases as well, to prevent risks of abrasive damage or tears of any of the second layer 420 or third layer 425, which may in turn expose portions of the first layer 410.

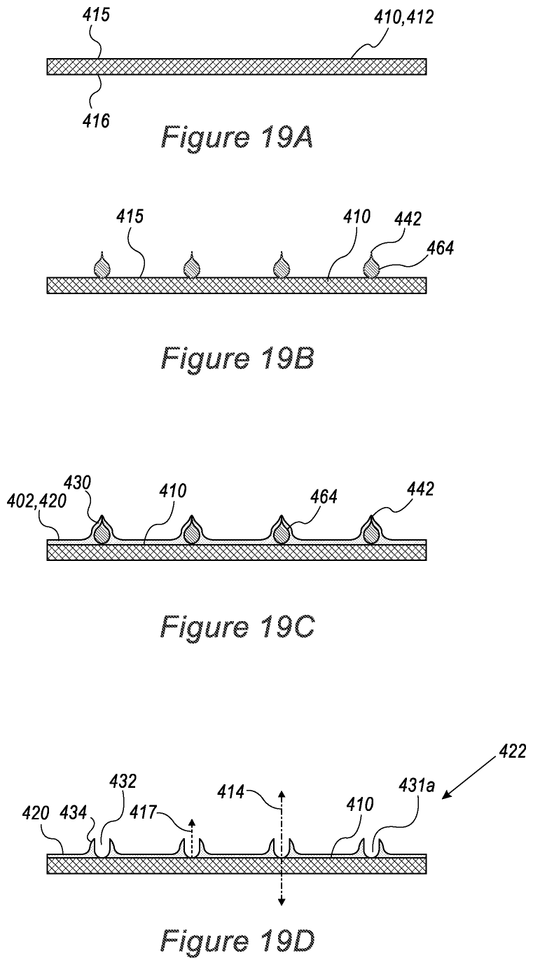

According to some examples, the sealing member 422 comprises a plurality of divided protrusions 434, extending away from a first surface 402 of the sealing member 422 and spaced from each other, wherein each one of the plurality of divided protrusions 434 defines an inner space 431a therein. According to some examples, each inner space 431a extends from an opening 432 thereof toward the first layer 410 (see for example FIG. 19D).

According to some examples, at least one of the second layer 420, the plurality of flaps 438, the at least one helical protrusion 430a, the plurality of protrusions 430, the plurality of divided protrusions 434, and optionally the fourth layer 445, comprise the same materials as described herein above for each one of the second layers 220 and/or 320. According to some examples, the second layer 420, the plurality of flaps 438, the at least one helical protrusion 430a, the plurality of protrusions 430, the plurality of divided protrusions 434, and optionally the fourth layer 445, are adapted to contact the implantation site tissue (i.e., the inner surface of the annular or arterial wall 105) and therefore are made from at least one elastic biocompatible material. Furthermore, it may be advantageous for the second layer 420, the plurality of protrusions 430 and/or divided protrusions 434 to be made of materials that may prevent/resist and/or reduce the extent of tissue ingrowth around or over the sealing member 422, according to some examples, such that if and when an explant procedure is required, the valve 100 can be easily removed from the site of implantation, as detailed above.

According to some examples, the first surface 402 of the sealing member 422 (i.e., the second layer 420), and optionally the fourth layer 445, is characterized by having a smooth and/or a low-friction surface, adapted to reduce friction with tissue of the implantation site, thereby reducing tissue ingrowth therearound and enabling easier removal of the previously implanted valve from the site of implantation. According to some examples, any one of the plurality of flaps 438, the at least one helical protrusion 430a, the plurality of protrusions 430 and/or the plurality of divided protrusions 434, are characterized by having a smooth and/or a low-friction outer surface, adapted to reduce friction with tissue of the implantation site, for the reasons described herein above.

According to some examples, the second layer 420 and/or the plurality of protrusions 430 are continuous in a manner which is devoid of yarns and/or strands (including texturized yarns and/or strands).

According to some examples, at least one of the second layer 420, the plurality of flaps 438, the at least one helical protrusion 430a, the plurality of protrusions 430, the plurality of divided protrusions 434, and optionally the fourth layer 445, can be made of various suitable biocompatible synthetic materials, such as, but not limited to, a thermoplastic material. According to some examples, the thermoplastic material is selected from the group consisting of: polyamides, polyesters, polyethers, polyurethanes, polyolefins, polytetrafluoroethylenes, and combinations and copolymers thereof.

According to some examples, at least one of the second layer 420, the plurality of flaps 438, the at least one helical protrusion 430a, the plurality of protrusions 430, the plurality of divided protrusions 434, and optionally the fourth layer 445, can be made of various suitable biocompatible synthetic materials, such as, but not limited to, thermoplastic material, including thermoplastic elastomers (TPE). According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example.

According to some examples, at least one of the second layer 420, the flaps 438, the at least one helical protrusion 430a, the plurality of plurality of protrusions 430, the plurality of divided protrusions 434, and optionally the fourth layer 445, comprise at least one thermoplastic thromboresistant material, wherein the thermoplastic thromboresistant material comprises at least one thermoplastic elastomer, optionally comprising TPU. According to further examples, the second layer 420 and any of the plurality of flaps 438, the at least one helical protrusion 430a, the plurality of protrusions 430, and/or the plurality of divided protrusions 434, together define the 3D shape of the sealing member 422 in a folded cylindrical state, which is adapted to improve PVL sealing between the prosthetic heart valve 100 and the inner surface of the annular or arterial wall 105, and optionally prevent and/or reduce tissue ingrowth thereover.

According to some examples, the second layer 420, any of the plurality of flaps 438, the at least one helical protrusion 430a, the plurality of protrusions 430, the plurality of divided protrusions 434, and optionally the fourth layer 445, comprise TPU. According to some examples, the third and second layers 425 and 420, respectively, are made of the same material, preferably TPU.

According to some examples, each one of the plurality of divided protrusions 434 forms the inner space 431a therein, wherein an external surface of each one of the plurality of divided protrusions 434 comprises the at least one thermoplastic thromboresistant material as described herein above, optionally comprising TPU.

According to some examples, each one of the plurality of protrusions 430 defines the hollow lumen 431 therein, wherein an external surface of each one of the plurality of protrusions 430 (e.g., the fourth layer 445) comprises the at least one thermoplastic thromboresistant material as described herein above, optionally comprising TPU.

According to some examples, each one of the hollow lumens 431 comprises the elastic material 433 therein, wherein the elastic material 433 is configured to be compressible or squeezable without experiencing irreversible deformation. The elastic material 433 can comprise an elastic foam and/or an elastic metallic cylinder, as specified herein above. The elastic material 433 can be different from the at least one thermoplastic thromboresistant material forming the plurality of protrusions 430.

According to some examples, the sealing member 422 comprises the first layer 410, the second layer 420, any of the plurality of flaps 438, the at least one helical protrusion 430a, the plurality of protrusions 430 or the plurality of divided protrusions 434, extending away from the second layer 420 that coats at least the first surface 402, and optionally at least one of the third layer 425 and/or the fourth layer 445.

According to some examples, the first layer 410 is configured to provide mechanical stability and tear resistance and support the structure thereof. According to some examples, the second layer 420 and any of the plurality of flaps 438, the at least one helical protrusion 430a, the plurality of protrusions 430 or the plurality of divided protrusions 434 (and optionally at least one of the third layer 425 and/or the fourth layer 445), are configured to form and maintain the 3D shape thereof, and optionally further configured to prevent and/or reduce tissue ingrowth thereover. It is contemplated that the second layer 420 on its own, may lack the ability to maintain a successful durable attachment the sealing member 422 to the outer surface of the frame 106. Specifically, the sealing member 422 may have a low tear resistance, which does not enable sewing it to the frame 106 in a durable manner.

Advantageously, the combination between the first layer 410, the second layer 420 on its own or together with the optional third layer 425 and fourth layer 445, and any one of the plurality of flaps 438, the at least one helical protrusion 430a, the plurality of protrusions 430 or the plurality of divided protrusions 434, enables to provide the required features of the sealing member 422. According to some examples, the second layer 420 comprising TPU, on its own or together with the optional third layer 425 and fourth layer 445, and any of the plurality of flaps 438, the at least one helical protrusion 430a, the plurality of protrusions 430 or the plurality of divided protrusions 434, are reinforced by the first layer 410 comprising PET to provide the strength required to retain the sutures.

It is contemplated that the utilization of thermoplastic elastomer material(s), such as TPU, as a layer of sealing member 422 and/or a component within the plurality of protrusions 430 or divided protrusions 434, enables to fabricate it in a manner which allows formation of a desired 3D-shaped sealing member 422 having a plurality of elastic protrusions 430 or divided protrusions 434. In some examples, advantageously, the plurality of elastic flaps 438, the at least one elastic helical protrusion 430a, the plurality of elastic protrusions 430 or the plurality of elastic divided protrusions 434 of the sealing member 422, are adapted to contact, and become compressed against, the annular or arterial wall 105 at the implantation site, following expansion of the prosthetic heart valve 100 therein, thereby improving PVL sealing between the prosthetic heart valve 100 and the inner surface of the annular or arterial wall 105.

Thus, according to some examples, each one of the plurality of flaps 438, the at least one helical protrusion 430a, the plurality of protrusions 430 or the plurality of divided protrusions 434, is elastic and compressible. The elastic and compressible characteristics of the plurality of flaps 438, the at least one helical protrusion 430a, the plurality of protrusions 430 or the plurality of divided protrusions 434, can improv retention of the sealing member 422 against the tissues of the native heart valve at the implantation site. According to some examples, the sealing member 422 has a resilient 3D shape, wherein said resilient 3D shape is configured to deform when an external force is applied thereto (e.g., when compressed against the annular or arterial wall 105, or against inner walls of a shaft or a retaining capsule), and further configured to revert to its original shape (i.e., the shape of its relaxed state) when the external force is no longer applied thereto (e.g., when a valve is released from the shaft or capsule prior to expansion thereof).

It is to be understood that the compressibility of the protrusions 430 does not contradict the resilient 3D structure of the second layer 420, on which any of the flaps 438, helical protrusion 430a, protrusions 430, and/or divided protrusions 434, are formed, or to which they are connected, as upon the ceasing of compression of squeezing thereof, their structure will be reinstated (i.e., revert back to its relaxed state configuration, extending radially outward).

According to some examples, the sealing member 422 includes at least the first layer 410 comprising a tear resistant material and the second layer 420 comprising a thermoplastic thromboresistant material, and any of the plurality of flaps 438, the at least one helical protrusion 430a, the plurality of protrusions 430 or the plurality of divided protrusions 434, extending away from the second layer 420 that coats at least the first surface 402 thereof.

According to some examples, the sealing member 422 further comprises the third layer 425 and/or and the fourth layer 445, each comprising a thermoplastic thromboresistant material. According to further examples, the sealing member 422 includes the first layer 410 comprising a tear resistance material comprising a PET fabric, and the second layer 420 comprising the plurality of protrusions 430 or divided protrusions 434 extending therefrom and comprising thermoplastic thromboresistant material comprising TPU. According to further examples, the sealing member 422 includes the third layer 425 and/or and fourth layer 445, each comprising a thermoplastic thromboresistant material comprising TPU.

The utilization of thermoplastic elastomer material(s), such as TPU, enables to fabricate the sealing member 422 in a manner which enables formation of a desired 3D-shaped sealing member 422 having the plurality of protrusions 430, wherein each one of the plurality of protrusions 430 forms the hollow lumen 431 disposed therein. In some examples, advantageously, the plurality of the protrusions 430 of the sealing member 422 are adapted to contact, and become compressed against, the annular or arterial wall 105 at the implantation site, following the expansion of the prosthetic heart valve 100 therein, so as to improve PVL sealing between the prosthetic heart valve 100 and the inner surface of the annular or arterial wall 105.

Since each one of the plurality of protrusions 430 forms the hollow lumen 431 therein and comprises the thermoplastic material, when the valve is retained in a crimped state within a sheath or a capsule, each one of the plurality of the hollow thermoplastic protrusions 430 can become compressed against the inner walls of a retaining sheath or capsule, without experiencing irreversible deformation, and may spring back to outward when the valve is released, to extend toward the surrounding anatomical wall at the site of implantation and improve PVL sealing after deployment. A configuration comprising hollow thermoplastic protrusions 430 can be advantageous, as it may provide enhanced compressibility compared to full-matter thermoplastic protrusions made from the same material (e.g., non-hollow protrusions).

The utilization of thermoplastic elastomer material(s), such as TPU, enables to fabricate the sealing member 422 in a manner which enables formation of a desired 3D-shaped sealing member 422 having the plurality of protrusions 430, wherein each one of the plurality of protrusions 430 forms the hollow lumen 431 disposed therein, and wherein each one of the hollow lumens 431 comprises the porous elastic material 433 therein. Since each one of the plurality of protrusions 430 forms the hollow lumen 431 comprising the porous elastic material 433 therein and comprises the thermoplastic material, when the valve is retained in a crimped state within a sheath or a capsule, each one of the plurality of the hollow thermoplastic protrusions 430 can become compressed against the arterial wall 105, without experiencing irreversible deformation, and may spring back to outward when the valve is released, to extend toward the surrounding anatomical wall at the site of implantation and improve PVL sealing therebetween. A configuration comprising hollow thermoplastic protrusions 430 filled with porous elastic material 433 can be advantageous, since as it may provide enhanced compressibility compared to full-matter (i.e., non-hollow) thermoplastic protrusions made from a uniform material.

According to some examples, the nonfibrous outer surface(s) (280, 380, 480) of the sealing member(s) (222, 322, 422) of the present invention are formed of a material inherently shaped to define the plurality of elevated portions (230, 330, 430) and the plurality of non-elevated portions (250, 350, 450). According to some examples, the outer surface(s) (280, 380, 480) are defined as the second layer(s) (220, 320, 420) and the elevated portions (230, 330, 430) comprising the thermoplastic elastomer material(s), such as TPU, as disclosed herein above. According to some examples, the inherent properties of the thermoplastic elastomer material(s) forming the outer surface(s) (280, 380, 480) enable the formation of the resilient 3D structure of the sealing members as presented herein above.

Thus, the term “inherently shaped”, as used herein, refers to a material or a layer comprising a material that is pre-shaped to assume a specific non-flat shape (e.g., so as to define an outer surface with elevated portions), such as a thermoplastic material that can be formed to a specific shape under elevated heat, and retain such shape when cooled. A material that is inherently shaped to form a specific non-flat outer surface will assume the same shape in a relaxed state thereof (for example, when no pressure exceeding a predefined threshold is applied to deform it), as opposed to flexible materials or layers that may assume randomized, non-specific, non-flat configurations, for example due to simply being folded, bunched, or inflated/expanded (for example, when internal pressure is applied thereto) to assume such shapes.

According to some examples, at least one of the plurality of protrusions 430, or the at least one helical protrusion 430a, of sealing member 422, defines a hollow lumen 431 therein, which contain a pharmaceutical composition 436 disposed therein (see FIGS. 12F and 12G). According to further examples, such protrusions 430, 430a comprise a plurality of apertures 435 spaced from each other (see FIGS. 12F and 12G), wherein each aperture 435 is configured to provide fluid communication between the hollow lumen 431 and the external environment outside of the apertures 435, i.e., the tissues and/or fluids (e.g., blood flow) at the implantation site. According to some examples, at least some of the lumens 431 of the plurality of protrusions 430 contain a pharmaceutical composition 436 disposed therein. According to some examples, the lumens 431 of all of the plurality of protrusions 430 contain a pharmaceutical composition 436 disposed therein.

It is to be understood that inclusion of the apertures 435 along the protrusions 430 does not contradict their definition of being continuous, as the term “continuous”, with respect to the peak portions described herein, refers to such peak portions being devoid of discontinuities that extend along the entire width of each protrusion 430 (i.e., extending across the entire dimension of the protrusion 430 between the adjacent inter-protrusion gaps 450 on both of its sides).

According to some examples, when the sealing member 422 comprising the plurality of apertures 435 is in a folded state mounted on the prosthetic heart valve 100 (see FIG. 15), the plurality of apertures 435 are configured to allow release of the pharmaceutical composition 436 disposed within the corresponding drug-containing hollow lumen 431, therethrough, for example toward the tissues and/or blood flow at the implantation site, thereby enabling the sealing member 422 to act as a drug-eluting PVL skirt. According to some examples, a sealing member 422 comprising the plurality of apertures 435 can have various configurations and orientation of the protrusions 430, as described above.

According to some examples, each one of the plurality of apertures 435 is sealed by a biodegradable membrane 437 (see FIG. 12G). According to some examples, the biodegradable membrane 437 decomposes slowly over time within the implantation site, and thus enables controlled release of the pharmaceutical composition 436 from within the respective hollow lumens 431 therethrough.

Biodegradable membrane 437 is made of a biodegradable biocompatible material, according to some examples. According to some examples, the biodegradable membrane 437 comprise a biodegradable material, wherein said biodegradable material is configured to controllably degrade over time upon contact with the fluids and/or tissues residing within in the implantation site (e.g., blood). Suitable biodegradable materials can be selected from, but not limited to, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid), and combinations and variations thereof. Each possibility represents a separate example of the present invention.

According to some other examples, each one of the plurality of protrusions 430 of sealing member 422 can be coated or covered externally by a biodegradable coating, thereby covering each one of the plurality of apertures 435, wherein said biodegradable coating can comprise the same materials and have the same properties and/or functionalities as the biodegradable membrane 437 as disclosed herein.

According to some examples, at least some of the hollow lumens 431 contain the pharmaceutical composition 436 disposed therein. According to some examples, each one of the hollow lumens 431 contains the pharmaceutical composition 436 disposed therein. The pharmaceutical composition 436 can be entangled, embedded, incorporated, encapsulated, bound, or attached to an inner surface of each one of the hollow lumens 431, according to some examples, in any way known in the art.

According to some examples, each one of the plurality of protrusions 430 comprises the elastic material 433 disposed therein, wherein the elastic material 433 is a porous elastic element/member comprising the pharmaceutical composition 436 as described herein above. According to some examples, the elastic material 433 comprises a sponge. The pharmaceutical composition 436 can be entangled, embedded, incorporated, encapsulated, bound, or attached to an inner surface of each one of the pores of the porous elastic material 433, in any way known in the art.

According to some examples, the plurality of apertures 435 are configured to allow release of the pharmaceutical composition 436 disposed within the porous elastic material 433 of the respective protrusions 430, therethrough, and toward the tissues and/or fluids (e.g., blood flow) at the implantation site, thereby allowing the sealing member 422 to further act as a drug-eluting PVL skirt. According to some examples, the porous elastic material 433 can be coated or covered by a biodegradable coating, wherein said biodegradable coating can comprise the same materials and have the same properties and/or functionalities as disclosed herein for the biodegradable membrane 437.

According to some examples, the pharmaceutical composition 436 may be in a form selected from solid (such as in a pill or a tablet), gel, absorbed on a solid article, suspension and/or liquid. Each possibility represents a separate example of the present invention.

According to some examples, the pharmaceutical composition 436 comprises at least one pharmaceutical active agent which is selected from the group consisting of antibiotics, antivirals, antifungals, antiangiogenics, analgesics, anesthetics, anti-inflammatory agents including steroidal and non-steroidal anti-inflammatories (NSAIDs), corticosteroids, antihistamines, mydriatics, antineoplastics, immunosuppressive agents, anti-allergic agents, metalloproteinase inhibitors, tissue inhibitors of metalloproteinases (TIMPs), vascular endothelial growth factor (VEGF) inhibitors or antagonists or intraceptors, receptor antagonists, RNA aptamers, antibodies, hydroxamic acids and macrocyclic anti-succinate hydroxamate derivatives, nucleic acids, plasmids, siRNAs, vaccines, DNA binding compounds, hormones, vitamins, proteins, peptides, polypeptides and peptide-like therapeutic agents, anesthetizers and combinations thereof. Each possibility represents a separate example of the present invention.

According to some examples, the pharmaceutical composition 436 comprises thromboresistant pharmaceutical agents and/or pharmaceutical agents configured to prevent or reduce tissue ingrowth.

According to some examples, the pharmaceutical composition 436 further comprises at least one pharmaceutical carrier. Pharmaceutical carriers that may be used in the context of the present invention include various organic or inorganic carriers including, but not limited to, excipients, lubricants, binders, disintegrants, water-soluble polymers and basic inorganic salts. The pharmaceutical compositions of the present invention may further include additives such as, but not limited to, preservatives, antioxidants, coloring agents, etc.

According to some examples, the sealing members of the present invention (222, 322, 422) may comprise at least one ripstop fabric. According to some examples, the first layer(s) of the sealing member(s) of the present invention (e.g., first layer 210, 310, or 410) comprise a tear resistant ripstop fabric, wherein the fabric is optionally a PET fabric. According to further examples, the first layer(s) comprise a ripstop fabric comprising fibers made from polyethylene terephthalate (PET).

According to some examples, the sealing members of the present invention (222, 322, 422) may comprise at least one radiopaque material. Radiopaque materials are understood to be capable of producing a relatively bright image on a fluoroscopy screen or another imaging technique, during the prosthetic valve 100 implantation procedure. Radiopaque materials can include, but are not limited to, gold, platinum, tantalum, tungsten alloy, platinum iridium alloy, palladium, and the like. According to some examples, the at least one radiopaque material can be formed by means of radiopaque inks and adhesives, and applied on at least a portion of the sealing members or on at least one layer thereof, in a number of ways, such as screen printing, high speed roller printing, coating, dipping, etc.

According to some examples, at least a portion of the first layer(s) (e.g., first layers 210, 310, and 410) of the sealing member(s) (e.g., sealing members 222, 322, and 422) of the present invention comprises the at least one radiopaque material. According to further examples, the at least one radiopaque material can be formed by means of radiopaque inks and adhesives, and applied on at least a portion of the first layers, in a number of ways, such as screen printing, high speed roller printing, coating, dipping, etc.

According to some examples, at least a portion of the second layer(s) (e.g., second layers 220, 320, and 420) of the sealing member(s) (e.g., sealing members 222, 322, and 422) of the present invention comprises the at least one radiopaque material. According to further examples, the at least one radiopaque material can be formed by means of radiopaque inks and adhesives, and applied on at least a portion of the second layers, in a number of ways, such as screen printing, high speed roller printing, coating, dipping, etc.

According to some examples, at least a portion of the plurality of protrusions (e.g., protrusions 330, 430 and divided protrusions 434) or ridges (e.g., ridges 230) of the sealing members of the present invention (e.g., sealing members 222, 322, and 422) comprises the at least one radiopaque material. According to further examples, the at least one radiopaque material can be formed by means of radiopaque inks and adhesives, and applied on at least a portion of the plurality of protrusions or ridges, in a number of ways, such as screen printing, high speed roller printing, coating, dipping, etc.

According to some examples, at least a portion of the elastic material 433 (e.g., an elastic foam and/or an elastic metallic cylinder) disposed within each one of the hollow lumens 431 comprises the at least one radiopaque material. According to further examples, the at least one radiopaque material can be formed by means of radiopaque inks and adhesives, or can be an integral component thereof.

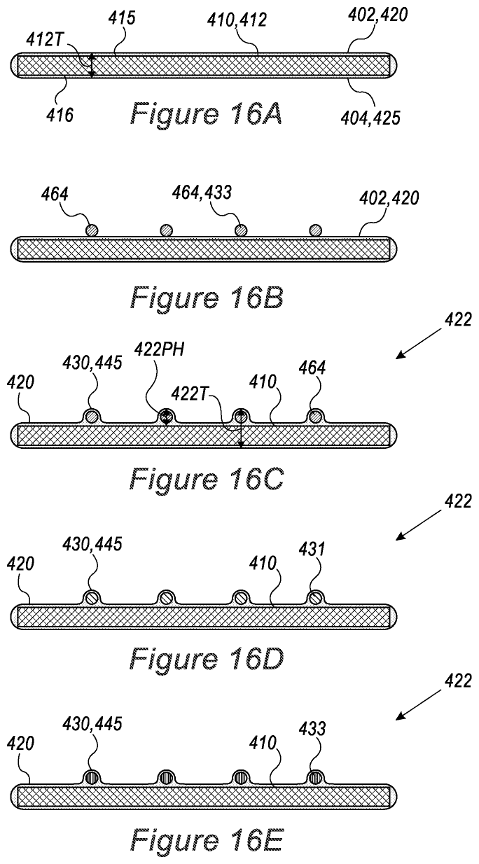

Reference is now made to FIGS. 16A-16E, showing various stages of processing steps for the manufacture of sealing member 422 utilizing a plurality of mandrels 464, according to some examples.

According to some examples, there is provided a PVL skirt 422 prepared by the methods of the present invention. According to some examples, there is provided a PVL skirt 422 in a folded state prepared by the methods of the present invention.

According to some examples, there is provided a method for fabricating the sealing member 422 as described herein above, in a cost-effective and simple manner, the method comprising: (i) providing a tear resistant flat sheet 412; (ii) treating the sheet in a thermal shape-forming process to assume a 3D shape in a spread relaxed state, by: placing a plurality of elongated molding members 464 on the tear resistant flat sheet 412; depositing a thermoplastic layer 445, at an elevated temperature, on the plurality of the elongated molding members 464, thereby forming a plurality of protrusions 430 and causing the sheet to assume a 3D shape; and (iii) connecting two opposite edges of the sheet 412 of step (ii) to form a cylindrical sealing member (or PVL skirt) in a cylindrical folded state.

The terms “elongated molding members” and mandrels are interchangeable, and may refer to elongated members in the form of rods, tubes, pipes, and the like. According to some examples, the elongated molding members 464 are made of a thermo-resistant material. It is to be understood that thermo-resistant materials are material which remain substantially unchanged upon exposure to standard thermal shape-forming temperatures (e.g. below 300° C.). According to some examples, the elongated molding members 464 are made of metal or a metal alloy.

According to some examples, the thermoplastic layer 445 is made of various suitable biocompatible synthetic materials, such as, but not limited to, a thermoplastic material. According to some examples, the thermoplastic layer is made of a thermoplastic material. Suitable thermoplastics biocompatible materials are selected from, but not limited, polyamides, polyesters, polyethers, polyurethanes, polyolefins (such as polyethylene and/or polypropylenes), polytetrafluoroethylenes, and combinations and copolymers thereof. Each possibility represents a different example. Thus, according to some examples, the thermoplastic layer is made of a thermoplastic material. According to some examples, thermoplastic layer comprises a thermoplastic material. According to some examples, thermoplastic layer consists of a thermoplastic material. According to some examples, the thermoplastic material is selected from the group consisting of: polyamides, polyesters, polyethers, polyurethanes, polyolefins, polytetrafluoroethylenes, and combinations and copolymers thereof.

According to some examples, the thermoplastic layer 445 can be made of various suitable biocompatible synthetic materials, such as, but not limited to, thermoplastic material, including thermoplastic elastomers (TPE). According to some examples, the thermoplastic material is a thermoplastic elastomer. According to some examples, the thermoplastic material comprises a thermoplastic elastomer (TPE).

According to some examples, the thermoplastic elastomer is selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations and variations thereof. Each possibility represents a different example. According to some examples, the thermoplastic elastomer is TPU. According to some examples, the thermoplastic elastomer comprises TPU.

According to some examples, the thermoplastic layer 445 comprises at least one thromboresistant material, adapted to prevent the formation of blood clots (thrombus) therearound, in order to prevent and/or reduce tissue ingrowth around the implanted prostatic heart valve, thereby enabling easily and safe explant thereof from the surrounding tissue when required, preferably devoid of complex surgical procedures. According to some examples, the second layer 220 comprises at least one thermoplastic elastomer thromboresistant material. According to some examples, the thermoplastic layer comprises at least one thermoplastic elastomer thromboresistant material, which is adapted to prevent and/or reduce tissue ingrowth therearound. Such material include TPU, according to some examples.

According to some examples, the thermoplastic layer comprises TPU.

According to some examples, depositing the thermoplastic layer 445 in step (ii) is performed at an elevated temperature.

According to some examples, step (ii) comprises removing the plurality of elongated molding members 464 from within the plurality of protrusions 430 after the formation thereof.

According to some examples, step (i) comprises providing a tear resistant flat sheet 412 comprising the first layer 410 that comprises at least one tear resistant material as described herein above, wherein the tear resistant material optionally comprises a PET fabric.

According to some examples, step (i) comprises providing a flat flexible sheet 412, which comprises a tear resistant first layer 410 and a thermoplastic second layer 420. According to some examples, step (i) comprises providing a flat flexible sheet 412, which comprises a tear resistant first layer 410 disposed between a thermoplastic second layer 420 and a thermoplastic third layer 425 of the flat flexible sheet 412 (see FIG. 16A).

According to some examples, step (i) comprises providing a flat flexible sheet 412, which comprises a tear resistant first layer 410, and coating at least a first surface 415 of the first layer 410 with a thermoplastic coating, thereby forming the thermoplastic second layer 420. According to some examples, step (i) comprises providing a flat flexible sheet 412, which comprises a tear resistant first layer 410, and coating a first surface 415 and a second surface 416 of the first layer 410 with a thermoplastic coating, thereby forming the thermoplastic second and third layers 420 and 425, respectively. The coating of the tear resistant first layer 410 can be performed utilizing a coating technique selected from brushing, spray-coating, dip coating, dipping, immersing, chemical deposition, vapor deposition, chemical vapor deposition, physical vapor deposition, roller printing, stencil printing, screen printing, inkjet printing, lithographic printing, 3D printing, and combinations thereof. Each possibility represents a different example.

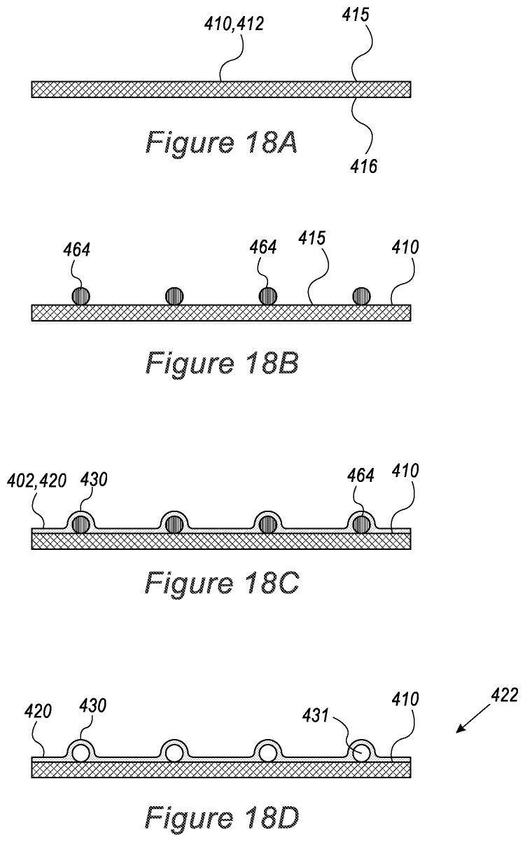

It is to be understood that since step (ii) includes depositing a thermoplastic layer over the flat flexible sheet 412, the flat flexible sheet 412 is not required to be coated in advance (see, for example, FIG. 18A). However, the option of providing a coated flexible sheet 412 in step (i) is contemplated, as detailed herein (see, e.g. FIG. 16A).

It is to be understood that any of the properties introduced above for each one of the layers (i.e., the first layer 410, the second layer 420 and the third layer 425) similarly apply to the respective layers when referring to the method of the present aspect of the invention. According to some examples, the first layer 410 comprises a tear resistant PET fabric. According to some examples, the second layer 420, the third layer 425, or both, comprises at least one thermoplastic material. According to some examples, the second layer 420, the third layer 425, or both, comprises at least one thromboresistant thermoplastic elastomer material comprising TPU. According to some examples, the second layer 420 and the third layer 425 are made from the same material. According to some examples, the third layer 425 is united with the second layer 420 as detailed herein.

According to some examples, the sheet 412 has four substantially right angle vertices, and two sets of two opposing edges (a set of first lateral edge 406 and second lateral edge 408, and a set of outflow edge 407 and an inflow edge 409).

According to some examples, step (ii) comprises placing/positioning a plurality of mandrels 464 on the first surface 415 of the first layer 410 of the tear resistant flat sheet 412. According to some examples, the plurality of mandrels 464 are spaced from each other. According to some alternative examples, step (ii) comprises placing/positioning a plurality of mandrels 464 on the surface 402 of the second layer 420 of the tear resistant flat sheet 412, wherein the plurality of mandrels 464 are spaced from each other therealong (see FIG. 16B). According to some examples, the mandrels 464 are equally spaced from each other.

The mandrels 464 can be positioned over the surface 402 such that each mandrel 464 extends from the first lateral edge 406 to the second lateral edge 408; from the inflow edge 409 to the outflow edge 407 of the sheet; diagonally along at least a portion of a surface of the sheet 412, or any combination thereof.

According to some examples, step (ii) further comprises depositing a thermoplastic layer 445, on the plurality of mandrels 464. According to some examples, the plurality of mandrels 464 are positioned between the flat sheet 412 and the thermoplastic layer 445, to facilitate formation of a plurality of 3D shaped protrusions 430 thereon. According to some examples, step (ii) comprises depositing a thermoplastic layer 445 at an elevated temperature on the surface 402 of the second layer 420 of the tear resistant flat sheet 412, wherein the surface 402 comprises the plurality of mandrels 464 placed thereon during step (ii). According to some examples, step (ii) comprises depositing a thermoplastic layer 445 at an elevated temperature on the plurality of mandrels 464 and on the surface 402 which spaces between adjacent mandrels 464. The deposition of the thermoplastic layer 445, at an elevated temperature, on the plurality of mandrels 464, and optionally the surface 402 which spaces between adjacent mandrels 464, causes the sheet to assume a 3D shape, thereby forming the plurality of protrusions 430 as described herein above.

According to some examples, step (ii) comprises coating the plurality of mandrels 464 and optionally the surface 402 which spaces between adjacent mandrels 464 with a thermoplastic coating, at an elevated temperature, thereby forming the thermoplastic layer 445 thereon (e.g., a fourth layer 445), as can be seen in FIG. 16C. Coating the plurality of mandrels 464, and optionally the surface 402 which spaces between adjacent mandrels 464, with the fourth thermoplastic layer 445, causes the sheet to assume a 3D shape by forming the plurality of protrusions 430 as described herein above, wherein each one of the plurality of protrusions 430 is formed over each mandrel 464, according to some examples. It is to be understood that although layer 445 is indicated as the “fourth layer” or as “thermoplastic fourth layer”, neither sealing member 422 nor the method of the present invention necessarily requires more than two layers. For example, sealing member 422 may include, according to some examples, only the first layer 410 and the fourth layer 445.

It is to be understood that the plurality of mandrels 464 are configured to support the formation of the fourth layer 445 thereover, in order to facilitate the formation of the plurality of protrusions 430 of the sealing member 422. According to some examples, each one of the plurality of mandrels 464 has an elongated structure, and is positioned to extend between two opposing edges of the sheet 412 (the first lateral edge 406 to the second lateral edge 408, or the outflow edge 407 to the inflow edge 409). According to some examples, each one of the plurality of mandrels 464 has an elongated structure, characterized by having various cross-sectional shapes, selected from circle, inverse U-shape, square, rectangle, any other polygon, and combinations thereof. Each possibility represents a different example.

The fourth thermoplastic layer 445 (or the thermoplastic layer 445) can comprise the same materials as the second layer 420 and optionally the third layer 425. The fourth layer 445 can comprise at least one thromboresistant thermoplastic elastomer material comprising TPU.

Coating the plurality of mandrels 464, and optionally the surface 402 which spaces between adjacent mandrels 464, with the fourth layer 445 can be performed at an elevated temperature. The elevated temperature is a temperature sufficient to enable a pliable relatively soft state of the fourth layer 445, as was disclosed herein above in the context of thermoplastic properties of thermoplastic materials. According to some examples, the elevated temperature in step (iii) is above about 60° C., 100° C., 125° C., 150° C., 175° C., 200° C., 225° C., 250° C., 275° C., 300° C., or more. Each possibility represents a different example.

After coating the plurality of mandrels 464, and optionally the surface 402 which spaces between adjacent mandrels 464, with the fourth layer 445, thereby forming the 3D shape of the sheet, the formed 3D shaped sheet can be cooled, thereby stabilizing the 3D shape in the spread relaxed state of the sealing member. According to some examples, step (ii) further comprises cooling (i.e., lowering the temperature of) the sheet 412 to a temperature below 40° C. According to further examples, the lowering of the temperature in step (ii) is cooling the sheet 412 to room temperature.

While cooling the 3D shaped sheet, the fourth layer 445 transitions to a semi-rigid or resilient relatively rigid state, wherein the shape of the coated mandrels 464 can transition to the shape of the plurality of the protrusions 430. The transition from the pliable relatively soft state at elevated temperatures, to the resilient relatively rigid state at lower temperatures, is as explained herein above in the context of thermoplastic properties of thermoplastic materials.

According to some examples, removing the plurality of mandrels 464 from within the plurality of protrusions 430 in step (ii) comprises extracting each mandrel 464 through at least one protrusion edge located at the first lateral edge 406 and/or the second lateral edge 408 of the sheet 412 (or alternatively, at any of the outflow edge 407 or the inflow edge 409), resulting in a plurality of hollow lumens 431 formed therein thus resulting in the sealing member 422 as described herein above (see FIG. 16D). It is to be understood that each hollow lumen 431 corresponds to a previously placed elongated molding member 464, and has a similar cross-sectional profile.

According to some examples, step (ii) further comprises perforating/puncturing a plurality of apertures 435 in the plurality of protrusions 430. The apertures 435 may be formed on a surface of at least one protrusion 430 (e.g. by puncturing or melting, for example, using a focused laser beam) such that the resulting opening of the aperture is flush with the external surface of the protrusion. In further examples, step (ii) comprises perforating/puncturing a plurality of apertures 435 at each protrusion 430, wherein the plurality of apertures 435 are spaced from each other therealong, and are configured to provide fluid communication between the hollow lumen 431 and the external environment outside of the apertures 435, as disclosed herein above, thereby forming the sealing member 422 as illustrated at FIGS. 12F and 12G. According to some examples, step (ii) further comprise inserting a pharmaceutical composition 436, as disclosed herein above, into at least part of the hollow lumens 431. The pharmaceutical composition 436 can be entangled, embedded, incorporated, encapsulated, bound, or attached to an inner surface of each one of the hollow lumens 431. According to some examples, step (ii) further comprise sealing at least part of the apertures 435 with a biodegradable membrane 437, as described above.

According to some examples, the sheet 412 of step (i) has a first surface 402 and a second surface 404, wherein the distance between the first surface 402 and a second surface 404 of the sheet 412 of step (i) constitutes the initial thickness 412T of the sheet 412 of step (i) (see FIG. 16A). According to some examples, the sheet 412 of step (i) is flat and substantially two dimensional. This means that the initial thickness 412T of the sheet 412 of step (i) is substantially shorter that an initial width and/or an initial length of the sheet 412. According to some examples, the initial thickness 412T corresponds to, or is identical to, the total layer thickness 403, as described above.

According to some examples, upon performing the method of the present invention, protrusions 430 are formed, wherein the protrusions 430 have protrusion height 422PH, being part of the thickness 422T of sealing member 422 in its spread relaxed state (see FIG. 16C).

According to some examples, the thickness 422T of sealing member 422 in its spread relaxed state, following the formation of the plurality of protrusions 430 at step (ii), is configured to assume the 3D shape thereof, and is at least 1000% greater than the initial thickness 412T of the sheet 412. According to further examples, the thickness 422T of sealing member 422 in its spread relaxed state is at least 2000%, at least 3000%, at least 4000%, at least 5000%, or at least 6000% greater than the initial thickness 412T of the sheet 412. Each possibility represents a different example.

According to some examples, the thickness modification of the sheet 412 following the method as described herein (412T to 422T) is configured to convert the initial 2D structure of the sheet 412 to a 3D structure in sealing member 422. In some implementation, the resulting sheet 412 after step (ii) has dimensions that are greater than any of a desired final width and/or length, and the method can include an additional step of cutting the sheet 412 to a desired final width and/or length, after step (ii) and prior to step (iii).

According to some examples, step (iii) comprises connecting two opposite edges (i.e., first lateral edge 406 and second lateral edge 408) of the sheet of step (ii) to form a cylindrical sealing member (or PVL skirt) in a cylindrical folded state (see for example, FIG. 20). The connection between the opposite edges can be performed by using at least one of adhesives, sutures, or heating and optionally melting the edges thereof. Alternatively, step (iii) comprises coupling the sealing member 422 to an outer surface of the frame 106, utilizing at least one of adhesives, sutures, or heating and optionally melting the edges of the sealing member 422 therearound.

According to some examples, for the sealing member configuration illustrated in FIGS. 13D and 14D, the fabrication method comprises: (i) providing the tear resistant flat sheet 412 in a folded cylindrical state extending from an inflow edge 409 toward an outflow edge 407; (ii) treating the sheet in a thermal shape-forming process to assume a 3D shape in a spread relaxed state, by: placing at least one helical mandrel (not shown) on the tear resistant flat sheet 412 in a helical configuration therearound; depositing a thermoplastic layer as described herein above, at an elevated temperature, on the at least one helical mandrel, thereby forming the at least one helical 3D protrusion 430a thereon, extending radially away at a helical configuration therearound; and removing the at least one helical mandrel from within the at least one helical protrusion 430a through at least one open-ended helical protrusion edge located at the inflow edge 409 or the outflow edge 407, thereby forming the folded sealing member 422a as described herein above.

According to some examples, step (i) comprises providing a flat flexible sheet 412, which comprises a tear resistant first layer 410 and a thermoplastic second layer 420. According to some examples, step (ii) entails placing the at least one helical mandrel around the thermoplastic second layer 420 of the flat flexible sheet 412, and depositing a thermoplastic layer as described herein above, at an elevated temperature, on the at least one helical mandrel, wherein the helical mandrel is positioned between the thermoplastic second layer 420 of the flat flexible sheet 412 and the thermoplastic layer, thereby forming at least one 3D shaped helical protrusion 430a.

According to some examples, step (ii) further comprises lowering the temperature, thereby maintaining a resilient 3D structure of the thermoplastic layer, wherein the thermoplastic layer is thermally shape-formable at the elevated temperature and resilient at the lowered temperature, as disclosed herein above. According to further examples, the removal of the at least one helical mandrel from within the at least one helical protrusion 430a through at least one helical protrusion edge forms a helical hollow lumen therein. The helical mandrel can be made from the same materials and have similar properties to each mandrel 464 as described herein.

According to some examples, step (ii) further comprises perforating/puncturing a plurality of apertures 435 in the helical protrusion. In further examples, step (ii) comprises perforating/puncturing a plurality of apertures 435 at the helical protrusion, wherein the plurality of apertures 435 are spaced from each other therealong, and are configured to provide fluid communication between the helical hollow lumen and the external environment outside of the apertures 435, as disclosed herein above. According to some examples, step (ii) further comprise inserting a pharmaceutical composition 436, as disclosed herein above, into at least a part of the helical hollow lumen. The pharmaceutical composition 436 can be entangled, embedded, incorporated, encapsulated, bound, or attached to an inner surface of the helical hollow lumen. According to some examples, step (ii) further comprise sealing at least a part of the apertures 435 with a biodegradable membrane 437, as described above.

According to some examples, a method for fabrication of the sealing member 422 configuration illustrated in FIG. 16E comprises: (i) providing a tear resistant flat sheet 412 as described herein above; (ii) treating the sheet in a thermal shape-forming process to assume a 3D shape in a spread relaxed state, by: placing a plurality of mandrels 464 which are a plurality of elongated elastic porous members 433 on the tear resistant flat sheet 412; depositing a thermoplastic layer as described herein above, at an elevated temperature, on the plurality of elongated elastic porous members 433, thereby forming a plurality of protrusions 430, causing the sheet to assume a 3D shape, and obtaining the sealing member 422 as described herein above; and (iii) connecting two opposite edges of the sheet 412 of step (ii) to form a cylindrical sealing member (or PVL skirt) in a cylindrical folded state.

According to some examples, step (ii) comprises placing/positioning a plurality of elastic porous members 433 on the first surface 415 of the first layer 410 of the tear resistant flat sheet 412, wherein the plurality of elastic porous members 433 are spaced from each other. According to some alternative examples, step (ii) comprises placing/positioning a plurality of elastic porous members 433 on the surface 402 of the second layer 420 of the tear resistant flat sheet 412, wherein the plurality of elastic porous members 433 are spaced from each other therealong.

According to some examples, the plurality of elongated elastic porous members are a plurality of elongated porous sponges 433. According to some examples, the elastic porous members 433 comprises an elastic foam, such as an elastic sponge. According to some examples, step (ii) comprises impregnating the plurality of elastic porous members 433 with a pharmaceutical composition 436 as described herein above, prior to the deposition thereof on the flat sheet 412.

According to some examples, step (ii) comprises coating the plurality of elastic porous members 433 and optionally the surface 402 which spaces between adjacent elastic porous members 433 with a thermoplastic coating, at an elevated temperature, thereby forming the fourth layer 445 thereon (see FIG. 16E). According to some examples, coating the plurality of elastic porous members 433, and optionally the surface 402 which spaces between adjacent elastic porous members 433, with the fourth layer 445 causes the sheet to assume a 3D shape by forming the plurality of protrusions 430 as described herein above, wherein each one of the plurality of protrusions 430 is formed over each elastic porous members 433, as can be appreciated from the figures by the skilled in the art.

It is to be understood that the plurality of elastic porous members 433 are configured to support the formation of the fourth layer 445 thereon, in order to facilitate the formation of the plurality of protrusions 430 of the sealing member 422. Thus, the plurality of elastic porous members 433 may contribute to the 3D shape of the sealing member 422. According to some examples, each one of the plurality of elastic porous members 433 has an elongated structure. According to some examples, some of the plurality of elastic porous members 433 have elongated structures.

According to some examples, the elastic porous members 433 extend between two opposing edges of the sheet 412 (the first lateral edge 406 to the second lateral edge 408, or the outflow edge 407 to the inflow edge 409).

According to some examples, the elastic porous members 433 extend between the first lateral edge 406 and the second lateral edge 408, and are spaced apart from each other along the axis between the outflow edge 407 and the inflow edge 409. According to some examples, the elastic porous members 433 are placed to extend between the outflow edge 407 and the inflow edge 409, and are spaced apart from each other along the axis between the first lateral edge 406 and the second lateral edge 408. It is to be understood that sponges 433 which extend between two opposing edges of the sheet 412 (the first lateral edge 406 to the second lateral edge 408, or the outflow edge 407 to the inflow edge 409) are typically elongated.

Nevertheless, it is not required, according to some examples, for the sponges 433 to extend is this manner, as they may be placed in a broken (i.e., non-continuous) or fragmented fashion. In such broken configuration the sponges 433 are not required to be elongated. According to some examples, the elastic porous members 433 are placed to be spaced apart one from the other along the axis the first lateral edge 406 and the second lateral edge 408, and to be spaced apart one from the other along the axis between the outflow edge 407 and the inflow edge 409.

According to some examples, in order to form the sealing member configuration illustrated in FIG. 16E, each one of the plurality of protrusions 430 of the sealing member 422 comprises the elastic porous member 433 disposed therein, wherein the fabrication method thereof is devoid of extracting the elastic porous member 433 from within the plurality of protrusions 430. As such, the elastic porous member 433 remains within the sealing member 422 formed by this specific method, in both the spread and folded states thereof.

According to some examples, step (iii) comprises connecting two opposite edges (i.e., first lateral edge 406 and second lateral edge 408) of the sheet of step (ii) to form a cylindrical sealing member (or PVL skirt) in a cylindrical folded state (see for example, FIG. 20). The connection between the opposite edges can be performed by using at least one of adhesives, sutures, or heating and optionally melting the edges thereof. Alternatively, step (iii) comprises coupling the sealing member 422 to an outer surface of the frame 106, utilizing at least one of adhesives, sutures, or heating and optionally melting the edges of the sealing member 422 therearound.

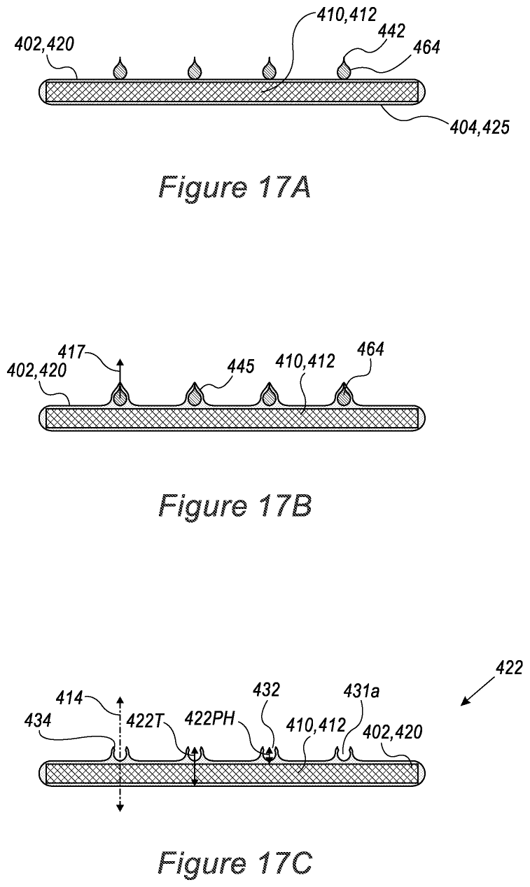

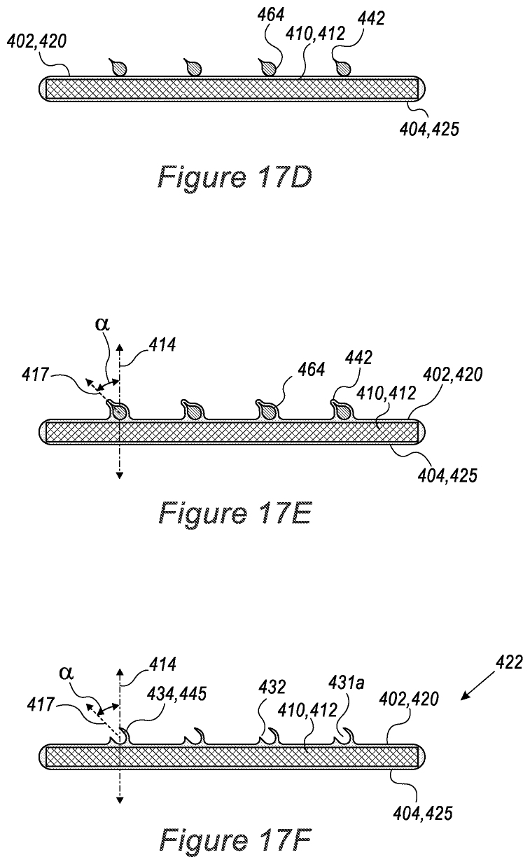

Reference is now made to FIGS. 17A-17F, showing various stages of processing steps for the manufacture of sealing member 422, utilizing a plurality of mandrels 464 comprising sharp tips 442, according to some examples.

According to some examples, there is provided a method for fabricating the sealing member 422 as described herein above, in a cost-effective and simple manner, the method comprising: (i) providing a tear resistant flat sheet 412; (ii) treating the sheet in a thermal shape-forming process to assume a 3D shape in a spread relaxed state, by: placing a plurality of mandrels 464 on the tear resistant flat sheet 412, wherein each one of the plurality of mandrels 464 comprises a sharp tip 442 (FIG. 17A); depositing a thermoplastic layer, at an elevated temperature, on the plurality of elongated molding members 464, thereby forming a plurality of protrusions 430 and causing the sheet to assume a 3D shape (FIG. 17B); removing the plurality of elongated molding members 464 through the plurality of protrusions 430, thereby forming the plurality of divided protrusions 434 (FIG. 17C); and (iii) connecting two opposite edges of the sheet 412 of step (ii) to form a cylindrical sealing member (or PVL skirt) in a cylindrical folded state.

According to some examples, the elongated molding members 464 are made of a thermo-resistant material. It is to be understood that thermo-resistant materials are material which remain substantially unchanged upon exposure to standard thermal shape-forming temperatures (e.g. below 300° C.). According to some examples, the elongated molding members 464 are made of metal or a metal alloy. According to some examples, the elongated molding members 464 are mandrels.

It is to be understood that removing the plurality of elongated molding members 464 through the plurality of protrusions 430, thereby forming the plurality of divided protrusions 434 entails dislocating the elongated molding members 464 in a direction, which is not parallel to the surface of tear resistant flat sheet 412 (i.e. surfaces 402 and 404). As discussed herein, the direction may be substantially perpendicular to form a sealing member as shown in FIG. 17C, or rotated with an angle with respect to the surface of tear resistant flat sheet 412, as shown in FIG. 17F.

According to some examples, depositing the thermoplastic layer (i.e., thermoplastic layer 445 as described above) on the plurality of mandrels 464 entails contacting the thermoplastic layer with the sharp tips 442 of the elongated molding members 464.

According to some examples, step (i) comprises providing a flat flexible sheet 412, which comprises a tear resistant first layer 410. According to some examples, step (i) comprises providing a flat flexible sheet 412, which comprises a tear resistant first layer 410 and a thermoplastic second layer 420. According to some examples, step (i) comprises providing a flat flexible sheet 412, which comprises a tear resistant first layer 410 located between a thermoplastic second layer 420 and a thermoplastic third layer 425 of the flat flexible sheet 412 (see FIG. 17A).

It is to be understood that any of the properties introduced above for each one of the layers (i.e. the first layer 410, the second layer 420 and the third layer 425) similarly apply to the respective layers when referring to the method of the present invention. According to some examples, the first layer 410 comprises a tear resistant PET fabric. According to some examples, the second layer 420, the third layer 425, or both, comprises at least one thermoplastic material. According to some examples, the second layer 420, the third layer 425, or both, comprises at least one thromboresistant thermoplastic elastomer material comprising TPU. According to some examples, the second layer 420 and the third layer 425 are made from the same material. According to some examples, the third layer 425 is united with the second layer 420 as detailed herein above. According to some examples, the material forming the second layer 420 and the third layer 425, if incorporated into the sealing member 422, is the same as the material forming the thermoplastic layer of step (ii).

According to some examples, step (ii) comprises placing/positioning the plurality of elongated molding members 464 on the surface 402 of the second layer 420 of the tear resistant flat sheet 412, wherein the plurality of elongated molding members 464 are spaced from each other, and wherein each one of the plurality of elongated molding members 464 comprises the sharp tip 442. According to further examples, the plurality of elongated molding members 464 are placed on surface 402 so that the sharp tip 442 is facing in the opposite direction relative to the surface 402.

According to some alternative examples, step (ii) comprises placing/positioning the plurality of elongated molding members 464 on the surface 402 of the second layer 420 of the tear resistant flat sheet 412, wherein the plurality of elongated molding members 464 are spaced from each other, and wherein each one of the plurality of elongated molding members 464 is narrow/slim and does not comprise the sharp tip 442. Such narrow-elongated molding members 464 can contain wires. Due to their small size, the removal of the narrow-elongated molding members 464 through the plurality of protrusions 430 can be performed without the sharp tips 442, in order to from the plurality of divided protrusions 434.

According to some examples, the elongated molding members 464 are placed in step (ii) to extend between the first lateral edge 406 and the second lateral edge 408, and to be spaced apart one from the other along the axis between the outflow edge 407 and the inflow edge 409. According to some examples, the elongated molding members 464 are placed in step (ii) to extend between the outflow edge 407 and the inflow edge 409, and to be spaced apart one from the other along the axis between the first lateral edge 406 and the second lateral edge 408. It is to be understood that elongated molding members 464 which extend between two opposing edges of the sheet 412 (the first lateral edge 406 to the second lateral edge 408, or the outflow edge 407 to the inflow edge 409) are typically elongated.

According to some examples, step (ii) comprises coating the plurality of elongated molding members 464 and optionally the surface 402 which spaces between adjacent elongated molding members 464 with a thermoplastic coating, at an elevated temperature, thereby forming the fourth layer 445 thereon (see FIG. 17B). Coating the plurality of elongated molding members 464 comprising the sharp tips 442, and optionally the surface 402 which spaces between adjacent elongated molding members 464, with the fourth layer 445 causes the sheet to assume a 3D shape by forming the plurality of protrusions 430 as described herein above, wherein each one of the plurality of protrusions 430 is formed over each elongated molding member 464 having a sharp tip 442.

It is to be understood that the plurality of elongated molding members 464 are configured to assist the formation of the fourth layer 445, in order to facilitate the formation of the plurality of protrusions 430 of the sealing member 422. According to some examples, each one of the plurality of elongated molding members 464 has an elongated structure comprising an elongated sharp tip 442, configured to extend between two opposing edges of the sheet 412 (the first lateral edge 406 to the second lateral edge 408, or the outflow edge 407 to the inflow edge 409). According to further examples, each one of the plurality of elongated molding members 464 have an elongated cylinder shape comprising an elongated sharp tip 442.

The fourth layer 445 can comprise the same materials as the second layer 420 and optionally the third layer 425. The fourth layer 445 can comprise at least one thromboresistant thermoplastic elastomer material comprising TPU. The fourth layer 445 can further comprise various adhesives or additives, configured to enhance the attachment between the plurality of mandrels 464, and optionally the surface 402 which spaces between adjacent mandrels 464.

Coating the plurality of elongated molding members 464, and optionally the surface 402 which spaces between adjacent elongated molding members 464, with the fourth layer 445 can be performed at an elevated temperature, as described herein above. After coating the plurality of mandrels 464, and optionally the surface 402 which spaces between adjacent elongated molding members 464, with the fourth layer 445, thereby forming the 3D shape of the sheet, the formed 3D shaped sheet can be cooled, thereby stabilizing the 3D shape in the spread relaxed state of the sealing member 422. While cooling the 3D shaped sheet, the fourth layer 445 transitions to a semi-rigid or resilient relatively rigid state, wherein the shape of the coated elongated molding member (e.g. mandrel) 464 can transition to the shape of the plurality of the protrusions 430. The transition from the pliable relatively soft state at elevated temperatures, to the resilient relatively rigid state at lower temperatures was previously explained herein above, in the context of thermoplastic properties of thermoplastic materials.

According to some examples, step (ii) of removing the plurality of elongated molding members 464 through the plurality of protrusions 430 comprises attracting/pulling each sharp tip 442 of each mandrel 464, through the fourth layer 445, in the direction of pulling arrow 417 (see FIG. 17B), thereby forming the plurality of divided protrusions 434. According to further examples, step (ii) comprises pulling each sharp tip 442 of each elongated molding member 464 through the fourth layer 445, wherein the interaction between each sharp tip 442 and the fourth layer 445 coating each protrusion 430 causes the fourth layer 445 to be cut or torn, resulting in the plurality of divided protrusions 434.

According to some alternative examples, step (ii) of removing the plurality of elongated molding members 464 through the plurality of protrusions 430 comprises pressing the fourth layer 445 against the sharp tips 442 (not shown) of the elongated molding members 464 (i.e., in a direction opposite to the pulling arrow 417), thereby forming the plurality of divided protrusions 434. According to further examples, the pressing of the fourth layer 445 against the sharp tips 442 causes the fourth layer 445 to be cut or torn, resulting in the plurality of divided protrusions 434.

According to further examples, each sharp tip 442 of each elongated molding member 464 is pulled along the axis 414 extending through the middle of each divided protrusion 434, in the direction of pulling arrow 417, thereby forming a symmetric inner space 431a therein (see FIG. 17C) and obtaining the sealing member 422 as described herein above. According to further examples, each inner space 431a is extending between an opening 432 of each divided protrusion toward the first surface 402 of the sealing member 422 (i.e., the second layer 420).

According to some examples, step (iii) comprises connecting two opposite edges (i.e., first lateral edge 406 and second lateral edge 408) of the sheet of step (ii) to form a cylindrical sealing member (or PVL skirt) in a cylindrical folded state. The connection between the opposite edges can be performed by using at least one of adhesives, sutures, or heating and optionally melting the edges thereof. Alternatively, step (iii) comprises coupling the sealing member 422 to an outer surface of the frame 106, utilizing at least one of adhesives, sutures, or heating and optionally melting the edges of the sealing member 422 therearound.

According to some alternative examples, step (ii) comprises placing/positioning the plurality of elongated molding members 464 on the surface 402 of the second layer 420 of the tear resistant flat sheet 412, wherein the plurality of elongated molding members 464 are spaced from each other, and wherein the plurality of mandrels 464 are placed on surface 402 so that the sharp tip 442 is diverted at an angle α relative to the axis 414, as can be seen at FIGS. 17D and 17E. According to further such examples, step (ii) comprises pulling the sharp tip 442 of each elongated molding member 464 through the fourth layer 445, wherein the interaction between each sharp tip 442 and the fourth layer 445 coating each protrusion 430 causes the fourth layer 445 to be cut or torn, resulting in a plurality of divided protrusions 434 as a result thereof, wherein the sharp tip 442 of each elongated molding members 464 is pulled in the direction of pulling arrow 417 which is diverted at the angle α relative to the axis 414, thereby forming an asymmetric inner space 431a therein, as can be seen at FIG. 17F.

According to some examples, upon performing the method of the present invention, divided protrusions 434 are formed, wherein the divided protrusions 434 have protrusion height 422PH, being part of the thickness 422T of sealing member 422 in its spread relaxed state (see FIGS. 12D and 17C). According to further examples, the thickness 422T of sealing member 422 in its spread relaxed state (see FIG. 17C) is at least 1000%, 2000%, at least 3000%, at least 4000%, at least 5000%, or at least 6000% greater than the initial thickness 412T of the sheet 412 (see FIG. 17A). Each possibility represents a different example.

Reference is now made to FIGS. 18A-18D, showing various stages of processing steps for the manufacture of sealing member 422 utilizing a plurality of mandrels 464, according to some examples.

As can be appreciated by the skilled in the art, the method illustrated in FIGS. 18A-D are similar to the method described above in conjunction with FIGS. 16A-E, except that in the method illustrated in FIGS. 18A-D, the initial tear resistant flat sheet 412 comprises the first layer 410 as a sole layer (FIG. 18A), while in the method of FIGS. 16A-E the initial tear resistant flat sheet 412 comprises the first layer 410 disposed between a thermoplastic second and third layers 420 and 425, respectively (FIG. 16A). As such, some of the examples describing the method of FIGS. 16A-E similarly apply to the method of FIGS. 18A-D and may be used to describe and define steps of the method of FIGS. 18A-D.

According to some examples, there is provided a method for of fabricating the sealing member 422 as described herein above, in a cost-effective and simple manner, the method comprising: (i) providing a tear resistant flat sheet 412 comprising the first layer 410 comprising at least one tear resistant material as described herein above (FIG. 18A), wherein the tear resistant material optionally comprises a PET fabric; (ii) treating the sheet in a thermal shape-forming process to assume a 3D shape in a spread relaxed state, by: placing a plurality of mandrels 464 as described herein above on the first surface 415 of the first layer 410 of the tear resistant flat sheet 412, wherein the plurality of mandrels 464 are spaced from each other therealong (FIG. 18B); coating the plurality of mandrels 464 and the first surface 415 which spaces between adjacent mandrels 464 with a thermoplastic coating as described herein above, at an elevated temperature, thereby forming the second layer 420 and the plurality of protrusions 430 thereon (see FIG. 18C), and causing the sheet to assume a 3D shape; removing the plurality of mandrels 464 from within the plurality of protrusions 430; and (iii) connecting two opposite edges of the sheet 412 of step (iv) to form a cylindrical sealing member (or PVL skirt) in a cylindrical folded state.

According to some examples, the tear resistant flat sheet 412 of step (i) further comprises a thermoplastic third layer 425 coating the second surface 416 of the first layer 410 (not shown).

It is to be understood that any of the properties introduced above for each one of the layers (i.e. the first layer 410, the second layer 420 and the third layer 425) similarly apply to the respective layers when referring to the method of the present invention.

According to some examples, step (ii) of removing the plurality of mandrels 464 from within the plurality of protrusions 430 comprises extracting each mandrel 464 through at least one protrusion edge located at the first lateral edge 406 and/or the second lateral edge 408 of the sheet 412, thereby forming a plurality of hollow lumens 431 therein and obtaining the sealing member 422 as described herein above (see FIG. 18D).

According to some examples, step (ii) comprises connecting two opposite edges (i.e., first lateral edge 406 and second lateral edge 408) of the sheet of step (ii) to form a cylindrical sealing member (or PVL skirt) in a cylindrical folded state. The connection between the opposite edges can be performed by using at least one of adhesives, sutures, or heating and optionally melting the edges thereof. Alternatively, step (iii) comprises coupling the sealing member 422 to an outer surface of the frame 106, utilizing at least one of adhesives, sutures, or heating and optionally melting the edges of the sealing member 422 therearound.

Reference is now made to FIGS. 19A-19D, showing various stages of processing steps for the manufacture of sealing member 422, utilizing a plurality of mandrels 464 comprising sharp tips 442, according to some examples.

As can be appreciated by those skilled in the art, the method illustrated in FIGS. 19A-D are similar to that of the method illustrated in FIGS. 17A-E, except that in the method of FIGS. 19A-D, the initial tear resistant flat sheet 412 comprises the first layer 410 as a sole layer (FIG. 19A), while in the method of FIGS. 17A-E the initial tear resistant flat sheet 412 comprises the first layer 410 disposed between a thermoplastic second and third layers 420 and 425, respectively (FIG. 17A). As such, some of the examples describing the method of FIGS. 17A-E similarly apply to the method of FIGS. 19A-D and may be used to describe and define steps of the method of FIGS. 19A-D.

According to some examples, there is provided a method for of fabricating the sealing member 422 as described herein above, in a cost-effective and simple manner, the method comprising: (i) providing a tear resistant flat sheet 412 comprising the first layer 410 comprising at least one tear resistant material as described herein above (FIG. 19A), wherein the tear resistant material optionally comprises a PET fabric; (ii) treating the sheet in a thermal shape-forming process to assume a 3D shape in a spread relaxed state, by: placing a plurality of mandrels 464 as described herein above on the first surface 415 of the first layer 410 of the tear resistant flat sheet 412, wherein the plurality of mandrels 464 are spaced from each other therealong, and wherein each one of the plurality of mandrels 464 comprises a sharp tip 442 (FIG. 19B); coating the plurality of mandrels 464 and the first surface 415 which spaces between adjacent mandrels 464 with a thermoplastic coating as described herein above, at an elevated temperature, thereby forming the second layer 420 and the plurality of protrusions 430 thereon (see FIG. 19C), and causing the sheet to assume a 3D shape; removing the plurality of mandrels 464 from within the plurality of protrusions 430, thereby forming the plurality of divided protrusions 434 (FIG. 19D); and (iii) connecting two opposite edges of the sheet 412 of step (iv) to form a cylindrical sealing member (or PVL skirt) in a cylindrical folded state.

According to some examples, the tear resistant flat sheet 412 of step (i) further comprises a thermoplastic third layer 425 coating the second surface 416 of the first layer 410 (not shown).

It is to be understood that any of the properties introduced above for each one of the layers (i.e. the first layer 410, the second layer 420 and the third layer 425) similarly apply for the respective layers when referring to the method of the present invention.

According to some examples, the plurality of mandrels 464 are placed on the first surface 415 of the first layer 410 so that the sharp tip 442 is facing in the opposite direction relative to the surface 415 (see FIG. 19B). According to further such examples, step (iv) comprises pulling each sharp tip 442 of each mandrel 464 through the second layer 420, wherein the interaction between each sharp tip 442 and the second layer 420 coating each protrusion 430 causes the second layer 420 to be cut or torn, thereby obtaining the plurality of divided protrusions 434 as a result thereof. Each sharp tip 442 of each mandrel 464 is pulled along the axis 414 extending through the middle of each divided protrusion 434, in the direction of pulling arrow 417, thereby forming a symmetric inner space 431a therein (see FIG. 19D) and achieving the configuration of the sealing member 422 as described herein above. According to still further examples, each symmetric inner space 431a is extending between an opening 432 of each divided protrusion toward the first surface 415 of the first layer 410.

According to some alternative examples, the plurality of mandrels 464 are placed on the first surface 415 of the first layer 410 so that the sharp tip 442 is diverted at an angle α relative to the axis 414 (not shown). According to further such examples, step (ii) comprises pulling each sharp tip 442 of each mandrel 464 through the second layer 420, wherein the interaction between each sharp tip 442 and the second layer 420 coating each protrusion 430 causes the second layer 420 to be cut or torn, thereby resulting in a plurality of divided protrusions 434. Each sharp tip 442 of each mandrel 464 is pulled in the direction of pulling arrow 417 which is diverted at the angle α relative to the axis 414, thereby forming an asymmetric inner space 431a therein (not shown). According to still further examples, each asymmetric inner space 431a is extending between an opening 432 of each divided protrusion toward the first surface 415 of the first layer 410.

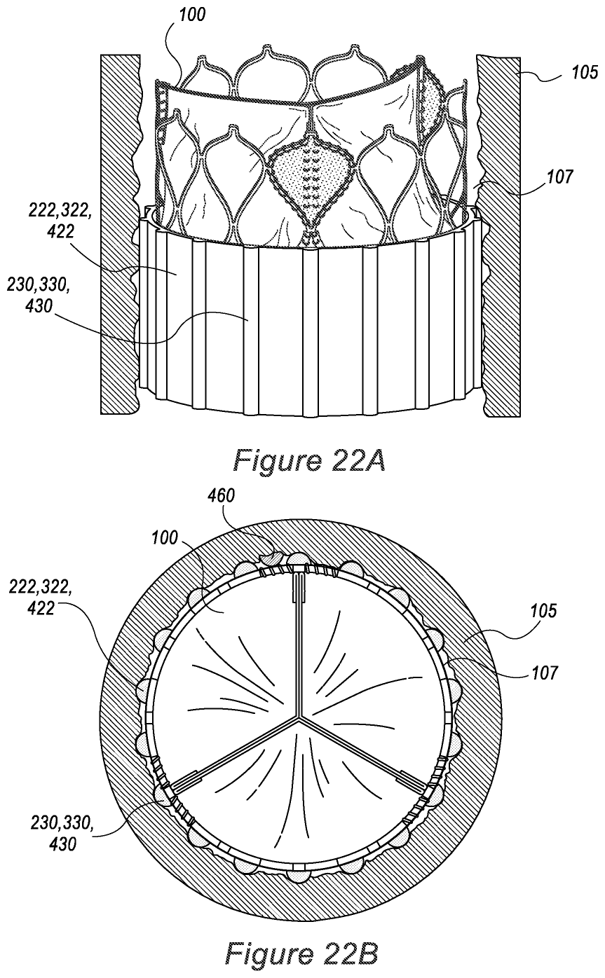

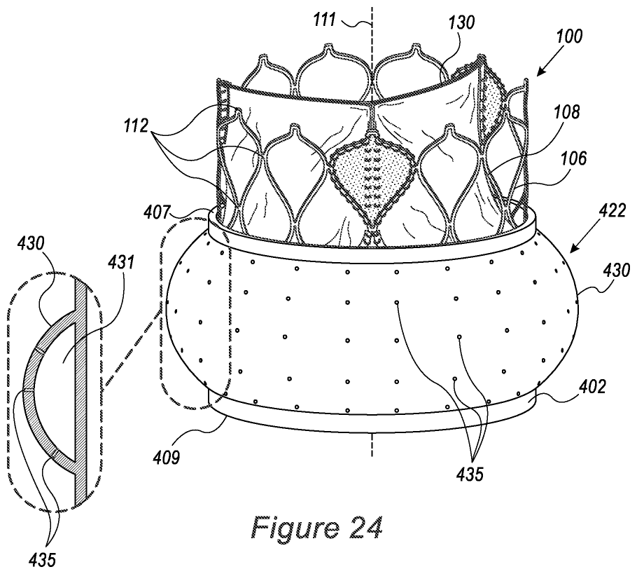

Reference is ow made to FIGS. 20-24. FIG. 20 show a view in perspective of a sealing member, which can correspond to any of the various configurations of the sealing members of the present invention, during transitioning thereof to a cylindrical folded state, according to some examples. FIGS. 21A-21B show a side view and a top view, respectively, of the prosthetic valve 100 comprising various sealing members at a specific configuration, positioned at a target implantation site, according to some examples. FIGS. 22A-22B show a side view and a top view, respectively, of the prosthetic valve 100 comprising various sealing members at a specific configuration, positioned at a target implantation site, according to some examples. FIGS. 23A-23B show an additional configuration of sealing member 422 comprising a single protrusion, mounted on the frame 106 of prosthetic valve 100, in an expanded state (FIG. 23A), and in a crimped state (FIG. 23B), according to some examples. FIG. 24 shows another example of a sealing member 422 comprising a single protrusion, mounted on the frame 106 of prosthetic valve 100.

FIG. 20 show a 3D sealing member (e.g., sealing member 322 or 422) being folded so as to assume a cylindrical folded configuration, by bending the two opposite lateral edges (such as the first lateral edge 306 and a second lateral edge 308 of the sealing member 322) into contacting each other to form a cylindrical shape. The connection between the opposite lateral edges (first lateral edge 306 and second lateral edge 308, or first lateral edge 406 and second lateral edge 408) can be performed by using at least one of adhesives, clipping, sutures, or heating and optionally melting the edges thereof as described herein above.

The circumferential configurations of the plurality of protrusions (e.g., 330 and 430) or ridges (e.g., 230) of the sealing members of the present invention (e.g., sealing members 222, 322, and 422 as shown in FIGS. 5A, 9A, and 14A), relative to the axial flow direction across the annular or arterial wall 105 and/or the sealing member centerlines (when the sealing member is coupled to the outer surface of the frame 106 of the prosthetic valve 100) is advantageous, since this configuration can improve PVL sealing between the sealing members and the annular or arterial wall 105, by preventing or at least significantly reducing perivalvular leakage (PVL) of blood around the valve 100 through the gaps 107 (see FIGS. 21A-21B). The circumferential configurations of the plurality of protrusions (see, for example, FIGS. 9A and 14A) or ridges (see, for example, FIG. 5A) of the sealing members of the present invention as described herein are advantageous, due to their potential to form a physical barrier that prevents valvular leakage (PVL) around the valve 100 through the gaps 107.

Heart valve calcification is a condition in which calcium deposits can form on various sections of aortic heart valves. Calcifications (i.e., the calcium deposits) may become embedded and/or superimposed on the aortic valve leaflets, which are connected to the aortic wall just below the coronary ostia, making the leaflets thicker and less pliable. Calcification may occur at the base of the leaflet, i.e. where the leaflet connects to the annulus or aortic wall, which can significantly impair the mobility of the leaflet, and thus result in issues such as valve stenosis, blood flow restriction and possibly valve malfunction. For example, the arterial wall 105 may comprise at least one calcification 460, as illustrated in FIG. 22B.

According to some examples, the axial (see FIGS. 5B, 9B, and 14B) and/or diagonal (see FIGS. 5C, 9C, and 14C) configurations of the plurality of protrusions (e.g., protrusions 330 and 430), divided protrusions (e.g., protrusions 434), or ridges (e.g., ridges 230), of the sealing members of the present invention (e.g., sealing members 222, 322, and 422), relative to the axial flow direction across the annular or arterial wall 105 (when the sealing member is coupled to the outer surface of the frame 106 of the prosthetic valve 100) can be advantageous when the implantation site includes significant calcifications, as can be seen at FIG. 22A, since such sealing member configurations can be angularly oriented and positioned within the site of implantation, relative to calcium deposits, in a manner that can improve PVL sealing.

During implantation of the prostatic heart valve, the axial and/or diagonal configurations of the plurality of protrusions or ridges of the sealing members of the present invention, can be angularly adjusted within the site of implantation, such that following implantation, the calcification(s) (e.g., calcification 460) are positioned between adjacent protrusions or ridges of the sealing members (see FIG. 22B). This adjustment of the parallel and/or diagonal configurations can potentially improve PVL sealing.

According to some additional examples, the axial (see FIGS. 5B, 9B, and 14B) and/or diagonal (see FIGS. 5C, 9C, and 14C) configurations of the plurality of protrusions (e.g., protrusions 330 and 430), divided protrusions 434, or ridges 230, of the various sealing members of the present invention (e.g., sealing members 222, 322, and 422), relative to the axial flow direction across the annular or arterial wall 105 (when the sealing member is coupled to the outer surface of the frame 106 of the prosthetic valve 100) can be advantageous, since these sealing member configurations can be positioned within the site of implantation so that the plurality of protrusions (e.g., protrusions 330 and 430), divided protrusions 434 or ridges 230 of the sealing members can angularly adjusted within the site of implantation, such that following implantation, the native commissures are positioned between adjacent protrusions or ridges of the sealing members.

Reference is now made to FIGS. 23A-24. According to some examples, there is provided an additional configuration of the sealing member 422 coupled to the outer surface of the frame 106 of the prosthetic valve 100, wherein the sealing member 422 comprises a single protrusion 430. In further examples, the single protrusion 430 extends away and around the first surface 402, in parallel to any one of the outflow edge 407 and the inflow edge 409. In still further examples, the length of the single protrusion 430 in a direction extending between the outflow and inflow edges 407 and 409, respectively, (e.g., parallel to centerline 111) is at least as great as the distance between two junctions 112 aligned and distanced axially from each other along at least one cell 108 covered by the sealing member 422.

According to some examples, one inter-protrusion gap 450 is formed between the outflow edge 407 and one side of the single protrusion 430, while another inter-protrusion gaps 450 is formed between the inflow edge 409 and another opposite side of the single protrusion 430.

According to some examples, the sealing member 422 is characterized by having a nonfibrous outer surface, comprising the single protrusion 430, similar to the nonfibrous outer surface 480 as disclosed herein.

It is to be understood that the various characteristics of the plurality of protrusions 430, as disclosed herein above, similarly apply to the single protrusion 430. According to some examples, the single protrusion 430 is elastic and comprise a thermoplastic elastomer material, such as TPU, as disclosed herein above. According to some examples, the sealing member 422 comprises the single protrusion 430 and has a resilient 3D shape/structure, wherein said resilient 3D shape is configured to deform when an external force is applied thereto (e.g., when compressed against the annular or arterial wall 105 or within a delivery system), and further configured to revert to its original shape when the external force is no longer applied thereto (e.g., when a valve is released from the shaft or capsule prior to expansion thereof).

As mentioned herein above, an important design parameter of a transcatheter prosthetic heart valve is the diameter of the folded or crimped state. The diameter of the crimped profile is important because it directly influences the user's (e.g., medical personnel) ability to advance the transcatheter prosthetic heart valve through the femoral artery or vein. More particularly, a smaller profile allows for treatment of a wider population of patients, with enhanced safety. When the prosthetic valve 100 is radially compressed or crimped to a radially compressed state for delivery into the patient's body, the frame 106 elongates in the direction of its centerline 111. Because the sealing member 422, comprising the single protrusion 430, is coupled to the outer surface of the frame 106 of the prosthetic valve 100, such that the protrusion 430 spans across at least two axially opposing junctions 112, the first layer 410 elongates therewith, stretching in turn the protrusion 430 in a manner that reduces the profile of the protrusion (see FIG. 23B) resulting in a lower crimped profile, when compared, for example, to the crimped profile of a valve 100 that includes a sealing member having a different 3D structure. This lower profile permits the user to more easily navigate the delivery apparatus (including crimped valve 100) through a patient's vasculature to the implantation site. The lower profile of the crimped valve is particularly advantageous when navigating through portions of the patient's vasculature which are particularly narrow, such as the iliac artery.

Advantageously, a prosthetic valve 100 that includes the sealing member 422 comprising the single protrusion 430 is characterized by having a lower profile of the crimped state (see FIG. 23B), relative to the expanded state (see FIG. 23A). Specifically, a prosthetic valve 100 that includes the sealing member 422 comprising the single protrusion 430 is characterized by having a lower crimped state profile within a delivery system, relative to a valve 100 comprising a sealing member having a plurality of smaller protrusion in the same state. The lower profile of the crimped state of valve 100 is achieved due to the 3D shape of the single protrusion 430 having a length which is at least as great as the distance between two junctions 112, causing it to assume a relatively flattened configuration (FIG. 24B) as the frame elongated during crimping thereof, distancing the inflow and outflow ends of the single protrusion 430 away from each other.

According to some examples, the single protrusion 430 defines a single hollow lumen 431 therein, as illustrated for example in the side cross-sectional enlarged views of the protrusion 430 in FIGS. 23A-B. According to some examples, the single hollow lumen 431 comprise a gas disposed therein. According to further examples, the gas does not affect the single protrusion's 430 elastic and compressible characteristics and/or abilities as was disclosed herein above. The gas can be a non-flammable, non-toxic gas, selected from but not limited to, air, nitrogen, argon, carbon dioxide, helium, etc. According to some examples, the gas is injected into the hollow lumen 431. In further examples, the gas is configured to replace a previous gas residing within the hollow lumen 431 prior to the injection thereof. For example, air can be extracted out of the hollow lumen 431 and replaced by nitrogen, optionally utilizing a needle tip to pierce the protrusion 430 and inject the gas, wherein the pierced protrusion 430 can be sealed afterwards by a biocompatible sealing additive.

According to some examples, the single protrusion 430 comprise a plurality of apertures 435 spaced from each other (see FIG. 24), wherein each aperture 435 is configured to provide fluid communication between the single hollow lumen 431 and the external environment outside of the apertures 435, i.e., the tissues and/or fluids (e.g., blood) within in the implantation site (e.g., the inner surface of the annular or arterial wall 105). According to some examples, the hollow lumen 431 contains a pharmaceutical composition 436 disposed therein, as disclosed herein above. According to some examples, at least a portion of the apertures 435 are sealed with a membrane (e.g., biodegradable membrane 437), as disclosed herein above. In further examples, each aperture 435 is sealed with a membrane.

According to some examples, the membrane can be a semi-permeable membrane, configured to enables diffusion of fluids (e.g., blood) therethrough into the hollow lumen 431, but not in the opposite direction. According to some examples, the hollow lumen 431 contains an aqueous solution disposed therein. According to further examples, the aqueous solution comprises at least one divalent ion and/or a salt thereof. The at least one divalent ion can be selected from calcium (Ca²⁺), magnesium (Mg²⁺), iron (Fe²⁺), combinations and/or salts thereof, or any other suitable divalent ion known in the art.

According to some examples, the semi-permeable membrane is configured to enable diffusion of fluids therethrough into the hollow lumen 431, thereby enabling to achieve equalized concentrations of salts between the fluids within in the implantation site and the hollow lumen 431, due to a gradient in the salt concentration. The term “diffusion” as used herein, refers to the movement of substances from a region of higher concentration to a region of lower concentration, driven by a gradient in concentration.

The diffusion of fluids into the hollow lumen 431 can cause it to swell or expand, thereby causing the elastic single protrusion 430 to expand. Expansion of the single protrusion 430 is possible due to its elastic characteristics, which stems from the thermoplastic elastomer material it's made from, as was disclosed herein above. Advantageously, the expansion of the single protrusion 430 can enhance the compression thereof against the annular or arterial wall 105 at the implantation site, and thus to enable an enhanced PVL sealing between the prosthetic heart valve 100 and the inner surface of the annular or arterial wall 105.

According to some examples, at least a portion of the protrusion 430 comprises or is made of a semi-permeable material, wherein the semi-permeable material is structured as, and is configured to perform according to, any of the examples described above for the semi-permeable membrane. According to some examples, the entire protrusion comprises or is made of a semi-permeable material, wherein the semi-permeable material is structured as, and is configured to perform according to, any of the examples described above for the semi-permeable membrane.

As mentioned above, air (or other gas) entrapped within an enclosed lumen of the protrusion 430 may pose a risk to the patient if the protrusions 430 are degraded or accidentally torn in a manner that may release the entrapped air and result in undesirable cavitation. When the protrusion 430 is provided with apertures 435, as shown in FIG. 24, the prosthetic valve 100 can be crimped by a crimper to the radially compressed state in a manner that flattens the protrusion 430 to the configurations similar to that shown in FIG. 23B, such that no air is trapped therein, and restrained in the crimped state as described herein above (for example, by being placed within a bounding sheath or a capsule), up until and during the implantation process, thus reducing risks of introducing entrapped air to the patient's body.

According to some examples, the prosthetic valve 100 comprising the sealing member 422 comprising the single protrusion 430, is configured to be advanced within a delivery system in the crimped state toward the implantation site, wherein the single protrusion 430 is compressed against an inner wall of the retaining sheath or capsule. When the valve is released from its crimped state and expanded against the anatomy, the inner lumen of the single protrusion 430 can be filled, through the apertures 435, with blood, allowing it to resiliently revert to its expanded released state, similar to that shown in FIG. 24.

According to another aspect, there is provided a method for delivering the prosthetic valve 100 comprising various possible configurations of the 3D sealing members of the present invention as described herein above (e.g., sealing members 222, 322, 422, or the folded sealing member 422a) to a site of implantation (e.g., the aortic annulus in the case of aortic valve replacement) within a patient's body, the method comprising: (a) providing a prosthetic heart valve 100 in a crimped state, the valve 100 comprising a frame 106 and a leaflet assembly 130 mounted within the frame, the frame comprising a plurality of intersecting struts 110, wherein the frame is movable between a radially compressed state and a radially expanded state, wherein the valve 100 further comprises a 3D sealing member as described herein above (e.g., sealing members 222, 322, 422, or the folded sealing member 422a) coupled to an outer surface of the frame 106, wherein the sealing member has a three-dimensional (3D) shape in a spread relaxed state.

According to some examples, the sealing member of step (a) comprises a plurality of protrusions or ridges, extending away from a first surface of the sealing member, wherein the plurality of protrusions or ridges are spaced apart from each other along the first surface thereof, wherein the plurality of protrusions or ridges form the 3D shape of the sealing member in its spread relaxed state. According to further examples, the sealing member, in a folded state thereof, extends from an inflow edge toward an outflow and is coupled to the outer surface of the frame 106 of the prosthetic valve 100, such that the plurality of protrusions or ridges are oriented to extend radially away from the centerline 111.

According to some examples, the sealing member of step (a) (e.g., sealing members 222, 322, 422, or the folded sealing member 422a) can have various configurations and/or structures, as specified herein above. For example, the sealing member can comprise circumferential configurations of the plurality of protrusions (e.g., protrusions 330, 430, or divided protrusions 434) as can be seen for example, at FIG. 9A or 14A. The sealing member can comprise circumferential configurations of the plurality of ridges 230, as can be seen for example, at FIG. 5A. The sealing member can comprise axial configurations of the plurality of protrusions (e.g., protrusions 330 and 430), divided protrusions (e.g., protrusions 434) or ridges (e.g., ridges 230), as can be seen for example, at FIGS. 5B, 9B, and 14B. The sealing member can comprise diagonal configurations of the plurality of protrusions (e.g., protrusions 330 and 430), divided protrusions (e.g., protrusions 434) or ridges (e.g., ridges 230), as can be seen for example, at FIGS. 5C, 9C, and 14C. The sealing member can be the folded sealing member 422a comprising the at least one helical protrusion 430a, as can be seen for example, at FIG. 14D.

According to some examples, the frame of the prosthetic heart valve 100 of step (a) is in a radially compressed state. According to further examples, while the frame is in the radially compressed state, the 3D sealing member of the present invention, which is coupled to the frame, is configured to become radially compressed therewith. According to still further examples, the radially compressed 3D sealing member is configured to maintain its ability to transition to a 3D shape in a cylindrical folded state as described herein above, when the frame is expanded, without experiencing irreversible deformation.

According to some examples, the method further comprises (b) percutaneously advancing through a patient's vasculature a distal end portion 54 of an elongate delivery system (e.g., catheter 50), wherein the prosthetic valve 100 of step (a) in a radially compressed state is disposed on the distal end portion thereof, and wherein the frame 106 of the valve 100 is coupled to a deployment mechanism disposed on the distal end portion of the elongate delivery system.

According to some examples, step (b) comprises providing a system for delivering and deploying an expandable heart valve. The main elements of the system can include a proximal operating handle; the elongate delivery system comprising a catheter 50 comprising an elongated shaft extending distally from the operating handle (not shown), and the heart valve deployment mechanism comprising the valve 100 to be delivered. The deployment mechanism may include an inflated balloon (e.g., inflatable balloon 52) coupled to the valve 100, wherein the deployment mechanism can be configured to inflate the balloon when actuated (see FIGS. 2A-B). The inflated balloon 52 coupled to the valve 100 can be provided on the distal end portion 54 of the shaft of the catheter 50 of the elongate delivery system.

Various examples of system for delivering and deploying an expandable heart valve can be used in the context of the present invention. For example, U.S. Pat. Nos. 6,730,118, 9,572,663, 9,827,093 and 10,603,165, each incorporated herein by reference, describe compressible transcatheter prosthetic heart valves that can be percutaneously introduced in a crimped state on a catheter and expanded in the desired position by balloon inflation, by utilization of a self-expanding frame or stent, or by utilization of a mechanical expansion and locking mechanism.

According to some examples, the method further comprises (c) positioning the prosthetic valve 100 in an annulus of a native aortic valve within the site of implantation.

A user (e.g., medical personnel) can advance and position the deployment mechanism and the valve 100 coupled thereto in proximity to the implantation site, in this case the aortic annulus, using visualization techniques or an endoscope. Visualization techniques such as fluoroscopy or another imaging technique can utilize radiopaque markers, located on the deployment mechanism and/or on the prosthetic valve 100 (e.g., on the sealing members of the present invention as described above), for the successful and safe advancement and positioning of the valve 100 in the required site.

According to some examples, the method further comprises (d) actuating the deployment mechanism, thereby expanding the frame of the prosthetic valve 100 to a final radially expanded state within the annulus of the native aortic valve. The deployment mechanism may comprise an inflated balloon coupled to the prosthetic valve 100, wherein actuating the deployment mechanism can be configured to inflate the balloon, thereby expanding the frame of the prosthetic valve 100. In alternative implementations, a mechanically expandable frame can be expanded by actuation a plurality of expansion and locking assemblies. According to some examples, when the frame 106 of the prosthetic valve 100 is radially expanded, the radially compressed 3D sealing member transitions to its 3D shape in a cylindrical folded state as described herein above, without sustaining any irreversible deformation, and becomes compressed against the annular or arterial wall 105.

According to some examples, the prosthetic valve 100 can be positioned within the annulus of the native aortic valve during step (c) so that when the frame of the prosthetic valve 100 is radially expanded during step (d), the sealing members of the present invention (e.g., sealing members 222, 322, and 422) will be positioned within the annulus relative to the annular arterial wall 105, such that at least one of the plurality of protrusions (e.g., protrusions 330 and 430), divided protrusions (e.g., protrusions 434) or ridges (e.g., ridges 230) of the sealing members of the present invention (e.g., sealing members 222, 322, and 422) extend circumferentially around the valve (see for example, FIGS. 21A-21B) and are compressed against the annular or arterial wall 105. Advantageously, this circumferential orientation can improve PVL sealing between the sealing member and the annular or arterial wall 105 by forming a physical barrier that prevents or significantly reduces paravalvular leakage (PVL) of blood around the valve 100 through the gaps 107.

According to some examples, the prosthetic valve 100 can be positioned within the annulus of the native aortic valve during step (c) so that when the frame of the prosthetic valve 100 is radially expanded during step (d), at least one of the plurality of protrusions (e.g., protrusions 330 and 430), divided protrusions (e.g., protrusions 434) or ridges (e.g., ridges 230) of the sealing members of the present invention (e.g., sealing members 222, 322, and 422) extend axially, substantially parallel to the direction of flow.

According to some examples, the prosthetic valve 100 can be positioned within the annulus of the native aortic valve during step (c) so that when the frame of the prosthetic valve 100 is radially expanded during step (d), at least one of the plurality of protrusions (e.g., protrusions 330 and 430), divided protrusions (e.g., protrusions 434) or ridges (e.g., ridges 230) of the sealing members of the present invention (e.g., sealing members 222, 322, and 422) extend diagonally with respect to the axial direction of blood flow.

According to some examples, the prosthetic valve 100 can be positioned within the annulus of the native aortic valve during step (c) such that when the frame of the prosthetic valve 100 is radially expanded during step (d), at least one helical protrusion 430a extends across a helical path over the valve, and is pressed against the annular or arterial wall 105.

As disclosed above, the annular or arterial wall 105, as well as the native leaflets, may comprise at least one calcification 460, as illustrated in FIG. 22B. According to some examples, the prosthetic valve 100 can be positioned within the annulus of the native aortic valve during step (c) such that when the frame of the prosthetic valve 100 is radially expanded during step (d), the at least one calcification 460 is positioned between axial and/or diagonal protrusions or ridges of the sealing members of the present invention as described above.

According to some examples, the method further comprises actuating locking mechanisms on the prosthetic valve 100 to lock the prosthetic valve in the final radially expanded state, pressed against the annulus, wherein the expanded valve 100 is typically retained in position due to the pressure applied thereby against the native anatomy. Various possible locking mechanisms known in the art, which can be used in the context of the present invention, were previously disclosed, such as for example in U.S. Pat. Nos. 6,733,525, 9,827,093, 10,603,165, and 10,806,573, U.S. Pat. Pub. No. 2018/0344456, and US Pat. App. Nos. 62/870,372 and 62/776,348, each incorporated herein by reference.

According to some examples, the method further comprises retracting the deployment mechanism and the elongate delivery system from the patient's body, leaving the prosthetic valve 100 implanted in the patient.

While the various sealing members are illustrated throughout the Figures to extend over the frame 106 in a manner that extends below its inflow end (which is hidden from view thereby), it is to be understood that this is for the purpose of illustration and not limitation, and that any of the sealing members can be positioned and/or sized to extend over different portion of the frame 106 than the illustrated configuration. For example, any of the sealing members can be coupled to the frame 10-6 such that it is axially spaced from the inlet end (e.g., from the inflow apices) of the frame.

Although the present disclosure illustrates the present sealing members in connection to specific prosthetic heart valves intended for implantation in humans, such as the prosthetic heart valve 100 illustrated throughout the Figures, it is to be understood that the sealing members can be configured for use on other prosthetic valves or other types of prosthetic devices intended for implantation at any of the native valve of an animal or patient (e.g., the aortic, pulmonary, mitral, tricuspid, and Eustachian valve, etc.).

As used herein, the term “about”, when referring to a measurable value such as an amount, a temporal duration, and the like, is meant to encompass variations of +/−10%, more preferably +/−5%, even more preferably +/−1%, and still more preferably +/−0.1% from the specified value, as such variations are appropriate to perform the disclosed devices and/or methods.

### ADDITIONAL EXAMPLES OF THE DISCLOSED TECHNOLOGY

In view of the above-described implementations and/or examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a frame comprising a plurality of intersecting struts, wherein the frame is movable between a radially compressed state and a radially expanded state; a leaflet assembly mounted within the frame; and a sealing member coupled to an outer surface of the frame, wherein the sealing member extends from an inflow edge toward an opposing outflow edge, wherein the sealing member comprises a first layer and a second layer coating the first layer, wherein a nonfibrous outer surface of the sealing member is formed of a material inherently shaped to define a plurality of elevated portions with peaks and a plurality of non-elevated portions, and wherein said first and second layers are disposed externally to the outer surface of the frame.

Example 2. The prosthetic heart valve of any example herein, particularly example 1, wherein the elevated portions are configured to deform when an external pressure exceeding a predefined threshold is applied thereto in a direction configured to press them against the frame, and to revert to a relaxed state thereof when the external pressure is no longer applied thereto, and wherein the distance of the peaks from the frame is greater than the distance of the non-elevated portions from the frame in the relaxed state.

Example 3. The prosthetic heart valve of any example herein, particularly example 2, wherein the predefined threshold of the external pressure is 300 mmHg.

Example 4. The prosthetic heart valve of any example herein, particularly any one of examples 1 to 3, wherein the nonfibrous outer surface is a smooth surface.

Example 5. The prosthetic heart valve of any example herein, particularly any one of examples 1 to 4, wherein the sealing member comprises a third layer, wherein the second layer and the third layer collectively form a coating which covers the first layer.

Example 6. The prosthetic heart valve of any example herein, particularly any one of examples 1 to 5, wherein the first layer comprises at least one tear resistant fabric.

Example 7. The prosthetic heart valve of any example herein, particularly example 6, wherein the tear resistant fabric comprises a ripstop fabric.

Example 8. The prosthetic heart valve of any example herein, particularly any one of examples 1 to 7, wherein the first layer comprises a biocompatible material.

Example 9. The prosthetic heart valve of any example herein, particularly any one of examples 1 to 8, wherein the first layer comprises at least one elastic material.

Example 10. The prosthetic heart valve of any example herein, particularly any one of examples 6 to 9, wherein the first layer comprises a PET fabric.

Example 11. The prosthetic heart valve of any example herein, particularly any one of examples 6 to 10, wherein the first layer is having a tear resistance of at least 5N.

Example 12. The prosthetic heart valve of any example herein, particularly any one of examples 6 to 10, wherein the first layer is having a tear resistance of at least 15N.

Example 13. The prosthetic heart valve of any example herein, particularly any one of examples 1 to 12, wherein the second layer comprises a biocompatible material.

Example 14. The prosthetic heart valve of any example herein, particularly any one of examples 1 to 13, wherein the second layer is made of a thermoplastic material selected from the group consisting of: polyamides, polyesters, polyethers, polyurethanes, polyolefins, polytetrafluoroethylenes, and combinations and copolymers thereof.

Example 15. The prosthetic heart valve of any example herein, particularly any one of examples 13 to 14, wherein the second layer is made of a thermoplastic elastomer.

Example 16. The prosthetic heart valve of any example herein, particularly example 15, wherein the second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof.

Example 17. The prosthetic heart valve of any example herein, particularly example 16, wherein the second layer comprises TPU.

Example 18. The prosthetic heart valve of any example herein, particularly any one of examples 1 to 17, wherein the second layer comprises at least one thromboresistant material.

Example 19. The prosthetic heart valve of any example herein, particularly any one of examples 5 to 18, wherein the third layer comprises a biocompatible material.

Example 20. The prosthetic heart valve of any example herein, particularly any one of examples 5 to 19, wherein the third layer is made of a thermoplastic material.

Example 21. The prosthetic heart valve of any example herein, particularly example 20, wherein the third layer is made of a thermoplastic material selected from the group consisting of: polyamides, polyesters, polyethers, polyurethanes, polyolefins, polytetrafluoroethylenes, and combinations and copolymers thereof.

Example 22. The prosthetic heart valve of any example herein, particularly any one of examples 20 to 21, wherein the third layer is made of a thermoplastic elastomer.

Example 23. The prosthetic heart valve of any example herein, particularly example 22, wherein the third layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof.

Example 24. The prosthetic heart valve of any example herein, particularly example 23, wherein the third layer comprises TPU.

Example 25. The prosthetic heart valve of any example herein, particularly any one of examples 5 to 24, wherein the third layer comprises at least one thromboresistant material.

Example 26. The prosthetic heart valve of any example herein, particularly any one of examples 5 to 25, wherein the second layer and the third layer are made from the same material.

Example 27. The prosthetic heart valve of any example herein, particularly any one of examples 1 to 26, wherein the elevated portions of the sealing member comprise a plurality of ridges, wherein the plurality of ridges are spaced apart from each other along a first surface of the sealing member, and wherein the second layer forms the first surface of the sealing member.

Example 28. The prosthetic heart valve of any example herein, particularly example 27, wherein each one of the plurality of ridges extends outward from the outer surface of the frame.

Example 29. The prosthetic heart valve of any example herein, particularly any one of examples 27 to 28, wherein the sealing member comprises a plurality of inner channels, wherein each channel is formed at a second surface of the sealing member.

Example 30. The prosthetic heart valve of any example herein, particularly example 29, wherein the number of channels is identical to the number of ridges, wherein each one of the plurality of channels is formed by a respective one of the plurality of ridges at an opposing surface of the sealing member.

Example 31. The prosthetic heart valve of any example herein, particularly any one of examples 29 to 30, wherein each one of the plurality of channels is facing inward.

Example 32. The prosthetic heart valve of any example herein, particularly any one of examples 29 to 31, wherein the non-elevated portions of the sealing member comprise a plurality of inter-ridge gaps formed over the surface of the first layer between each two adjacent ridges of the sealing member.

Example 33. The prosthetic heart valve of any example herein, particularly any one of examples 27 to 32, wherein the plurality of ridges follow parallel path-lines extending along the first surface of the sealing member.

Example 34. The prosthetic heart valve of any example herein, particularly example 33, wherein the plurality of ridges follow parallel path-lines extending substantially in parallel to at least one of the inflow edge and/or the outflow edge.

Example 35. The prosthetic heart valve of any example herein, particularly example 33, wherein the plurality of ridges follow parallel path-lines extending substantially perpendicular to at least one of the inflow edge and the outflow edge.

Example 36. The prosthetic heart valve of any example herein, particularly example 33, wherein the plurality of ridges follow parallel path-lines extending substantially diagonally with respect to at least one of the inflow edge and the outflow edge.

Example 37. The prosthetic heart valve of any example herein, particularly any one of examples 27 to 36, wherein the plurality of ridges are compressible.

Example 38. The prosthetic heart valve of any example herein, particularly any one of examples 32 to 37, wherein the sealing member has a total layer thickness measured between the first surface and the second surface of the sealing member, at one of the inter-ridge gaps, and a sealing member thickness measured by the height of the ridges of the sealing member, wherein the sealing member thickness is greater by at least 1000% than the total layer thickness.

Example 39. The prosthetic heart valve of any example herein, particularly example 38, wherein the sealing member thickness is greater by at least 2000% than the total layer thickness.

Example 40. The prosthetic heart valve of any example herein, particularly example 38, wherein the sealing member thickness is greater by at least 3000% than the total layer thickness.

Example 41. The prosthetic heart valve of any example herein, particularly any one of examples 1 to 26, wherein the elevated portions of the sealing member comprise a plurality of protrusions extending around and outward from a first surface of the sealing member, wherein said plurality of protrusions are spaced apart from each other along the first surface, and wherein each one of the plurality of protrusions is compressible.

Example 42. The prosthetic heart valve of any example herein, particularly example 41, wherein the sealing member comprises a flat second surface located opposite to the first surface, when in its relaxed state.

Example 43. The prosthetic heart valve of any example herein, particularly any one of examples 41 to 42, wherein the non-elevated portions of the sealing member comprise a plurality of inter-protrusion gaps, wherein each gap is located between two adjacent protrusions, wherein the plurality of inter-protrusion gaps are facing the same direction as the protrusions face.

Example 44. The prosthetic heart valve of any example herein, particularly any one of examples 41 to 43, wherein each one of the plurality of protrusions extends around and away from the first surface and forms 3D shapes thereon, wherein the 3D shapes can be selected from the group consisting of: inverse U-shapes, half-spheres, domes, cylinders, pyramids, triangular prisms, pentagonal prisms, hexagonal prisms, flaps, polygons, and combinations thereof.

Example 45. The prosthetic heart valve of any example herein, particularly example 44, wherein the plurality of protrusions form elongated 3D shapes and extend substantially in parallel to at least one of: the inflow edge, the outflow edge, or both.

Example 46. The prosthetic heart valve of any example herein, particularly example 44, wherein the plurality of protrusions form elongated 3D shapes and extend substantially perpendicular to at least one of: the inflow edge, the outflow edge, or both.

Example 47. The prosthetic heart valve of any example herein, particularly example 44, wherein the plurality of protrusions form elongated 3D shapes and extend substantially diagonally with respect to at least one of: the inflow edge, the outflow edge, or both.

Example 48. The prosthetic heart valve of any example herein, particularly any one of examples 42 to 47, wherein the sealing member has a total layer thickness measured between the first surface and the second surface at one of the inter-protrusion gaps, and a sealing member thickness defined as the distance between the protrusions to the second surface, wherein the sealing member thickness is greater by at least 1000% than the total layer thickness.

Example 49. The prosthetic heart valve of any example herein, particularly example 48, wherein the sealing member thickness is greater by at least 2000% than the total layer thickness.

Example 50. The prosthetic heart valve of any example herein, particularly example 48, wherein the sealing member thickness is greater by at least 3000% than the total layer thickness.

Example 51. The prosthetic heart valve of any example herein, particularly any one of examples 41 to 50, wherein the plurality of protrusions comprises the same material as the second layer.

Example 52. The prosthetic heart valve of any example herein, particularly any one of examples 41 to 51, wherein each protrusion is made of a biocompatible material.

Example 53. The prosthetic heart valve of any example herein, particularly any one of examples 41 to 52, wherein each protrusion is made of a thermoplastic material.

Example 54. The prosthetic heart valve of any example herein, particularly example 53, wherein each protrusion is made of a thermoplastic material selected from the group consisting of: polyamides, polyesters, polyethers, polyurethanes, polyolefins, polytetrafluoroethylenes, and combinations and copolymers thereof.

Example 55. The prosthetic heart valve of any example herein, particularly any one of examples 53 to 54, wherein each protrusion is made of a thermoplastic elastomer.

Example 56. The prosthetic heart valve of any example herein, particularly example 55, wherein each protrusion is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof.

Example 57. The prosthetic heart valve of any example herein, particularly example 56, wherein each protrusion comprises TPU.

Example 58. The prosthetic heart valve of any example herein, particularly any one of examples 52 to 57, wherein each protrusion comprises at least one thromboresistant material.

Example 59. The prosthetic heart valve of any example herein, particularly any one of examples 41 to 58, wherein each one of the plurality of protrusions defines a non-hollow structure.

Example 60. The prosthetic heart valve of any example herein, particularly any one of examples 41 to 59, wherein each one of the plurality of protrusions defines a hollow lumen therein.

Example 61. The prosthetic heart valve of any example herein, particularly example 60, wherein each hollow lumen comprise two lumen edges, wherein each hollow lumen is open at one or both lumen edges.

Example 62. The prosthetic heart valve of any example herein, particularly any one of examples 60 to 61, wherein each one of the plurality of protrusions comprises a plurality of apertures spaced from each other therealong, wherein each aperture is configured to provide fluid communication between the hollow lumen and an external environment outside of the apertures.

Example 63. The prosthetic heart valve of any example herein, particularly example 62, wherein each one of the plurality of apertures is sealed by a biodegradable membrane, configured to enable a controlled release of a pharmaceutical composition from within the each one of the hollow lumens therethrough.

Example 64. The prosthetic heart valve of any example herein, particularly any one of examples 62 to 63, wherein each one of the hollow lumens contains a pharmaceutical composition disposed therein.

Example 65. The prosthetic heart valve of any example herein, particularly any one of examples 60 to 64, wherein each one of the hollow lumens contains an elastic porous element disposed therein.

Example 66. The prosthetic heart valve of any example herein, particularly example 65, wherein the elastic porous element comprises a pharmaceutical composition disposed therein.

Example 67. The prosthetic heart valve of any example herein, particularly any one of examples 65 to 66, wherein the elastic porous element is a sponge.

Example 68. The prosthetic heart valve of any example herein, particularly example 64 or 66, wherein the pharmaceutical composition comprises at least one pharmaceutical active agent selected from the group consisting of antibiotics, antivirals, antifungals, antiangiogenics, analgesics, anesthetics, anti-inflammatory agents including steroidal and non-steroidal anti-inflammatories (NSAIDs), corticosteroids, antihistamines, mydriatics, antineoplastics, immunosuppressive agents, anti-allergic agents, metalloproteinase inhibitors, tissue inhibitors of metalloproteinases (TIMPs), vascular endothelial growth factor (VEGF) inhibitors or antagonists or intraceptors, receptor antagonists, RNA aptamers, antibodies, hydroxamic acids and macrocyclic anti-succinate hydroxamate derivatives, nucleic acids, plasmids, siRNAs, vaccines, DNA binding compounds, hormones, vitamins, proteins, peptides, polypeptides and peptide-like therapeutic agents, anesthetizers and combinations thereof.

Example 69. The prosthetic heart valve of any example herein, particularly any one of examples 41 to 58, wherein each one of the plurality of protrusions is a divided protrusion, wherein each one of the plurality of divided protrusions forms an inner space between the divided protrusions.

Example 70. The prosthetic heart valve of any example herein, particularly example 65, wherein said inner space extends between an opening of each divided protrusion toward the first surface of the sealing member.

Example 71. The prosthetic heart valve of any example herein, particularly example 65, wherein said inner space extends between an opening of each divided protrusion toward a first surface of the first layer.

Example 72. The prosthetic heart valve of any example herein, particularly any one of examples 70 to 71, wherein the opening of each one of the plurality of divided protrusions is symmetric relative to an axis extending through the middle of each divided protrusion, thereby forming a symmetric inner space therein.

Example 73. The prosthetic heart valve of any example herein, particularly any one of examples 70 to 71, wherein the opening of each one of the plurality of divided protrusions is diverted at an angle relative to an axis extending through the middle of each divided protrusion, thereby forming an asymmetric inner space therein.

Example 74. A prosthetic heart valve comprising: a frame comprising a plurality of intersecting struts, wherein the frame is movable between a radially compressed state and a radially expanded state; a leaflet assembly mounted within the frame; and a sealing member coupled to an outer surface of the frame, wherein the sealing member is in a folded state, wherein the sealing member extends from an inflow edge toward an opposing outflow edge, wherein the sealing member comprises a first layer and a second layer coating the first layer, wherein a nonfibrous outer surface of the sealing member is formed of a material inherently shaped to define at least one helical protrusion extending radially outward in a helical configuration around the second layer, between the inflow edge and the outflow edge of the sealing member, and wherein said first and second layers are disposed externally to the outer surface of the frame.

Example 75. The prosthetic heart valve of any example herein, particularly example 74, wherein the first layer comprises at least one tear resistant fabric.

Example 76. The prosthetic heart valve of any example herein, particularly example 75, wherein the tear resistant fabric comprises a ripstop fabric.

Example 77. The prosthetic heart valve of any example herein, particularly any one of examples 74 to 76, wherein the first layer comprises a biocompatible material.

Example 78. The prosthetic heart valve of any example herein, particularly any one of examples 74 to 77, wherein the first layer comprises a PET fabric.

Example 79. The prosthetic heart valve of any example herein, particularly any one of examples 74 to 78, wherein the first layer is having a tear resistance of at least 5N, or optionally a tear resistance of at least 15N.

Example 80. The prosthetic heart valve of any example herein, particularly any one of examples 74 to 79, wherein the second layer is made of a biocompatible thermoplastic material selected from the group consisting of: polyamides, polyesters, polyethers, polyurethanes, polyolefins, polytetrafluoroethylenes, and combinations and copolymers thereof.

Example 81. The prosthetic heart valve of any example herein, particularly example 80, wherein the second layer is made of a thermoplastic elastomer.

Example 82. The prosthetic heart valve of any example herein, particularly example 81, wherein the second layer is made of a thermoplastic elastomer selected from the group consisting of: thermoplastic polyurethane (TPU), styrene block copolymers (TPS), Thermoplastic polyolefinelastomers (TPO), thermoplastic vulcanizates (TPV), thermoplastic copolyester (TPC), thermoplastic polyamides (TPA), and combinations thereof.

Example 83. The prosthetic heart valve of any example herein, particularly example 82, wherein the second layer comprises TPU.

Example 84. The prosthetic heart valve of any example herein, particularly any one of examples 74 to 83, wherein the second layer comprises at least one thromboresistant material.

Example 85. The prosthetic heart valve of any example herein, particularly any one of examples 74 to 84, wherein the distance of the helical protrusion from the frame is greater by at least 1000% than the distance of the second layer from the frame.

Example 86. The prosthetic heart valve of any example herein, particularly example 85, wherein the distance of the helical protrusion from the frame is greater by at least 2000% than the distance of the second layer from the frame.

Example 87. The prosthetic heart valve of any example herein, particularly example 85, wherein the distance of the helical protrusion from the frame is greater by at least 3000% than the distance of the second layer from the frame.

Example 88. A prosthetic heart valve comprising: a frame comprising a plurality of intersecting struts defining a plurality of junctions, wherein the frame is movable between a radially compressed state and a radially expanded state; a leaflet assembly mounted within the frame; and a sealing member coupled to an outer surface of the frame, wherein the sealing member extends from an inflow edge toward an opposing outflow edge, wherein the sealing member comprises a tear resistant first layer and a thermoplastic second layer coating the first layer and defining a first surface of the sealing member, wherein a nonfibrous outer surface of the sealing member is formed of a material inherently shaped to define a single compressible protrusion extending away and around said first surface of the sealing member, in parallel to any one of the outflow and the inflow edges, wherein the length of the single protrusion in a direction extending between the outflow and inflow edges of the sealing member is at least as great as the distance between two junctions of the frame, which are aligned and distanced axially from each other, and wherein said first and second layers are disposed externally to the outer surface of the frame.

Example 89. The prosthetic heart valve of any example herein, particularly example 88, wherein the single compressible protrusion defines a single hollow lumen therein.

Example 90. The prosthetic heart valve of any example herein, particularly any one of examples 88 to 89, wherein the distance of the protrusion from the frame is greater by at least 1000% than the distance of the first surface of the sealing member from the frame.

Example 91. The prosthetic heart valve of any example herein, particularly example 90, wherein the distance of the protrusion from the frame is greater by at least 3000% than the distance of the first surface of the sealing member from the frame.

Example 92. The prosthetic heart valve of any example herein, particularly any one of examples 89 to 91, wherein the single compressible protrusion comprises a plurality of apertures spaced from each other therealong, wherein each aperture is configured to provide fluid communication between the hollow lumen and an external environment outside of the apertures.

Example 93. The prosthetic heart valve of any example herein, particularly example 92, wherein the single hollow lumen contains a pharmaceutical composition disposed therein.

Example 94. The prosthetic heart valve of any example herein, particularly example 93, wherein at least a portion of the apertures are sealed with a semi permeable membrane, configured to enable a controlled release of the pharmaceutical composition from within the hollow lumen therethrough.

Example 95. The prosthetic heart valve of any example herein, particularly any one of examples 88 to 94, wherein the tear resistant first layer comprises a ripstop fabric.

Example 96. The prosthetic heart valve of any example herein, particularly any one of examples 88 to 95, wherein the tear resistant first layer comprises a PET fabric.

Example 97. The prosthetic heart valve of any example herein, particularly any one of examples 88 to 96, wherein the tear resistant first layer is having a tear resistance of at least 5N, or optionally a tear resistance of at least 15N.

Example 98. The prosthetic heart valve of any example herein, particularly any one of examples 88 to 97, wherein the thermoplastic second layer comprises TPU.

Example 99. A method for producing a perivalvular leakage (PVL) skirt, the method comprising: (i) providing a tear resistant flat sheet, comprising a tear resistant first layer and a thermoplastic second layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, wherein the treatment comprises contacting the flat sheet with a mold at an elevated temperature; lowering the temperature, thereby maintaining a resilient structure of the thermoplastic second layer, wherein the second layer is located distally to the mold; and removing the mold from the sheet after the temperature was lowered; and (iii) connecting two opposite edges of the sheet to form a cylindrical sealing member in a cylindrical folded state.

Example 100. The method of any example herein, particularly example 99, wherein the flat sheet in step (i) comprises a tear resistant first layer located between a thermoplastic second layer and a thermoplastic third layer of the flat sheet.

Example 101. The method of any example herein, particularly example 100, step (ii) entails contacting the flat sheet with the mold, wherein the third layer is contacting the mold.

Example 102. The method of any example herein, particularly any one of examples 99 to 101, wherein step (ii) comprises contacting the flat sheet with the mold at an elevated temperature thereby forming a plurality of ridges thereon.

Example 103. The method of any example herein, particularly any one of examples 99 to 102, wherein the second layer is thermally shape-formable at the elevated temperature and resilient at the lowered temperature.

Example 104. The method of any example herein, particularly any one of examples 99 to 103, wherein the elevated temperature in step (ii) is at least 60° C.

Example 105. The method of any example herein, particularly any one of examples 99 to 104, wherein the lowered temperature in step (ii) is below 40° C.

Example 106. The method of any example herein, particularly any one of examples 99 to 105, wherein the thickness of sealing member in its spread relaxed state following step (ii) is at least 1000% greater than the initial thickness of the sheet provided in step (i).

Example 107. The method of any example herein, particularly example 106, wherein the thickness of sealing member in its spread relaxed state following step (ii) is at least 2000% greater than the initial thickness of the sheet provided in step (i).

Example 108. The method of any example herein, particularly example 107, wherein the thickness of sealing member in its spread relaxed state following step (ii) is at least 3000% greater than the initial thickness of the sheet provided in step (i).

Example 109. The method of any example herein, particularly any one of examples 99 to 108, wherein step (ii) entails placing the flat sheet on a mold, wherein the second layer is located distally to the mold.

Example 110. The method of any example herein, particularly any one of examples 100 to 108, wherein step (ii) entails placing the flat sheet on the mold, wherein the third layer is contacting the mold.

Example 111. The method of any example herein, particularly any one of examples 99 to 110, wherein step (ii) comprises placing the flat sheet on a mold at an elevated temperature and gravitationally submerging the heated sheet, thereby forming a plurality of ridges thereon, wherein the mold is selected from a plurality of rods, tubes, pipes, and combinations thereof.

Example 112. The method of any example herein, particularly any one of examples 99 to 110, wherein the mold comprises a base, a plurality of protrusions and a vacuum source comprising a plurality of apertures, wherein the plurality of protrusions extend away from the base and are spaced from each other along the base, and wherein the plurality of apertures are formed at the base, at the protrusions, or at both.

Example 113. The method of any example herein, particularly example 112, wherein step (ii) comprises positioning the flat sheet above the mold; heating the flat sheet to a thermoformable temperature; and bringing the sheet towards said mold, to effectively engage said flat sheet with the protrusions of mold, thereby to enable the sheet to conform to said protrusions, wherein the engagement of the sheet with the plurality of protrusions forms a plurality of ridges, while the engagement of the sheet with the base forms a plurality of inter-ridge gaps of the sealing member.

Example 114. The method of any example herein, particularly any one of examples 99 to 110, wherein step (ii) includes application of force using mold over two opposite edges of the sheet, wherein the mold comprises a first mold and a second mold, wherein the first mold comprises a first base and plurality of first mold protrusions and the second mold comprises a second base and plurality of second mold protrusions.

Example 115. The method of any example herein, particularly example 114, wherein step (ii) comprises placing the flat sheet between the plurality first mold protrusions and the plurality of second mold protrusions, so that the plurality first mold protrusions and the plurality second mold protrusions are disposed at a zipper-like configuration; and pressing the second mold against the first mold at an elevated temperature, thereby effectively engaging the flat sheet therebetween to enable the sheet to conform to said molds.

Example 116. A method for producing a perivalvular leakage (PVL) skirt, the method comprising: (i) providing a tear resistant flat sheet consisting of a tear resistant first layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, the treatment comprising placing the flat sheet on a mold, thereby forming a plurality of ridges thereon over the mold, wherein the mold comprises a base and a plurality of protrusions; heat coating the sheet at an elevated thermoformable temperature with a thermoplastic material, thereby forming a thermoplastic second layer thereon; and lowering the temperature, thereby forming a resilient structure of the thermoplastic second layer; and (iii) connecting two opposite edges of the sheet to form a cylindrical sealing member in a cylindrical folded state.

Example 117. The method of any example herein, particularly example 116, wherein the elevated thermoformable temperature in step (ii) is at least 60° C.

Example 118. The method of any example herein, particularly example 116, wherein the lowered temperature in step (ii) is below 40° C.

Example 119. The method of any example herein, particularly any one of examples 116 to 118, wherein the thickness of sealing member in its spread relaxed state is at least 1000% greater than the initial thickness of the sheet provided in step (i).

Example 120. The method of any example herein, particularly example 119, wherein the thickness of sealing member in its spread relaxed state following step (ii) is at least 3000% greater than the initial thickness of the sheet provided in step (i).

Example 121. A method for producing a perivalvular leakage (PVL) skirt, the method comprising: (i) providing a tear resistant flat sheet, comprising a tear resistant first layer and a thermoplastic second layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, wherein the treatment comprises: extruding a plurality of members on the thermoplastic second layer of the flat sheet, wherein each member comprises a molten composition at an elevated temperature, and wherein the members are spaced from each other; and lowering the temperature, resulting in the transition of each extruded member to a resilient state, thereby forming a plurality of protrusions thereon; and (iii) connecting two opposite edges of the sheet to form a cylindrical sealing member in a cylindrical folded state.

Example 122. The method of any example herein, particularly example 121, wherein the flat sheet in step (i) comprises a tear resistant first layer located between a thermoplastic second layer and a thermoplastic third layer of the flat sheet.

Example 123. The method of any example herein, particularly example 121 or 122, wherein the molten composition is made of a biocompatible thermoplastic material selected from the group consisting of: polyamides, polyesters, polyethers, polyurethanes, polyolefins, polytetrafluoroethylenes, and combinations and copolymers thereof.

Example 124. The method of any example herein, particularly any one of examples 121 to 123, wherein the molten composition is made of a thermoplastic elastomer, and optionally wherein the molten composition comprises TPU.

Example 125. The method of any example herein, particularly any one of examples 121 to 124, wherein the molten composition comprises at least one thromboresistant material.

Example 126. The method of any example herein, particularly any one of examples 121 to 125, wherein the elevated temperature in step (ii) is at least 60° C.

Example 127. The method of any example herein, particularly any one of examples 121 to 126, wherein the lowered temperature in step (ii) is below 40° C.

Example 128. The method of any example herein, particularly any one of examples 121 to 127, wherein the thickness of sealing member in its spread relaxed state following step (ii) is at least 1000% greater than the initial thickness of the sheet provided in step (i).

Example 129. The method of any example herein, particularly examples 128, wherein the thickness of sealing member in its spread relaxed state following step (ii) is at least 3000% greater than the initial thickness of the sheet provided in step (i).

Example 130. The method of any example herein, particularly any one of examples 121 to 129, wherein each one of the plurality of protrusions formed in step (ii) is in a 3D shape selected from the group consisting of: inverse U-shapes, half-spheres, domes, cylinders, pyramids, triangular prisms, pentagonal prisms, hexagonal prisms, flaps, polygons, and combinations thereof.

Example 131. A method for producing a perivalvular leakage (PVL) skirt, the method comprising: (i) providing a tear resistant flat sheet, comprising a tear resistant first layer and a thermoplastic second layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, wherein the treatment comprises: placing a mold comprising a plurality of masking elements spaced apart one from the other on the thermoplastic second layer of the flat sheet; depositing a thermoplastic material at an elevated temperature in the spaces formed between adjacent masking elements; and lowering the temperature, resulting in the transition of the thermoplastic material to a resilient state, thereby forming a plurality of protrusions on the flat sheet; and (iii) connecting two opposite edges of the sheet to form a cylindrical sealing member in a cylindrical folded state.

Example 132. The method of any example herein, particularly examples 131, wherein the flat sheet in step (i) comprises a tear resistant first layer located between a thermoplastic second layer and a thermoplastic third layer of the flat sheet.

Example 133. The method of any example herein, particularly any one of examples 131 to 132, wherein the thermoplastic material is biocompatible and is selected from the group consisting of: polyamides, polyesters, polyethers, polyurethanes, polyolefins, polytetrafluoroethylenes, and combinations and copolymers thereof.

Example 134. The method of any example herein, particularly examples 133, wherein the thermoplastic material is a thermoplastic elastomer, and optionally wherein the thermoplastic material comprises TPU.

Example 135. The method of any example herein, particularly any one of examples 131 to 134, wherein the thermoplastic material comprises at least one thromboresistant material.

Example 136. The method of any example herein, particularly any one of examples 131 to 135, wherein each one of the plurality of protrusions formed in step (ii) is in a 3D shape selected from the group consisting of: inverse U-shapes, half-spheres, domes, cylinders, pyramids, triangular prisms, pentagonal prisms, hexagonal prisms, flaps, polygons, and combinations thereof.

Example 137. The method of any example herein, particularly any one of examples 131 to 136, wherein the deposition of the thermoplastic material at step (ii) is performed by a technique selected from the group consisting of extrusion, brushing, spray-coating, chemical deposition, liquid deposition, vapor deposition, chemical vapor deposition, physical vapor deposition, roller printing, stencil printing, screen printing, inkjet printing, lithographic printing, 3D printing, and combinations thereof.

Example 138. The method of any example herein, particularly any one of examples 131 to 137, wherein the deposition of the thermoplastic material at step (ii) comprises depositing a monomer composition in the spaces formed between adjacent masking elements and polymerizing the composition, resulting in a transition of the monomer composition to a polymerized resilient state, thereby forming a plurality of protrusions on the flat sheet.

Example 139. The method of any example herein, particularly any one of examples 131 to 138, wherein a thickness of sealing member in its spread relaxed state following step (ii) is at least 1000%, optionally at least 2000%, or alternatively at least 3000% greater than an initial thickness of the sheet provided in step (i).

Example 140. A method for producing a perivalvular leakage (PVL) skirt, the method comprising: (i) providing a tear resistant flat sheet, comprising a tear resistant first layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, wherein the treatment comprises: placing a plurality of elongated molding members on the tear resistant flat sheet; depositing a thermoplastic layer, at an elevated temperature on the plurality of elongated molding members, thereby forming a plurality of protrusions; lowering the temperature, thereby forming a resilient 3D structure of the protrusions; and removing the plurality of elongated molding members from within the plurality of protrusions; and (iii) connecting two opposite edges of the sheet to form a cylindrical sealing member in a cylindrical folded state.

Example 141. The method of any example herein, particularly example 140, wherein the flat sheet in step (i) consists of a single tear resistant first layer.

Example 142. The method of any example herein, particularly example 141, wherein the flat sheet in step (i) further comprises a thermoplastic second layer.

Example 143. The method of any example herein, particularly example 140, wherein the flat sheet in step (i) comprises a tear resistant first layer located between a thermoplastic second layer and a thermoplastic third layer of the flat sheet.

Example 144. The method of any example herein, particularly any one of examples 140 to 143, wherein step (ii) comprises placing the plurality of elongated molding members on the tear resistant flat sheet; and depositing the thermoplastic layer, at the elevated temperature, on the tear resistant flat sheet, such that the plurality of elongated molding members are positioned between the tear resistant flat sheet and the thermoplastic layer, thereby forming a plurality of 3D shaped protrusions thereon.

Example 145. The method of any example herein, particularly any one of examples 140 to 144, wherein the elevated temperature in step (ii) is at least 60° C.

Example 146. The method of any example herein, particularly any one of examples 140 to 145, wherein the lowered temperature in step (ii) is below 40° C.

Example 147. The method of any example herein, particularly any one of examples 140 to 146, wherein a thickness of sealing member in its spread relaxed state following step (ii) is at least 1000%, optionally at least 2000%, or alternatively at least 3000% greater than an initial thickness of the sheet provided in step (i).

Example 148. The method of any example herein, particularly any one of examples 140 to 147, wherein the thermoplastic layer of step (ii) is made of a biocompatible thermoplastic material, and is selected from the group consisting of: polyamides, polyesters, polyethers, polyurethanes, polyolefins, polytetrafluoroethylenes, and combinations and copolymers thereof.

Example 149. The method of any example herein, particularly examples 148, wherein the thermoplastic layer comprise a thermoplastic elastomer, and optionally wherein the thermoplastic layer comprises TPU.

Example 150. The method of any example herein, particularly any one of examples 140 to 149, wherein the thermoplastic layer comprises at least one thromboresistant material.

Example 151. The method of any example herein, particularly any one of examples 140 to 150, wherein the plurality of elongated molding members are made of a temperature resilient metal or a metal alloy, and are selected from rods, tubes, pipes, and combinations thereof.

Example 152. The method of any example herein, particularly any one of examples 140 to 151, wherein removing the plurality of elongated molding members from within the plurality of protrusions in step (ii) comprises extracting each elongated molding member through at least one protrusion edge located at the first lateral edge or the second lateral edge of the sheet, thereby forming a plurality of hollow lumens therein.

Example 153. The method of any example herein, particularly examples 152, wherein step (ii) further comprises perforating a plurality of apertures in the plurality of protrusions.

Example 154. The method of any example herein, particularly examples 153, wherein step (ii) further comprise inserting a pharmaceutical composition into at least part of said hollow lumens.

Example 155. A method for producing a perivalvular leakage (PVL) skirt, the method comprising: (i) providing a tear resistant flat sheet, comprising a tear resistant first layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, wherein the treatment comprises: placing a plurality of elastic porous members on the tear resistant flat sheet; depositing a thermoplastic layer, at an elevated temperature on the plurality of elastic porous members, thereby forming a plurality of protrusions; and lowering the temperature, thereby forming a resilient 3D structure of the protrusions; and (iii) connecting two opposite edges of the sheet to form a cylindrical sealing member in a cylindrical folded state.

Example 156. The method of any example herein, particularly example 155, wherein the flat sheet in step (i) is identical to the sheet as depicted at any one of examples 141 to 143.

Example 157. The method of any example herein, particularly any one of examples 155 to 156, wherein step (ii) comprises placing the plurality of elastic porous members on the tear resistant flat sheet; and depositing the thermoplastic layer, at the elevated temperature, on the tear resistant flat sheet, such that the plurality of elastic porous members are positioned between the tear resistant flat sheet and the thermoplastic layer, thereby forming a plurality of 3D shaped protrusions comprising the elastic porous members there-within.

Example 158. The method of any example herein, particularly any one of examples 155 to 157, wherein the elevated temperature in step (ii) is at least 60° C. and/or wherein the lowered temperature in step (ii) is below 40° C.

Example 159. The method of any example herein, particularly any one of examples 155 to 158, wherein a thickness of sealing member in its spread relaxed state following step (ii) is at least 1000%, optionally at least 2000%, or alternatively at least 3000% greater than an initial thickness of the sheet provided in step (i).

Example 160. The method of any example herein, particularly any one of examples 155 to 159, wherein the thermoplastic layer of step (ii) is made of the same material(s) as depicted at any one of examples 148 to 150.

Example 161. The method of any example herein, particularly any one of examples 155 to 160, wherein each elastic porous member is made of a temperature resilient biocompatible sponge.

Example 162. The method of any example herein, particularly any one of examples 155 to 161, wherein step (ii) further comprises perforating a plurality of apertures in the plurality of protrusions.

Example 163. The method of any example herein, particularly any one of examples 155 to 162, wherein step (ii) further comprises impregnating the plurality of elastic porous members with a pharmaceutical composition.

Example 164. A method for producing a perivalvular leakage (PVL) skirt, the method comprising: (i) providing a tear resistant flat sheet, comprising a tear resistant first layer, wherein the sheet extends between a first lateral edge and a second lateral edge, and between an inflow edge and an outflow edge; (ii) treating the sheet in a thermal shape-forming process to assume a resilient structure comprising a plurality of elevated portions and a plurality of non-elevated portions, in a spread relaxed state, wherein the treatment comprises: placing a plurality of elongated molding members on the tear resistant flat sheet, wherein each of the plurality of elongated molding members comprises a sharp tip; depositing a thermoplastic layer, at an elevated temperature on the plurality of elongated molding members, thereby forming a plurality of protrusions; lowering the temperature, thereby forming a resilient 3D structure thereof; and removing the plurality of elongated molding members through the plurality of protrusions, thereby forming a plurality of divided protrusions; and (iii) connecting two opposite edges of the sheet to form a cylindrical sealing member in a cylindrical folded state.

Example 165. The method of any example herein, particularly example 164, wherein the flat sheet in step (i) is identical to the sheet as depicted at any one of examples 141 to 143.

Example 166. The method of any example herein, particularly any one of examples 164 to 165, wherein depositing the thermoplastic layer on the plurality of elongated molding members at step (ii) entails contacting the thermoplastic layer with the sharp tips of the elongated molding members.

Example 167. The method of any example herein, particularly any one of examples 164 to 166, wherein step (ii) comprises pulling the sharp tip of each elongated molding member through the thermoplastic layer, wherein the sharp tip of each elongated molding member is pulled along an axis extending through the middle of each divided protrusion, in a direction perpendicular to the flat sheet, thereby forming a symmetric inner space therein.

Example 168. The method of any example herein, particularly any one of examples 164 to 166, wherein step (ii) comprises pulling the sharp tip of each elongated molding member through the thermoplastic layer, wherein the sharp tip of each elongated molding member is pulled in the direction of a pulling arrow which is diverted at the angle relative to a direction perpendicular to the flat sheet, thereby forming an asymmetric inner space therein.

Example 169. The method of any example herein, particularly any one of examples 164 to 168, wherein the elevated temperature in step (ii) is at least 60° C. and/or wherein the lowered temperature in step (ii) is below 40° C.

Example 170. The method of any example herein, particularly any one of examples 164 to 169, wherein a thickness of sealing member in its spread relaxed state following step (ii) is at least 1000%, optionally at least 2000%, or alternatively at least 3000% greater than an initial thickness of the sheet provided in step (i).

Example 171. The method of any example herein, particularly any one of examples 164 to 170, wherein the thermoplastic layer of step (ii) is made of the same material(s) as depicted at any one of examples 148 to 150.

Example 172. The method of any example herein, particularly any one of examples 164 to 171, wherein the plurality of elongated molding members and sharp tips are made of a temperature resilient metal or a metal alloy.

Example 173. A method for producing a perivalvular leakage (PVL) skirt, the method comprising: (i) providing a tear resistant flat sheet in a folded cylindrical state extending from an inflow edge towards an outflow edge; and (ii) treating the sheet in a thermal shape-forming process to assume a resilient structure comprising a plurality of elevated portions and a plurality of non-elevated portions, in the folded cylindrical state, wherein the treatment comprises: placing at least one helical mandrel around the tear resistant flat sheet; depositing a thermoplastic layer, at an elevated temperature, on the at least one helical mandrel, thereby forming at least one helical protrusion thereon extending radially away at a helical configuration therearound; lowering the temperature, thereby maintaining a resilient structure of the thermoplastic layer; and removing the at least one helical mandrel from within the at least one helical protrusion through at least one helical protrusion edge located at the inflow edge or the outflow edge, thereby forming a helical hollow lumen therein.

Example 174. The method of any example herein, particularly example 173, wherein the flat sheet in step (i) is identical to the sheet as depicted at any one of examples 141 to 142.

Example 175. The method of any example herein, particularly any one of examples 173 to 174, wherein step (ii) entails placing the at least one helical mandrel around the thermoplastic second layer of the flat sheet.

Example 176. The method of any example herein, particularly any one of examples 173 to 175, wherein the elevated temperature in step (ii) is at least 60° C. and/or wherein the lowered temperature in step (ii) is below 40° C.

Example 177. The method of any example herein, particularly any one of examples 173 to 176, wherein a thickness of sealing member in its spread relaxed state following step (ii) is at least 1000%, optionally at least 2000%, or alternatively at least 3000% greater than an initial thickness of the sheet provided in step (i).

Example 178. The method of any example herein, particularly any one of examples 173 to 177, wherein the thermoplastic layer of step (ii) is made of the same material(s) as depicted at any one of examples 148 to 150.

Example 179. The method of any example herein, particularly any one of examples 173 to 178, wherein step (ii) further comprise perforating a plurality of apertures in the helical protrusion.

Example 180. The method of any example herein, particularly example 179, wherein step (ii) further comprise inserting a pharmaceutical composition into at least a part of the helical hollow lumen.

Example 181. The method of any example herein, particularly any one of examples 99 to 172, wherein the tear resistant flat sheet comprises a first layer made from at least one biocompatible tear resistant material.

Example 182. The method of any example herein, particularly example 181, wherein the first layer comprises a ripstop fabric.

Example 182. The method of any example herein, particularly example 181, wherein the first layer comprises a PET fabric.

Example 183. The method of any example herein, particularly any one of examples 99 to 115, 121 to 139, and 142 to 154, wherein the thermoplastic second layer is made of the same material(s) as depicted at any one of examples 80 to 84.

Example 184. A prosthetic heart valve comprising: a frame comprising a plurality of intersecting struts defining a plurality of junctions, wherein the frame is movable between a radially compressed state and a radially expanded state; a leaflet assembly mounted within the frame; and a sealing member coupled to an outer surface of the frame, wherein the sealing member extends from an inflow edge toward an opposing outflow edge, wherein the sealing member comprises a tear resistant first layer and a second layer coating the first layer and defining a first surface of the sealing member, wherein a nonfibrous outer surface of the sealing member is formed of a semi-permeable material shaped to define a compressible protrusion extending away and around said first surface of the sealing member, in parallel to any one of the outflow and the inflow edges, wherein the length of the single protrusion in a direction extending between the outflow and inflow edges of the sealing member is at least as great as the distance between two junctions of the frame, which are aligned and distanced axially from each other, and wherein said first and second layers are disposed externally to the outer surface of the frame.

Example 185. The prosthetic heart valve of any example herein, particularly example 184, wherein the single compressible protrusion defines a single hollow lumen therein.

Example 186. The prosthetic heart valve of any example herein, particularly any one of examples 184 to 185, wherein the distance of the protrusion from the frame is at least 1000%, optionally at least 2000%, or alternatively at least 3000% greater than the distance of the first surface of the sealing member from the frame.

Example 187. The prosthetic heart valve of any example herein, particularly any one of examples 184 to 186, wherein the tear resistant first layer is made of the same material(s) as depicted at any one of examples 76 to 78.

Example 188. The prosthetic heart valve of any example herein, particularly any one of examples 184 to 187, wherein the tear resistant first layer is having a tear resistance of at least 5N, or optionally a tear resistance of at least 15N.

It is appreciated that certain features of the invention, which are, for clarity, described in the context of separate examples, may also be provided in combination in a single example. Conversely, various features of the invention which are, for brevity, described in the context of a single example, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention includes both combinations and sub-combinations of the various features described hereinabove as well as variations and modifications thereof which would occur to persons skilled in the art upon reading the foregoing description.

1. A prosthetic heart valve comprising:
a frame comprising a plurality of intersecting struts, wherein the frame is movable between a radially compressed state and a radially expanded state;
a leaflet assembly mounted within the frame; and
a sealing member coupled to an outer surface of the frame, wherein the sealing member extends from an inflow edge toward an opposing outflow edge, wherein the sealing member comprises a first layer and a second layer coating the first layer, wherein a nonfibrous outer surface of the sealing member is formed of a material inherently shaped to define a plurality of elevated portions with peaks and a plurality of non-elevated portions, and
wherein said first and second layers are disposed externally to the outer surface of the frame.

2. The prosthetic heart valve of claim 1, wherein the elevated portions are configured to deform when an external pressure exceeding a predefined threshold is applied thereto in a direction configured to press them against the frame, and to revert to a relaxed state thereof when the external pressure is no longer applied thereto, and wherein the distance of the peaks from the frame is greater than the distance of the non-elevated portions from the frame in the relaxed state.

3. The prosthetic heart valve of claim 1, wherein the nonfibrous outer surface is a smooth surface.

4. The prosthetic heart valve of claim 1, wherein the sealing member comprises a third layer, wherein the second layer and the third layer collectively form a coating which covers the first layer.

5. The prosthetic heart valve of claim 1, wherein the first layer comprises at least one tear resistant polyethylene terephthalate (PET) fabric.

6. The prosthetic heart valve of claim 1, wherein the second layer is made of biocompatible thermoplastic polyurethane (TPU).

7. The prosthetic heart valve of claim 1, wherein the elevated portions of the sealing member comprise a plurality of ridges, wherein the plurality of ridges are spaced apart from each other along a first surface of the sealing member, wherein the second layer forms the first surface of the sealing member, wherein each one of the plurality of ridges extends outward from the outer surface of the frame, wherein the sealing member comprises a plurality of inner channels, wherein each channel is formed at a second surface of the sealing member, and wherein each one of the plurality of channels is facing inward.

8. The prosthetic heart valve of claim 7, wherein the number of channels is identical to the number of ridges, wherein each one of the plurality of channels is formed by a respective one of the plurality of ridges at an opposing surface of the sealing member.

9. The prosthetic heart valve of claim 7, wherein the non-elevated portions of the sealing member comprise a plurality of inter-ridge gaps formed over the surface of the first layer between each two adjacent ridges of the sealing member.

10. The prosthetic heart valve of claim 7, wherein the plurality of ridges follow parallel path-lines extending along the first surface of the sealing member, and wherein the plurality of ridges are compressible.

11. The prosthetic heart valve of claim 10, wherein the plurality of ridges follow parallel path-lines extending substantially in parallel to at least one of the inflow edge or the outflow edge.

12. The prosthetic heart valve of claim 10, wherein the plurality of ridges follow parallel path-lines extending substantially diagonally with respect to at least one of the inflow edge or the outflow edge.

13. The prosthetic heart valve of claim 9, wherein the sealing member has a total layer thickness measured between the first surface and the second surface of the sealing member, at one of the inter-ridge gaps, and a sealing member thickness measured by the height of the ridges of the sealing member, wherein the sealing member thickness is greater by at least 1000% than the total layer thickness.

14. The prosthetic heart valve of claim 1, wherein the elevated portions of the sealing member comprise a plurality of protrusions extending around and outward from a first surface of the sealing member, wherein said plurality of protrusions are spaced apart from each other along the first surface, wherein each one of the plurality of protrusions is compressible.

15. The prosthetic heart valve of claim 14, wherein the sealing member comprises a flat second surface located opposite to the first surface, when in its spread relaxed state.

16. The prosthetic heart valve of claim 14, wherein the non-elevated portions of the sealing member comprise a plurality of inter-protrusion gaps, wherein each gap is located between two adjacent protrusions, wherein the plurality of inter-protrusion gaps are facing the same direction as the protrusions face.

17. The prosthetic heart valve of claim 14, wherein each one of the plurality of protrusions extends around and away from the first surface and forms 3D shapes thereon, wherein the 3D shapes can be selected from the group consisting of: inverse U-shapes, half-spheres, domes, cylinders, pyramids, triangular prisms, pentagonal prisms, hexagonal prisms, flaps, polygons, and combinations thereof.

18. The prosthetic heart valve of claim 17, wherein the plurality of protrusions form elongated 3D shapes and extend substantially in parallel to at least one of the inflow edge or the outflow edge.

19. The prosthetic heart valve of claim 17, wherein the plurality of protrusions form elongated 3D shapes and extend substantially diagonally with respect to at least one of the inflow edge or the outflow edge.

20. The prosthetic heart valve of claim 16, wherein the sealing member has a total layer thickness measured between the first surface and the second surface at one of the inter-protrusion gaps, and a sealing member thickness defined as the distance between the protrusions to the second surface, wherein the sealing member thickness is greater by at least 1000% than the total layer thickness.

21. The prosthetic heart valve of claim 14, wherein each one of the plurality of protrusions defines a non-hollow structure.

22. The prosthetic heart valve of claim 14, wherein each one of the plurality of protrusions defines a hollow lumen therein.

23. The prosthetic heart valve of claim 22, wherein each one of the plurality of protrusions comprises a plurality of apertures spaced from each other therealong, wherein each aperture is configured to provide fluid communication between the hollow lumen and an external environment outside of the apertures, and wherein each one of the hollow lumens contains a pharmaceutical composition disposed therein.

24. The prosthetic heart valve of claim 22, wherein each one of the hollow lumens contains an elastic porous element disposed therein, wherein the elastic porous element comprises a pharmaceutical composition disposed therein, and wherein each one of the plurality of protrusions comprises a plurality of apertures spaced from each other therealong.

25. The prosthetic heart valve of claim 14, wherein each one of the plurality of protrusions is a divided protrusion, wherein each one of the plurality of divided protrusions forms an inner space between the divided protrusions, wherein said inner space extends between an opening of each divided protrusion toward the first surface of the sealing member or toward a first surface of the first layer.

26. The prosthetic heart valve of claim 25, wherein the opening of each one of the plurality of divided protrusions is symmetric relative to an axis extending through the middle of each divided protrusion, thereby forming a symmetric inner space therein; or wherein the opening of each one of the plurality of divided protrusions is diverted at an angle relative to an axis extending through the middle of each divided protrusion, thereby forming an asymmetric inner space therein.
